SEQUENCE LISTING (110 sequences)

SEQ ID NO: 1 - protein, 9 aa, synthetic construct
  VARIANT 1: note = Any non-natural amino acid
  SITE 1: note = Any non-natural amino acid
  CROSSLNK 1..5: note = Intramolecular staple (Xaa-Xaa)
  VARIANT 5: note = Any non-natural amino acid
  SITE 5: note = Any non-natural amino acid
XSLIXSIAS

SEQ ID NO: 2 - protein, 9 aa, synthetic construct
  VARIANT 3: note = Any non-natural amino acid
  SITE 3: note = Any non-natural amino acid
  CROSSLNK 3..7: note = Intramolecular staple (Xaa-Xaa)
  VARIANT 7: note = Any non-natural amino acid
  SITE 7: note = Any non-natural amino acid
GSXIASXAS

SEQ ID NO: 3 - protein, 9 aa, synthetic construct
  VARIANT 4: note = Any non-natural amino acid
  SITE 4: note = Any non-natural amino acid
  CROSSLNK 4..8: note = Intramolecular staple (Xaa-Xaa)
  VARIANT 8: note = Any non-natural amino acid
  SITE 8: note = Any non-natural amino acid
GSLXASIXS

SEQ ID NO: 4 - protein, 10 aa, synthetic construct
  VARIANT 1: note = Any non-natural amino acid
  SITE 1: note = Any non-natural amino acid
  CROSSLNK 1..9: note = Intramolecular staple (Xaa-Xaa)
  VARIANT 9: note = Any non-natural amino acid
  SITE 9: note = Any non-natural amino acid
XGSLIASIXS

SEQ ID NO: 5 - protein, 9 aa, synthetic construct
GSLIASIAS

SEQ ID NO: 6 - protein, 11 aa, synthetic construct
  VARIANT 1: note = Any non-natural amino acid
  SITE 1: note = Any non-natural amino acid
  CROSSLNK 1..5: note = Intramolecular staple (Xaa-Xaa)
  VARIANT 5: note = Any non-natural amino acid
  SITE 5: note = Any non-natural amino acid
XNKVXRELVKH

SEQ ID NO: 7 - protein, 11 aa, synthetic construct
  VARIANT 5: note = Any non-natural amino acid
  SITE 5: note = Any non-natural amino acid
  CROSSLNK 5..9: note = Intramolecular staple (Xaa-Xaa)
  VARIANT 9: note = Any non-natural amino acid
  SITE 9: note = Any non-natural amino acid
SNKVXRELXKH

SEQ ID NO: 8 - protein, 11 aa, synthetic construct
  VARIANT 1: note = Any non-natural amino acid
  SITE 1: note = Any non-natural amino acid
  CROSSLNK 1..8: note = Intramolecular staple (Xaa-Xaa)
  VARIANT 8: note = Any non-natural amino acid
  SITE 8: note = Any non-natural amino acid
XNKVLREXVKH

SEQ ID NO: 9 - protein, 11 aa, synthetic construct
  VARIANT 2: note = Any non-natural amino acid
  SITE 2: note = Any non-natural amino acid
  CROSSLNK 2..9: note = Intramolecular staple (Xaa-Xaa)
  VARIANT 9: note = Any non-natural amino acid
  SITE 9: note = Any non-natural amino acid
SXKVLRELXKH

SEQ ID NO: 10 - protein, 11 aa, synthetic construct
  VARIANT 4: note = Any non-natural amino acid
  SITE 4: note = Any non-natural amino acid
  CROSSLNK 4..11: note = Intramolecular staple (Xaa-Xaa)
  VARIANT 11: note = Any non-natural amino acid
  SITE 11: note = Any non-natural amino acid
SNKXLRELVKX

SEQ ID NO: 11 - protein, 11 aa, synthetic construct
SNKVLRELVKH

SEQ ID NO: 12 - protein, 552 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLSSRQVVESVGNQMGVGKESDIYIVANEEGQQFALKLHRLGRTSFRNLKNKRDYHKHRHNVSWLYLSRLSAMKEFAYMKALYERKFPVPKPIDYNRHAVVMELINGYPLCQIHHVEDPASVYDEAMELIVKLANHGLIHGDFNEFNLILDESDHITMIDFPQMVSTSHPNAEWYFDRDVKCIKDFFMKRFSYESELFPTFKDIRREDTLDVEVSASGYTKEMQADDELLHPLGPDDKNIETKEGSEFSFSDGEVAEKAEVYGSENESERNCLEESEGCYCRSSGDPEQIKEDSLSEESADARSFEMTEFNQALEEIKGQVVENNSVTEFSEEKNRTENYNRQDGQRVQGGVPAGSDEYEDECPHLIALSSLNREFRPFRDEENVGAMNQYRTRTLSITSSGSAVSCSTIPPELVKQKVKRQLTKQQKSAVRRRLQKGEANIFTKQRRENMQNIKSSLEAASFWGE

SEQ ID NO: 13 - protein, 474 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLSSRQVVESVGNQMGVGKESDIYIVANEEGQQFALKLHRLGRTSFRNLKNKRDYHKHRHNVSWLYLSRLSAMKEFAYMKALYERKFPVPKPIDYNRHAVVMELINGYPLCQIHHVEDPASVYDEAMELIVKLANHGLIHGDFNEFNLILDESDHITMIDFPQMVSTSHPNAEWYFDRDVKCIKDFFMKRFSYESELFPTFKDIRREDTLDVEVSASGYTKEMQADDELLHPLGPDDKNIETKEGSEFSFSDGEVAEKAEVYGSENESERNCLEESEGCYCRSSGDPEQIKEDSLSEESADARSFEMTEFNQALEEIKGQVVENNSVTEFSEEKNRTENYNRQDGQRVQGGVPAGSDEYEDECPHLIALSSLNREFRPFRYRLLSIAF

SEQ ID NO: 14 - DNA, 2174 bp, Homo sapiens
  source 1..2174: mol_type = genomic DNA
gccttacgctttgcggctgtcgtcggagaggcatctgggttcggactggggccgccatggggaaagtgaatgtggccaagttgcgttacatgagccgagatgacttcagggtcttgaccgcggttgaaatgggcatgaagaaccatgaaattgttcccggcagtttgattgcttctatagccagccttaaacatggtggctgtaataaagttttaagagaattagtgaaacataaactcatagcttgggagcgtaccaaaactgtccagggctatcggttgacaaatgcaggatatgattacctagctttgaaaacactttcttctaggcaagtagttgagtctgttggaaaccagatgggtgttggcaaagaatcagatatttacattgttgcaaatgaagaaggacaacaatttgcattaaagcttcacagactaggaagaacctcgtttcgaaatttgaaaaacaaacgcgattatcataaacataggcacaatgtgtcatggctatatttatctcgtctctctgccatgaaggaatttgcctatatgaaggcattgtatgagaggaaatttccagttccaaagccaattgattacaatcgtcatgcagtggtcatggaactcataaatggttatccactatgtcagatacaccatgttgaagatcctgcatcagtatatgatgaagctatggaactaattgtcaaacttgcaaatcatgggctgattcatggagattttaatgaatttaatctcattttggatgaaagtgaccatatcaccatgattgattttccacagatggtttcaacttctcatcccaatgctgagtggtattttgacagagatgttaaatgcattaaagatttctttatgaaacgtttcagctacgaaagtgagctttttccaacttttaaggatatcaggagagaagacactcttgatgtggaggtttctgccagtggctacacaaaggaaatgcaggcagatgatgaactgcttcatccattaggtccagatgataaaaatattgaaacaaaagagggatctgaattctcattttcagatggagaagtggcagaaaaagcagaggtttacgggtcagaaaatgaaagtgaacggaactgtctagaagaatcagagggctgctattgcagatcatctggagaccctgaacaaataaaggaagacagtttatcagaagagagtgctgatgcacggagttttgaaatgactgaattcaatcaagctttagaagaaataaaagggcaggttgttgaaaacaactctgtaactgaattttctgaggagaaaaacagaactgaaaattacaacaggcaagatggtcagagagttcaaggaggagtccctgctggctctgacgagtatgaagatgaatgccctcatctaattgccttgtcgtcattaaatagagaattcaggcctttcaggtatagacttctgtcgattgctttttaactttagtcttgagactgatactttgttagattctagtaaaagaatgtttttttcaaatgatgtgacaaaattgtattctgccaccttgatgtataaaatgaaagccaaataattttttgttgttaaaaactcaaaatagcttccttttcaattgacctgattctaggcagcctattgactaaatttcaagtaattctgccttaaaatacaaactgatggctgggcacagtggctcgctcatgcctgtaatctcagcacttctgggaggccaaggcaggcaaatcacctgaggtcaggagtttgagaccagccaggccaacatggcaaaaccctgtctctactaaaaatataaaaattaaccgggcatggtggcctgtgcttgtaatcccagctacttgggagactgaggcaggagaatcacttgaacctgggaggcagaggttgcagtaagccaaagatcgtaccactgcactccagcctgggtgacgagcaaaaaactccgtctcaaaaatatatatatatatacaaactgatgattttaaaattattgcaggtctctggatacattgatcaggttttagatggatgagttattcctcatatattttcctaattacacatctgatgtgtgtttgagttatgtaaaggcatgtaaatattaaaacatgcaataaatgtactttctatatgaaa

SEQ ID NO: 15 - DNA, 3909 bp, Homo sapiens
  source 1..3909: mol_type = genomic DNA
gccttacgctttgcggctgtcgtcggagaggcatctgggttcggactggggccgccatggggaaagtgaatgtggccaagttgcgttacatgagccgagatgacttcagggtcttgaccgcggttgaaatgggcatgaagaaccatgaaattgttcccggcagtttgattgcttctatagccagccttaaacatggtggctgtaataaagttttaagagaattagtgaaacataaactcatagcttgggagcgtaccaaaactgtccagggctatcggttgacaaatgcaggatatgattacctagctttgaaaacactttcttctaggcaagtagttgagtctgttggaaaccagatgggtgttggcaaagaatcagatatttacattgttgcaaatgaagaaggacaacaatttgcattaaagcttcacagactaggaagaacctcgtttcgaaatttgaaaaacaaacgcgattatcataaacataggcacaatgtgtcatggctatatttatctcgtctctctgccatgaaggaatttgcctatatgaaggcattgtatgagaggaaatttccagttccaaagccaattgattacaatcgtcatgcagtggtcatggaactcataaatggttatccactatgtcagatacaccatgttgaagatcctgcatcagtatatgatgaagctatggaactaattgtcaaacttgcaaatcatgggctgattcatggagattttaatgaatttaatctcattttggatgaaagtgaccatatcaccatgattgattttccacagatggtttcaacttctcatcccaatgctgagtggtattttgacagagatgttaaatgcattaaagatttctttatgaaacgtttcagctacgaaagtgagctttttccaacttttaaggatatcaggagagaagacactcttgatgtggaggtttctgccagtggctacacaaaggaaatgcaggcagatgatgaactgcttcatccattaggtccagatgataaaaatattgaaacaaaagagggatctgaattctcattttcagatggagaagtggcagaaaaagcagaggtttacgggtcagaaaatgaaagtgaacggaactgtctagaagaatcagagggctgctattgcagatcatctggagaccctgaacaaataaaggaagacagtttatcagaagagagtgctgatgcacggagttttgaaatgactgaattcaatcaagctttagaagaaataaaagggcaggttgttgaaaacaactctgtaactgaattttctgaggagaaaaacagaactgaaaattacaacaggcaagatggtcagagagttcaaggaggagtccctgctggctctgacgagtatgaagatgaatgccctcatctaattgccttgtcgtcattaaatagagaattcaggcctttcagagatgaagaaaatgtgggagctatgaatcagtatagaacaagaactctgagtatcacttcttcaggcagtgctgtaagctgttcaacaattcctccagaactggtgaaacagaaggtgaaacgtcagttgacaaaacagcaaaaatcagctgtcagacgtcgattgcagaaaggagaagcaaatatatttaccaagcaacgtagggaaaacatgcaaaatatcaaatcaagtttggaagcagccagcttttggggagaataatatatttaggatcttggatatgtttaatatattttttaaagttactgtaattcctttttgagccctcatttgtcttttttgagccaaggctatcatatattaataaataaaccctctttcatctataaatttggtcattcagtatgtgtcttgcaaagaataattacctgccaaagcagtccatttttttcaaagtaatttgggtaactacagttacgtaactttattagagacatttgcatatttttaaaaatctaaataacatttttgttgttttctgtttgttgaggatgatatgctacaatgcacataaagcaattagaacaatgtttagcacattcttaagcattcaataaaacatggctattagaatcataatgattctgaaattctgctgacaatttggttgcattttatttggcaactggaggacttcactgtcttatttttctttagaagtaaagataagcctttggaagtaaagataagccagtgatgcacaatttgcttaattaattgattaataatctgttttgtgccacttacattggtaatgtggtcatgagaatggaaacttacttgagatagtctaccacaatttttacagttggcagcttgtaagcaggcctgagtagcagaagagttaggggttggggaaggtggagtagaatgaggaatgaaatgttctttctcatatgcttattatataagtgggtccggaacatgttctcacctcaaggttaacaaatacctaatgcaggaaagaaaatacatagccatggtcccttcctcaaggactttataattcagtgagatgtaaagatgagtgcttaagtgtatgcacctgattattgtctcttcattgccgcataagtcggaaaaggggatcacttgcagcgaaaaatggccactagatggcaatatcaccccatagtcaccaaacaggattacttgcatccctaattatgttctaccagagggaatatagtacagacttttccaagattgataaagacagcagattccctttcacctttgccaaattaactctactagctatttgcaagacctgtaattatctaataaattggcttaaatatatagaagctagaaaatgccaattcctcttccctcaaaagttacctgacattcaaatatttaaatacccttgctttgcattttagatatatctttgaattcaggtattcattttgtataatctctcaacttttaagcatttttataatgtaaagttgaaaacagcttaatagaaccacagaatgtttattttggagtcattttcttatattcagagctatgaaagcaaattgggaataaagccttatcctgctcagctttacctagttgactcagttgacttttgagtatctactacataaaaagcccattactattaagaaactcaagacaaacatgatttttacctttaatagcctaagatttcttgtgggccaaggaattaattattccaatcagaaatggaggccttacctcatttgggcaaagattagtgctagttattgaagatttactaataaatgatctgttaaggaatttagttttttttggatatgttgttttggttgttgaaaactaaggtataattataatagttattttttgaagatatgtttggctagccatcaattagaagttagagagtttgtcctaacagataagctataaccctcacattttaaaacatgcatagtgaacaatgaacaaaaacaaattcaaaattttactaatgtaaagctgtatattgtgattgttaaaacacttttttttgcctaggagttccagatttaaatcctggttctacacttcccaaacctcaggtaccttctgtggcctctctaacctcaatttttttacttgtaaaatggggacatttaatagtatacctcagtgtcgtgaagactaaatgaagtaccacttgtaaagagtttattacagtgcctgacatatactgtatggttagtactgctaaaatgacttcttaaggttaccaaaaggaaaattccacaagctgaccacctttcctattgtgatggtcactggtatttcagctatgatcctttgttgtgtatttatccagactgaaagatgtttttctagtaaccatcttgcatcgtcttagtctccccattaaataaacttttcctacaaacta

SEQ ID NO: 16 - protein, 547 aa, Mus musculus
MGKVNVAKLRYMSRDDFRVLTAVEMGMKNHEIVPCSLIASIASLKHGGCNKILRELVKHKLIAWERTKTVQGYRLTNAGYDYLALKTLSSRQVVESVGNQMGVGKESDIYIVANEAGQQLALKLHRLGRTSFRNLKNKRDYHKHRHNVSWLYLSRLSAMKEFAYMKALYERKFPVPKPIDYNRHAVIMELINGYPLCQIHHVEDPASVYDEAMELIVKLGNHGLIHGDFNEFNLMLDKDDHITMIDFPQMVSTSHPNAEWYFDRDVKCIREFFMKRFSYESELYPTFSDIRKEDSLDVEVSASGYTKEMQADDELLHPVGPDDKITETEEDSDFTFSDEEMLEKAKVWRSELEKEADPADESGGSWCCSSTDSKQIKDGGLPEESAHVSSFEVTALSQAVEEMERQVLPHRSVTEFSEESRRTENDGQPGQRSPAGSEDCDDEPPHLIALSSVNREFRPFRDEESMSSVTRHRTRTLSVTSAGSALSCSTIPPELVKQKVKRQLTRQQKAAARRRLQKGEANVFTKQRRENMQNIKSSLEAASFWGD

SEQ ID NO: 17 - DNA, 3201 bp, Mus musculus
  source 1..3201: mol_type = genomic DNA
gtggcatactatgtaaaacatttgcaacttgccaaatcagtcaaacgtaaattatttatttaggggctgaaaaatcacttctgcctgatttttagctacaaaaacgagaaaaaaatagcatttttctactgcttcttgagcacgtgttcttacctagcgataagcaccttaaatacagggcgcgtgcgcgggcaggtcgccgccgaaagcctgcgagattacccagcaactttgcgtcttcccagagagcgtccgtggtggaactgggcaagcatggggaaggtgaatgtagccaagcttaggtacatgagccgcgatgacttcagagttctgactgcggttgaaatgggcatgaaaaaccatgagattgttccctgcagtttgattgcttctatagccagcctgaaacatggtggctgtaataagattctaagagaattggtgaagcataaacttatagcgtgggaacgtactaaaactgtgcagggctatcggttgacaaatgctggctatgattacctagctctgaaaacgctgtcttctagacaggtagttgaatctgttggaaaccagatgggtgtgggcaaagagtcagacatttacattgttgccaatgaagccgggcagcagctcgcactgaagctccacagactcggaagaacttcctttcgaaatctgaaaaacaagcgcgattaccacaagcacaggcacaatgtttcttggctttacttatctcggctctcggccatgaaggaatttgcctatatgaaggcattatatgagaggaaatttccagttccaaagccaattgattataatcgccatgcagtgatcatggagcttattaatggttatcccctatgtcagatacatcatgttgaagaccctgcatcagtatatgatgaagctatggaactgatagtcaaactgggaaaccatggactgattcatggagatttcaatgaattcaatctcatgttggacaaagatgaccacatcaccatgattgattttccgcagatggtttccacatcccacccaaatgctgaatggtattttgacagagatgttaaatgcattcgagaattcttcatgaaacgttttagctatgaaagtgagctctacccaaccttcagtgatatcaggaaggaggattctcttgatgttgaggtctctgccagtggttacacaaaggaaatgcaagcagatgatgaactgctacatccagtaggcccagatgataaaattactgaaacagaagaggactctgacttcacgttttctgatgaagaaatgttagaaaaagccaaagtgtggaggtcagaattggaaaaggaagcagacccagcagatgaatcaggtggatcctggtgctgctcgtctacagacagcaaacaaataaaggatggcggcttaccagaggagagtgctcatgtgtccagtttcgaagtgacggcattaagccaagctgtagaagagatggaaaggcaggttcttccccacaggtcggttactgagttctctgaggagagcaggagaactgaaaatgatgggcagccaggtcagagaagccctgctggctctgaggactgtgacgatgaacccccccatctgattgccttgtcatcagtgaacagggagttcaggcctttcagagacgaagagagtatgagtagtgttactcggcatagaacaagaacattgagtgttacgtctgcgggcagtgccctaagctgttcaacaatccctccagaactggtgaagcagaaggtgaagcgtcagctgacaaggcagcagaaggcggctgccagacgccggctgcagaaaggagaagccaatgtgtttaccaagcagcggagggaaaacatgcagaatattaaatcgagtttggaagcagccagcttttggggagactaatagatttctgatatgtattatttttttagatgctataattttatttttagccagtatattggtcttttttggaccaaagccagtaaattatggaaataaagcctctttagactcagagcttccaaggtctgattaatagtaaatctgccaagcactctgtttcttaaaatttttcaggtaactaatttaaatataaaataacgtgtgaaaaaaacttcattggcagtaattgcaaaacatggacatcatttttatttcactgtgatgttcagactctgccttaaacaggtagaaaaatgacttgcctcagattctggatttacatgtaataaataatagaacaacttaatgcaatctgtagttctctcttttctccttcagtcggtcacacttgggcttttactacattggtgagttggctttaggatcacctgagacagtcagtacacagttatttgaccattggagtgaaactgatcgtcttctgtttctttagacttctggagtggagatgaatcagtaatgggtagacgagcagcagcgggtcttttggtgccgttctcattggccctgtggtcgtgggaaaaggaagtttaggttcctaccagtatttttgcagttgtcagctttccccaggtttcagcagaagtggggggagggagggagggagggagggagggagggagagagagagagagagagagagagagagagagagagagagagagagagagaatgcatgactccaggatgtgttcctacctcagtgttaacaagtctctagaacaacactctggtgtgattcatccctcaagcgcattctaattcagtcagtggaagtgagtgtaagtatacaagcactgtcccttcagcttccctgagcagatctcgaatagaagatcgtcttcagcaagaagtgaccaccagatggcagtgtttgccagcagtgacatgataaaaacaccagcttcccttttaccttagcaaaattcattctgactatttgtaactgatcactatctaagcgcatttaaatgtttggatatatgaatatgtcaagtgcagtttctttgaaaattagactgatttttggaagtgttgatatagccctgttttaaaatgtagtttaagctcagatactcattttttatactctgctaaaagtgtttctataacataaactgatgttgaagatagtttaataagaccacagaatgtttattttgtaatacgtttttcttatgtccaaagcagattgggaataaaagtgtttgacctcaaaaa

SEQ ID NO: 18 - protein, 551 aa, Rattus norvegicus
MGKVNVAKLRYMSRDDFRVLTAVEMGMKNHEIVPCSLVASIASLKHGGCNKILRELVKHKLIAWERTKTVQGYRLTNAGYDYLALKTLSSRQVVDSVGNQIGVGKESDIYIVANEEGQQFALKLHRLGRTSFRNLKNKRDYHKHRHNVSWIYLSRLSAMKEFAYMKALHERKFPVPKPVDYNRHAVIMELINGYPLCQIHHVEDPASVYDEAMELIVKLGNHGLIHGDFNEFNLMLDKDDHITMIDFPQMVSTSHPNAEWYFDRDVKCIREFFLKRFNYESELYPTFSDIRREDSLDVEVSASGYTKEMQADDELLHPVGPDDKITETEEESDLSLSDEEMLERAKVQRSELENEPNPADESGSSCCFSSADSKQMKEDGLPEESADVSSFEVTKLHQAVEEVGRQVLTHKSVAEVSEESTRTEDDPRHDGQPGQSSPAGCAEGEDELPDLIALSSLNKEFRPFRDEDSVSSVTQHRTRTLSVTSTGSVLSCSTIPPELVKQKVKRQLTRQQKAAARRRLQKGEANIFTKQRRENMQNIKSSLEADSFWGD

SEQ ID NO: 19 - DNA, 1814 bp, Rattus norvegicus
  source 1..1814: mol_type = genomic DNA
gctgggcaagcatggggaaggtgaatgtggccaagctaaggtacatgagtcgagatgacttcagagttctgactgcggttgaaatgggcatgaaaaaccatgagatcgttccctgcagtttggttgcttctatagccagcctgaaacacggtggctgtaataaaatcttaagagaattggtgaagcataaacttatagcatgggaacgtactaaaactgtccagggctatcggttgacaaatgctggctatgattacctagctttgaaaaccctgtcttccagacaggtagttgattctgttggaaaccagataggtgtcggcaaagagtcagatatttacattgttgcaaatgaagaagggcagcagtttgcactgaagctgcaccgactggggagaacctcctttcgaaatctgaaaaacaagcgtgattaccataagcacagacataatgtttcttggatttatttatctcgtctctctgccatgaaggaatttgcctatatgaaggcattacatgagcggaaattcccagttccaaagccagttgactacaatcgccacgcagtgatcatggagcttatcaatggctaccccctgtgtcagatacatcatgttgaagaccctgcatcagtgtatgacgaagctatggaactgatagtcaaactgggaaaccatgggctgattcatggagatttcaatgaattcaacctcatgttggacaaagatgaccacatcaccatgattgattttccacagatggtttccacttcccacccgaacgctgaatggtattttgacagagatgttaaatgcattcgagagttcttcctgaaacgttttaactatgaaagtgagctctacccaaccttcagtgatatcaggagagaggattctctcgatgttgaggtctctgccagtggttacacgaaggaaatgcaggcagatgatgaactgctacatccagtaggtccggatgataaaattactgaaacagaagaggaatctgacttgtcactctctgatgaagaaatgttagaaagagccaaagttcagaggtcagaactggaaaatgaaccaaacccagcggatgaatcaggtagctcctgttgcttctcatctgcagacagcaaacagatgaaggaagacggcttacctgaggagagtgctgatgtgtccagttttgaagtaactaagttacatcaagctgtagaagaagtgggaaggcaggttcttacccacaagtctgttgctgaggtttctgaggagagcactagaactgaggatgatcccaggcatgatggccagccgggtcagagcagccctgctggctgtgcagagggggaggatgaactccccgatctcattgccctgtcatcattgaacaaggagttcaggcctttcagagatgaagacagtgtgagcagtgttactcagcatagaacaagaaccttgagtgttacgtctaccggcagtgtcctgagctgctccacaatccctccagaactggtgaagcagaaggtgaagcgtcagctgacgaggcagcagaaggcagctgccagacgccggctgcagaaaggagaagccaatatatttaccaagcagcggagggaaaacatgcagaatatcaaatcaagtttggaagcagacagcttttggggagactaataaatttttgatctgtatatttttttagatactgtaattctatttttagccagtatattggtctttttaagaccaaagccaatatttatggaaattatggaaataaacccttattagactcaaaaaaaaaaaaaaaaaaaaaaaaaa

SEQ ID NO: 20 - protein, 539 aa, Homo sapiens
MPENVAPRSGATAGAAGGRGKGAYQDRDKPAQIRFSNISAAKAVADAIRTSLGPKGMDKMIQDGKGDVTITNDGATILKQMQVLHPAARMLVELSKAQDIEAGDGTTSVVIIAGSLLDSCTKLLQKGIHPTIISESFQKALEKGIEILTDMSRPVELSDRETLLNSATTSLNSKVVSQYSSLLSPMSVNAVMKVIDPATATSVDLRDIKIVKKLGGTIDDCELVEGLVLTQKVSNSGITRVEKAKIGLIQFCLSAPKTDMDNQIVVSDYAQMDRVLREERAYILNLVKQIKKTGCNVLLIQKSILRDALSDLALHFLNKMKIMVIKDIEREDIEFICKTIGTKPVAHIDQFTADMLGSAELAEEVNLNGSGKLLKITGCASPGKTVTIVVRGSNKLVIEEAERSIHDALCVIRCLVKKRALIAGGGAPEIELALRLTEYSRTLSGMESYCVRAFADAMEVIPSTLAENAGLNPISTVTELRNRHAQGEKTAGINVRKGGISNILEELVVQPLLVSVSALTLATETVRSILKIDDVVNTR

SEQ ID NO: 21 - protein, 509 aa, Homo sapiens
MPENVAPRSGATAGAAGGRGKGAYQDRDKPAQIRFSNISAAKAVADAIRTSLGPKGMDKMLVELSKAQDIEAGDGTTSVVIIAGSLLDSCTKLLQKGIHPTIISESFQKALEKGIEILTDMSRPVELSDRETLLNSATTSLNSKVVSQYSSLLSPMSVNAVMKVIDPATATSVDLRDIKIVKKLGGTIDDCELVEGLVLTQKVSNSGITRVEKAKIGLIQFCLSAPKTDMDNQIVVSDYAQMDRVLREERAYILNLVKQIKKTGCNVLLIQKSILRDALSDLALHFLNKMKIMVIKDIEREDIEFICKTIGTKPVAHIDQFTADMLGSAELAEEVNLNGSGKLLKITGCASPGKTVTIVVRGSNKLVIEEAERSIHDALCVIRCLVKKRALIAGGGAPEIELALRLTEYSRTLSGMESYCVRAFADAMEVIPSTLAENAGLNPISTVTELRNRHAQGEKTAGINVRKGGISNILEELVVQPLLVSVSALTLATETVRSILKIDDVVNTR

SEQ ID NO: 22 - DNA, 2376 bp, Homo sapiens
  source 1..2376: mol_type = genomic DNA
cttctccgcctccgcctcctcccgacgccggcgccgctttctggaaggttcgtgaaggcagtgagggcttaccgttattacactgcggccggccagaatccgggtccatccgtccttcccgagccaacccagacacagcggagtttgccatgcccgagaatgtggcaccccggagcggggcgactgccggggctgccggcggccgcgggaaaggcgcctatcaggaccgcgacaagccagcccagatccgcttcagcaacatttccgccgccaaagcggttgctgatgctattagaacaagccttggaccaaaaggaatggataaaatgattcaagatggaaaaggtgatgtaaccattacaaatgatggtgctaccattctgaaacaaatgcaagtattacatccagcagccagaatgctggtggagctgtctaaggctcaagatatagaagcaggagatggcaccacatcagtagtcatcattgctggctccctcttagattcttgtaccaagcttcttcagaaagggattcatccaaccatcatttctgagtcattccagaaggccctggaaaagggcattgaaatcttgactgacatgtctcgacctgtggaactgagtgacagagaaactttgttaaatagtgcaaccacttcactgaactcaaaggtggtttctcagtattcaagtctgctttctccaatgagtgtaaatgcagtgatgaaagtgattgacccagccacagccaccagtgtagatcttagagatattaaaatagttaagaagcttggtgggacaattgatgactgtgagttggtggaagggctggttctcacccaaaaagtgtcaaattctggcataaccagagttgaaaaggccaagattgggcttattcagttttgcttatctgctcccaaaacagacatggataatcaaatagtggtttctgactatgcccagatggaccgagtgctgcgagaagagagagcctatattttaaatttagtgaagcaaattaaaaaaacaggatgtaatgtccttctcatacagaaatctattctaagagatgctcttagtgatcttgcattacactttctgaataaaatgaagatcatggtgattaaggatattgaaagagaagacattgaattcatttgtaagacaattggaaccaagccagttgctcatattgaccaatttactgctgacatgctgggttctgctgagttagctgaggaggtcaatttaaatggttctggcaaactgctcaagattacaggctgtgccagccctggaaaaacagttacaattgttgttcgtggttctaacaaactggtgattgaagaagctgagcgctccattcatgatgccctatgtgttattcgttgtttagtgaagaagagggctcttattgcaggaggtggtgctccagaaatagagttggccctacgattaactgaatattcacgaacactgagtggtatggaatcctactgcgttcgtgcttttgcagatgctatggaggtcattccatctacactagctgaaaatgccggcctgaatcccatttctacagtaacagaactaagaaaccggcatgcccagggagaaaaaactgcaggcattaatgtccgaaagggtggtatttccaacattttggaggaactggttgtccagcctctgttggtatcagtcagtgctctgactcttgcaactgaaactgttcggagcattctgaaaatagatgatgtggtaaacactcgataatctggataactgactagcaccattatgatcaccagtattgtggctggaatggaagaagatcaccttggtgttccttgtttggaagattatttcctctgaatttctgggcttggtcttccagttggcatttgcctgaagttgtattgaaacaatttaatgaaaatattaaatatttggtttcaaaaggcagatttatcttctcccaacattctgttatttctgatacttttgaaaaactaataaaaactaataaaagaagcgtaaaaagtgagtttacatgttgaggaaaaaaatggcccaatatgctcatcactgataaatgctccctggccttaaaaactaccaacatataatatatatgctgtcttaaaagttaatgatccaagtggcacctctctgaacctactttggcttgggaggctgcccagttaaaacaaaaataagttaatggtacagaaagagaatgaaaaatgaaagcctccttttatcctatcatcctaattccttttcccagtaataatgactgctgtgttggattccttctgcaaataaaagtgtatacatatatgtagcaaatcttacttaaacaaaagggtttcttaactaa

SEQ ID NO: 23 - DNA, 2262 bp, Homo sapiens
  source 1..2262: mol_type = genomic DNA
ggcaaggaaggcccccttctccgcctccgcctcctcccgacgccggcgccgctttctggaaggttcgtgaaggcagtgagggcttaccgttattacactgcggccggccagaatccgggtccatccgtccttcccgagccaacccagacacagcggagtttgccatgcccgagaatgtggcaccccggagcggggcgactgccggggctgccggcggccgcgggaaaggcgcctatcaggaccgcgacaagccagcccagatccgcttcagcaacatttccgccgccaaagcggttgctgatgctattagaacaagccttggaccaaaaggaatggataaaatgctggtggagctgtctaaggctcaagatatagaagcaggagatggcaccacatcagtagtcatcattgctggctccctcttagattcttgtaccaagcttcttcagaaagggattcatccaaccatcatttctgagtcattccagaaggccctggaaaagggcattgaaatcttgactgacatgtctcgacctgtggaactgagtgacagagaaactttgttaaatagtgcaaccacttcactgaactcaaaggtggtttctcagtattcaagtctgctttctccaatgagtgtaaatgcagtgatgaaagtgattgacccagccacagccaccagtgtagatcttagagatattaaaatagttaagaagcttggtgggacaattgatgactgtgagttggtggaagggctggttctcacccaaaaagtgtcaaattctggcataaccagagttgaaaaggccaagattgggcttattcagttttgcttatctgctcccaaaacagacatggataatcaaatagtggtttctgactatgcccagatggaccgagtgctgcgagaagagagagcctatattttaaatttagtgaagcaaattaaaaaaacaggatgtaatgtccttctcatacagaaatctattctaagagatgctcttagtgatcttgcattacactttctgaataaaatgaagatcatggtgattaaggatattgaaagagaagacattgaattcatttgtaagacaattggaaccaagccagttgctcatattgaccaatttactgctgacatgctgggttctgctgagttagctgaggaggtcaatttaaatggttctggcaaactgctcaagattacaggctgtgccagccctggaaaaacagttacaattgttgttcgtggttctaacaaactggtgattgaagaagctgagcgctccattcatgatgccctatgtgttattcgttgtttagtgaagaagagggctcttattgcaggaggtggtgctccagaaatagagttggccctacgattaactgaatattcacgaacactgagtggtatggaatcctactgcgttcgtgcttttgcagatgctatggaggtcattccatctacactagctgaaaatgccggcctgaatcccatttctacagtaacagaactaagaaaccggcatgcccagggagaaaaaactgcaggcattaatgtccgaaagggtggtatttccaacattttggaggaactggttgtccagcctctgttggtatcagtcagtgctctgactcttgcaactgaaactgttcggagcattctgaaaatagatgatgtggtaaacactcgataatctggataactgactagcaccattatgatcaccagtattgtggctggaatggaagaagatcaccttggtgttccttgtttggaagattatttcctctgaatttctgggcttggtcttccagttggcatttgcctgaagttgtattgaaacaatttaatgaaaatattaaatatttggtttcaaaaggcagatttatcttctcccaacattctgttatttctgatacttttgaaaaactaataaaaactaataaaagaagcgtaaaaagtgagtttacatgttgaggaaaaaaatggcccaatatgctcatcactgataaatgctccctggccttaaaaactaccaacatataatatatatgctgtcttaaaagttaatgatccaagtggcacctctctgaacctactttggcttgggaggctgcccagttaaaacaaaaataagttaatggtacagaaagagaatgaaaaatgaaagcctccttttatcctatcatcctaattccttttcccagtaataatgactgctgtgttggattccttctgcaaataaaagtgtatacataaaa

SEQ ID NO: 24 - protein, 524 aa, Homo sapiens
MADAEVIILPKKHKKKKERKSLPEEDVAEIQHAEEFLIKPESKVAKLDTSQWPLLLKNFDKLNVRTTHYTPLACGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWKQEYVDYSESAKKEVVAEVVKAPQVVAEAAKTAKVSGMLSSVWNRKRESESESDETPPAAPQLIKKEKKKSKKDKKAKAGLESGAEPGDGDSDTTKKKKKKKKAKEVELVSE

SEQ ID NO: 25 - protein, 509 aa, Mus musculus
MADAEVITFPKKHKKKKDRKPLQEDDVAEIQHAEEFLIKPESKVAQLDTSQWPLLLKNFDKLNVRTAHYTPLPCGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLCFVLGSGCQMQELRRVRSGVMSEKDHMVTMHDVLDAQWLYDNHKDESYLRRVVYPLEKLLTSHKRLVMKDSAVNAICYGAKIMLPGLLRYEDGIEVNQEIVVITTKGEAICMAIALMTTAVISTCDHGIVAKIKRVIMERDTYPRKWGLGPKASQKKMMIKQGLLDKHGKPTDNTPATWKQDYIDYSDSGKNTLVTEAVQAPQLAAEAVNVIKRKRDSESESDETPTVPQLKEKKKKKDKKPKTVLESGGETGDGDNDTTKKKKKKKVKVVEEMSE

SEQ ID NO: 26 - DNA, 10918 bp, Homo sapiens
  source 1..10918: mol_type = genomic DNA
gagcagcgcggcctgacgggaccaaggcggcgggagtctgcggtcgttccctcggctgtggaccgggcggcacgcacgcggtgcagggtaacatggcggatgcggaaggtaagggctgcaggcttccgggccgtgctaactccgggcgactcggggaacgggggtggggggatggtatcggggcccgcgcacgcctccctctggttcccgccggcgtgtcggcctcttcaccgcccggccttaagccggccttgtctctcggccctggctccgtggcaagggcgcgagagtgggagcccctcgcggtgatttggcatgatggggctcggccctctcacctccctggggtccgggtcgccgtcgaaagacatctgccgtgctgcccaccccgcggtggcgtgggacgtggcagtgtctgtgctgctgccagcccggacgccgccgttacagctccatgcgactaggggagcaagggtagtggaaacgggccgagaagaaaaggagcggagggaccccggagcagggatagagctccgagttgcaagaaagttctagaggccacaagcccgcggggacagtcgagtggtcgcttatggaactttccggccccctgcgtgagcgcttctgtgggatgtttaagctctgccatagccacgtggtgaaccggaagggaagccgacacgtgcagtagcctacagtcagttacccagccatgggacccatacatttggccttacacaccttgttttctcgcttacctacggatatacgttctaagaaatgggtcgttaggcgatttcatcattgtgtgaacatcagagggctcttacacaaacctaggtggcataggctactacacacctaggctgtatatggtgtagcgtatggctcctaggcttcaaacctggacagcatggtattgtgcggaatgctgtaggcagttgggacacagcgtcgagagtttgtgtacttaaacatgtctaagcagaaaaaggacagtaaaaatatagcacagggccgggcgcggtggctcaggcctgtgatcccagcccaggctggcggatcacctgcggtcaggagttcaagaccagcctggccaacatggtgaaaccccgtctctactaaaaatacaaaaaatttaggtgggcgtggtggtatacgcctgtaatcccagctactcgggaggctgaggcaggagaattgcttgaacctggagaaggaggttgcagtgagccgagtttctgccactgcactccagcctggggaaagagtgtgaccgtctcaaaaaaaaaaaattatatatatataaatatgtgtgtgtatatatgtatattatatatgtatgtattatatgtatgtatattatatatatatagtacacaaggttataagtggtgcacccgaagaggagggcacttaccatgaatggagcttgctgagctggaagttgccctggttgagtcggtgactgaatgtgaaggcctcagacattacactactggagactttataatctgtacacctaggctacactaaatttataaaaatatatttttcttcagaataaatcaaccttagcttactgtaactttataaactttttacttcgactgttgtaataatacttagcttaaaacaaacacattatttagctgtacgtaaatattttctctgcttatctccttatttataagttttctattttaaaattttttaatttttttcttaaaaaatttttttttgttaactaagacacaaacacagcacacacagtagccttgacctacacagggtcaggatcatcaagacatcactaggagataggaatttttcagctctgttataatcttatgggggccaccatcacatacgcagtctgtacttgcaaaacattatgtggcatgtgactgtaattgtagacctaaaatccatttcctacctgccctaatttcggtctttattttaaacccttgtttcatttattcttggggaaaattcccaaatcctgtgtttgtttttttagtaattattttgccaaagaaacataagaagaaaaaggagcggaagtcattgccagaagaagatgtagccgtgagtagtaatgtgtttgacttcactttgactaaaaagaaagtttacttaaataacaaactaaaggaaagaagtgtttttaaaggtttccaaggaatgggccagcattggtagtcattgtgtagattcgaaaaagcactgaaatgacacagccccagacagtccaagtgtccttcaggcatagcttactaaatggtgctggccttaccaactgctgtctctgaatttgtcttccttatgccacccacagacccgccatccccctttctctttaaatgtttggacccatcttagtgacatttcacttcccctctgtactcctggttcatttcttctctctctttcccttggaggtcccagtgaaaaggcataggaagccttgttttctgtttaaaaggcatacatttccatggcagtaatggaattgttcaaaatcgggtgggaagtttaataggttatatttctgtcctgtcgaaggaaatacaacacgctgaagaatttcttatcaaacctgaatccaaagttgctaagttggacacgtctcagtggccccttttgctaaaggtatgtggttaaaatcgtgcatcagatgaatgcctgcttttacgttatttccaagtgttaaacaattgattgagaatgcttactgctggcatccttgactttgggaagtgcccactgatacagtttaaagcaggattccttaaggtatctgaaatgagaaccaagtttttaaaaaattggcagtgcactcaacactgttgtagaacacatgtccccacaatgttaaattgctgagtttgtcaacagttctcagtttttgtacttagtccatcttgcagacctatgccagtttgtgggccacatagtggtactgactctttttttacatgtgctcacgacatggtattttgttttgtgttgtgtcgttgctttgtagaattttgataagctgaatgtaaggacaacacactatacacctcttgcatgtggttcaaatcctctgaagagagagattggggactatatcaggtaagtgttgggaggaggcactgtgcaaacttactttcttttgaaaatgctttcacatcagctattcaggaaggcagtggcatgccagaatagctttgtcttgaatatataacctgtacccgcagtgaattatttttctttttaacttttcagatttgttgtttcactggagcattaagagtgggtgatacattaattttttttttttccattccaggacaggtttcattaatcttgacaagccctctaacccctcttcccatgaggtggtagcctggattcgacggatacttcgggtggagaagacagggcacagtggtactctggatcccaaggtgactggttgtttaatcgtgtgcatagaacgagccactcgcttggtgaagtcacaacagagtgcaggtatgtgggagagggagggaaggactggctagagtgaaaagctttctgtatttcccttctatgttttgtctgctggaaactatttcttcattaagaaaaaaaaaaatcctaaagcaaggatattgttttggtttctgtgtacgttctgtcccctgccagatcttggtggtccgcgctttttggaattccacatactaagcatgtattcatcaagctgactggatccatttgtccgggttcagctctcagcttgtcactccaggcttctcactctgtgctaccgcttctgcctaaagtatccttcgttcatttttatctgagaaaagcctgcttatcttgcaaggctttttccacgcagcttctacactgagtactcccaactgtccctgttagtacattgaattcccagtgagaaagtgtcttcattctttcccacatcatcaccatgaaatctctcttaatgcaaattttatagaaacacaaggtcaaactgccgcttttcctgctgtgcaaaatgggagtgactgagcatataagaaatatggaaaacagaaaactcagtggccacacctgactactcttttgtcatttttcaggcaaagagtatgtggggattgtccggctgcacaatgctattgaaggggggacccagctttctagggtaagtctgcaattgtagggaggacaccctttgttgacttggatctctgagcccctaaacatctggagatgggttagctgtcctgactgactccttttgtccccttttcagtcggctgctgcagccagcctggaccagtgactgcacatgtacattctgatgaggtgtttgttttcatttgtgtttgttcttaggccctagaaactctgacaggtgccttattccagcgacccccacttattgctgcagtaaagaggcagctccgagtgaggaccatctacgagagcaaaatgattgaatacgatcctgaaagaagattaggtgagtcattaggcctgtgagtgggtatgaacacattcttcaggattctgttctcttattaaaagtagatgcccttgaagttgaagactttctaaaacctgccctacagctgggatattgtctgtgggcgatagtttttgttatcttttgttgaaaaagtgaacagtgcctagtaaagtgagatggcttttacaccttttatgatgactgcttctgctggagttgaaatgttttcattctgcatttgtgtagttcggtgctttgttcaaaggtaagtgttttcaccaaagtatgttttgcatgtgttttttatttgctgcaattttgactgcttagaagtttctgttgtcagaagtaaatttaagacaatgccaattttcctcccatccatgaaacataccatgctgaatatacctgcaacttaagtgttaaaaattctgctaatatcagaattacagatctttttttttttttttttttttttgagacggagtctcgctctgtcgcccaggctggagtgcggtggcatgatctcggctcactgcaaactctgtctcccgggttcacaccattttcctgcctcagcctcccaagtagctgggactataggcgcccgccatcatgcccggctaattttttgtacatttagtagagacggggtttcaccatgttagccaggatggtcttgacctcctgacgtcgtgatccacccgcctctgcctcccaaagtgctggaattacaggcgtgagccaccgtgcctggccagaattacagatctttacagcaaatgcttatattttaagaagtgtgtctgtcacctctagtcttggtggctcagatgaaggataactgcatttctcaaccagtgatgtatttacagtaggtgctgcttttcttttgtgtgtgtgtatcttaggaatcttttgggtgagttgtgaggctggcacctacattcggacattatgtgtgcaccttggtttgttattgggagttggtggtcagatgcaggagcttcggagggttcgttctggagtcatgagtgaaaaggtatgtgttacggggctagaagttttagagctggttcttaccatctgccagcctttgatggcactccacttgcttcatgtcgtgggaaagatgctttccaaagtgttgagttcagtccagggcagcttccctgttctgttaattaaactttgggacattaaaatgggctaagggagatgattgggtagaaagtattattctattcatttgcctcccagcctacaaaaatgcctgcttggggtctaatacttcaacggttaaagatgcctggaagagggcgcggtggctcacgcctgtaatcccagcactttgggaggccgaggcgggtggatcatgaggtcaggagatcgagaccatcctggctaacaaggtgaaaccccgtctctactaaaaatacaaaaaaaattagccgggcgcggtggcgggcgcctgtagtcccagctactcgggaggctgaggcaggagaatggcgtgaacccgggaagcggagcttgcagtgagccgagattgcgccactgcagtccgcagtcccacctgggcgacagagcgagactccgtctcaaaaaaaaaaaaaaaaaaaaaaaaagatgcctggaagagccaggactgaagaggctgaaaacataatgagcttggactagtaaacaagtgacattcagttttaggaagtttggggtctgatgggctgagatacaaataaactgaattattttcatacatggctgcttttcaggaccacatggtgacaatgcatgatgtgcttgatgctcagtggctgtatgataaccacaaggatgagagttacctgcggcgagttgtttaccctttggaaaagctgttgacatctcataaacggctggttatgaaagacagtgcagtaagttccgggttaggagtttatatttggaaagaacgtactccaaagatgacgcttgctctttttattgttttccttgggttttattttatttttttcaagtccaaaccaagtatattaatttaaagtaaaagtttaatggtaggcatacttaaaaataaggcacattaggccaggcacagtggctgatgcctataatcctagcactttggcaggctgaggcaagaggatcacttgagtccaggaaggagtttgagaccagcttgagcccaggagatcaaggctgtgagctgtgtttttatgatgatcagagagttggccttttataaattaaaacattgtctttttctctaacttgagggataaaaatttctggatatttaaaatgtttgtagttggtaattatttatattcaaatcttgtatagatgttcacattttttgtatgtgtataggctgtaataaatactgaaagatggcaccataatagttgaaaaaactcagtcacaaaacagtggacaacataaatgtgatgtcctctccttatcaaaccattggcttctgggtgaatgtaaagggaggggagaatgtgatcaaaagaggtctaggtgagtggagatggtatgaggcagatgggtgcattgggtaccattgaacctctggagaggtagccttaacttgtacccactgacctataaattctgcaccaagacttttgtgtgattggttcaacagggctcagtaatcttgttctcaagttctttaagagcctgttattgcctggatctccagtaaatctgttctttctcctcataaggaggagagaggagtaattaaggaatgccaaggctatttttatgtttcaaatgaaaatatggaccacgtgcggtggctcacacccataatcccagcgctttgggaggccgggccaggcaaattgcttgagctcaggagtttgagaccagcctgggcaatatggtgaaaccctgtctctattaaaaatacaaaaattagctgggcgtggtggcacgtgcctgtaatcccagctccttgggagactgagaatcacttgaacttgggagatggaggttgcagtgagccgagaatgtgccactgcactccaccctggacaacagagggaggttctgcctgaaaattaaaaaaaaaaaaaaaaaaaagaaaatacttgtgagtctctagaacctgaaaacctgaaagtcttctttactacttttgagtcgtgtgtaatgtgcggagagtggtacctatagcacagtgaggtggctcttctgagtaccactcatgccccttgcagctagtgggctataagtgtcatccctgtttcctggtgtcaggccccactcccttgttgtcctcctttactctggtgtgggagtggggtggagggcagcagctgggccccttacacttggtgccatctctgcaggtaaatgccatctgctatggggccaagattatgcttccaggtgttcttcgatatgaggacggcattgaggtcaatcaggagattgtggttatcaccaccaaaggagaagcaatctgcatgggtaagaggggatgtttattttttaaattctaaatgtttgtggttacatactaggtgtatatatttatggggttcatgagatgttttgatacaggcatgcaatacataataatcacatcatggagaatggggcatccatcttaagcatttatcctttgtgttacaaacaatcctattttactgttagttatttatcaccttagttattttaaaatatacacttaagttattattgattatagtcaccccgttgtgctattaaatagtctcactcttcttgttcattcttttggttttttggtggtggtgttttttttttttttttttttttgagacagtcttgctcttttcacccagactgcagtgcagagacgtgacctcggctcactgcaacttctgcttcccaggttcaagcgattctcgtgcctcagccttccgagtagctaggattacaggtgcacaccatgacacctggctaattttttgtatttttagtagagacagggttttgtcatgttggtcaggctggtctcgaacttctgacctcaggtgatctgccctcccaaagtgctgggattacaggtgtgagccaccatgccgggcctgattttttttttttttgtacccattaaccatccctacctccctgccagccccccactaccctcccagtctctggtaaccattcttttactcttctctgtccatgagttcaattgtttcgattcttagctcccacaaataagtgagaacatgcaatgtttgtctttctgtgactggccgattacacttaatataacgatcttcagttccatccatgttgttggaaatgacaggatctcattcttttttaaggctgaatagtaatccatcgtgtatatgtaccacattttctttatccatttatctgtcgatgggcacttcggttgcttccaaatcttggctactgtgatctctttgataacactgatttcctttatttggggtatatacccagcagtgggattgctggatcctatggtagctcaatttagtttttcgaggaacctccaaactgttcttcatagtggttatactaatttacattcccatcaacaatgtacaaaggttcccttttctccacatccttgccagcacttgtcattgcctgccttttggatataaaccattttaactggagtgaaatgatatttcattgtagttttgatttgcatttctctcgtgatcagcattgagcactttttcatatgcctgtttgccatttatttgtcttcttttgagaagtgtctgttcaaatcttttgcctgttttttgatcagattattagattttttttcctatagtgctgagttccttatatattctggttattaatcccttgtcagatgggtagtttgcggatattttctcccattcttatgcatatggatatctagttttcccagcaccatttattgaagagactggtttttacccagtgtatgttcttggcacctttgtcaaaaatgagttaaatgtaggtgtgtgcatttgcttctgggttccttattctgttccattggtttatgtgcctgtttttatgccagtaccatactgttttggatactgtagctctatactataatttgaagtcaggtaatgtgattcctccaggtttgttctttttgcttaggatagctttggctattttgagtcttttgtggttccatgtaaagtaccagtgttttttaacttgtgaaagcttagctactgttggattgtctcttaagactttaaatgctctgcgcaccctcttagtgaatgaaccttagagctattcatttaatgggtctaatttgtaccagaagtaagcatgtcatcaggattccttagctctgtgagcaggcaaggcagccacggcttatggacagcagcagaaaaggaatccttaaaacttcaaaggttctgtggccccaaagggagtgtgcatgccgttcttatagacgtcttgagctgcaagcctgttatgtgtatcgttaggtgctttggttaggtgttttttaacttcagaagtttaacgtcatttcaaaggcctccttctcataatggagtatttcctccatatgcacatcctgagcaaaggtaaccccaattaaagtagtagctcagagttttaaaagttttaaagtggcatacaacagtagaattggggctcatcaattatcaattctttcacccttcaaataattcttttctttattcaatgcctgtagctattgcattaatgaccacagcggtcatctctacctgcgaccatggtatagtagccaagatcaagagagtgatcatggagagagacacttaccctcggaagtggggtttaggtccaaaggtaagtggtactgggttgcgtgccctgccaggttcttttttttttttttttttttttaaatttatttttttattgataatttttgggtgtttctcacagagggggatttggcagggtcataggacaatagtggagggaaggtcagcagataaacaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccatttaaccctgagtggacacagcacatgtttcagagagcacagggttgggggtaaggtcacagatcaacaggatcccaaggcagaagaatttttcttagtacagaacaaaatgaaaagtctcccatgtctacttctttccacacagacacggcaaccatccgatttctcaatcttttccccacctttcccccgctttctattccacaaaaccagcattgacaccattctaaa

SEQ ID NO: 27 - protein, 552 aa, Homo sapiens; identical to SEQ ID NO: 12

SEQ ID NO: 28 - protein, 451 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLSSRQVVESVGNQMGVGKESDIYIVANEEGQQFALKLHRLGRTSFRNLKNKRDYHKHRHNVSWLYLSRLSAMKEFAYMKALYERKFPVPKPIDYNRHAVVMELINGYPLCQIHHVEDPASVYDEAMELIVKLANHGLIHGDFNEFNLILDESDHITMIDFPQMVSTSHPNAEWYFDRDVKCIKDFFMKRFSYESELFPTFKDIRREDTLDVEVSASGYTKEMQADDELLHPLGPDDKNIETKEGSEFSFSDGEVAEKAEVYGSENESERNCLEESEGCYCRSSGDPEQIKEDSLSEESADARSFEMTEFNQALEEIKGQVVENNSVTEFSEEKNRTENYNRQDGQRVQGGVPAGSDEYEDECPH

SEQ ID NO: 29 - protein, 90 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLFF

SEQ ID NO: 30 - protein, 92 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKHFLLGK

SEQ ID NO: 31 - protein, 58 aa, Homo sapiens
MGKVNVAKLRYMSRDDFRVLTAVEMGMKNHEIVPGSLIASIASLKHGGCNSFKRISET

SEQ ID NO: 32 - protein, 103 aa, Homo sapiens
MGKVNVAKLRYMSRDDFRVLTAVEMGMKNHEIVPGSLIASIASLKHGGCNKVLRELVKHKLIAWERTKTKYFEAPFVIFQVIGFHQSKCSKSQEVEVARLVKH

SEQ ID NO: 33 - RNA, 20 nt, synthetic construct
  source 1..20: mol_type = other RNA
gaacggcgggtttcttaccg

SEQ ID NO: 34 - RNA, 20 nt, synthetic construct
  source 1..20: mol_type = other RNA
catttgtcaaccgatagccc

SEQ ID NO: 35 - RNA, 20 nt, synthetic construct
  source 1..20: mol_type = other RNA
tgacttcagggtcttgaccg

SEQ ID NO: 36 - RNA, 20 nt, synthetic construct
  source 1..20: mol_type = other RNA
tgattacaatcgtcatgcag

SEQ ID NO: 37 - RNA, 20 nt, synthetic construct
  source 1..20: mol_type = other RNA
ttctccgcgggaacaaacgg

SEQ ID NO: 38 - DNA, 22 bp, synthetic construct
  source 1..22: mol_type = other DNA
gctatcggttgacaaatgcagg

SEQ ID NO: 39 - DNA, 23 bp, synthetic construct
  source 1..23: mol_type = other DNA
gaaacgaggttcttcctagtctg

SEQ ID NO: 40 - DNA, 21 bp, synthetic construct
  source 1..21: mol_type = other DNA
tgcggcctctatcacaagatg

SEQ ID NO: 41 - DNA, 21 bp, synthetic construct
  source 1..21: mol_type = other DNA
ctgcccgtttactgacaatca

SEQ ID NO: 42 - DNA, 21 bp, synthetic construct
  source 1..21: mol_type = other DNA
actgacggagagcatgaagat

SEQ ID NO: 43 - DNA, 19 bp, synthetic construct
  source 1..19: mol_type = other DNA
ccggcacataggaggggta

SEQ ID NO: 44 - DNA, 21 bp, synthetic construct
  source 1..21: mol_type = other DNA
gtgccctatgacacggatcta

SEQ ID NO: 45 - DNA, 21 bp, synthetic construct
  source 1..21: mol_type = other DNA
agtcccagtaatggtcgctat

SEQ ID NO: 46 - DNA, 22 bp, synthetic construct
  source 1..22: mol_type = other DNA
aggcaatgacgagaactactcc

SEQ ID NO: 47 - DNA, 19 bp, synthetic construct
  source 1..19: mol_type = other DNA
cgaaggtcgttgaacctgg

SEQ ID NO: 48 - DNA, 19 bp, synthetic construct
  source 1..19: mol_type = other DNA
ggttgcggcaagagctaca

SEQ ID NO: 49 - DNA, 20 bp, synthetic construct
  source 1..20: mol_type = other DNA
gtcagagcgcgaaaaagcac

SEQ ID NO: 50 - DNA, 22 bp, synthetic construct
  source 1..22: mol_type = other DNA
caacttcaagctaagccactgc

SEQ ID NO: 51 - DNA, 19 bp, synthetic construct
  source 1..19: mol_type = other DNA
cggtgctcacagaagccag

SEQ ID NO: 52 - DNA, 22 bp, synthetic construct
  source 1..22: mol_type = other DNA
catgaaggatgatctgtggagc

SEQ ID NO: 53 - DNA, 21 bp, synthetic construct
  source 1..21: mol_type = other DNA
aatccgcaggttactggtgag

SEQ ID NO: 54 - DNA, 22 bp, synthetic construct
  source 1..22: mol_type = other DNA
acttgcagtgagaacacgtatg

SEQ ID NO: 55 - DNA, 21 bp, synthetic construct
  source 1..21: mol_type = other DNA
tcatccgccgaaagtcatgtg

SEQ ID NO: 56 - DNA, 21 bp, synthetic construct
  source 1..21: mol_type = other DNA
ctgaggagaagtctgccgtta

SEQ ID NO: 57 - DNA, 20 bp, synthetic construct
  source 1..20: mol_type = other DNA
agcatcaggagtggacagat

SEQ ID NO: 58 - DNA, 22 bp, synthetic construct
  source 1..22: mol_type = other DNA
ggagatgttcacgacttccttc

SEQ ID NO: 59 - DNA, 22 bp, synthetic construct
  source 1..22: mol_type = other DNA
gaatggtgccagcttattctga

SEQ ID NO: 60 - DNA, 19 bp, synthetic construct
  source 1..19: mol_type = other DNA
cgggtgctgacagctatcc

SEQ ID NO: 61 - DNA, 21 bp, synthetic construct
  source 1..21: mol_type = other DNA
acacacagatgtctcacaggg

SEQ ID NO: 62 - DNA, 20 bp, synthetic construct
  source 1..20: mol_type = other DNA
gcgacatcagtggagctagg

SEQ ID NO: 63 - DNA, 22 bp, synthetic construct
  source 1..22: mol_type = other DNA
cggcacactcattggcatattg

SEQ ID NO: 64 - DNA, 23 bp, synthetic construct
  source 1..23: mol_type = other DNA
ggctacttgggctattgtaaagg

SEQ ID NO: 65 - DNA, 23 bp, synthetic construct
  source 1..23: mol_type = other DNA
cagtttctccgacaactttctct

SEQ ID NO: 66 - DNA, 20 bp, synthetic construct
  source 1..20: mol_type = other DNA
gcgagaagatgacccagatc

SEQ ID NO: 67 - DNA, 19 bp, synthetic construct
  source 1..19: mol_type = other DNA
ccagtggtacggccagagg

SEQ ID NO: 68 - DNA, 30 bp, synthetic construct
  source 1..30: mol_type = other DNA
acacacctgtagtcccagctactcaggagg

SEQ ID NO: 69 - DNA, 30 bp, synthetic construct
  source 1..30: mol_type = other DNA
cctcctgagtagctgggactacaggtgtgt

SEQ ID NO: 70 - DNA, 30 bp, synthetic construct
  source 1..30: mol_type = other DNA
cttgggctttcctcccacccgggactggtg

SEQ ID NO: 71 - DNA, 30 bp, synthetic construct
  source 1..30: mol_type = other DNA
caccagtcccgggtgggaggaaagcccaag

SEQ ID NO: 72 - DNA, 30 bp, synthetic construct
  source 1..30: mol_type = other DNA
aaggggactatctcccagcggcaggccctt

SEQ ID NO: 73 - DNA, 30 bp, synthetic construct
  source 1..30: mol_type = other DNA
aagggcctgccgctgggagatagtcccctt

SEQ ID NO: 74 - DNA, 30 bp, synthetic construct
  source 1..30: mol_type = other DNA
ccgggcgccgccccctgcgccctgaggccc

SEQ ID NO: 75 - DNA, 30 bp, synthetic construct
  source 1..30: mol_type = other DNA
gggcctcagggcgcagggggcggcgcccgg

SEQ ID NO: 76 - DNA, 30 bp, synthetic construct
  source 1..30: mol_type = other DNA
acacacctgtagttttagctactcaggagg

SEQ ID NO: 77 - DNA, 30 bp, synthetic construct
  source 1..30: mol_type = other DNA
cctcctgagtagctaaaactacaggtgtgt

SEQ ID NO: 78 - DNA, 30 bp, synthetic construct
  source 1..30: mol_type = other DNA
aaggggactatcttttagcggcaggccctt

SEQ ID NO: 79 - DNA, 30 bp, synthetic construct
  source 1..30: mol_type = other DNA
aagggcctgccgctaaaagatagtcccctt

SEQ ID NO: 80 - DNA, 30 bp, synthetic construct
  source 1..30: mol_type = other DNA
ccgggcgccgccttttgcgccctgaggccc

SEQ ID NO: 81 - DNA, 30 bp, synthetic construct
  source 1..30: mol_type = other DNA
gggcctcagggcgcaaaaggcggcgcccgg

SEQ ID NO: 82 - DNA, 37 bp, synthetic construct
  source 1..37: mol_type = other DNA
ggtttttgagggtgagggtgagggtgagggtgagggt

SEQ ID NO: 83 - DNA, 39 bp, synthetic construct
  source 1..39: mol_type = other DNA
tcccgactatccctatccctatccctatccctatcccta

SEQ ID NO: 84 - DNA, 23 bp, synthetic construct
  source 1..23: mol_type = other DNA
cagcaagtgggaaggtgtaatcc

SEQ ID NO: 85 - DNA, 25 bp, synthetic construct
  source 1..25: mol_type = other DNA
cccattctatcatcaacgggtacaa

SEQ ID NO: 86 - protein, 92 aa, Homo sapiens
MGKVNVAKLRYMSRDDFRVLTAVEMGMKNHEIVPGSLIASIASLKHGGCNKVLRELVKHKLIAWERTKTVQGYRLTNAGYDYLALKTLSSRQ

SEQ ID NO: 87 - DNA, 1047 bp, Homo sapiens
  source 1..1047: mol_type = other DNA
cacgtgtcaccttaaactcccaccccaaccagcgcaggcgcacttcgaagcctcctagaccaacctaagagaagtactgagcaagcgcatgagttccattaaccacgccctccggagctgccggagcaggcgcaattaggagtagagggcgtgcttgtgtgattacagtccactttacctttaacagatttggttgtctggactctagctcaggttcttttacccctgtcttgttggaagtcactttgcctagagtagaagttgtttgcgggaaaaatttgctcctcccattaagtaaccactttgctctctgaggcggaacgtgtcattgaggcagcaagtcctgacagatatggaaaaaaaaaggcacattgtagagaatatctacactccgccaactcagtcacaacgcaaaaattacacaaaaataaaacgttcacttgttacatactatgtgtttcataagacatacaagtagttttattttcctatatcaatgagaacagcacttctaactattaacaaggacctatactaagacgcagcgcgtgcgcgagccatctggcgctgtaaagcgcttagaggctggaataacgacctgccttacgctttgcggctgtcgtcggagaggcatctgggttcggactggggccgccatggggaaagtgaatgtggccaagttgcgttacatgagccgagatgacttcagggtcttgaccgcggtaagaaacccgccgttcctgttctcccttccccttaacactgtgtgacctcttcctgttcctgcaagttttctgggatctgagcaggcacactctgggacccgggacgtggcagaagctgggaatggagaagagagcattcagaaagtcggatttttaaaaaggttttgtttgggagcttaccttcccccagatagagcaaaggtcccgtcttctaataattgttttcttttctgctgctattcgctttgcgctgaagagaaacgcttgcatctctgtggcctggaaacttctctgatcctgagcttccgaccagctgtgaaag

SEQ ID NO: 88 - DNA, 12 bp, synthetic construct
  source 1..12: mol_type = other DNA
nvaaadrvaraa

SEQ ID NO: 89 - DNA, 11 bp, synthetic construct
  source 1..11: mol_type = other DNA
nchbcmwgvcm

SEQ ID NO: 90 - DNA, 16 bp, synthetic construct
  source 1..16: mol_type = other DNA
nacakrbnnrnrccmy

SEQ ID NO: 91 - DNA, 449 bp, Homo sapiens
  source 1..449: mol_type = other DNA
ctagccaggcctggtggcacacacctgtagtcccagctactcaggaggctgaggcaggaggatcactgccgggaggcagtggttgcagtgagccgagatcgtgccactgcactccagcctggcaacagaggagaccctgcctcaaaaaaaagtgtgtgtggatggggggggaaggatttctgtgtctgaggaccccttctgtcctcgcaggttaatccccagaggctccatggagttccctggcctggggtccctggggacctcagagcccctcccccagtttgtggatcctgctctggtgtcctccacaccagaatcaggggttttcttcccctctgggcctgagggcttggatgcagcagcttcctccactgccccgagcacagccaccgctgcagctgcggcactggcctactacagggacgctgaggcctacagacactccccag

SEQ ID NO: 92 - DNA, 437 bp, Homo sapiens
  source 1..437: mol_type = other DNA
gccttcctttcgttttgagccttgggctttcctcccacccgggactggtgctctttctcgccggatctgggctggggctccgtggcgtgcgggacacctcgtggtgggactttggggggtgtcaggcgctggcggcacgcctcactcccccttcctcgcgcagggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacg

SEQ ID NO: 93 - DNA, 822 bp, Homo sapiens
  source 1..822: mol_type = other DNA
aggcagtgccttggcgaagctgtccgcggtccctgcggcgcagccggagcgcacgggcccaagaagaagtggggttggaccccgccccgcgggccccgggggtactaaccccgcgcgggcggccgcggccccgccacttgattctggaggatttgttctggggctgcggccgcggagtcggggcggccgcgggcgagcttcggggcgggaggcggcggcagcggcacagccccgcgcgggccccgccgcggcccaggcagccgggacagccacgaggggcggccgcacgcggggccgcgcgccgaggatgcgggactagccgggcaggctgcgggcggccgtcgggccagcgaggcctcgcagcgggcgggccctggcgagtagtggccgggcgccgccccctgcgccctgaggcccgggccccgccgcttctgctttcccgcttctcgcggcagcggcggccgaggaggcgcccgcgccggccgcccccgggggaagccgcgccgtctccgcctgcccggcgccctgacggccgctgttatgcgtattcccgtagacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaag

SEQ ID NO: 94 - DNA, 336 bp, Homo sapiens
  source 1..336: mol_type = other DNA
agagacttcctgtatgtagcgcaagagatttatgcaaacgggttggggcggtgatgtcaccccaaggggactatctcccagcggcaggcccttcgataaaatcaggaacttgtgctggccctgcaatgtcaagggagggggctcacccagggctcctgtagctcagggggcaggcctgagccctgcacccgccccacgaccgtccagcccctgacggggcaccccatcctgaggggctctgcattggcccccaccgaggcaggggatctgaccgactcggagcccggctggatgttacaggcgtgcaaaatggaagggtttcccctcgtcccccct

SEQ ID NO: 95 - DNA, 321 bp, Homo sapiens
  source 1..321: mol_type = other DNA
ctgctgcagggctgagaccctgggaggtcccaacccaggcaaattgaacgccaggctaatttgaagacccaactcccagccctccccttcaccggaggacagagctctagctggcctgggcccccacctgatagcagcctccaacgtctggggtgtctgataatcacagcctcagagttcacgaggcagccgaggaagaggaggcttgaggcccagggtgggcaccagccagccatggccacagccgagaccgccttgccctccatcagcacactgaccgccctgggccccttcccggacacacaggatgacttcctcaag

SEQ ID NO: 96 - DNA, 297 bp, Homo sapiens
  source 1..297: mol_type = other DNA
caattttaaaatttctggctgggcacagtggctcatgcctgtaatcccagcactttgggagtctgaggtgggcggatcgcttgaggtcaggagtttgagaccagcctgggcaacatggtgccgctgcggcccaaggcttgccgtttgacgaggagcagtcgcggtaggcggtgggcaaggctgccctgggcggaggccgaggcgcggctcggactccagcatggcgaccgcggtgcgcgctgtgggctgcctccccgtgctgtgtagcgggacggcaggtgagagctgggacgccgg

SEQ ID NO: 97 - DNA, 230 bp, Homo sapiens
  source 1..230: mol_type = other DNA
gcccaggaccccgggccctgacccctcgcgtgccccgcccccgcagggaggtcccagccgcctggggacagcgctgtggctcctccgggcctgtaggcggcactgcggccccgcgcggatggcgacggaaggggaggccgctcggcctggcctggagggagacctcgctctgccccgcgtcccagccatggcgacatcctcggccgcgctgccccgaatactcggcgcgg

SEQ ID NO: 98 - DNA, 293 bp, Homo sapiens
  source 1..293: mol_type = other DNA
tgcagtcccagctactcgggaggctgaggcaggagaatggcgtgaacccaggaggcggacactccagaactgcccacttatgggcgggggtcactccttcaggcttaacagtcattggctgaattgggccagagaggtctcattggctgaattcctgcaccggctcgtcggaggcggcgaggctccagtggccttgacctcccgcggcgtgggaggctgcgcggcgatgctgcagttcgtccgggccggggcgcgggcctggcttcggcctaccggcagccaggtgaggccag

SEQ ID NO: 99 - DNA, 299 bp, Homo sapiens
  source 1..299: mol_type = other DNA
ggacaggcgcgtcccagcccacgcagccggaacgcctgagcgctgggcgcctggtctgccggacctcagcagggggcaccgcgggccggactgtggaggggcgcacgcccggaagcggcgagggtagccatgacggcctccgtgctgcgaagtatctcgctagccctgcgcccgactagcgggtgagtgcggacgctggccggatagcggagtgcccagggcgcgtgcggggtcttcgcgactgtccgccaggactcgcctgtgctccctgctcgcctcccagtccacgaatatctcag

SEQ ID NO: 100 - DNA, 415 bp, Mus musculus
  source 1..415: mol_type = other DNA
agccaatgggattgagtagacagtccgcgggttcgtagagattcctgcccccgtaccccgtcctgtcctcctcctcctcctcctcttctctacgtcccttttttatatcagcaagctaggggcggtgcaacggtgggcgggtcctaatgacgtgcccggacgggtctcacaccataggagtctacagttcttagtttgtggcggcttgggcgggacgcacgcggtgcagttcaagatggcggatgcagaaggtaagaggttctggtggcagtgccatgagacctgtggggcctcctacacgacccggagctagttatagtgagattgcatcgtgacccgcggttccccgtgttcccgctcgcagtttggctgtctcaccactcggctctggctctacctgtgaatgcacgcattc

SEQ ID NO: 101 - protein, 11 aa, synthetic construct
CNKVLRELVKH

SEQ ID NO: 102 - protein, 11 aa, synthetic construct
  SITE 1: note = S5-pentenyl alanine
  CROSSLNK 1..5: note = Intramolecular staple (Ala-Ala)
  SITE 5: note = S5-pentenyl alanine
ANKVARELVKH

SEQ ID NO: 103 - protein, 11 aa, synthetic construct
  SITE 5: note = S5-pentenyl alanine
  CROSSLNK 5..9: note = Intramolecular staple (Ala-Ala)
  SITE 9: note = S5-pentenyl alanine
SNKVARELAKH

SEQ ID NO: 104 - protein, 11 aa, synthetic construct
  SITE 1: note = R5-octenyl alanine
  CROSSLNK 1..8: note = Intramolecular staple (Ala-Ala)
  SITE 8: note = S5-pentenyl alanine
ANKVLREAVKH

SEQ ID NO: 105 - protein, 11 aa, synthetic construct
  SITE 2: note = R5-octenyl alanine
  CROSSLNK 2..9: note = Intramolecular staple (Ala-Ala)
  SITE 9: note = S5-pentenyl alanine
SAKVLRELAKH

SEQ ID NO: 106 - protein, 11 aa, synthetic construct
  SITE 4: note = R5-octenyl alanine
  CROSSLNK 4..11: note = Intramolecular staple (Ala-Ala)
  SITE 11: note = S5-pentenyl alanine
SNKALRELVKA

SEQ ID NO: 107 - protein, 9 aa, synthetic construct
  SITE 1: note = S5-pentenyl alanine
  CROSSLNK 1..5: note = Intramolecular staple (Ala-Ala)
  SITE 5: note = S5-pentenyl alanine
ASLIASIAS

SEQ ID NO: 108 - protein, 9 aa, synthetic construct
  SITE 3: note = S5-pentenyl alanine
  CROSSLNK 3..7: note = Intramolecular staple (Ala-Ala)
  SITE 7: note = S5-pentenyl alanine
GSAIASAAS

SEQ ID NO: 109 - protein, 9 aa, synthetic construct
  SITE 4: note = S5-pentenyl alanine
  CROSSLNK 4..8: note = Intramolecular staple (Ala-Ala)
  SITE 8: note = S5-pentenyl alanine
GSLAASIAS

SEQ ID NO: 110 - protein, 10 aa, synthetic construct
  SITE 1: note = R5-octenyl alanine
  CROSSLNK 1..9: note = Intramolecular staple (Ala-Ala)
  SITE 9: note = S5-pentenyl alanine
AGSLIASIAS

(19) United States
(12) Patent Application Publication
(10) Pub. No.: US 2025/0381241 A1
(43) Pub. Date: Dec. 18, 2025

(54) METHODS AND COMPOSITIONS TO MODULATE RIOK2

(71) Applicant: Dana-Farber Cancer Institute, Inc., Boston, MA (US)

(72) Inventors: Laurie H. Glimcher, Boston, MA (US); Shrestha Ghosh, Boston, MA (US)

(21) Appl. No.: 18/711,385

(22) PCT Filed: Nov. 29, 2022
(86) PCT No.: PCT/US22/51254

§ 371 Date: May 17, 2024

Related U.S. Application Data
(60) Provisional application No. 63/283,839, filed on Nov. 29, 2021.
(60) Provisional application No. 63/331,388, filed on Apr. 15, 2022.

(51) Int. Cl. A61K 38/08; A61K 38/18; A61K 38/20; C07K 7/06

(52) U.S. Cl. CPC A61K 38/08; A61K 38/1816; A61K 38/206; C07K 7/06

(57) ABSTRACT

The present invention provides methods of treating a disease or disorder in a subject comprising administering to the subject a therapeutically effective amount of an agent that modulates RIOK2.

### CROSS-REFERENCE TO RELATED APPLICATIONS

This application is a § 371 national-stage application based on PCT/US2022/051254, filed Nov. 29, 2022, which claims the benefit of priority to U.S. Provisional Application Ser. No. 63/283,839, filed on 29 Nov. 2021, and U.S. Provisional Application Ser. No. 63/331,388, filed on 15 Apr. 2022; the entire contents of each of said applications are incorporated herein in their entirety by this reference.

### REFERENCE TO A SEQUENCE LISTINM XML

This application contains a Sequence Listing which has been submitted electronically in XML format. The Sequence Listing XML is incorporated herein by reference. Said XML file, created on Mar. 3, 2023, is named DFS-32325_SL.xml and is 161,621 bytes in size.

### BACKGROUND OF THE INVENTION

Telomeres form the ends of chromosomes. Telomeres shorten with each round of cell division and this mechanism limits proliferation of cells containing chromosomal DNA to a finite number of cell divisions by inducing replicative senescence or apoptosis. There is growing evidence indicating that telomere shortening also limits other biological processes, such as stem cell function, regeneration, and organ maintenance during ageing. Telomere shortening during ageing and associated disease is also involved in increased cancer risk (Jiang, H. et al. Telomere shortening and ageing. Z Gerontol Geriatr 5, 314-24 (2007)). In addition, telomere shortening is also emerging as a characteristic feature of Idiopathic pulmonary fibrosis (IPF) (Stuart et al. Effect of telomere length on survival in patients with idiopathic pulmonary fibrosis: an observational cohort study with independent validation. Lancet Respir Med. 2: 557-65 (2014)).

Anemia is a hallmark of a plethora of hematologic disorders associated with aging, chronic diseases such as renal failure and inflammation, bone marrow failure and myeloid neoplasms (Palapar, L. et al. Anaemia and physical and mental health in the very old: An individual participant data meta-analysis of four longitudinal studies of ageing. Age Ageing 50, 113-119 (2021); Lopes, M. B. et al. A real-world longitudinal study of anemia management in non-dialysis-dependent chronic kidney disease patients: a multinational analysis of Coups. Sic Rep 11, 1784 (2021); Becktell, K. et al. Aplastic Anemia & MDS International Foundation (AA & MDSIF): Bone Marrow Failure Disease Scientific -1I- Symposium 2018. Leuk Res 80, 19-25 (2019)). Aberrant red blood cell differentiation (erythropoiesis) underlies anemias and can be accompanied by myeloid proliferation. For example, myelodysplastic syndromes (MDS), a heterogeneous group of clonal hematologic disorders, are classically characterized by anemia and myeloproliferation (Saygin, C. & Carraway, H. E. Current and emerging strategies for management of myelodysplastic syndromes. Blood Rev, 100791 (2020)). The average survival time following diagnosis of MDS is 3 years owing to few treatment options and roughly 20-30% of MDS patients progress to acute myeloid leukemia (Hong, S. et al. Survival following relapse after allogeneic hematopoietic cell transplantation for acute leukemia and myelodysplastic syndromes in the contemporary era. Hematol Oncol Stem Cell Ther S1658-3876(20) 30178-3 (2020); Garcia-Manero, G., Chien, K. S. & Montalban-Bravo, G. Myelodysplastic syndromes: 2021 update on diagnosis, risk stratification and management. Am J Hematol 95, 1399-1420 (2020)). Hence, the substantial risks of allogeneic bone marrow transplants in elderly patients, together with a dearth of effective FDA-approved drugs make it imperative to revisit the origins of blood cell differentiation (hematopoiesis) defects underlying anemia and myeloproliferation to identify new druggable targets (Feld, J., Navada, S. C. & Silverman, L. R. Myelo-deception: Luspatercept & TGF-Beta ligand traps in myeloid diseases & anemia. Leuk Res 97, 106430 (2020); Bhatt, V. R. & Steensma, D. P. Hematopoietic Cell Transplantation for Myelodysplastic Syndromes. J Oncol Pract 12, 786-792 (2016)).

Metabolic disorders refer to a group of disorders including, but not limited to, metabolic syndrome, obesity, and diabetes. Blood glucose levels rise after food intake, stimulating insulin secretion which in turn stimulates cells in peripheral tissues to uptake glucose from the blood. Loss of glucose homeostasis as a result of dysregulated insulin secretion and action can result in metabolic disorders such as diabetes, which are either induced simultaneously by obesity or worsened by obesity (Wilcox, Insulin and Insulin Resistance. Clin Biochem Rev. 2: 19-39 (2005)).

Polycythemia vera (PV) is the most common myeloproliferative neoplasm (MPN). It is also the MPN with the highest incidence of thromboembolic complications, which usually occur early in the course of the disease, and the only MPN in which erythrocytosis occurs. The classical presentation of PV is characterized by erythrocytosis, leukocytosis, and thrombocytosis, often with splenomegaly and occasionally with myelofibrosis, but it can also present as isolated erythrocytosis with or without splenomegaly, isolated thrombocytosis or isolated leukocytosis, or any combination of these (Spivak, Polycythemia Vera. Curr Treat Options Oncol. 2: 12 (2018)).

All diseases and disorders disclosed herein would benefit from the development of agents that treat such disorders.

### SUMMARY OF THE INVENTION

RIOK2 (right open reading frame kinase 2) is an atypical serine threonine kinase that plays important roles in the final maturation steps of the pre-40S ribosomal complex to facilitate cytoplasmic translation (Ferreira-Cerca, S. et al. ATPase-dependent role of the atypical kinase Rio2 on the evolving pre-40S ribosomal subunit. Nat Struct Mol Biol 19, 1316-1323 (2012); Zemp, I. et al. Distinct cytoplasmic maturation steps of 40S ribosomal subunit precursors require hRio2. J Cell Biol 185, 1167-1180 (2009); Ameismeier, M., Cheng, J., Berninghausen, O. & Beckmann, R. Visualizing late states of human 40S ribosomal subunit maturation. Nature 558, 249-253 (2018)).

Hematopoietic cell-specific heterozygous deletion of Riok2 leads to anemia and myeloproliferation in mice (Raundhal, M. et al. Blockade of IL-22 signaling reverses erythroid dysfunction in stress-induced anemias. Nat Immunol 22(4):520-529 (2021)), reminiscent of Myelodysplastic Syndrome (MDS)-associated phenotypes in patients (Pellagatti, A. & Boultwood, J. The molecular pathogenesis of the myelodysplastic syndromes. Eur JHaematol 95, 3-15 (2015); Garcia-Manero, G., Chien, K. S. & Montalban-Bravo, G. Myelodysplastic syndromes: 2021 update on diagnosis, risk stratification and management. Am J Hematol 95, 1399-1420 (2020)). Reduced RIOK2 mRNA levels in patients with the del(5q) subtype of MDS as compared to healthy individuals was also observed (Raundhal, M. et al. Blockade of IL-22 signaling reverses erythroid dysfunction in stress-induced anemias. Nat Immunol 22(4):520-529 (2021); Pellagatti, A. et al. Deregulated gene expression pathways in myelodysplastic syndrome hematopoietic stem cells. Leukemia 24, 756-764 (2010)).

Therefore, the present invention is based, in part, on the discovery that RIOK2 is a master transcriptional regulator of hematopoietic lineage commitment and that its ablation drives primary human stem and progenitor cells (HSPCs) towards MDS-associated hematopoietic differentiation defects. The transcriptomic profiling, structural modeling, ChIP sequencing, ATAC-sequencing and structure-function domain deletion mutants shown herein revealed that RIOK2 regulates specific genetic programs in hematopoiesis via its previously unappreciated winged helix-turn-helix DNA-binding domain and two transactivation domains. Mechanistically, RIOK2 transcriptionally modifies the expression of key lineage-specific transcription factors, such as GATA1, GATA2, SPI1, RUNX3 and KLF1 to fine-tune lineage fate determination in primary human hematopoietic stem cells. It is further demonstrated that GATA1 and RIOK2 function in a positive feedback loop to drive erythroid differentiation. These discoveries thus present therapeutic opportunities to correct hematopoietic differentiation defects in a range of hematologic disorders, such as anemia.

Therefore, provided herein are methods of treating a red blood cell disorder (e.g., anemia, such as anemia associated with MDS or acute myeloid leukemia (AML)) by stabilizing and/or increasing the copy number, expression, and or activity of RIOK2 in a subject. Also provided herein are methods of assessing the efficacy of an agent that stabilizes and/or increases the copy number, amount, and/or activity of RIOK2 for treating a red blood cell disorder (e.g., anemia, such as anemia associated with MDS or acute myeloid leukemia (AML)) in a subject, comprising: a) detecting in a sample at a first point in time the copy number, amount, and/or or activity of RIOK2; b) repeating step a) during at least one subsequent point in time after contacting the sample with the agent; and c) comparing the copy number, amount, and/or activity detected in steps a) and b), wherein a lack of change of, the slowing of a decrease in, or a significant increase in, the copy number, amount, and/or activity of, the RIOK2, in the subsequent sample as compared to the copy number, amount, and/or activity in the sample at the first point in time, indicates that the agent effectively treats the red blood cell disorder (e.g., anemia, such as anemia associated with MDS or AML).

Conversely, also provided herein are methods of treating polycythemia vera by decreasing the copy number, expression, and or activity of RIOK2 in a subject. Additionally, methods of assessing the efficacy of an agent that inhibits the copy number, amount, and/or activity of RIOK2 for treating a polycythemia vera, in a subject, comprising: a) detecting in a sample at a first point in time the copy number, amount, and/or or activity of RIOK2; b) repeating step a) during at least one subsequent point in time after contacting the sample with the agent; and c) comparing the copy number, amount, and/or activity detected in steps a) and b), wherein a significant decrease in, the copy number, amount, and/or activity of the RIOK2, in the subsequent sample as compared to the copy number, amount, and/or activity in the sample at the first point in time, indicates that the agent effectively treats the polycythemia vera.

The present invention is also based, in part, on the discovery that loss of RIOK2 leads to telomere shortening. CRISPR/Cas9-mediated knockdown or knockout of RIOK2 decreases telomere length in primary human cells dose-dependently, thus signifying that RIOK2 is critical for telomere maintenance. Telomeres play a critical role in aging, and therefore, provided herein are compositions and methods for stabilizing and/or increasing the copy number, expression, and or activity of RIOK2 in subjects afflicted by aging or disorders associated with telomere shortening such as dyskeratosis congenita (DC), aplastic anemia, a myelodysplastic syndrome, or idiopathic pulmonary fibrosis (IPF). Also provided herein are assays (e.g., cell-based assays or cell-free assays) for screening for agents that slow aging and/or telomere shortening, by first contacting a cell with a test agent selected from the group consisting of 1) a nucleic acid encoding a RIOK2 peptide, or biologically active fragment thereof, 2) a RIOK2 polypeptide, or biologically active fragment thereof, and/or 3) an internally cross-linked peptide that specifically binds to an amino acid sequence in a corepressor that binds to a transrepressor domain (TRD) of RIOK2, and determining telomere length within the cell relative to a control, thereby identifying the test agent to slow aging and/or telomere shortening. In some embodiments, the control is a cell not contacted with the test agent. In other embodiments, the control is a cell contacted with an anti-aging agent and/or a telomere-stabilizing agent. The cell may be isolated from an animal model of aging or a human patient afflicted with a disorder associated with telomere-shortening. In some embodiments, the step of contacting occurs in vivo, ex vivo, or in vitro. Determining telomere length within the cell may comprise any amplification reaction, such as PCR.

The inventors have also shown that TRiC and Dyskerin complex subunits are critical in maintaining telomerase activity and telomere length. RIOK2 transcriptionally regulates TRiC and Dyskerin complex subunit expression via its transcription factor (TF) activity.

Therefore, also provided herein are methods of assessing the efficacy of an agent that stabilizes and/or increases the copy number, amount, and/or activity of RIOK2 for slowing aging and/or telomere shortening comprising a) detecting in a sample at a first point in time the copy number, amount, and/or or activity of RIOK2, TRiC and/or a Dyskerin complex subunit, such as DKC1, NHP2, NOP10 and GAR1; b) repeating step a) during at least one subsequent point in time after contacting the sample with the agent; and comparing the copy number, amount, and/or activity detected in steps a) and b), wherein a lack of change of, the slowing of a decrease in, or a significant increase in, the copy number, amount, and/or activity of, the RIOK2, TRiC and/or a Dyskerin complex subunit, such as DKC1, NHP2, NOP10 and GAR1, in the subsequent sample as compared to the copy number, amount, and/or activity in the sample at the first point in time, indicates that the agent effectively treats aging and/or telomere shortening.

Provided herein are methods and compositions for slowing aging and/or telomere shortening in a subject in need thereof, the method comprising administering to the subject an effective amount of at least one agent that increases and/or stabilizes the copy number, the expression level, and/or the activity of RIOK2. In some embodiments, the subject is afflicted with a disease or disorder associated with telomere shortening, such as dyskeratosis congenita (DC), aplastic anemia, myelodysplastic syndrome, or idiopathic pulmonary fibrosis (IPF).

Therefore, also provided herein are methods and compositions for treating dyskeratosis congenita (DC), aplastic anemia, myelodysplastic syndrome, or idiopathic pulmonary fibrosis (IPF).

As used herein, “aging” may refer to an individual, such as an individual that is 60 years of age or older. Additionally, “aging” can refer to cellular aging and includes, but is not limited to, any cellular or physiological process in which a subject's telomeres shorten over time but does not include general deterioration in physiological, psychological and other biological characteristics. Therefore, aging may also refer to cells exhibiting senescence, apoptosis, and/or cell cycle arrest at S or G2/M phase in cells when compared to cells from an individual that is less than 60 years old and/or compared to a subject lacking a pathology associated with aging and/or telomere shortening. Additionally, it will be appreciated that any “disease or disorder disclosed herein” includes aging, any disease or disorder associated with aging and/or telomere shortening. In some embodiments, a subject that is afflicted by aging or is aged includes any subject that is at least about 60 years old, such as at least 65 years old, at least 70 years old, at least 75 years old, at least 80 years old, or at least 90 years old, or any range in between, inclusive, such as 60-75 years old.

In addition, RIOK2 levels can be low in subjects with metabolic disorders associated with mitochondrial defects (e.g., mitochondriopathies). Therefore, also provided herein are compositions and methods for preventing or treating a metabolic syndrome and/or disorders associated with mitochondrial defects in a subject by stabilizing and/or increasing the copy number, expression, and or activity of RIOK2 in the subject. Methods provided herein include methods of assessing the efficacy of an agent that stabilizes and/or increases the copy number, amount, and/or activity of RIOK2 for treating metabolic disorders (e.g., any metabolic disorder disclosed herein) comprising a) detecting in a sample at a first point in time the copy number, amount, and/or or activity of RIOK2; b) repeating step a) during at least one subsequent point in time after contacting the sample with the agent; and comparing the copy number, amount, and/or activity detected in steps a) and b), wherein a lack of change of, the slowing of a decrease in, or a significant increase in, the copy number, amount, and/or activity of, the RIOK2, in the subsequent sample as compared to the copy number, amount, and/or activity in the sample at the first point in time, indicates that the agent effectively treats the metabolic disorder. Metabolic disorders, include, but are not limited to, diabetes, obesity, pre-diabetes, mitochondriopathies, metabolic syndrome or metabolic disorders (e.g., metabolic disorders associated with mitochondrial defects). The metabolic disorder may be any metabolic disorder associated with low levels of RIOK2 (e.g., low levels of RIOK2 compared to a subject not afflicted with the metabolic disorder).

In some aspects, provided herein are methods and compositions for treating one or more red blood cell disorders in a subject (e.g., any red blood cell disorder disclosed herein), the method comprising administering to the subject an effective amount of at least one agent that increases and/or stabilizes the copy number, the expression level, and/or the activity of RIOK2. The red blood cell disorder may be anemia, optionally wherein the anemia is selected from the group consisting of macrocytic anemia, anemia associated with chronic kidney disease (CKD), anemia caused by insufficiency of serine/threonine-protein kinase RIOK2, anemia caused by one or more mutations and/or deletions in human chromosome 5 or in an ortholog thereof, stress-induced anemia, aplastic anemia, Diamond Blackfan anemia, and Schwachman-Diamond syndrome. The anemia may be an anemia associated with a cancer, optionally wherein the cancer is a hematologic malignancy (e.g., myelodysplastic syndromes (MDS) or acute myeloid leukemia (AML)). In some embodiments, the red blood cell disorder is associated with increased megakaryopoiesis and/or myelopoiesis. The anemia may be associated with treatment of cancer by chemotherapy or radiation. The anemia may be associated with chronic kidney disease or with inflammatory diseases such as rheumatoid arthritis or systemic lupus erythematosus (SLE). The methods provided herein may also include administering to the subject an effective amount of an erythropoiesis-stimulating agent (e.g., erythropoietin, epoetin alfa, epoetin beta, epoetin omega, epoetin zeta, IL-9, or darbepoetin alfa). The anemia may be an anemia associated with a bone marrow failure syndrome, such as aplastic anemia, Diamond Blackfan anemia, amegakaryocytic thrombocytopenia (Amega), dyskeratosis congenita (DC), fanconi anemia (FA), Pearson syndrome, severe congenital neutropenia (SCN), Shwachman-Diamond syndrome (SDS), thrombocytopenia absent radii (TAR) and others.

In some aspects, provided herein are methods of promoting differentiation of an erythroid progenitor cell toward a mature red blood cell in a subject, comprising administering an effective amount of at least one agent that increases the copy number, the expression level, and/or the activity of RIOK2.

Loss of RIOK2 results in myelodysplastic syndrome-associated phenotypes. Moreover, 20-30% MDS patients progress to AML. Without being bound by theory, it is believed that enhancing the expression and/or activity of RIOK2 improves MDS and/or AML, as well as reverses anemia associated with these hematologic malignancies.

Therefore, also provided herein are methods of preventing or treating a myelodysplastic syndrome in a subject, the method comprising administering to the subject an effective amount of at least one agent that increases the copy number, the expression level, and/or the activity of RIOK2.

In some embodiments, the subject has a mutation that is associated with a decreased copy number, expression level, and/or the activity of RIOK2. The subject may have a loss of function mutation in a RIOK2 gene. The subject may express any one of the RIOK2 variants listed in Table 3.

In some embodiments, at least one agent (e.g., the agent that increases and/or stabilizes the copy number, the expression level, and/or the activity of RIOK2) comprises an internally cross-linked peptide, small molecule, a peptide, a polypeptide, an aptamer, an antibody or a binding fragment thereof, an intrabody or a binding fragment thereof, and/or a nucleic acid. The internally cross-linked peptide may specifically bind to an amino acid sequence in a corepressor that binds to a transrepressor domain (TRD) of RIOK2, activate RIOK2, and/or stabilize the expression of or activity of RIOK2.

The internally cross-linked peptide may comprise any one of the following:
i) an amino acid sequence of XSLIXSIAS (SEQ ID NO: 1), wherein X is a non-natural amino acid and X1 and X5 are joined by an internal staple;
ii) an amino acid sequence of GSXIASXAS (SEQ ID NO: 2), wherein X is a non-natural amino acid, and X3 and X7 are joined by an internal staple;
iii) an amino acid sequence of GSLXASIXS (SEQ ID NO: 3), wherein X is a non-natural amino acid, and X4 and X8 are joined by an internal staple; or
iv) an amino acid sequence of 8GSLIASIXS (SEQ ID NO: 4), wherein X and 8 are non-natural amino acids, and 8 and X8 are joined by an internal staple.

In some embodiments, 8 is R5-octenyl alanine. In some embodiments, X is S5-pentenyl alanine.

In some embodiments, the stapled peptides bind to the corepressors of RIOK2 that bound to its TRD domain and hence activate RIOK2's activity and/or expression. In some embodiments, the stapled peptides do not bind to RIOK2, such as do not bind a domain of RIOK2.

In some embodiments, the at least one agent comprises a peptide having an amino acid sequence of GSLIASIAS (SEQ ID NO: 5) with at least one, at least two, at least three, at least four, at least five, at least six, or at least seven substitutions, additions, and/or deletions, as stated above. In some embodiments, the at least one agent comprises a peptide having an amino acid sequence of GSLIASIAS (SEQ ID NO: 5) with at most one, at most two, at most three, at most four, at most five, at most six, or at most seven, substitutions, additions, or deletions, as stated above. The at least one agent may comprise a nucleic acid (e.g., nucleic acid is operably linked to a promoter or a viral particle, such as a lentivirus particle, an adenovirus particle, or an adeno-associated virus particle). In some embodiments, the at least one agent comprises a cell-based agent. The cell based agent may comprise a cell that is modified to comprise an increased copy number, expression level, and/or activity of RIOK2 or a fragment thereof. The cell may be non-replicative. The cell may be autologous or allogeneic.

In some aspects, provided herein are compositions and methods for treating polycythemia vera, the method comprising administering to the subject an effective amount of at least one agent that decreases the copy number, the expression level, and/or the activity of RIOK2. The agent may comprise an inhibitory internally cross-linked peptide, small molecule, a peptide, a polypeptide, an aptamer, an antibody or a binding fragment thereof, an intrabody or a binding fragment thereof, and/or a nucleic acid. The at least one agent may be an inhibitory internally cross-linked peptide (e.g., an inhibitory internally cross-linked peptide that specifically binds to an amino acid sequence in a corepressor that binds to a transrepressor domain (TRD) of RIOK2; and/or an inhibitory internally cross-linked peptide that blocks RIOK2 binding to DNA, and/or decreases the copy number, expression, or activity of RIOK2).

The inhibitory internally cross-linked peptide may comprise:
i) an amino acid sequence of XNKVXRELVKH (SEQ ID NO: 6), wherein X is a non-natural amino acid and X1 and X5 are joined by an internal staple;
ii) an amino acid sequence of SNKVXRELXKH (SEQ ID NO: 7), wherein X is a non-natural amino acid, and X5 and X9 are joined by an internal staple;
iii) an amino acid sequence of 8NKVLREXVKH (SEQ ID NO: 8), wherein X and 8 are non-natural amino acids, and 8 and X8 are joined by an internal staple;
iv) an amino acid sequence of S8KVLRELXKH (SEQ ID NO: 9), wherein X and 8 are non-natural amino acids, and 8 and X9 are joined by an internal staple, and/or
v) an amino acid sequence of SNK8LRELVKX(SEQ ID NO: 10), wherein X and 8 are non-natural amino acids, and 8 and X1I are joined by an internal staple.

In some embodiments, 8 is R5-octenyl alanine and X is S5-pentenyl alanine.

In some embodiments, the at least one agent comprises a peptide having an amino acid sequence of SNKVLRELVKH (SEQ ID NO: 11) with at least one, at least two, at least three, at least four, at least five, or at least six, or at least seven substitutions, additions, or deletions, as stated above. In some embodiments, the at least one agent comprises a peptide having an amino acid sequence of SNKVLRELVKH (SEQ ID NO: 11) with at most one, at most two, at most three, at most four, at most five, at most six, or at most seven substitutions, additions, or deletions, as stated above. The at least one agent comprises an anti-RIOK2 antibody or antigen-binding fragment thereof. The agent may comprise a RIOK2 binding protein or a fragment thereof. The agent may comprise a cell-based agent, such as a cell based agent that comprises a cell that is modified to comprise a decreased copy number, expression level, and/or activity of RIOK2 or a fragment thereof. The cell may be autologous or allogeneic.

### BRIEF DESCRIPTION OF THE DRAWINGS

The patent of application file contains at least one drawing executed in color. Copies of this patent or patent application publication with color drawings will be provided by the office upon request and payment of the necessary fee.

FIG. 1A-FIG. 1L show that RIOK2 drives erythropoiesis and suppresses megakaryopoiesis and myelopoiesis. FIG. 1A shows a representative immunoblot showing RIOK2 knockdown in TF-1 cells; sh #1 and 2: shRIOK2 #1 and #2. FIG. 1B shows the ratio of erythroid (CD235a+) to non-erythroid (CD235a-) TF-1 cells after differentiation, scr: scrambled. FIG. 1C shows a western blot showing shRNA-mediated RIOK2 knockdown in K562 cells. FIG. 1D shows the ratio of erythroid (CD235a+) to non-erythroid (CD235a-) K562 cells after differentiation. FIG. 1E shows an immunoblot showing RIOK2 expression after KD and KO of RIOK2 in HSPCs; Ctrl: control. FIG. 1F shows flow plots depicting erythropoiesis (CD235a), megakaryopoiesis (CD41/61) and myelopoiesis (CD11b) in differentiating HSPCs after KD and KO of RIOK2. FIG. 1G shows the quantification of data presented in FIG. 1F. FIG. 1H shows cell pellets of RIOK2-sufficient and deficient HSPCs post 14 days of erythroid differentiation. FIG. 1I shows selective differentiation of Ctrl vs RIOK2 KD and KO HSPCs towards myeloid and megakaryocytic lineages. FIG. 1J and FIG. 1K shows BFU-E, CFU-E, CFU-GM and CFU-Mk colonies formed in Ctrl vs RIOK2 KD and KO HSPCs. FIG. 1L shows volcano plots showing differentially expressed proteins in control vs RIOK2-depleted HSPCs. n=4 independent donors in g, i-k, n=3 technical replicates in FIGS. 1B and 1D, * p<0.05, ** p<0.01, *** p<0.001, **** p<0.0001, one-way ANOVA with Tukey's or Dunnett's correction. Data represented as mean±SEM. FIG. 1A, 1C, 1E representative of 3 independent experiments.

FIG. 2A-FIG. 2K show that RIOK2 and GATA1 function in a positive feedback loop to regulate hematopoietic differentiation. FIG. 2A shows a graph showing relative binding of GATA1 to RIOK2 promoter using 2 different monoclonal antibodies against GATA1, negative control: anti-rabbit Immunoglobulins (IgG) and antibodies against RNA polymerase II. FIG. 2B shows an agarose gel picture of bands quantified in 2A. FIG. 2C shows a diagram showing incorporation of wild-type (WT: GATA) or mutant (CACA) RIOK2 promoter in basic pGL3.1 luciferase reporter plasmid. FIG. 2D shows quantification of wild-type (WT: GATA) and mutant (CACA) RIOK2-promoter driven luciferase activity in response to dose dependently increasing GATA1 expression in HEK293 cells. FIG. 2E shows RT-qPCR assessment of GATA1 and RIOK2 mRNA expression (normalized to actin) upon knockdown of GATA1 using 2 different crRNAs. FIG. 2F shows RIOK2 mRNA levels post overexpression of GATA1 in RIOK2 knockdown (KD) cells. FIG. 2G shows FACS plots showing erythroid differentiation (CD235a expression) in RIOK2 KD cells expressing either empty vector (EV) or ectopic GATA1. FIG. 2H shows quantification of data presented in FIG. 2G. FIG. 21 shows quantification of erythroid (CD235a), myeloid (CD1ib), and megakaryocytic (CD41/61) progression on day 5 of differentiating HSPCs after knockdown (KD) of GATA1 and RIOK2, respectively. FIG. 2J shows FACS plot depicting GATA1 protein expression in HSPCs upon knockdown (KD) or knockout (KO) of RIOK2. FIG. 2K shows quantification of data presented in j. n=3 independent experiments in a, e, f, h; n=4 technical replicates in d; n=4 and 3 independent donors in 21 and 2K respectively. * p<0.05, ** p<0.01, *** p<0.001, **** p<0.0001, ns: not significant, one-way ANOVA with Tukey's or Dunnett's correction. Data represented as mean±SEM. FIG. 2B representative of 3 independent experiments.

FIG. 3A-FIG. 3K show that RIOK2 regulates the expression of key transcription factors in hematopoiesis. FIG. 3A shows a heat map of differentially expressed genes (cut off: adj P value <0.05) from bulk RNA sequencing of primary human HSPCs with knockdown (KD) and knockout (KO) of RIOK2, n=3 donors. FIG. 3B shows principal component analysis (PCA) of data presented in a, lines joining data points indicate individual donors. FIG. 3C-3D shows gene set enrichment analysis (GSEA) plots of early erythropoiesis, megakaryopoiesis & myelopoiesis-associated genes in RIOK2-depleted vs control HSPCs, respectively. FIG. 3E shows GATA1, GATA2, SPIl, RUNX3 and KLF1 mRNA expression (normalized to actin) in HSPCs upon KD and KO of RIOK2, n=6 independent donors. FIG. 3F shows GATA1 mRNA level (normalized to actin) in GATA1 KD cells ectopically expressing empty vector (EV) or RIOK2; SCR: scrambled. FIG. 3G shows erythroid progression in GATA1 KD cells with ectopic expression of EV or RIOK2.

FIG. 3H shows SPIl mRNA level (normalized to actin) in control (Ctrl) vs RIOK2 KD cells after inhibition of SPIl with 2 different crRNAs. FIG. 31 shows erythroid progression in scrambled (SCR) vs RIOK2 KD cells with or without knockdown of SPIl using 2 different crRNAs. FIG. 3J shows RUNX3 mRNA level (normalized to actin) in control vs RIOK2 KD cells after knocking down RUNX3 with 2 different crRNAs. FIG. 3K shows erythroid progression in scrambled (SCR) vs RIOK2 KD cells with or without knockdown of RUNX3 using 2 different crRNAs. n=4 technical replicates in f-k. * p<0.05, ** p<0.01, *** p<0.001, **** p<0.0001, one-way ANOVA with Tukey's or Dunnett's correction. Data represented as mean±SEM.

FIG. 4A-FIG. 4G show that RIOK2 binds to the promoter regions of its targets and regulates chromatin accessibility. FIG. 4A shows a graph depicting ATAC-sequencing analysis showing number of peaks detected in control vs RIOK2 KO HSPCs at the promoter, exon, intron and intergenic regions. FIG. 4B and FIG. 4C TSS and gene plots depicting chromatin accessibility in control and RIOK2 KO HSPCs at the transcription start sites (TSS) of genes and in the entire gene body, respectively. FIG. 4D shows chromosome view plots depicting chromatin accessibility at the promoters of GATA1 and RUNX3 in control vs RIOK2 KO HSPCs. FIG. 4E shows gene section breakdown map of RIOK2 occupancy in the entire genome. FIG. 4F shows TSS plots showing enrichment of RIOK2 at the transcription start sites (TSS) of genes as compared to Input. FIG. 4G shows chromosome view plots depicting binding of RIOK2 at the promoters of GATA1 and RUNX3.

FIG. 5A-FIG. 51 show that RIOK2 binds to a specific de novo nucleotide motif at the promoter regions of its targets to regulate transcription. FIG. 5A shows de novo nucleotide motif bound by RIOK2 in the human genome. FIG. 5B show relative binding of RIOK2 to the promoter regions of GATA1, GATA2, SPIl, RUNX3 and KLF1 via ChIP using monoclonal (Mab) and polyclonal (Pab) antibodies against RIOK2. FIG. 5C shows electrophoretic mobility shift assay showing DNA migration in the presence of recombinant human RIOK2 and duplex DNA incorporating promoter regions of GATA1, GATA2, SPIl and RUNX3. NSD1, 2: non-specific duplex DNA. FIG. 5D shows incorporation of wild-type (WT: CCC) or mutant (MUT: TTT) promoters of RIOK2 targets in basic pGL3.1 luciferase reporter plasmid. FIG. 5E-I shows wild-type (WT: CCC) and mutant (MUT: TTT) GATA1, RUNX3, KLF1, SPIl and GATA2 promoter-driven luciferase activity in response to increasing RIOK2 expression, respectively. n=3 technical replicates in 5E-5I, all comparisons done with respect to (w.r.t) EV+++control; n=3 independent experiments in b. All comparisons done with respect to EV control in e-i. * p<0.05, ** p<0.01, *** p<0.001, **** p<0.0001, ns: not significant, one-way ANOVA with Tukey's or Dunnett's correction.

Data represented as mean±SEM. FIG. 5C representative of 4 independent experiments.

FIG. 6A-FIG. 6J show that the wHTH domain of RIOK2 harbors a DNA-binding domain that is critical in hematopoiesis. FIG. 6A shows structural modeling of the wHTH domain of RIOK2 (residues 1-92) associated with B-form of double stranded DNA. u-helices and R stands are marked in red and yellow, respectively; the helix interacting with DNA is marked in purple. FIG. 6B shows a diagram showing wild-type (WT), DNA-binding mutant (DBM) and N-terminal extension (NTE) plasmids of RIOK2 with an N-terminal HA-tag. FIG. 6C shows relative binding affinities of empty vector (EV), WT, DBM and NTE RIOK2 to promoters of GATA1, GATA2, SPIl and RUNX3, assessed by ChIP.

FIG. 6D shows GATA1, GATA2, SPIl, and RUNX3 promoter-driven luciferase reporter activity after expression of EV, WT, DBM or NTE RIOK2. FIG. 6E shows a western blot showing expression of ectopically expressed HA-tagged WT, DBM and NTE RIOK2 in RIOK2 KO HSPCs. FIG. 6F shows mRNA levels of GATA1, GATA2, SPIl and RUNX3 (normalized to actin) in RIOK2-KO HSPCs ectopically expressing EV, WT, DBM or NTE RIOK2, SCR: scrambled. FIG. 6G shows erythroid progression in RIOK2 KO HSPCs ectopically expressing EV, WT, DBM or NTE RIOK2. FIG. 6H shows O-propargyl-puromycin (OPP) incorporation in scrambled (SCR) vs RIOK2 KO cells after reconstitution of WT RIOK2 or EV. FIG. 61 shows OPP incorporation in RIOK2 KO cells ectopically expressing EV, WT or DBM RIOK2. FIG. 6J shows OPP incorporation in RIOK2 KO cells ectopically expressing EV, WT or NTE RIOK2. n=3 independent experiments in FIG. 6C, 6F, 6G; n=4 technical replicates in 6D; n=3 technical replicates in 6h-6j. All comparisons done w.r.t EV control in c, d * p<0.05, ** p<0.01, *** p<0.001, **** p<0.0001, ns: not significant, one-way ANOVA with Tukey's or Dunnett's correction. Data represented as mean±SEM. Figures are representative of 3 independent experiments.

FIG. 7A-FIG. 7G show that the two transactivation domains (TADs) of RIOK2 facilitate binding with a core transcriptional complex required for hematopoiesis. FIG. 7A shows 3-state prediction model showing residues forming helices, strands and coils in the wHTH domain of RIOK2 (1-92 residues) (SEQ ID NO: 86). DBD: DNA-binding domain, TRD: Transrepressor domain. FIG. 7B shows a diagram showing WT, transactivation domain 1 and 2 deleted (ATAD1 and ATAD2) RIOK2 plasmids with N-terminal HA-tag, aas: amino acids. FIG. 7C shows a western blot showing ectopically expressed HA-tagged WT, ATAD1 and ATAD2 RIOK2 in RIOK2 KO HSPCs. FIG. 7D shows mRNA expression of GATA1, GATA2, KLF1, and SPIl (normalized to actin) in RIOK2-depleted HSPCs ectopically expressing EV, WT, ATAD1 or ATAD2 RIOK2. FIG. 7E shows a crystal structure of residues 2-301 of human RIOK2 (PDB code 6hk6) docked onto B-form of DNA, 2-92: wHTH domain, 93-289: RIO domain. TAD1: yellow, TAD2: pale grey, DBD: red, RIO domain: chocolate, end of the RIO domain marked in red spheres. FIG. 7F shows relative spectral counts reflecting binding intensities of HA-WT vs HA-ATAD1, HA-ΔTAD2 and EV with POLR2A, WDR43 and DDX21. FIG. 7G shows erythroid (CD235a), myeloid (CD11b), and megakaryocytic (CD41/61) progression in differentiating HSPCs ectopically expressing EV, WT, ΔTAD1 or ΔTAD2 RIOK2 in RIOK2-KO setting, respectively. n=3 independent experiments in 7D, 7G. * p<0.05, ** p<0.01, *** p<0.001, **** p<0.0001, ns: not significant, one-way ANOVA with Tukey's or Dunnett's correction. Data represented as mean±SEM. FIG. 7C representative of 3 independent experiments.

FIG. 8A-FIG. 8F show mRNA expression of RIOK2 correlates with its targets and other hematopoietic genes in patients with hematologic disorders. FIG. 8A shows a correlation of the mRNA expression of RIOK2 with GATA1, KLF1, GATA2 and RUNX3 in MDS patient-derived bone marrow cells; n=183. FIG. 8B shows a correlation of the mRNA expression of RIOK2 with HBB, HBA1, ITGAM and ITGB3 in MDS patient-derived bone marrow cells; n=183. FIG. 8C shows a correlation of the mRNA expression of RIOK2 with GATA1, KLF1, SPIl and RUNX3 in AML patient-derived bone marrow samples; n=76.

FIG. 8D shows a correlation of the mRNA expression of RIOK2 with HBB, HBA1, ITGAM and ITGB3 in AML patient-derived bone marrow samples; n=76. FIG. 8E shows RIOK2 mRNA expression in the whole blood of healthy controls vs chronic kidney disease (CKD) patients; n=20 healthy controls, n=63 CKD patients. FIG. 8F shows a correlation of the mRNA expression of RIOK2 with HBB, HBA1 and TFRC in healthy controls vs CKD patients; n=20 healthy controls, n=63 CKD patients. Two-tailed Pearson's correlation performed in 8A-D and 8F. Pearson's correlation coefficients (r) and P values are shown in 8A-D and 8F; Unpaired non-parametric Mann-Whitney test in 8E.

FIG. 9A-FIG. 9F show that RIOK2 drives erythropoiesis and concomitantly suppresses megakaryopoiesis and myelopoiesis. FIG. 9A-FIG. 9D show frequency of CD71+CD235a- and CD71+CD235a+population in control vs RIOK2 depleted TF-1 and K562 cells, respectively. FIGS. 9C and 9D show FACS plots and frequency of megakaryocytes (CD41/CD61+) after RIOK2 knockdown in TF-1 and K562 cells, respectively; sh #1 and 2: shRIOK2 #1 and #2. FIG. 9E shows histogram plots depicting erythroblasts (CD235a), megakaryocytes (CD41/61) and myeloblasts (CD11b) in differentiating HSPCs after KD and KO of RIOK2. FIG. 9F shows quantification of data presented in e, n=4 independent donors. n=3 technical replicates in 9A-9D. ** p<0.01, *** p <0.001, **** p<0.0001, one-way ANOVA with Tukey's or Dunnett's correction in FIG. 9C, 9D, and 9F; unpaired two-tailed Student's t-test in FIG. 9A and FIG. 9B. Data represented as mean±SEM.

FIG. 10A-FIG. 10I show that RIOK2 drives erythropoiesis and inhibits megakaryopoiesis and myelopoiesis. FIG. 10A and FIG. 10B shows total no. of myeloid (CD11b+) and megakaryocytic (CD41/61+) cells after selective differentiation of control (Ctrl) vs RIOK2 KD and KO HSPCs to myeloid and megakaryocytic lineages, respectively.

FIG. 10C and FIG. 10D shows myeloid (CD13+CD14+) and megakaryocytic (CD41+CD42b+) cells after selective differentiation of control (Ctrl) vs RIOK2 KD and KO HSPCs to myeloid and megakaryocytic lineages, respectively. FIG. 10E shows images of blast forming unit-erythroid (BFU-E) and colony forming unit-granulocyte monocyte progenitors (CFU-GM) after KD and KO of RIOK2 in HSPCs, scale bar 750 μm. FIG. 10F shows quantification of BFU-E, CFU-E and CFU-GM colonies in control vs RIOK2 KD and KO HSPCs. FIG. 10G shows principal component analysis (PCA) of quantitative proteomic dataset showing control vs RIOK2 KD and KO primary human HSPCs, n=3 donors. FIG. 10H shows a volcano plot showing differentially expressed proteins in control vs RIOK2 KD HSPCs. FIG. 101 shows gene set enrichment analysis (GSEA) plot showing defective ribosome biogenesis in RIOK2 KO vs control HSPCs. n=4 independent donors in a-d, f. * p <0.05, ** p<0.01, *** p<0.001, **** p<0.0001, one-way ANOVA with Tukey's or Dunnett's correction. Data represented as mean±SEM. FIG. 10E representative of 4 independent experiments.

FIG. 11A-FIG. 11G show that RIOK2 and GATA1 form a positive feedback loop to regulate hematopoietic differentiation. FIG. 11A shows GATA binding motif (yellow) in the promoter region of RIOK2 (black) followed by the first exon (blue) and ATG start codon (red). FIG. 11A discloses SEQ ID NO: 87. FIG. 11B shows RIOK2 promoter-driven luciferase activity in response to increasing GATA1, GATA2 and GATA3 expression in HEK293 cells. FIG. 11C shows mRNA levels of RIOK2 (normalized to actin) after GATA1 overexpression (OE) and adjoining co-relation plot; EV: empty vector; Pearson's correlation coefficient (r) and P value are shown. FIG. 11D shows RIOK2 mRNA expression (normalized to actin) in RIOK2 KD vs control cells. FIG. 11E shows GATA1 mRNA expression (normalized to actin) in GATA1 KD vs control cells. FIG. 11F shows HBB, ITGAM and ITGB3 mRNA expression (normalized to actin) in RIOK2 KD vs GATA1 KD HSPCs, compared to control. FIG. 11G shows schema showing positive feedback loop between GATA1 and RIOK2 regulating hematopoietic differentiation. n=3 independent donors in d-f; n=4 and 6 technical replicates in 11B and 11C respectively. * p<0.05, ** p <0.01, *** p<0.001, one-way ANOVA with Tukey's correction in f, Unpaired two-tailed Student's t-test in FIG. 11C-I1E. Data represented as mean±SEM.

FIG. 12A-FIG. 12E show that RIOK2 regulates expression of transcription factors involved in hematopoietic lineage commitment. FIG. 12A shows volcano plots showing differentially expressed genes (cut off: adjusted P-Value<0.05) in control vs RIOK2 KD and control vs RIOK2 KO HSPCs; red: upregulated genes, blue: downregulated genes. FIG. 12B shows GATA2 mRNA level (normalized to actin) in control vs RIOK2 KD cells after suppression of GATA2 with 2 different crRNAs. FIG. 12C shows erythroid progression (CD235a) in scrambled vs RIOK2 KD cells with or without suppression of GATA2 with 2 different crRNAs. FIG. 12D shows megakaryocytic progression (CD41/61+cells) in scrambled vs RIOK2 KD cells with or without knockdown of SPI1/RUNX3/GATA2 using 2 crRNAs against each. FIG. 12E shows a graph showing comparable mapped reads in control and RIOK2 KO HSPCs in ATAC-sequencing. n=4 technical replicates in b-d. * p<0.05, ** p<0.01, *** p<0.001, **** p<0.0001, one-way ANOVA with Tukey's or Dunnett's correction. Data represented as mean±SEM.

FIG. 13A-FIG. 13D show that RIOK2 binds to a specific de novo nucleotide motif in the human genome. FIG. 13A shows relative binding intensity of RIOK2 at the transcription start site (TSS) in RIOK2 immunoprecipitated (RIOK2 IP) vs input sample. FIG. 13B shows chromosome view plots depicting binding of RIOK2 at the promoters of GATA2 and SPI1.

FIG. 13C shows de novo DNA binding sequences (SEQ ID NOS 88-90, respectively, in order of appearance) identified in the entire genome via ChIP sequencing using monoclonal antibodies of RIOK2. The sequence highlighted in red was identified in the promoters of RIOK2's target genes. FIG. 13D shows the presence of de novo nucleotide binding motif specific for RIOK2 in the promoter regions of its putative target genes: GATA1 (SEQ ID NO: 91), GATA2 (SEQ ID NO: 92), RUNX3 (SEQ ID NO: 93), SPIl (SEQ ID NO: 94), and KLF1 (SEQ ID NO: 95).

FIG. 14A-FIG. 14G show that RIOK2 binds DNA in vitro. FIG. 14A shows electrophoretic mobility shift assay (EMSA) showing WT (central CCC) or mutant (MUT: central CCC mutated to TTT) DNA migration in the presence of recombinant human RIOK2. FIG. 14B shows a quantitative band shift represented in 14A. FIG. 14C and FIG. 14D shows EMSA and adjoining quantification showing increasing DNA binding ability of RIOK2 over a time course; mins: minutes. FIGS. 14E and 14F shows EMSA and adjoining quantification showing dose-dependent increase in DNA binding ability of RIOK2 with increasing protein concentration. FIG. 14G shows mRNA levels of early erythroid genes (HBB, HBA1, FECH, TFRC, KEL: normalized to actin) in RIOK2-KO HSPCs ectopically expressing EV, WT, DBM or NTE RIOK2, SCR: scrambled. n=3 and 4 per group in d and f respectively. n=3 experimental replicates in 14B and 14G. ** p<0.01, **** p<0.0001, ns: not significant, Unpaired two-tailed Student's t-test in b, one-way ANOVA with Dunnett's correction in 14G. Data represented as mean±SEM. FIGS. 14A and 14C representative of 3 independent experiments and FIG. 14E representative of 4 independent experiments.

FIG. 15A-FIG. 15F show characterization of the transactivation (TAD) and transrepressor (TRD) domains of RIOK2. FIG. 15A shows ChIP of the promoter regions of GATA1, GATA2, RUNX3 and KLF1 by empty vector (EV) or HA-tagged wild-type (WT), ΔTAD1 (transactivation domain 1 deleted) and ΔTAD2 (transactivation domain 2 deleted) RIOK2 using anti-HA antibodies. FIG. 15B shows mRNA levels of early erythroid genes (HBB, HBA1, FECH, TFRC, KEL: normalized to actin) in RIOK2-KO HSPCs ectopically expressing EV, WT, ΔTAD1 or ΔTAD2 RIOK2, SCR: scrambled. FIG. 15C shows erythroid progression (CD235a) in RIOK2-KO HSPCs ectopically expressing EV, WT or ATRD (deletion of transrepressor domain) RIOK2. FIG. 15D shows mRNA levels of early erythroid genes (HBA1, HBA2, SPTA1, TFRC: normalized to actin) in RIOK2-KO HSPCs ectopically expressing EV, WT or ATRD RIOK2. All comparisons done with respect to KO+EV group in FIG. 15B-D. FIG. 15E show schema showing the 3 known domains of human RIOK2: N-terminal wHTH domain, central RIO domain and C-terminal domain. Positions of the DNA binding domain (DBD) and the transactivation domains (TAD1 and TAD2) of RIOK2 are shown. FIG. 15F shows a diagram illustrating RIOK2 as a master transcriptional regulator of key transcription factors (GATA1, KLF1, RUNX3, SPI1 and GATA2) in hematopoiesis. n=2 per group in a; n=3 independent donors in 15B-15D. Data represented as mean±SEM. * p <0.05, ** p<0.01, *** p<0.001, **** p<0.0001, ns: not significant, one-way ANOVA with Tukey's or Dunnett's correction in b-d. Data represented as mean±SEM.

FIG. 16A-FIG. 16G show that the kinase domain of RIOK2 is involved in erythropoiesis but does not affect RIOK2's transcriptional activities. FIG. 16A shows GATA1, SPIl and RUNX3 promoter-driven luciferase reporter activity after expression of EV, WT, K123A, DBM or ΔTAD1 RIOK2; all comparisons done with respect to EV group. FIG. 16B shows EMSA showing DNA-binding affinities of EV, WT, K123A, DBM, ΔTAD1 and ΔTAD2 RIOK2. FIG. 16C shows western blot showing expression of ectopically expressed HA-tagged WT, DBM, K123A, ΔTAD1 RIOK2 or EV in RIOK2 KO HSPCs. FIG. 16D shows O-propargyl-puromycin (OPP) incorporation in control (Ctrl) vs RIOK2 KO cells after reconstitution of WT, DBM, K123A RIOK2 or EV. FIG. 16E-16G shows selective progression of erythroid (CD235a), myeloid (CD11b), and megakaryocytic (CD41/61) differentiation in HSPCs ectopically expressing EV, WT, DBM, ΔTAD1 or K123A RIOK2 in RIOK2-KO setting, respectively. All comparisons done with respect to EV in a, and KO+EV group in FIG. 16D-G. n=3 technical replicates in a, 16D-16G. * p<0.05, ** p<0.01, *** p<0.001, **** p<0.0001, ns: not significant, one-way ANOVA with Tukey's or Dunnett's correction. Data represented as mean±SEM. FIGS. 16B and 16C representative of 2 independent experiments.

FIG. 17 shows a graphical illustration of RIOK2 functioning as a master transcription factor governing hematopoietic differentiation.

FIGS. 18A- FIG. 18C show flow cytometry plots showing gating strategies. FIG. 18A shows erythroid, myeloid and megakaryocytic lineages. FIG. 18B shows BFU-E and CFU-E formation, and FIG. 18C shows CFU-GM and CFU-Mk formation are shown.

FIG. 19 shows amino acid sequences of the 5 RIOK2 mutants and their roles in erythropoiesis. Amino acid sequences of the 5 loss-of-function (LOF) mutants (SEQ ID NOS 28-32, respectively, in order of appearance) identified in a consanguineous Pakistani cohort.

Amino acids labeled in green represent exclusive amino acids not encoded in wild-type (WT) RIOK2 (SEQ ID NO: 27).

FIG. 20 shows erythroid differentiation (CD235a+) of primary human HSPCs with endogenous RIOK2 knocked out and reconstituted with either empty vector (EV), wild-type (WT) or loss-of-function (LOF) mutants M1, M2, M3, M4, or M5.

FIG. 21 shows CRISPR/Cas9-mediated knockdown or knockout of RIOK2 decreases telomere length in primary human HSPCs dose-dependently, thus signifying that RIOK2 is critical for telomere maintenance.

FIG. 22 shows CRISPR/Cas9-mediated knockdown of RIOK2 decreases telomeric puncta (green dots) in TF-1 cells, thus signifying that loss of RIOK2 leads to significant telomere shortening.

FIG. 23 shows CRISPR/Cas9-mediated knockdown of RIOK2 decreases telomeric puncta (green dots) in K562 cells, thus signifying that loss of RIOK2 leads to significant telomere shortening.

FIG. 24A-FIG. 24B show that RIOK2 transcriptionally regulates TRiC and Dyskerin complex subunit expression via its transcription factor (TF) activity. FIG. 24A shows reduced TRiC complex gene expression while FIG. 24B shows downregulated Dyskerin complex gene expression upon RIOK2 deficiency in primary human hematopoietic stem and progenitor cells.

FIG. 25 shows that mRNA expression of RIOK2 is significantly reduced in PBMCs of young vs old individuals.

FIG. 26 shows that mRNA expression of RIOK2 positively correlates with TRiC and Dyskerin complex subunits in PBMCs of young vs old individuals.

FIG. 27A-FIG. 27B show stapled peptides modeled after DNA-binding domain (DBD) of RIOK2 to inhibit RIOK2. FIG. 27A shows helical wheel depiction of DBD peptides and their staple positions. FIG. 27A discloses SEQ ID NO: 101. FIG. 27B shows amino acid sequences (SEQ ID NOS 11 and 102-106, respectively, in order of appearance) of the DBD staple-scanning library generated by inserting all-hydrocarbon i, i+4 or i, i+7 staples sequentially along the length of the DBD peptides. X: S5-pentenyl alanine; 8: R5-octenyl alanine; SAH: stabilized u-helix.

FIG. 28 shows stapled peptides (Biotinylated peptides used for EMSA).

Electrophoretic mobility shift assay (EMSA) to detect binding of RIOK2 (recombinant human full-length RIOK2, Abcam) with duplex DNA (IDT). The binding of DNA with protein confers an increase in its molecular weight, thus resulting in decreased migration in agarose gel, leading to a shift in the DNA band. RIOK2 alone or in the presence of unstapled peptide (P6)/stapled peptide (P8) can efficiently bind DNA. However, stapled peptides P7, P9-11 can completely block RIOK2's interaction with DNA, as observed by no DNA shift that refers to blockade in RIOK2's DNA-binding ability. SAH-DBD=P6; SAH-DBD1=P7; SAH-DBD2-P8; SAH-DBD3=P9; SAH-DBD4=P10; SAH-DBD5=P11.

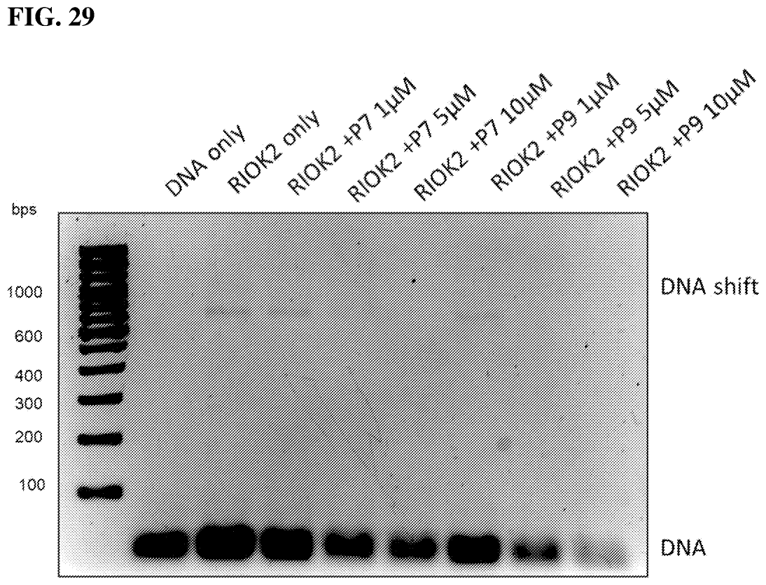

FIG. 29 shows dose-dependent impact of Stapled Peptides on RIOK2's DNA-binding activity. Both stapled peptides P7 and P9 marginally affect RIOK2's DNA binding ability at 1 μM, and completely block RIOK2's DNA binding ability at 10 μM. P9 potently blocks RIOK2's DNA binding ability at 5 μM also, whereas administration of P7 at 5 μM fails to fully inhibit DNA binding activity of RIOK2.

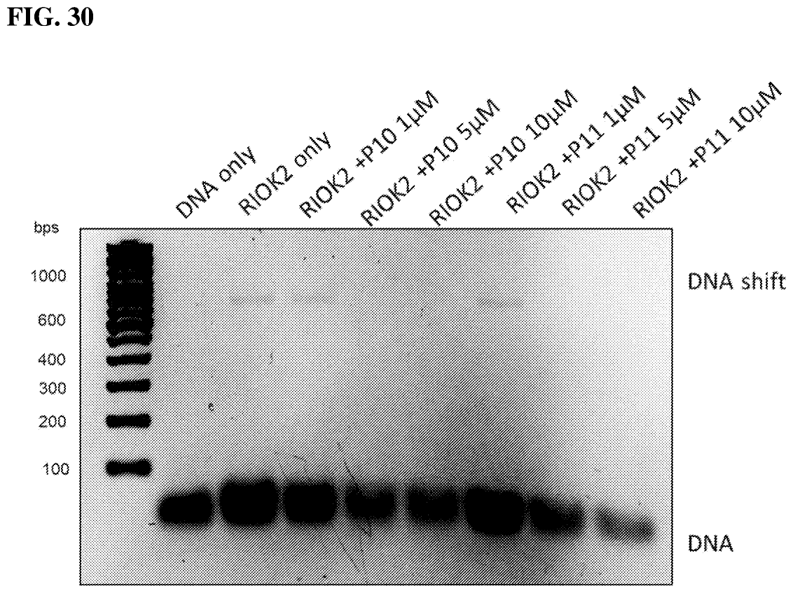

FIG. 30 shows dose-dependent impact of Stapled Peptides on RIOK2's DNA-binding activity. Both stapled peptides P10 and P11 marginally affect RIOK2's DNA binding ability at 1 μM, and completely block RIOK2's DNA binding ability at 10 μM. Both P10 and P11 potently block RIOK2's DNA binding ability at 5 μM also.

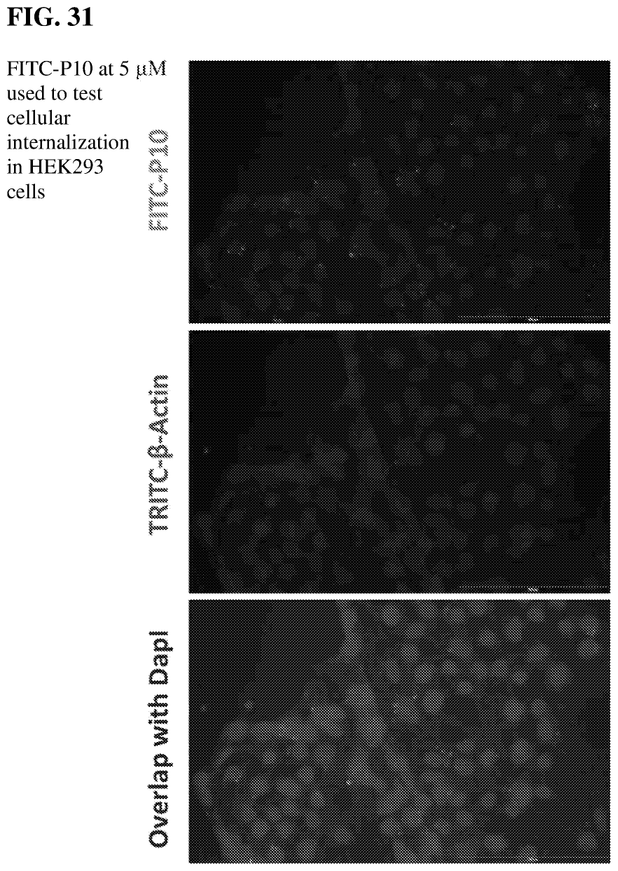

FIG. 31 shows FITC-P10 stapled peptide at 5 μM used to test cellular internalization in HEK293 cells.

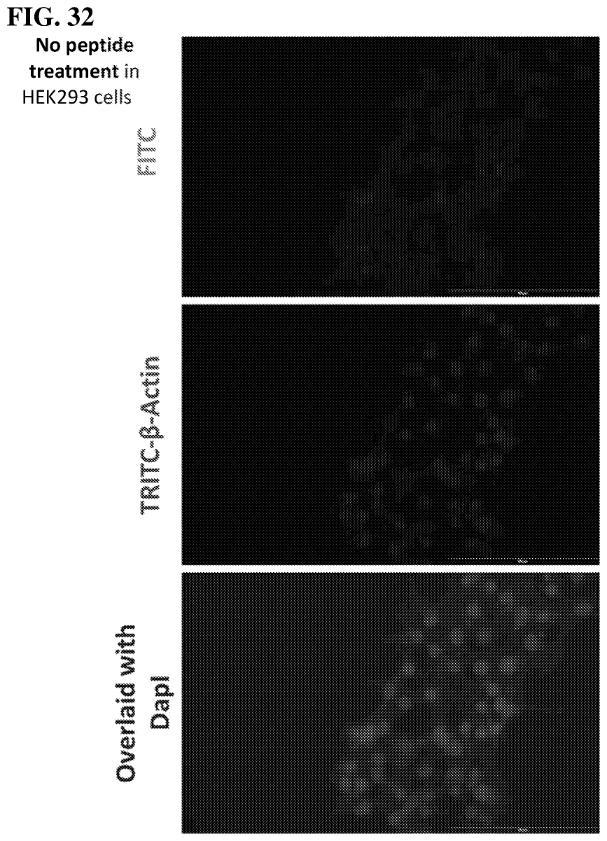

FIG. 32 shows no peptide treatment in HEK293 cells.

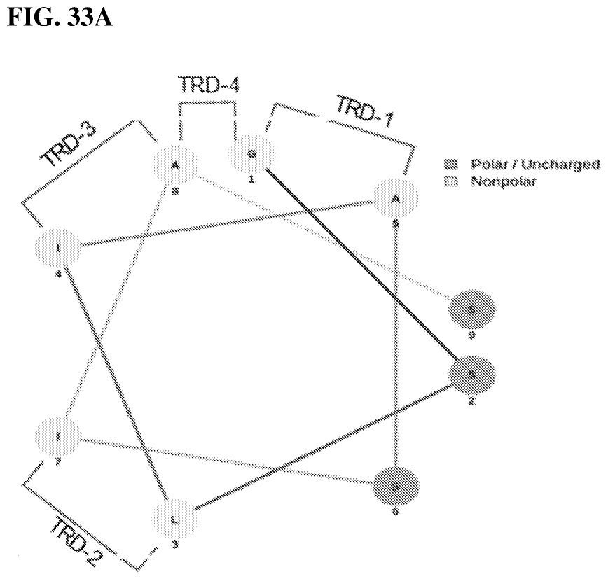

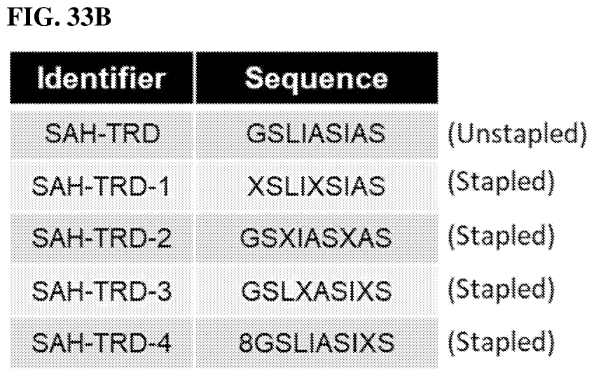

FIG. 33A-FIG. 33B show stapled peptides modeled after transrepressor domain (TRD) of RIOK2. FIG. 33A shows helical wheel depiction of TRD peptides and their staple positions. FIG. 33B shows amino acid sequences (SEQ ID NOS 5 and 107-110, respectively, in order of appearance) of the TRD staple-scanning library generated by inserting all-hydrocarbon i, i+4 or i, i+7 staples sequentially along the length of the TRD peptide (X: S5-pentenyl alanine; 8: R5-octenyl alanine; SAH: stabilized u-helix).

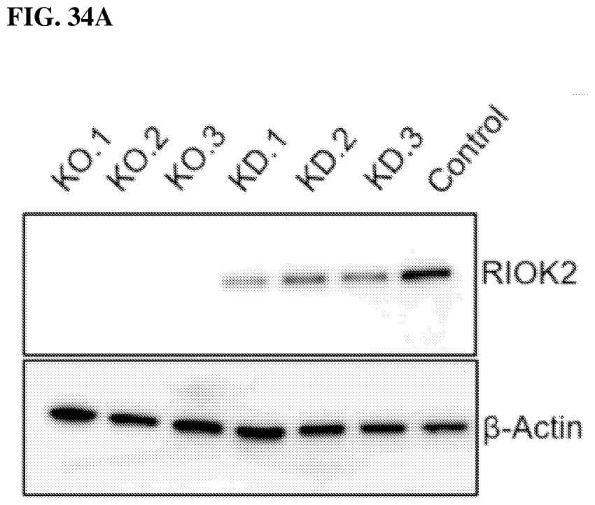

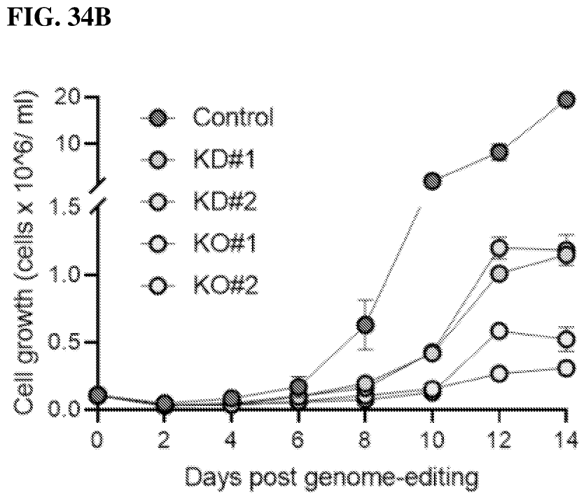

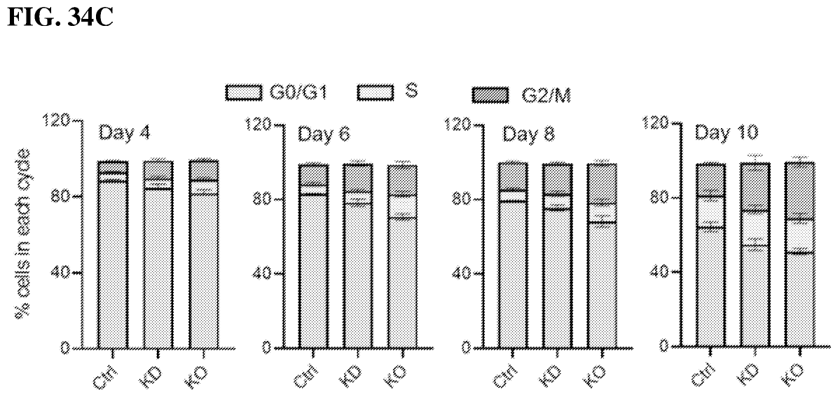

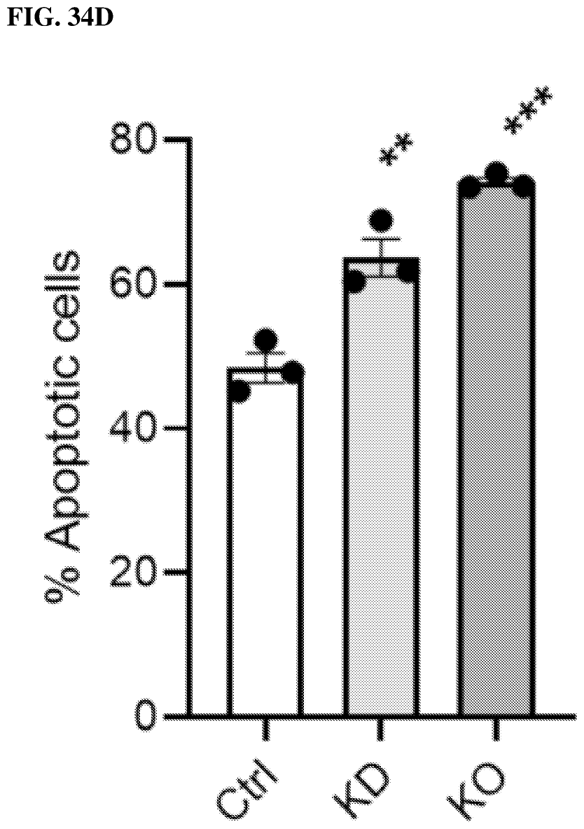

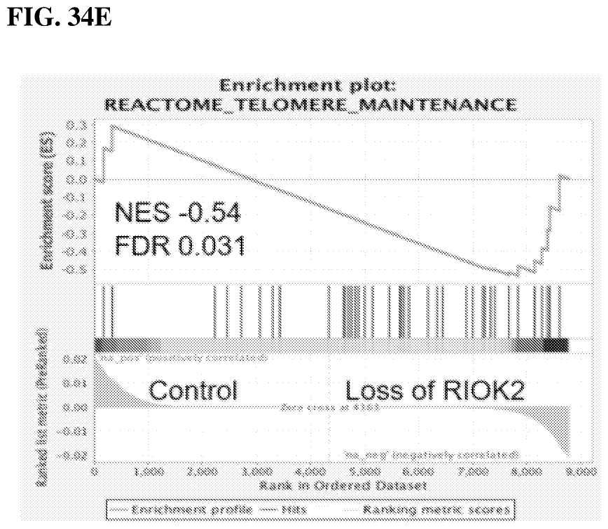

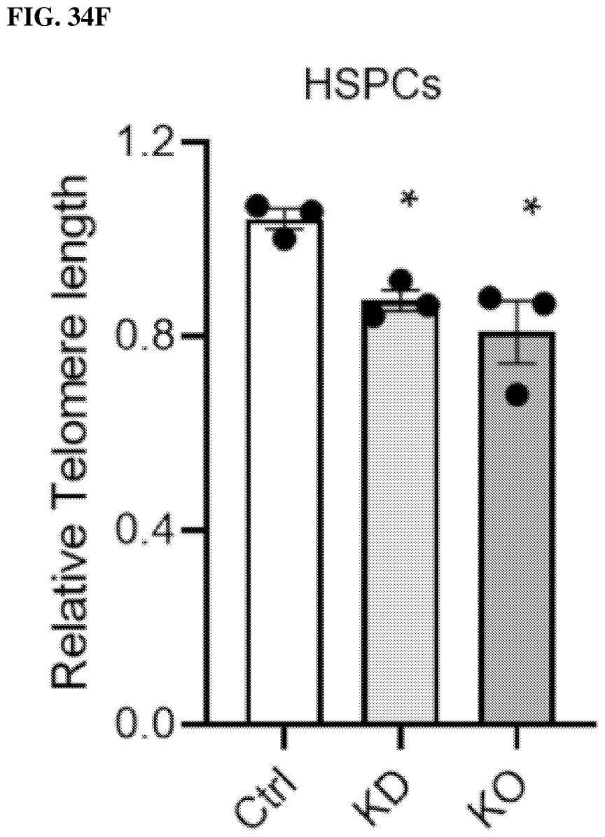

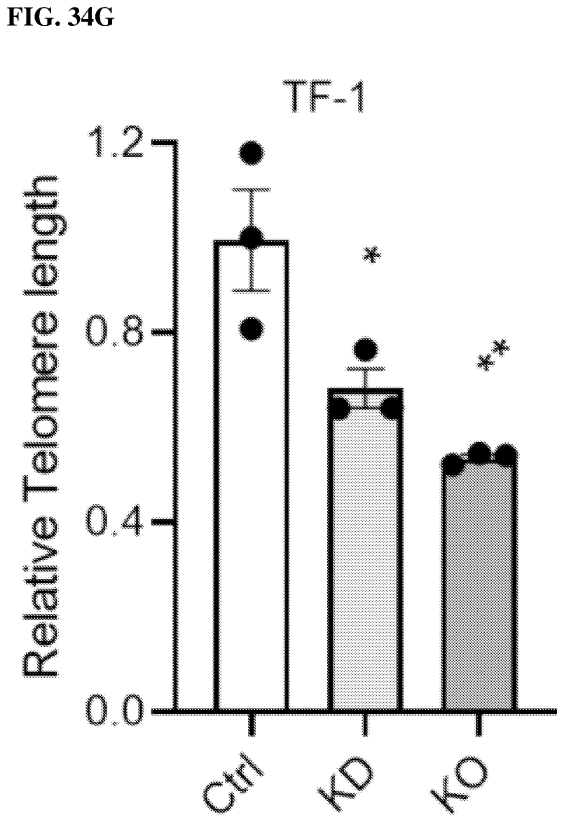

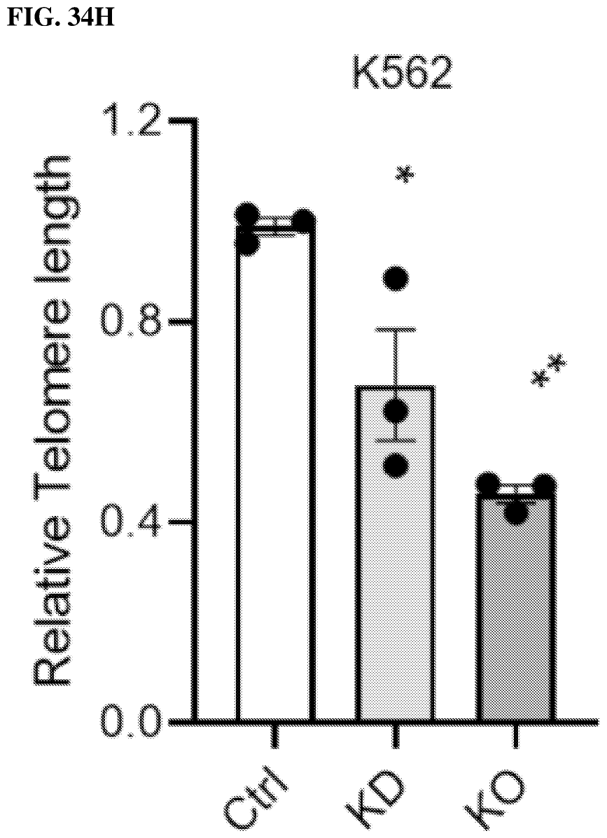

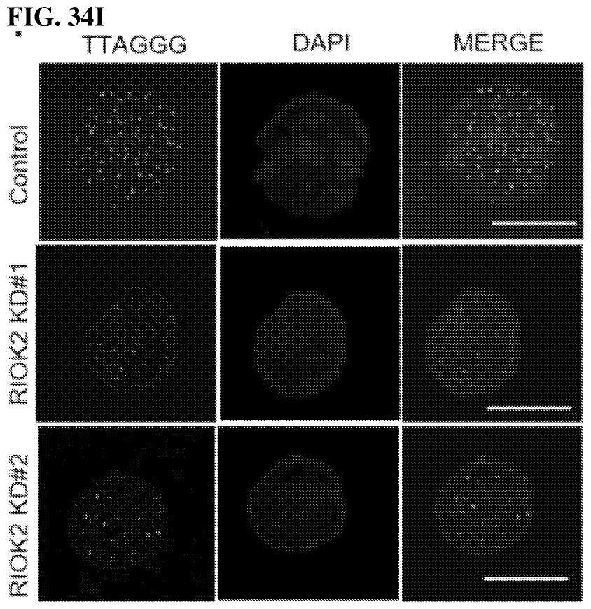

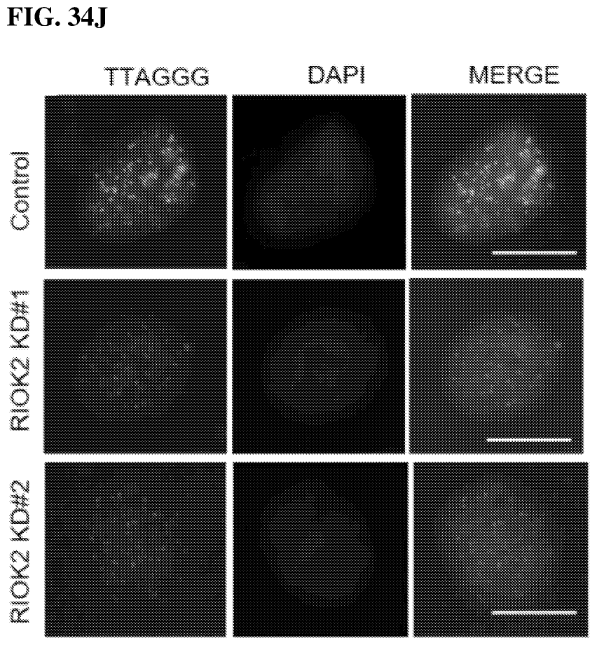

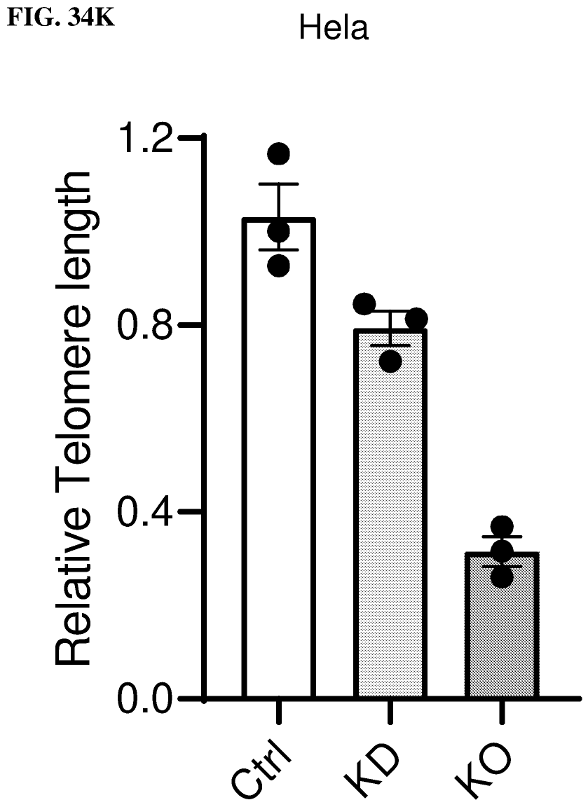

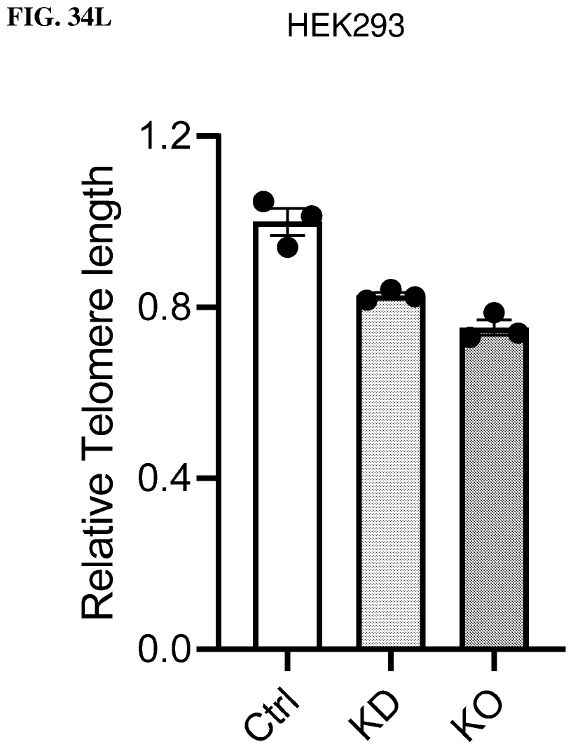

FIG. 34A-FIG. 34L show loss of RIOK2 results in telomere shortening. FIG. 34A shows an immunoblot showing RIOK2 expression after knockdown (KD) and knockout (KO) of RIOK2 in TF-1 cells and Ctrl (control). FIG. 34B shows a graph demonstrating cell proliferation in TF-1 cells after knockdown (KD) and knockout (KO) of RIOK2. FIG. 34C shows cell cycle analysis of control (Ctrl) vs RIOK2 KD and KO TF-1 cells post day 4, 6, 8 and 10 of gene-editing. FIG. 34D shows a graph showing percent apoptotic cells in RIOK2 proficient vs deficient TF-1 cells. FIG. 34E shows a gene set enrichment analysis (GSEA) plot of telomere maintenance-associated genes in RIOK2-depleted vs control HSPCs. FIG. 34F, FIG. 34G, and FIG. 34H shows quantitative PCR-based analysis of telomere lengths in primary human HSPCs, TF-1 and K562 cells respectively, upon RIOK2 deficiency. FIG. 341 and FIG. 34J shows fluorescence in-situ hybridization (FISH) of telomeric DNA in TF-1 and K562 cells upon RIOK2 deficiency. FIG. 34K and FIG. 34L shows quantitative PCR-based analysis of telomere lengths in control vs RIOK2-depleted Hela and HEK293 cells (* p <0.05, ** p<0.01, *** p<0.001, **** p<0.0001, one-way analysis of variance (ANOVA)). Data represented as mean±SEM. All comparisons are done w.r.t. control (Ctrl). Scale bar 10 μm.

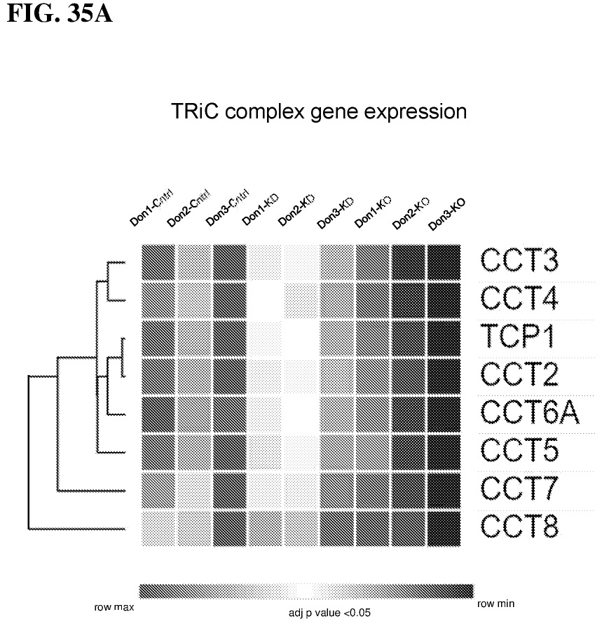

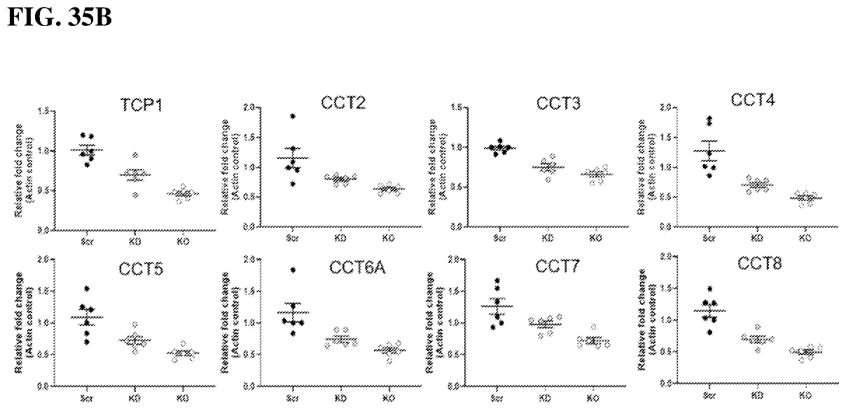

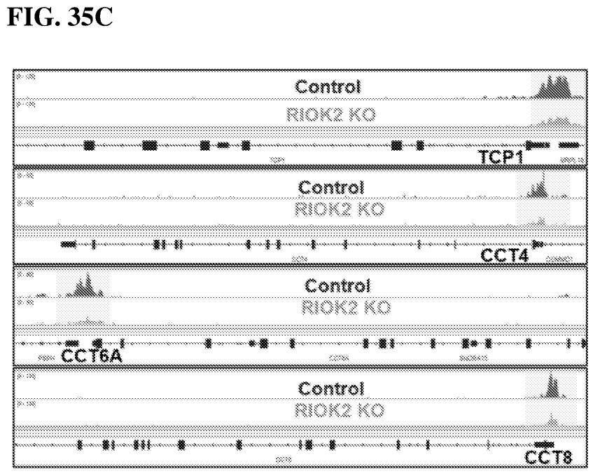

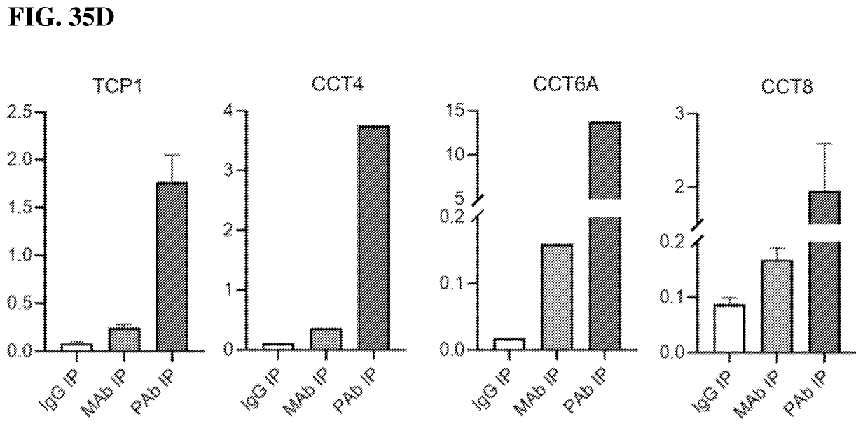

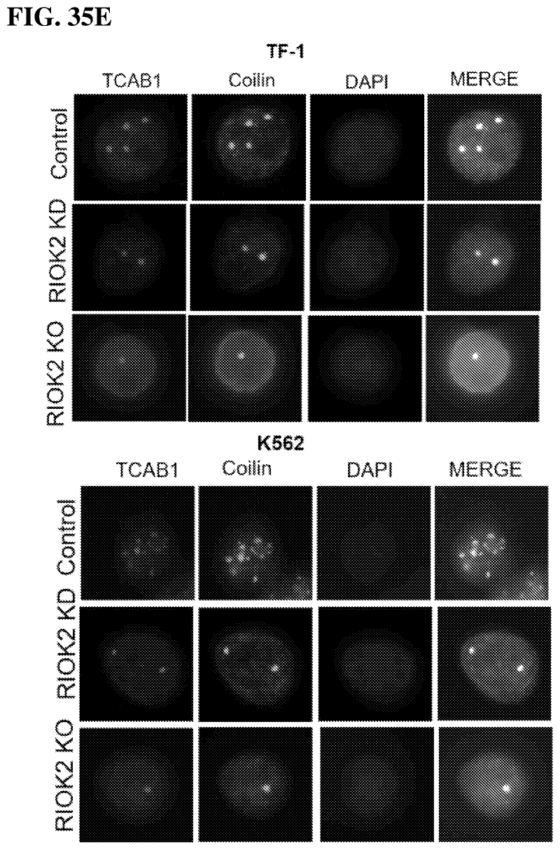

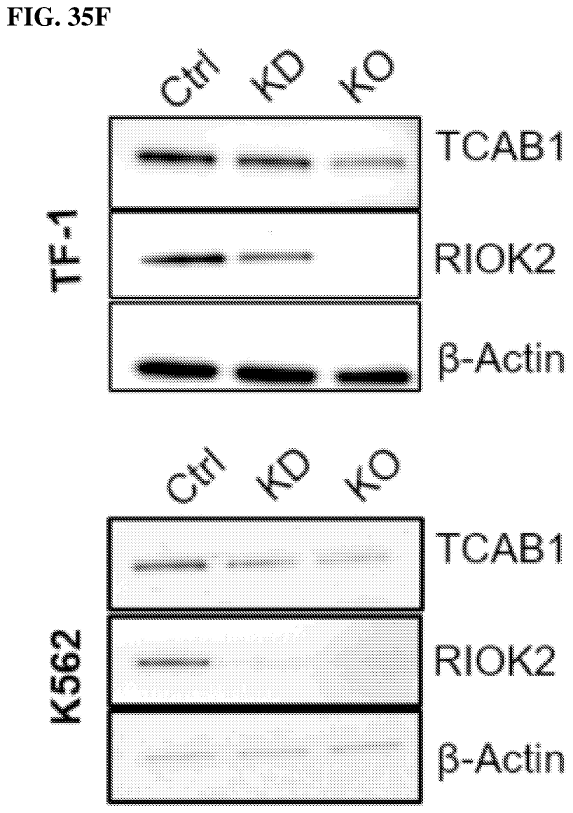

FIG. 35A-FIG. 35F show RIOK2 regulates mRNA expression of TRiC complex. FIG. 35A shows of a heat map of differentially expressed TRiC complex genes (cut off: adj P value<0.05) from bulk RNA sequencing of primary human HSPCs with knockdown (KD) and knockout (KO) of RIOK2 (n=3 donors). FIG. 35B shows TCP1 (CCT1)-CCT8 mRNA expression (normalized to P-actin) in HSPCs upon KD and KO of RIOK2 (n=6 donors). FIG. 35C shows chromosome view plots depicting chromatin accessibility at the promoters of TCP1, CCT4, CCT6A and CCT8 in control vs RIOK2 KO HSPCs. FIG. 35D shows relative binding of RIOK2 to the promoter regions of TCP1, CCT4, CCT6A and CCT8 via ChIP using monoclonal (Mab) and polyclonal (Pab) antibodies against RIOK2. FIG. 35E shows immunofluorescence staining showing expression of TCAB1 in control, RIOK2 knockdown (KD) and knockout (KO) TF-1 and K562 cells. FIG. 35F shows western blotting showing total protein expression of TCAB1 in TF-1 and K562 cells after knockdown (KD) and knockout (KO) of RIOK2 (* p<0.05, ** p<0.01, *** p<0.001, **** p<0.0001, one-way analysis of variance (ANOVA)). Data represented as mean±SEM. Scale bar 10 μm.

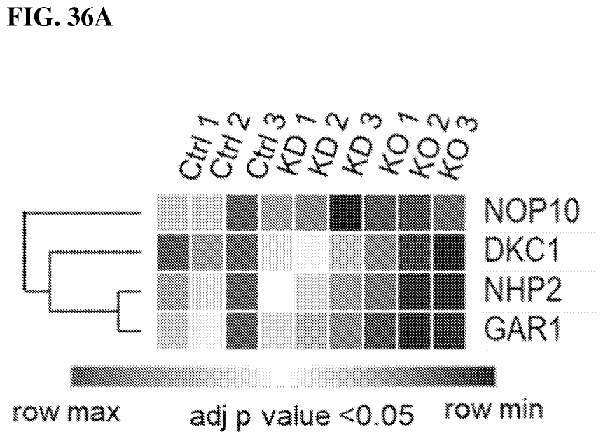

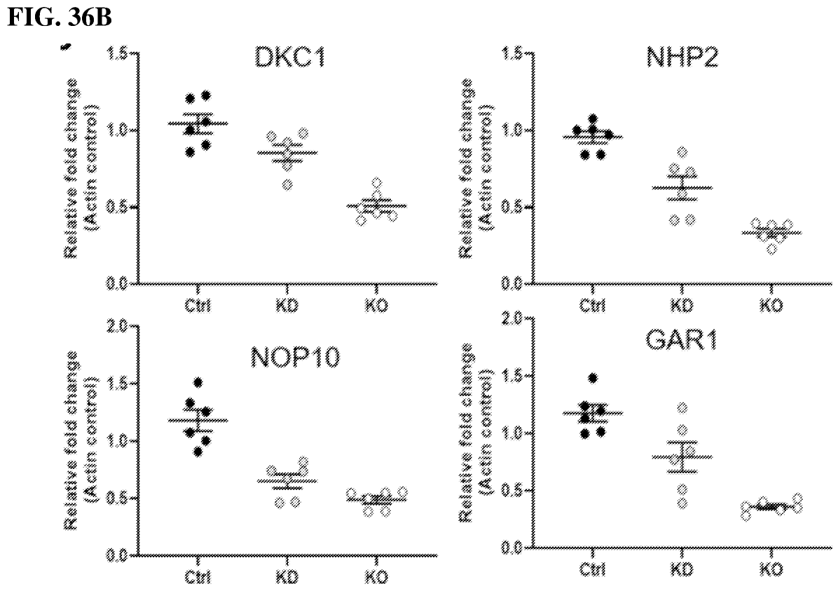

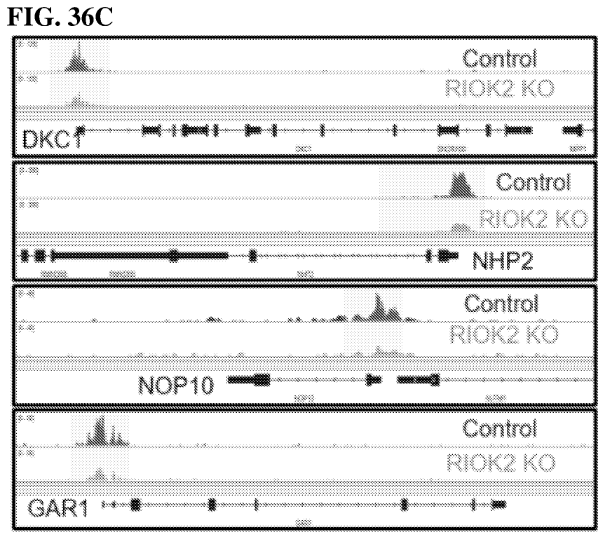

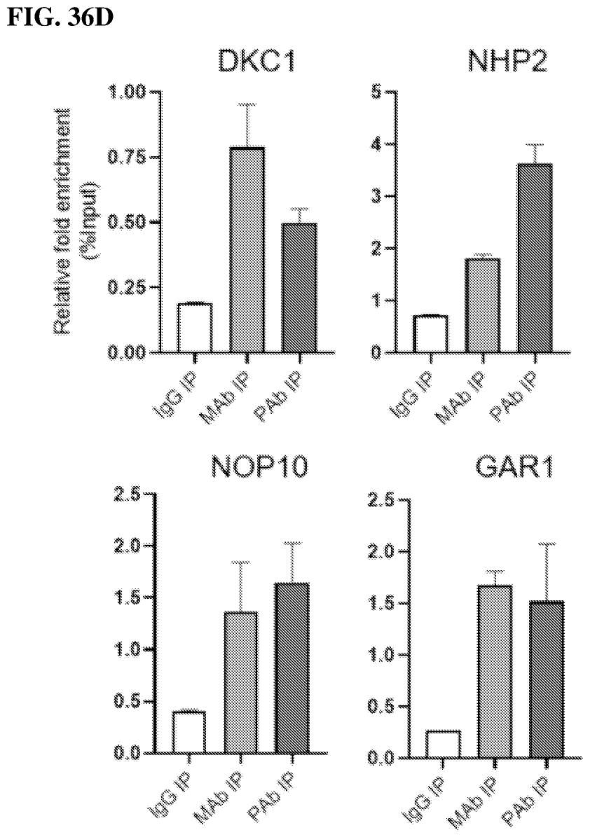

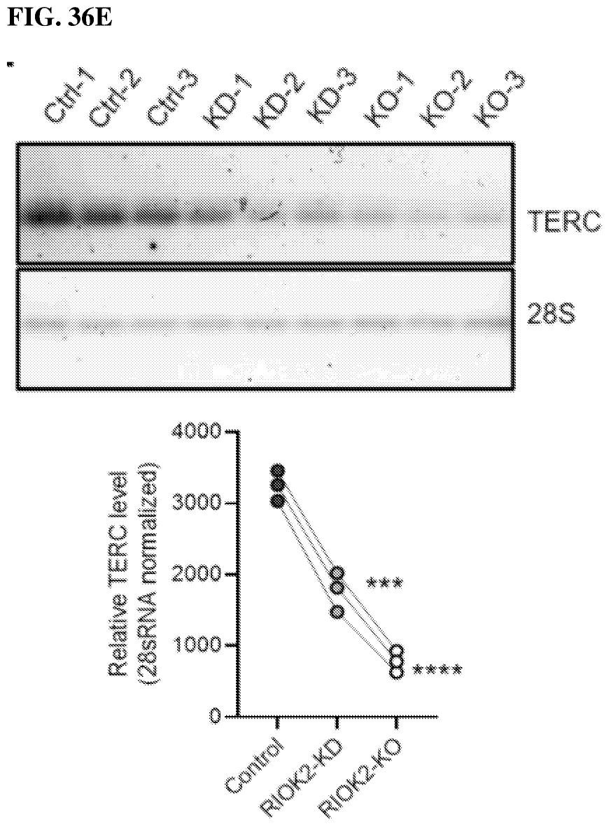

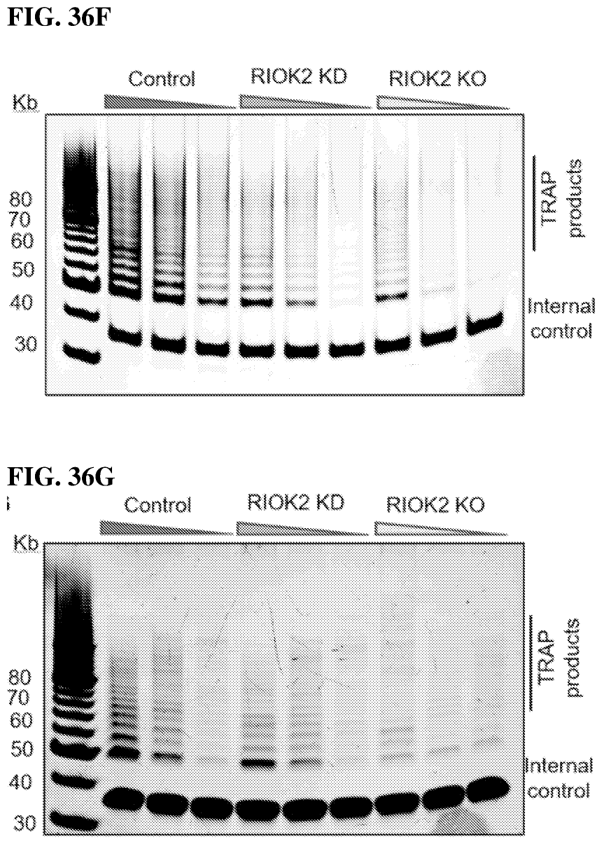

FIG. 36A-FIG. 36G show RIOK2 regulates mRNA expression of dyskerin complex.

FIG. 36A shows heat map of differentially expressed dyskerin complex genes (cut off: adj P value<0.05) from bulk RNA sequencing of primary human HSPCs with knockdown (KD) and knockout (KO) of RIOK2 (n=3 donors). FIG. 36B shows DKCI, NHP2, NOP10 and GAR1 mRNA expression (normalized to 0-actin) in HSPCs upon KD and KO of RIOK2 (n=6 donors). FIG. 36C shows chromosome view plots depicting chromatin accessibility at the promoters of DKCI, NHP2, NOP10 and GAR1 in control vs RIOK2 KO HSPCs. FIG. 36D shows relative binding of RIOK2 to the promoter regions of DKCI, NHP2, NOP10 and GAR1 via ChIP using monoclonal (Mab) and polyclonal (Pab) antibodies against RIOK2.

FIG. 36E shows agarose gel picture and adjoining quantification showing expression of TERC and 28s RNA in control, RIOK2 knockdown (KD) and knockout (KO) HSPCs. FIG. 36F and FIG. 36G shows TRAP assay showing telomerase activity in TF-1 and K562 cells after knockdown (KD) and knockout (KO) of RIOK2. 0.5-0.25-0.1 pg total protein containing lysates loaded for each condition (* p<0.05, ** p<0.01, *** p<0.001, **** p<0.0001, one-way analysis of variance (ANOVA)). Data represented as mean±SEM.

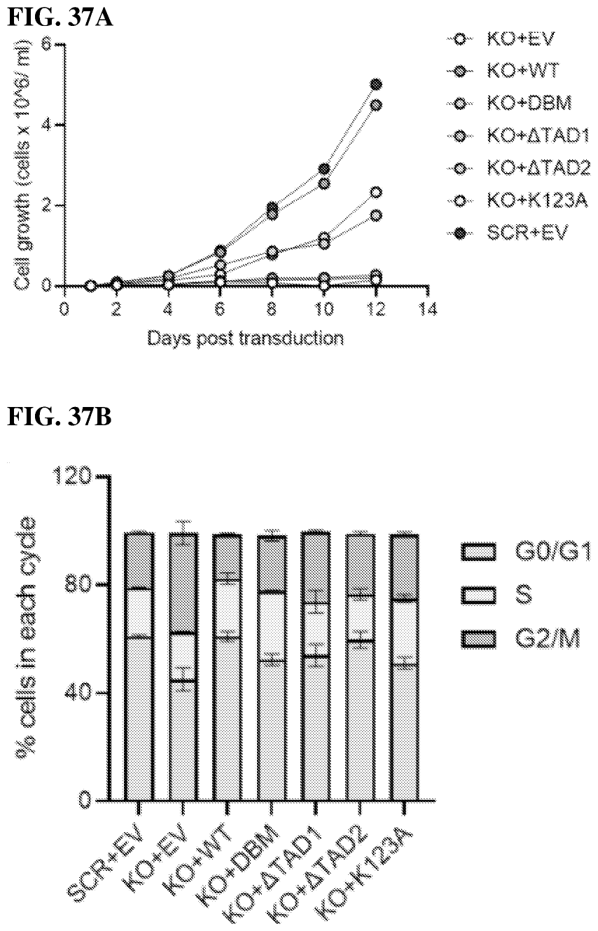

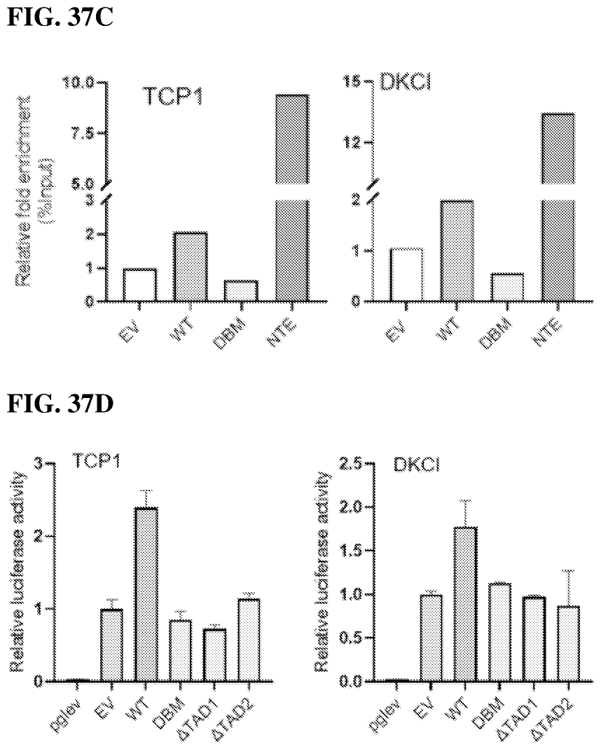

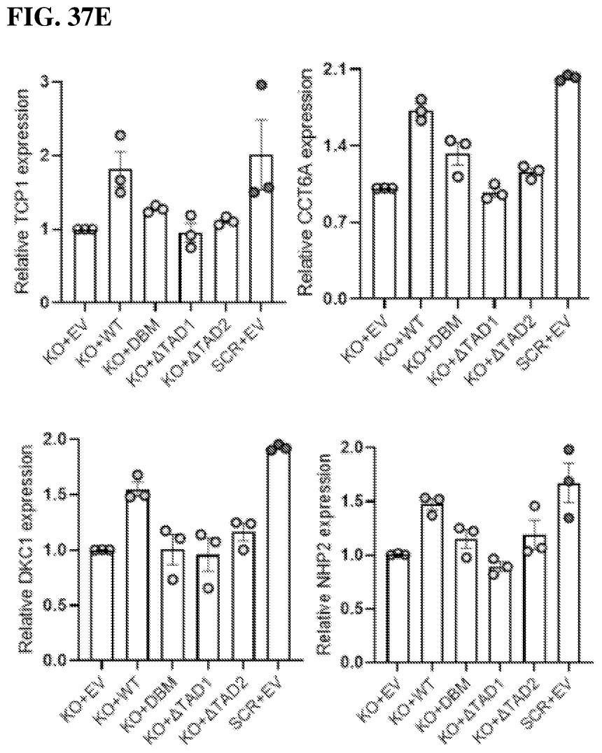

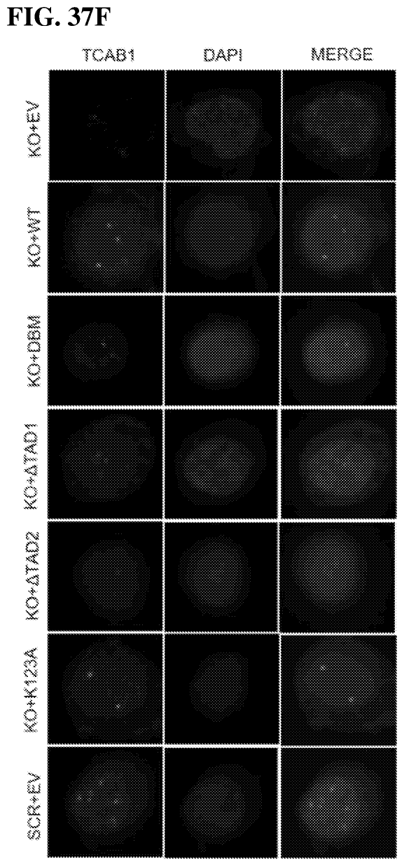

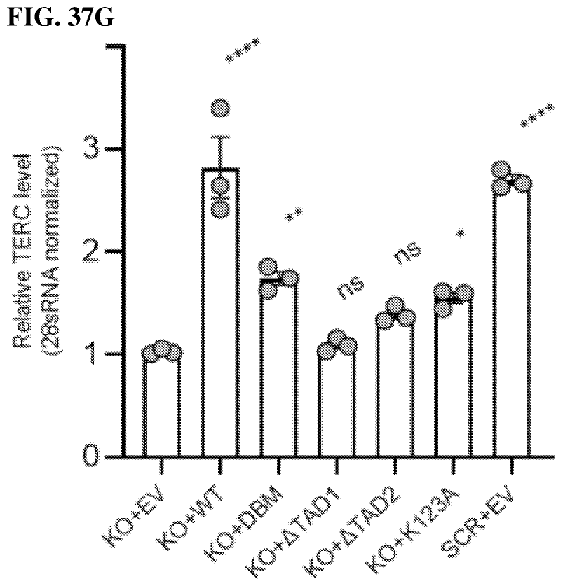

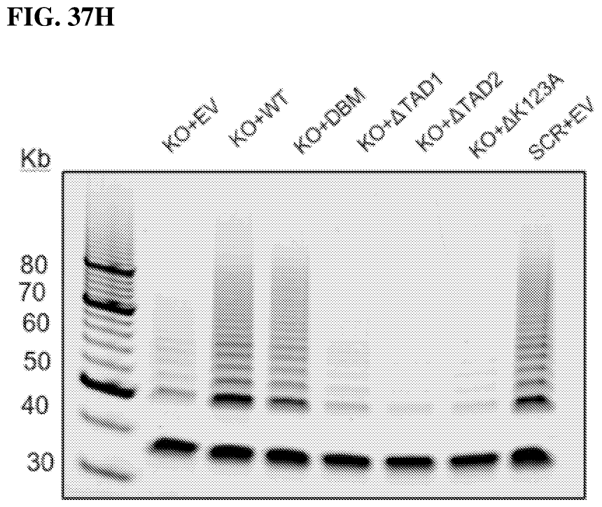

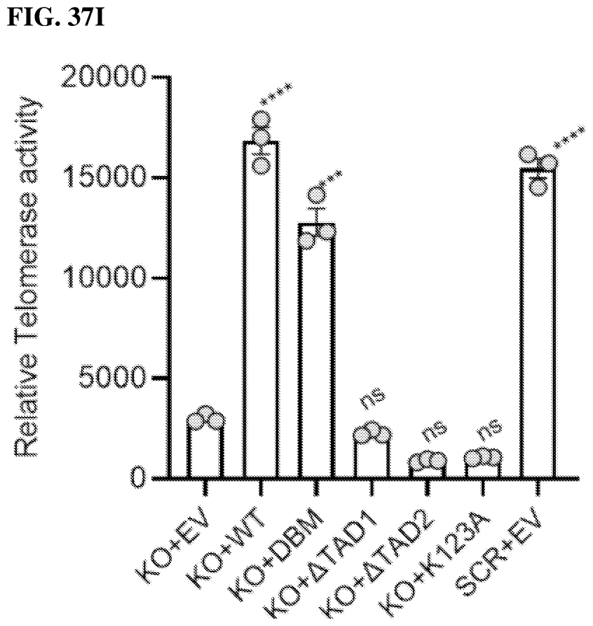

FIG. 37A-FIG. 371 show loss of RIOK2's transcriptional abilities results in telomere shortening. FIG. 37A shows a graph showing cell proliferation in RIOK2 knockout (KO) TF-1 cells ectopically expressing EV, WT, DBM, ΔTAD1, ΔTAD2 or K123A RIOK2. FIG. 37B shows cell cycle analysis of RIOK2 knockout (KO) TF-1 cells ectopically expressing EV, WT, DBM, ΔTAD1, ΔTAD2 or K123A RIOK2. FIG. 37C shows relative binding of EV, WT, DBM or NTE RIOK2 to the promoter regions of TCP1 and DKCI via ChIP using monoclonal antibodies against HA. FIG. 37D shows a luciferase reporter assay showing transactivation of TCP1 and DKC1 by EV, WT, DBM, ΔTAD1 and ΔTAD2 RIOK2. FIG. 37E shows TCP1, CCT6A, DKCI and NHP2 mRNA expression (normalized to 0-actin) in RIOK2 KO TF-1 cells ectopically expressing EV, WT, DBM, ΔTAD1 and ΔTAD2 RIOK2 (Scr: Scrambled). FIG. 37F shows immunofluorescence staining showing levels of TCAB1 in RIOK2 KO TF-1 cells ectopically expressing EV, WT, DBM, ΔTAD1, ΔTAD2 or K123A RIOK2. FIG. 37G shows the expression of TERC (normalized to 28s RNA) in RIOK2 KO TF-1 cells ectopically expressing EV, WT, DBM, ΔTAD1, ΔTAD2 or K123A RIOK2. FIG. 37H shows a TRAP assay showing telomerase activity in RIOK2 KO TF-1 cells ectopically expressing EV, WT, DBM, ΔTAD1, ΔTAD2, AN, or K123A RIOK2. FIG. 371 shows quantification of TRAP assay shown in FIG. 37H (p<0.05, ** p<0.01, *** p<0.001, **** p <0.0001, one-way analysis of variance (ANOVA)). Data represented as mean±SEM. Scale bar 10 μm.

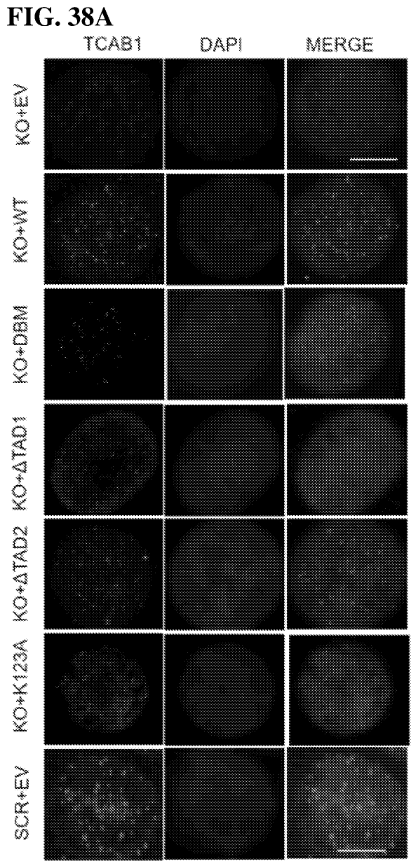

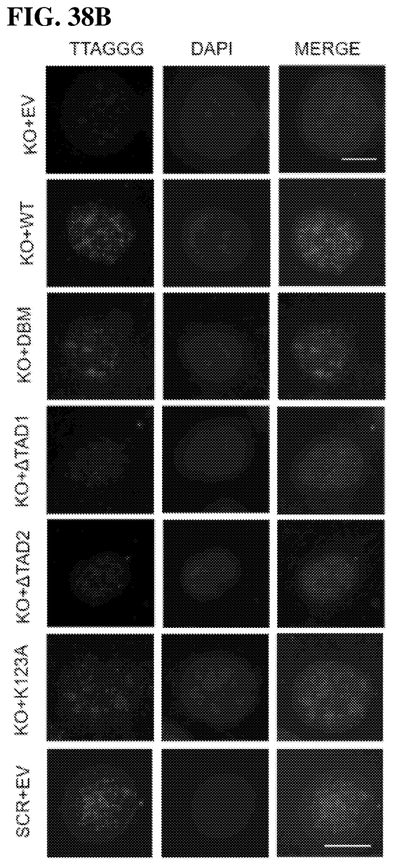

FIG. 38A-FIG. 38B show the transcriptional functions of RIOK2 are critical in preventing telomere shortening. FIG. 38A and FIG. 38B shows fluorescence in-situ hybridization of telomeric DNA in RIOK2 KO TF-1 and K562 cells respectively, each ectopically expressing EV, WT, DBM, ΔTAD1, ΔTAD2 or K123A RIOK2. Scale bar 10 μm.

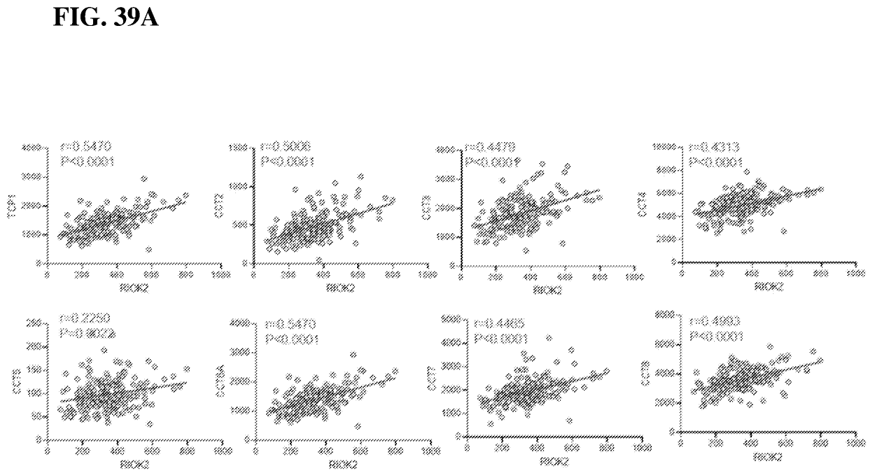

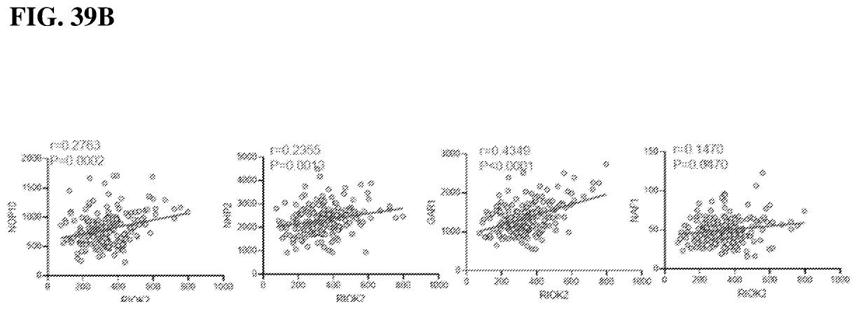

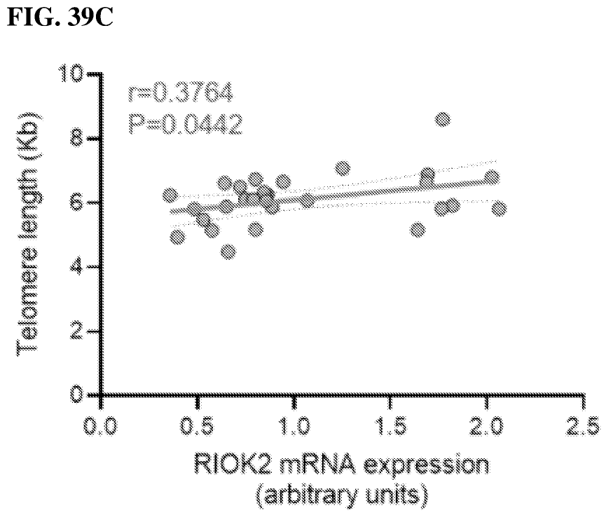

FIG. 39A-FIG. 39C show mRNA expression of RIOK2 correlates with telomere lengths, and mRNA expression of TRiC and dyskerin complexes in MDS patients. FIG. 39A shows the correlation of mRNA expression of RIOK2 with TRiC complex subunits in MDS patient-derived bone marrow cells; n=183. FIG. 39B shows the correlation of the mRNA expression of RIOK2 with dyskerin complex subunits in MDS patient-derived bone marrow cells; n=183. FIG. 39C shows the correlation of mRNA expression of RIOK2 with telomere lengths in MDS patient-derived bone marrow cells; n=29. Two-tailed Pearson's correlation performed in FIG. 39A-FIG. 29C. Pearson's correlation coefficients (r) and P values are shown in FIG. 39A-FIG. 39C.

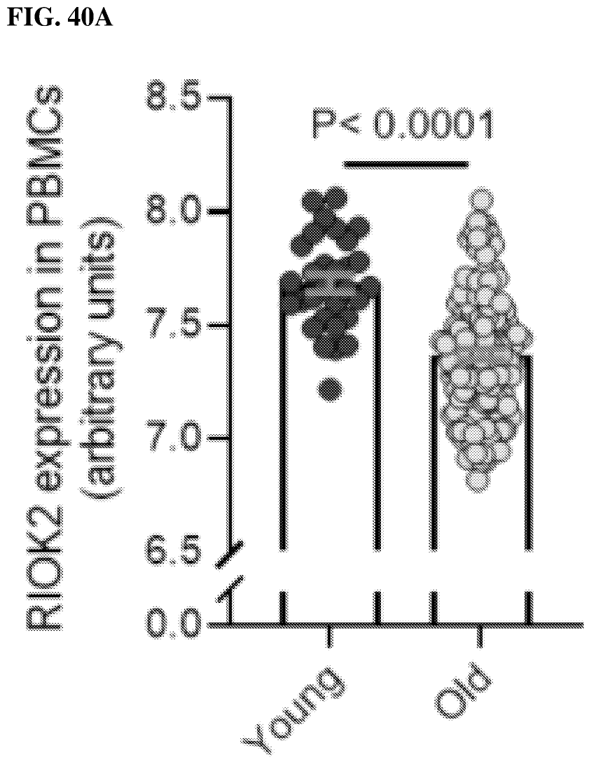

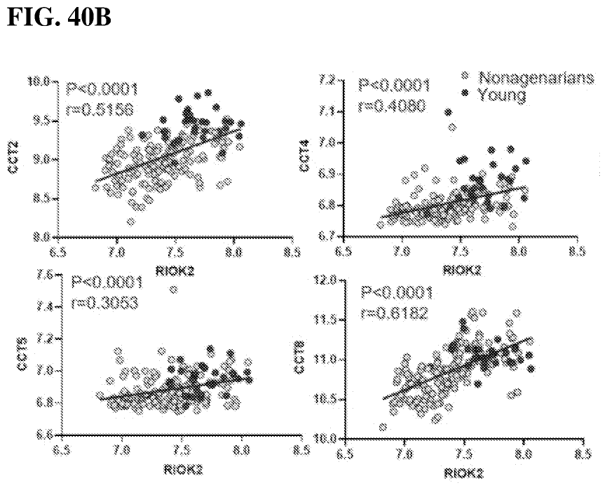

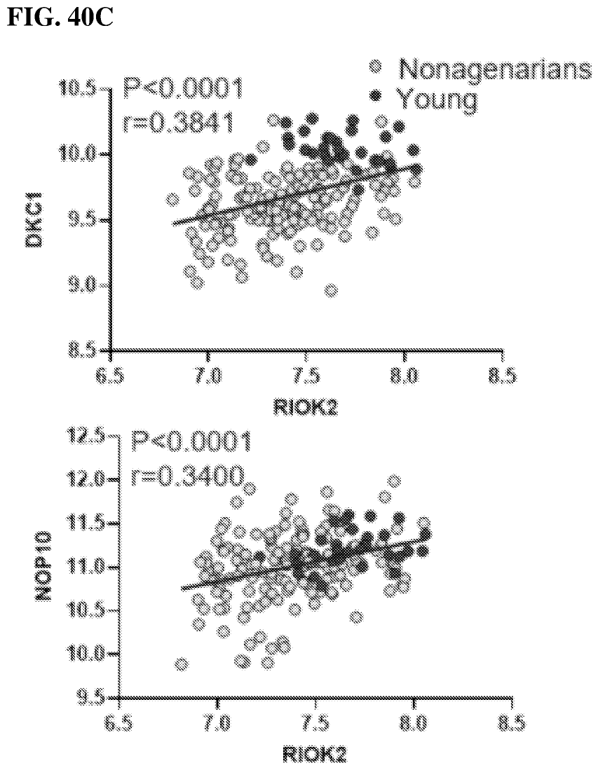

FIG. 40A-40C show mRNA expression of RIOK2 is reduced in aging individuals.

FIG. 40A shows quantification of mRNA expression of RIOK2 in PBMCs derived from 30 young (19-30 years) vs 146 nonagenarians (>90 years). FIG. 40B shows the correlation of mRNA expression of RIOK2 with TRiC complex subunits in PBMCs derived from 30 young (19-30 years) and 146 nonagenarians (>90 years). FIG. 40C shows the correlation of mRNA expression of RIOK2 with dyskerin complex subunits in PBMCs derived from 30 young (19-30 years) and 146 nonagenarians (>90 years). Two-tailed Pearson's correlation was performed and shown in FIG. 40B-FIG. 40C. Pearson's correlation coefficients (r) and P values are shown in FIG. 40B-FIG. 40C. Unpaired non-parametric Mann-Whitney test is shown in FIG. 40A.

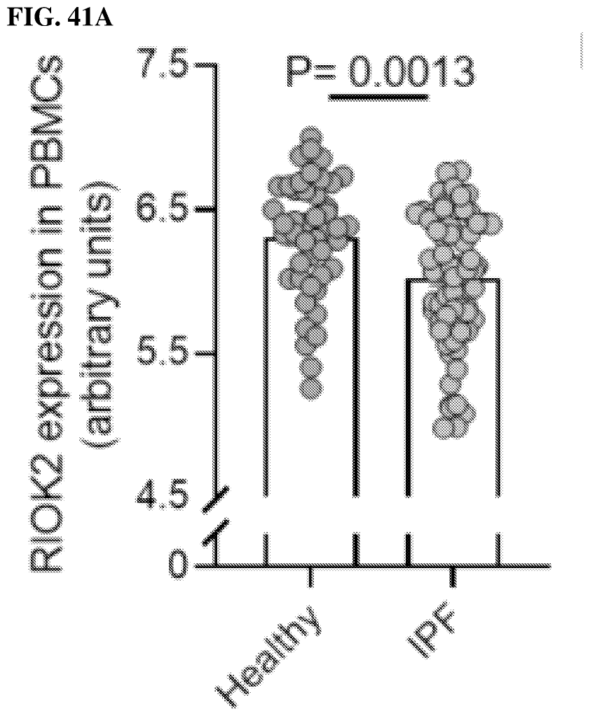

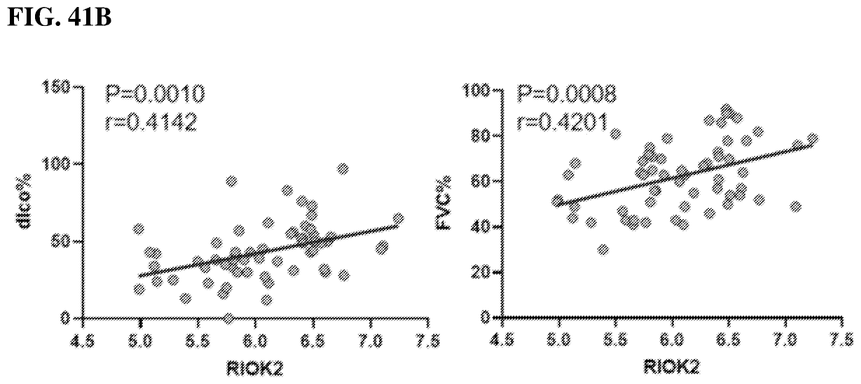

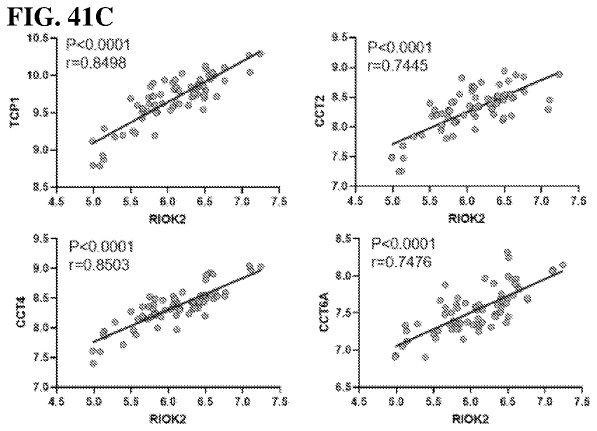

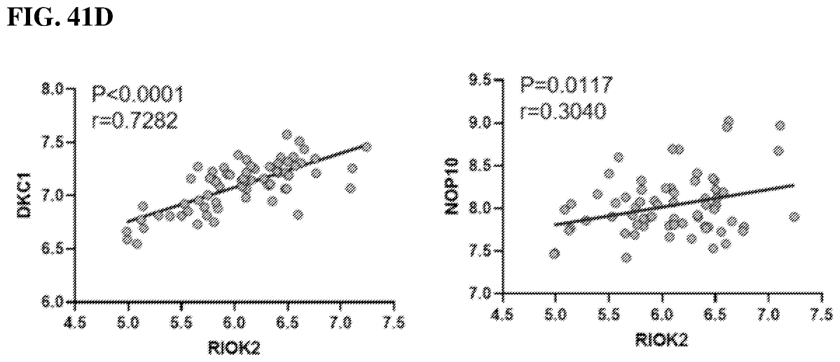

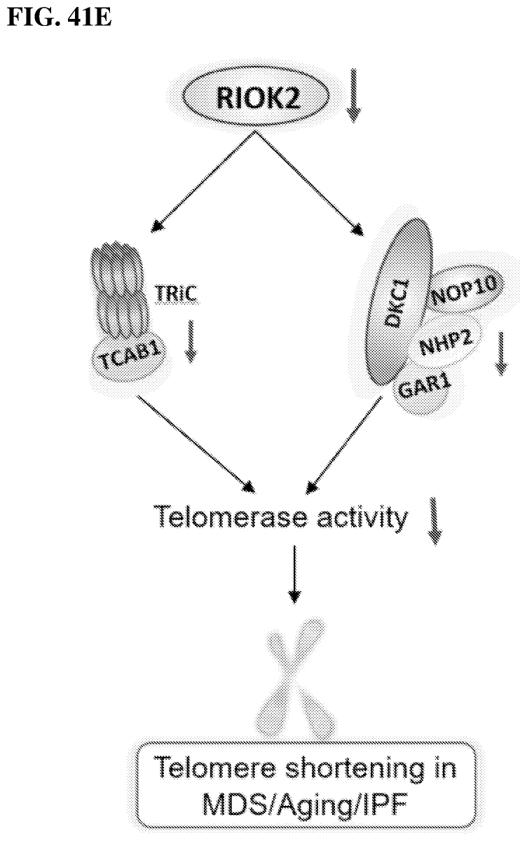

FIG. 41A-FIG. 41E show the mRNA expression of RIOK2 is decreased in IPF patients. FIG. 41A shows quantification of mRNA expression of RIOK2 in PBMCs derived from 45 healthy vs 70 IPF patients. FIG. 41B shows the correlation of mRNA expression of RIOK2 with dlco % and FVC % in PBMCs derived from 70 IPF patients. FIG. 41C shows the correlation of mRNA expression of RIOK2 with TRiC complex subunits in PBMCs derived from 70 IPF patients. FIG. 41D shows the correlation of mRNA expression of RIOK2 with dyskerin complex subunits in PBMCs derived from 70 IPF patients. FIG. 41E shows a graphical illustration depicting loss of RIOK2-mediated regulation of TRiC and dyskerin complexes underlies telomere shortening in MDS, IPF and aging individuals. Two-tailed Pearson's correlation was performed and shown in FIG. 41B-FIG. 41D. Pearson's correlation coefficients (r) and P values are shown in FIG. 41B-FIG. 41D. Unpaired non-parametric Mann-Whitney test is shown in FIG. 41A.

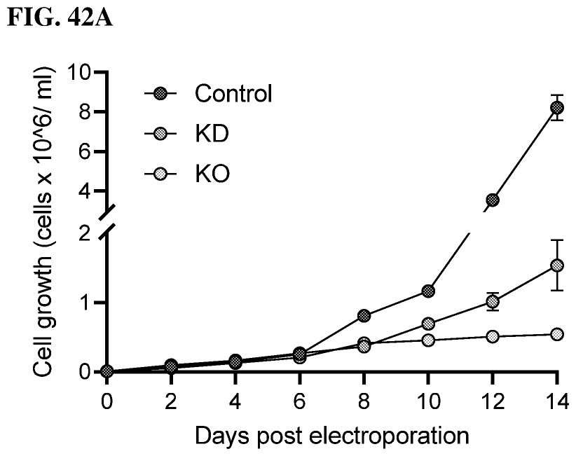

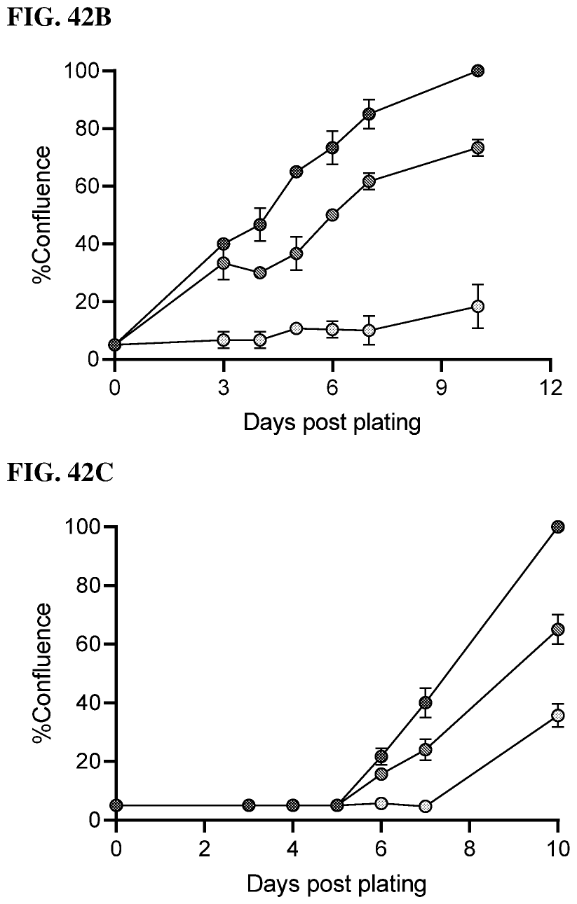

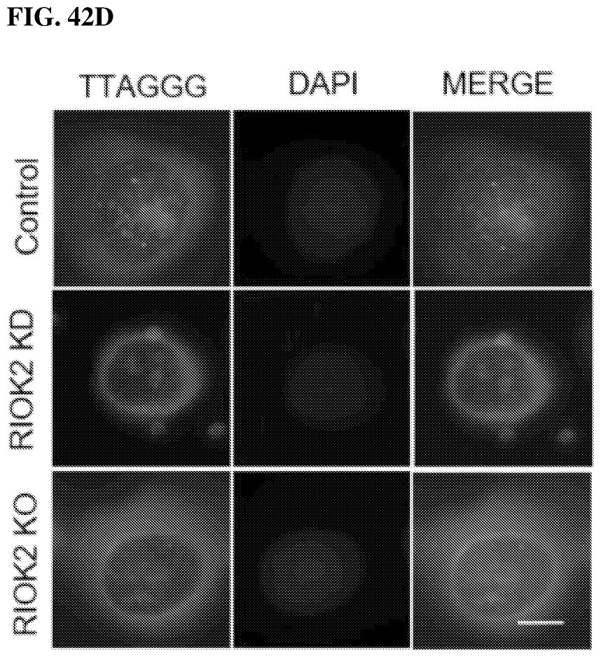

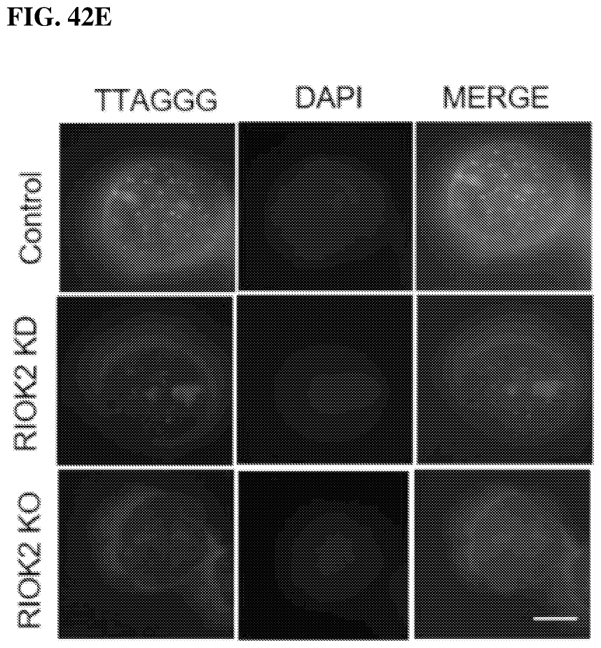

FIG. 42A-FIG. 42E show loss of RIOK2 in erythroid and non-erythroid cells results in telomere shortening. FIG. 42A shows a graph showing cell proliferation in K562 cells after knockdown (KD) and knockout (KO) of RIOK2. FIG. 42B shows a graph showing cell proliferation in Hela cells after knockdown (KD) and knockout (KO) of RIOK2. FIG. 42C is a graph showing cell proliferation in HEK293 cells after knockdown (KD) and knockout (KO) of RIOK2. FIG. 42D shows the fluorescence in-situ hybridization of telomeric DNA in Hela cells upon RIOK2 deficiency. FIG. 42E shows a fluorescence in-situ hybridization of telomeric DNA in HEK293 cells upon RIOK2 deficiency. Scale bar 10 μm.

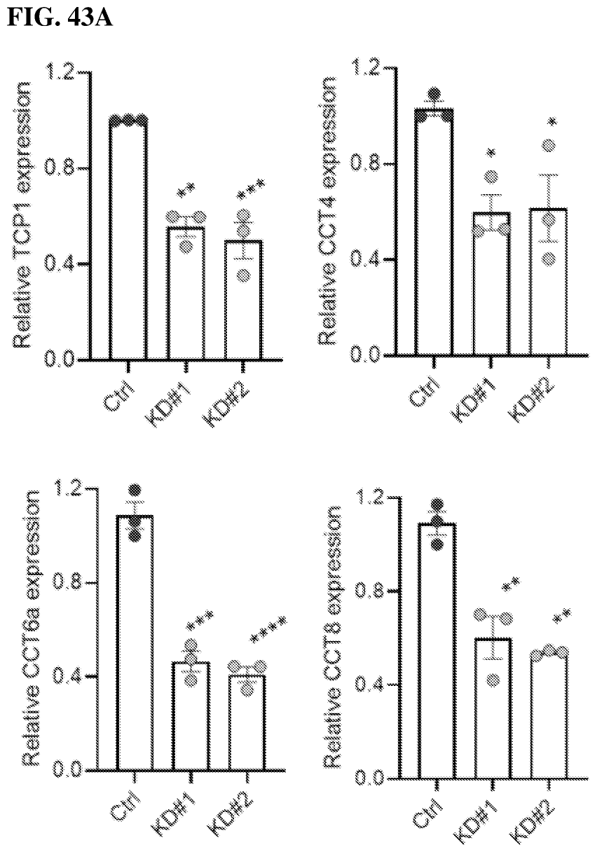

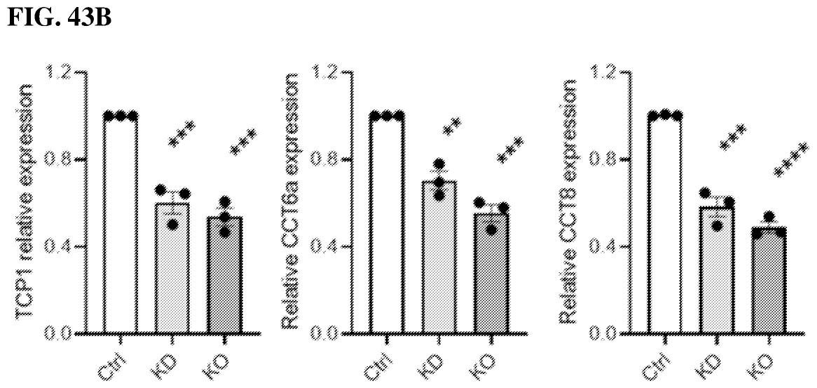

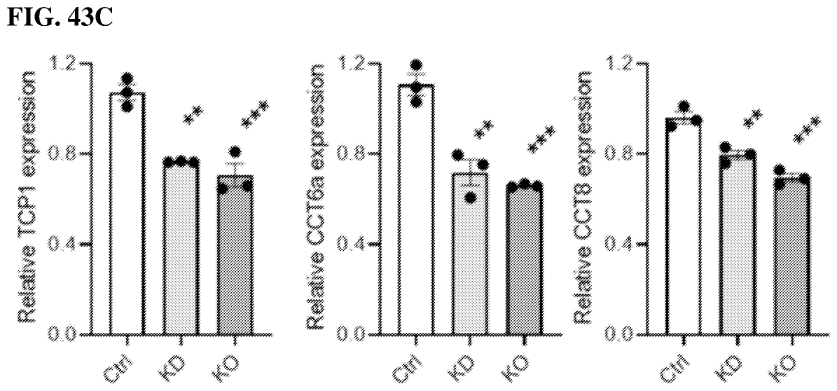

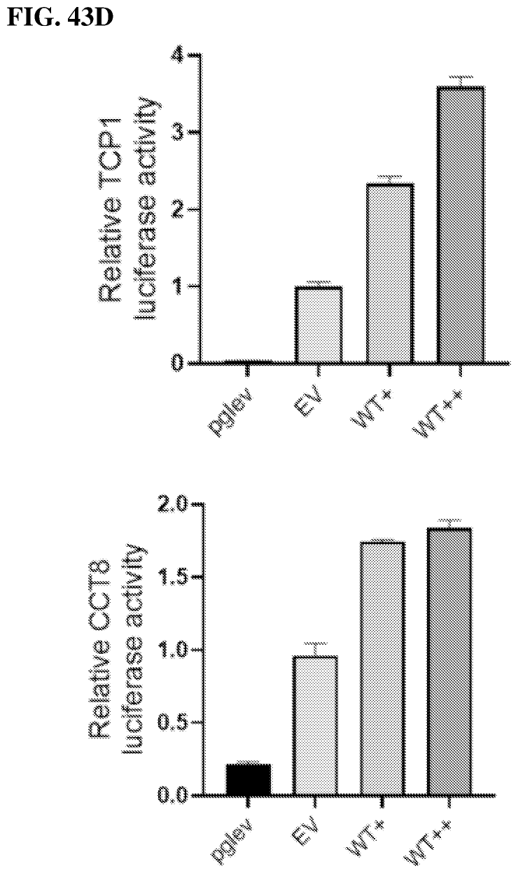

FIG. 43A-FIG. 43D show RIOK2 transcriptionally regulates TRiC complex expression. FIG. 43A shows TCP1 (CCT1), CCT4, CCT6, CCT8 mRNA expression (normalized to 0-actin) in TF-1 cells upon knockdown (KD) of RIOK2 using 2 different guide RNAs: KD #1, KD #2. FIG. 43B shows TCP1, CCT6A and CCT8 mRNA expression (normalized to 0-actin) in Hela cells upon KD and Knockout (KO) of RIOK2. FIG. 43C shows TCP1, CCT6A and CCT8 mRNA expression (normalized to 0-actin) in HEK293 cells upon KD and KO of RIOK2. FIG. 43D shows quantification of TCP1 and CCT8-promoter driven luciferase activity in response to dose dependently increasing RIOK2 expression in HEK293 cells; EV: empty vector. * p<0.05, ** p<0.01, *** p<0.001, **** p<0.0001, one-way analysis of variance (ANOVA). Data represented as mean±SEM. All comparisons are done w.r.t. control (Ctrl).

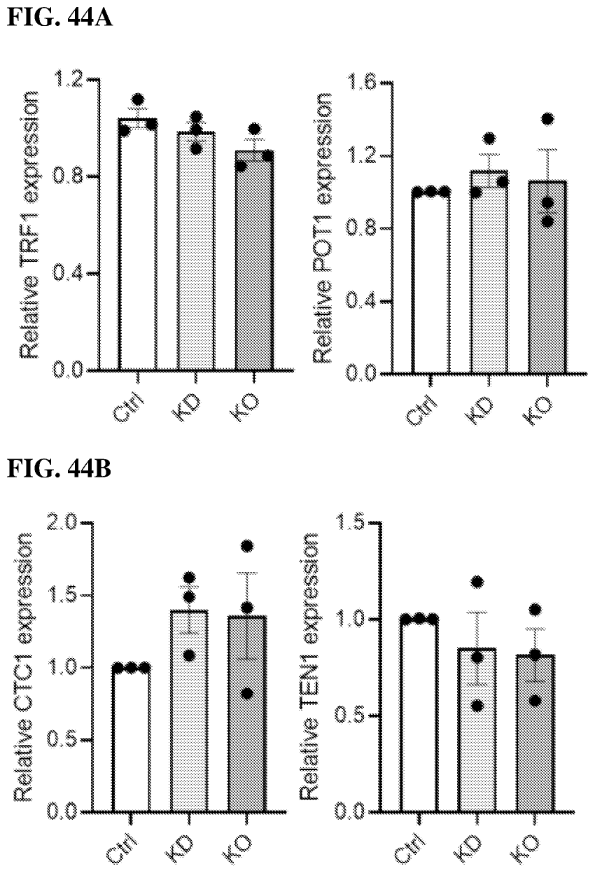

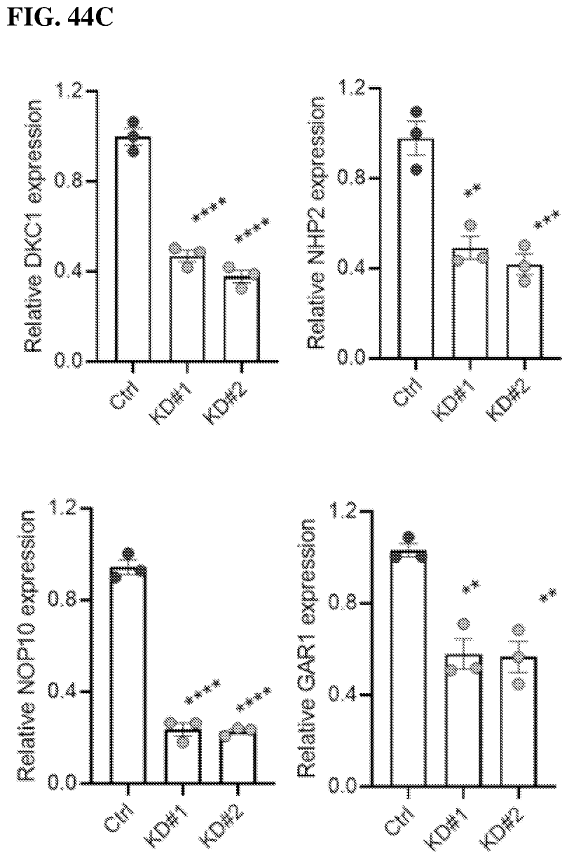

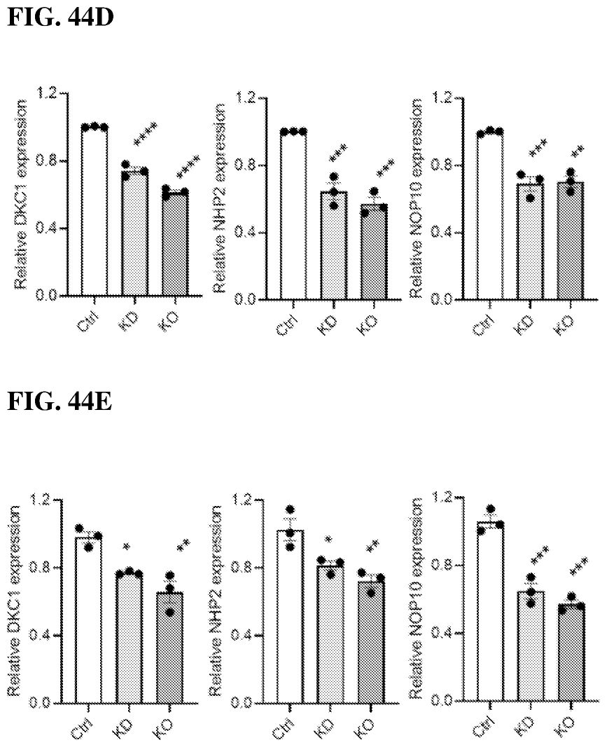

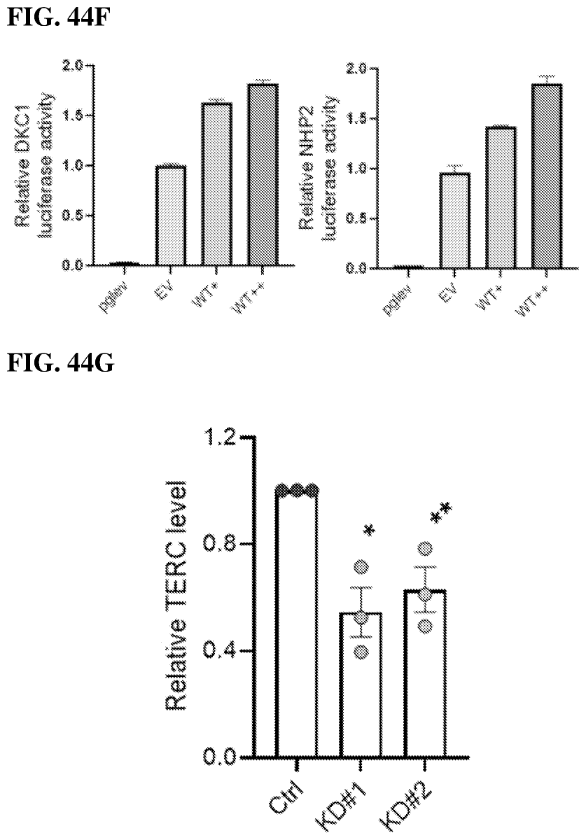

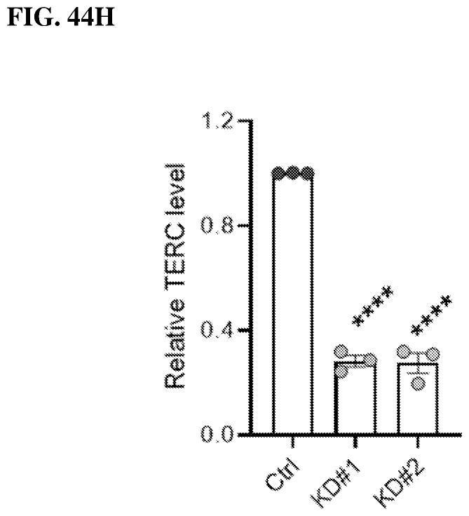

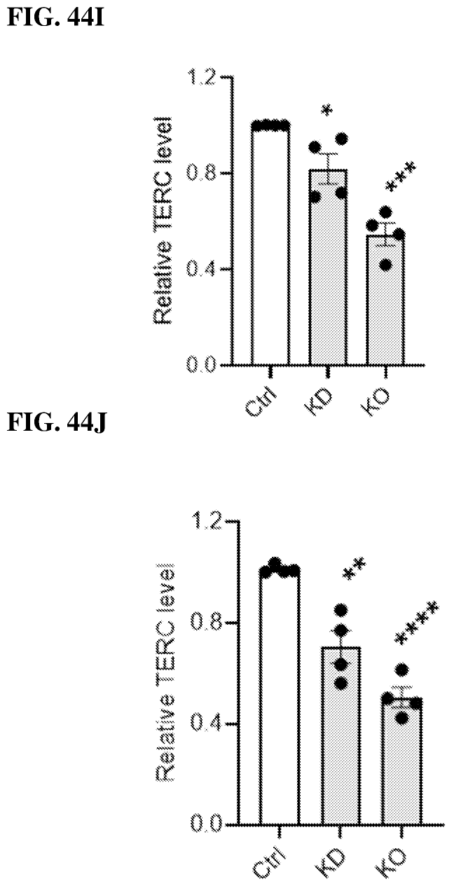

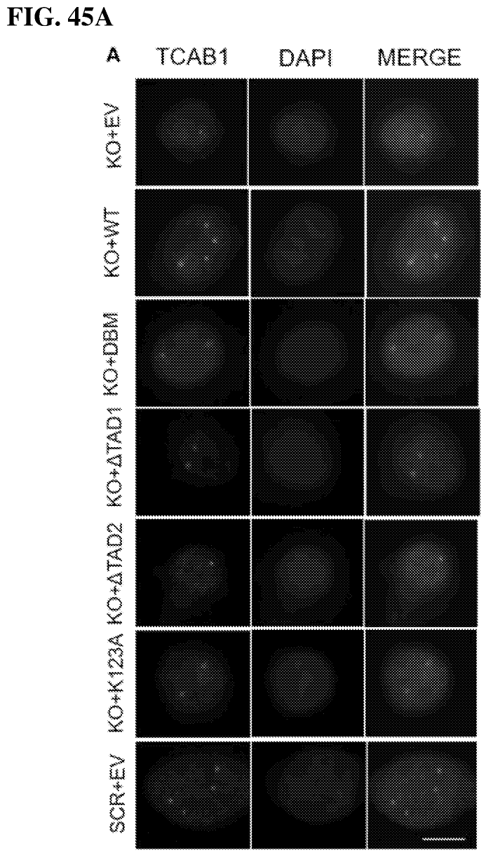

FIG. 44A-FIG. 44J show RIOK2 transcriptionally regulates dyskerin complex expression. FIG. 44A shows mRNA expression of shelterin complex subunits TRF1 and POT1 (normalized to 0-actin) in HSPCs upon knockdown (KD) and knockout (KO) of RIOK2. FIG. 44B shows mRNA expression of CST complex subunits CTC1 and TEN1 (normalized to 0-actin) in HSPCs upon knockdown (KD) and knockout (KO) of RIOK2. FIG. 44C shows mRNA expression of DKC1, NHP2, NOP10 and GAR1 (normalized to 0-actin) in TF-1 cells upon KD of RIOK2 using 2 different guide RNAs: KD #1, KD #2. FIG. 44D shows DKC1, NHP2 and NOP10 mRNA expression (normalized to 0-actin) in Hela cells upon KD and KO of RIOK2. FIG. 44E shows DKC1, NHP2 and NOP10 mRNA expression (normalized to 0-actin) in HEK293 cells upon KD and KO of RIOK2. FIG. 44F shows quantification of DKC1 and NHP2-promoter driven luciferase activity in response to dose dependently increasing RIOK2 expression in HEK293 cells; EV: empty vector. FIG. 44G and FIG. 44H shows quantification of expression of TERC (normalized to 28s RNA) upon KD of RIOK2 using 2 different guide RNAs: KD #1, KD #2, in TF-1 and K562 cells, respectively.

FIG. 441 and FIG. 44J show quantification of expression of TERC (normalized to 28s RNA) upon KD and KO of RIOK2 in Hela and HEK293 cells, respectively (* p<0.05, ** p<0.01, *** p<0.001, **** p<0.0001, one-way analysis of variance (ANOVA)). Data represented as mean±SEM. All comparisons are done w.r.t. control (Ctrl).

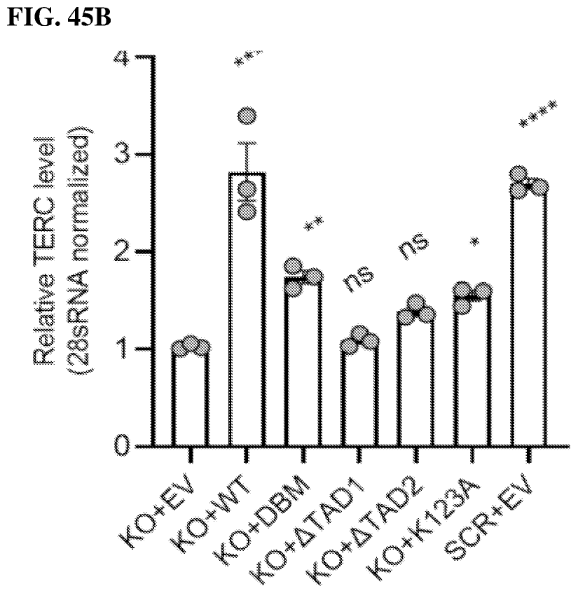

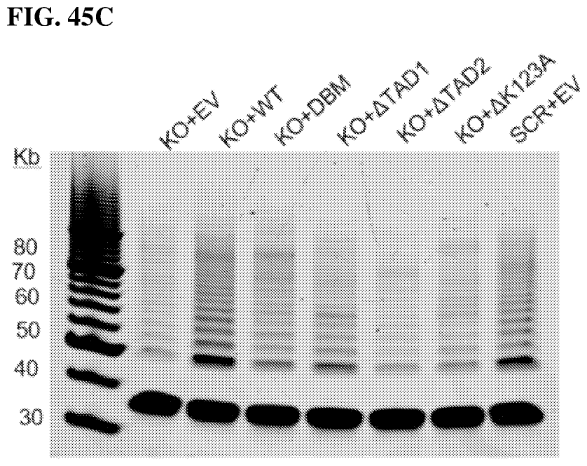

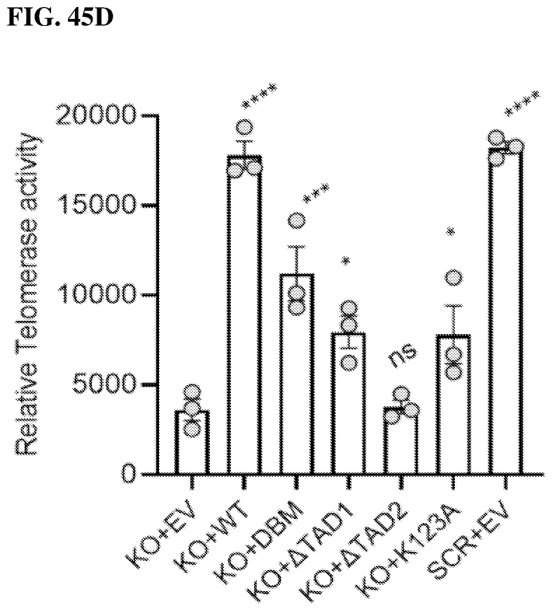

FIG. 45A-FIG. 45D show loss of transcriptional abilities of RIOK2 results in telomere shortening. FIG. 45A shows immunofluorescence staining showing expression of TCAB1 in RIOK2 KO K562 cells ectopically expressing EV, WT, DBM, ΔTAD1, ΔTAD2 or K123A RIOK2. FIG. 45B shows expression of TERC (normalized to 28s RNA) in RIOK2 KO K562 cells ectopically expressing EV, WT, DBM, ΔTAD1, ΔTAD2 or K123A RIOK2.

FIG. 45C shows a TRAP assay showing telomerase activity in RIOK2 KO K562 cells ectopically expressing EV, WT, DBM, ΔTAD1, ΔTAD2 or K123A RIOK2. FIG. 45D shows quantification of the TRAP assay shown in FIG. 45C (p<0.05, ** p<0.01, *** p<0.001, **** p<0.0001, one-way analysis of variance (ANOVA)). Data represented as mean±SEM. Scale bar 10 μm.

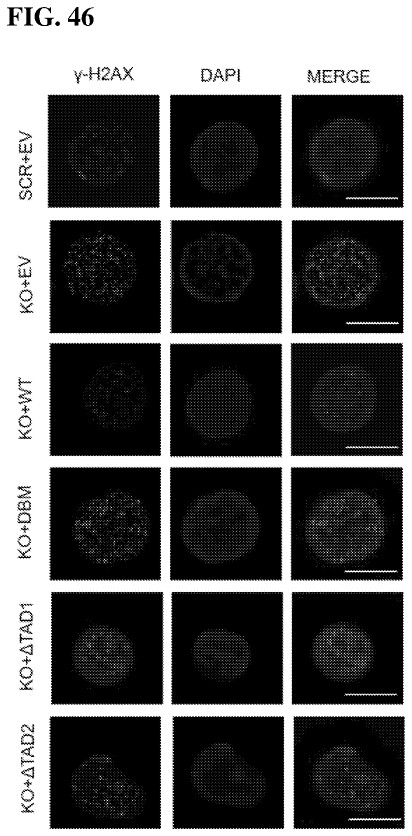

FIG. 46 shows loss of transcriptional abilities of RIOK2 triggers DNA-damage responses. Immunofluorescence images of γH2AX staining depicting DNA damage responses in RIOK2 KO TF-1 cells ectopically expressing EV, WT, DBM, ΔTAD1 or ΔTAD2 RIOK2. Scale bar 10 μm.

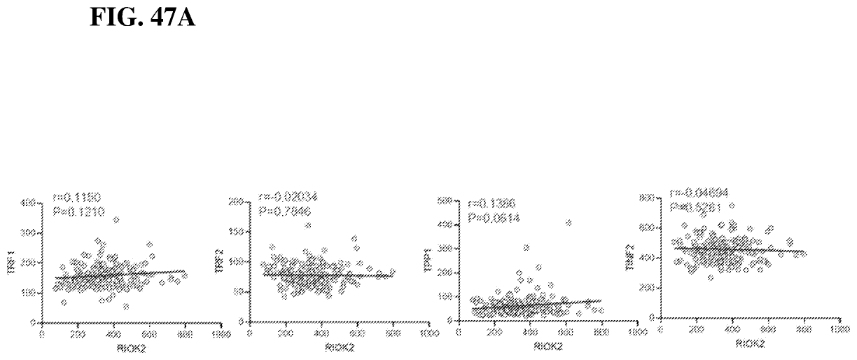

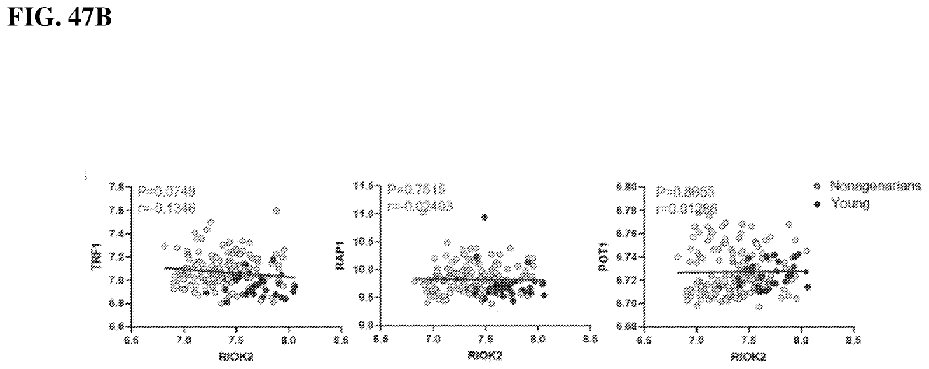

FIG. 47A-FIG. 47B show correlation of the mRNA expression of RIOK2 with shelterin complex subunits in MDS patients and aging individuals. FIG. 47A shows the correlation of mRNA expression of RIOK2 with shelterin complex subunits, TRF1, TRF2, TPP1, TINF2, in MDS patient-derived bone marrow cells; n=183. FIG. 47B shows the correlation of mRNA expression of RIOK2 with shelterin complex subunits, TRF1, RAP1, POT1, in PBMCs derived from 30 young (19-30 years) and 146 nonagenarians (>90 years).Two-tailed Pearson's correlation performed in FIG. 47A- FIG. 47B. Pearson's correlation coefficients (r) and P values are shown in FIG. 47A and FIG. 47B.

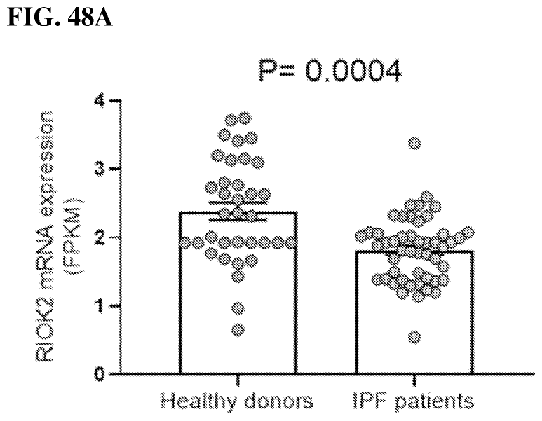

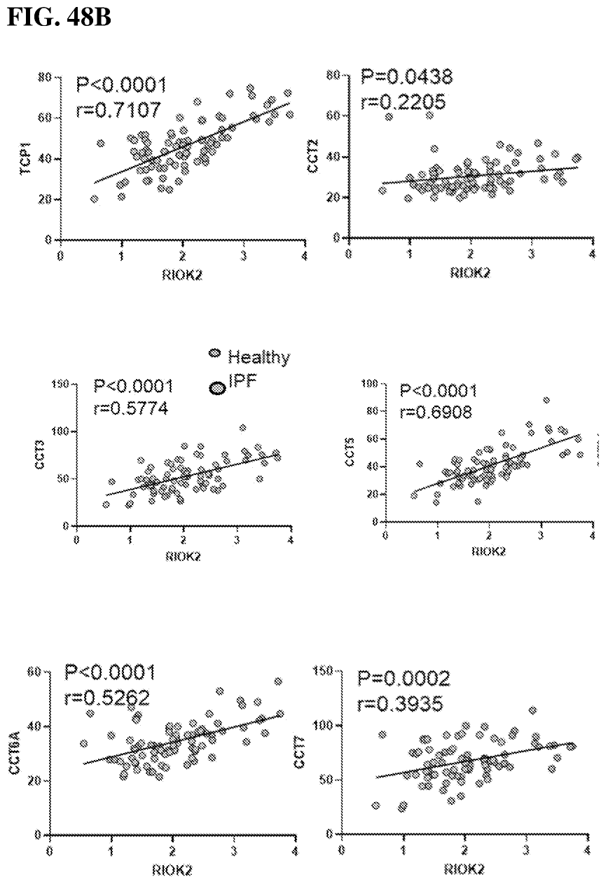

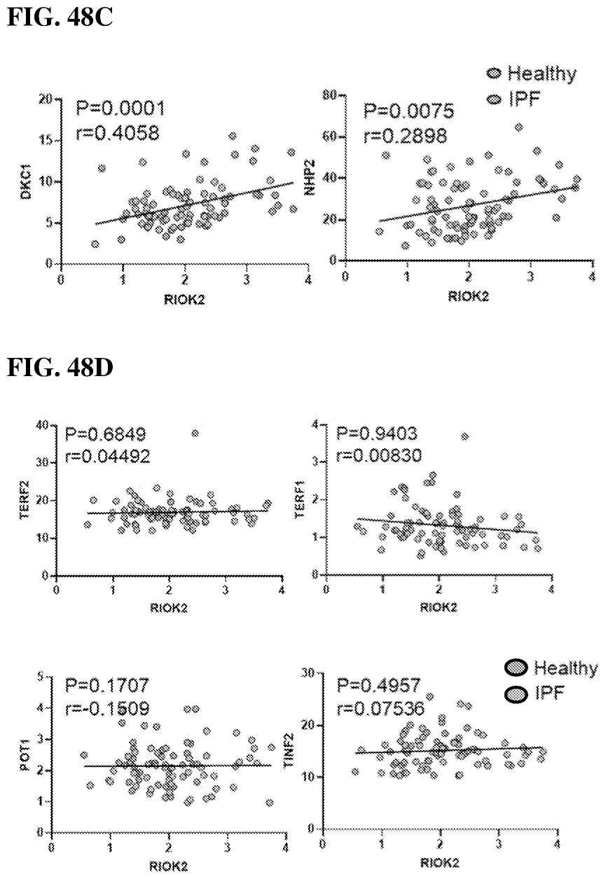

FIG. 48A-FIG. 48D show mRNA expression of RIOK2 is decreased in IPF patients.

FIG. 48A shows quantification of mRNA expression of RIOK2 in lung tissues derived from 35 healthy vs 49 IPF patients. FIG. 48B shows the correlation of mRNA expression of RIOK2 with TRiC complex subunits in lung tissues derived from 35 healthy and 49 IPF patients. FIG. 48C shows the correlation of mRNA expression of RIOK2 with dyskerin complex subunits in lung tissues derived from 35 healthy and 49 IPF patients. FIG. 48D shows the correlation of mRNA expression of RIOK2 with shelterin complex subunits in lung tissues derived from 35 healthy and 49 IPF patients. Two-tailed Pearson's correlation performed in FIG. 48B-FIG. 48D. Pearson's correlation coefficients (r) and P values are shown in FIG. 48B-FIG. 48D; Unpaired non-parametric Mann-Whitney test in FIG. 48A.

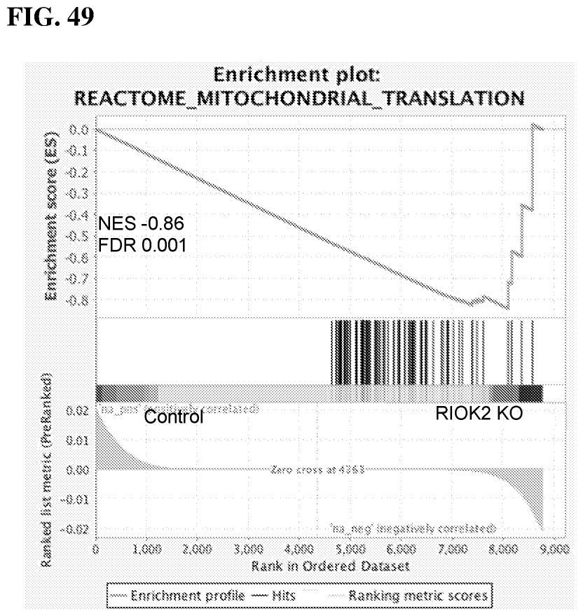

FIG. 49 shows a gene-set enrichment analysis of bulk RNA sequencing dataset showing reduced mitochondrial translation in RIOK2 knockout (KO) primary human HSPCs.

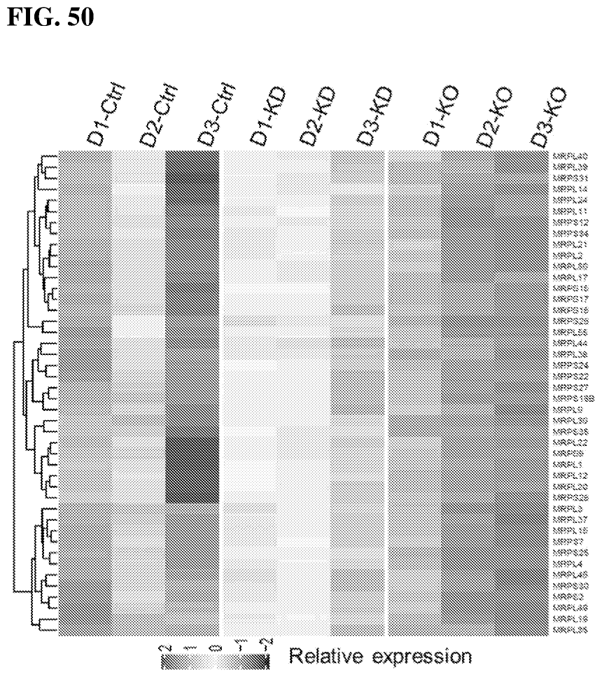

FIG. 50 shows a heat map showing dose-dependent differential expression of mitochondrial ribosomal protein (MRP) coding genes after knockdown (KD) and knockout (KO) of RIOK2 in primary human HSPCs; n=3 donors (D1,2,3), ctrl: control.

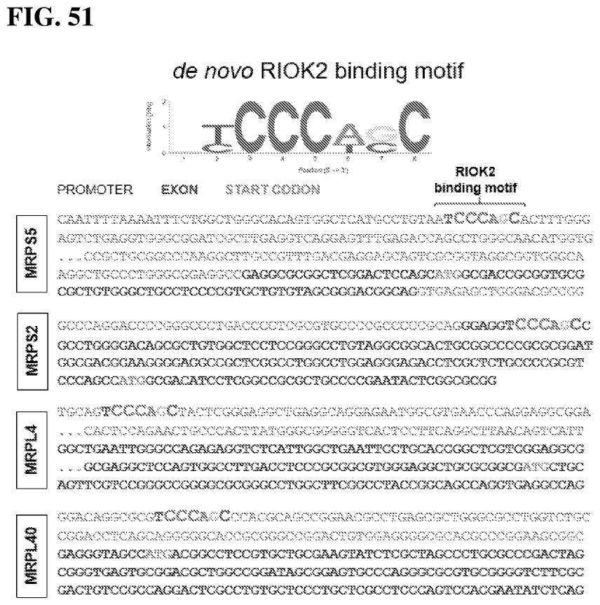

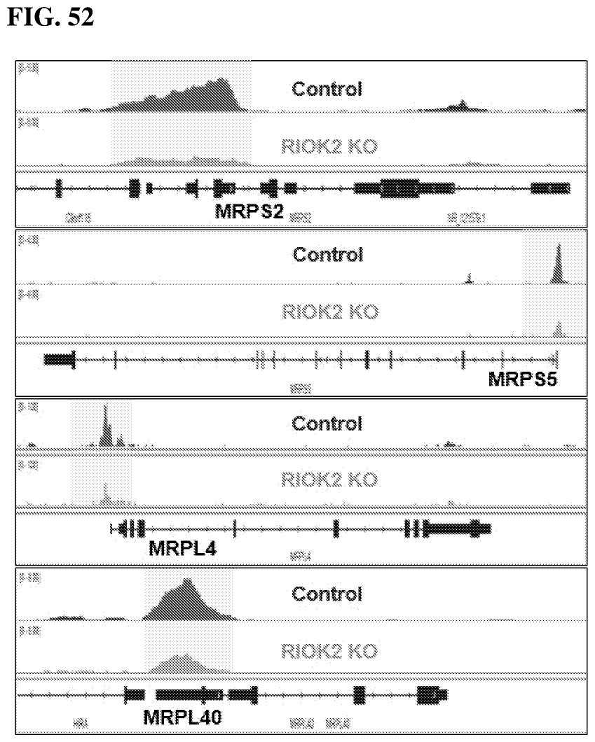

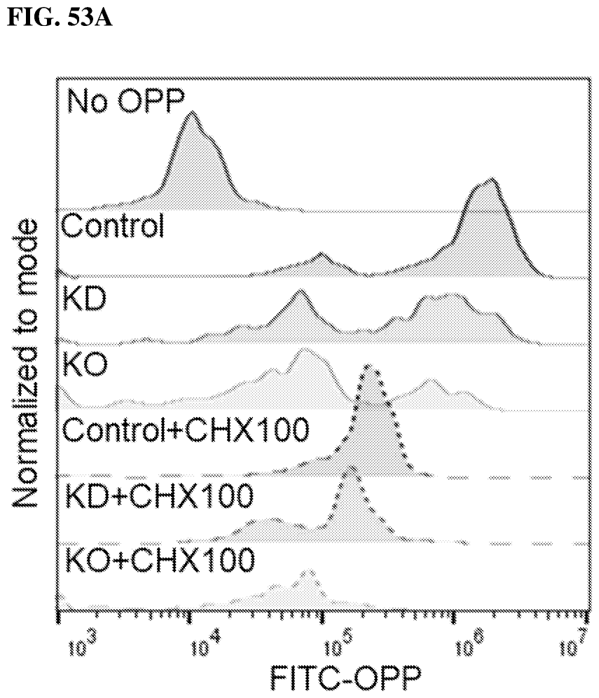

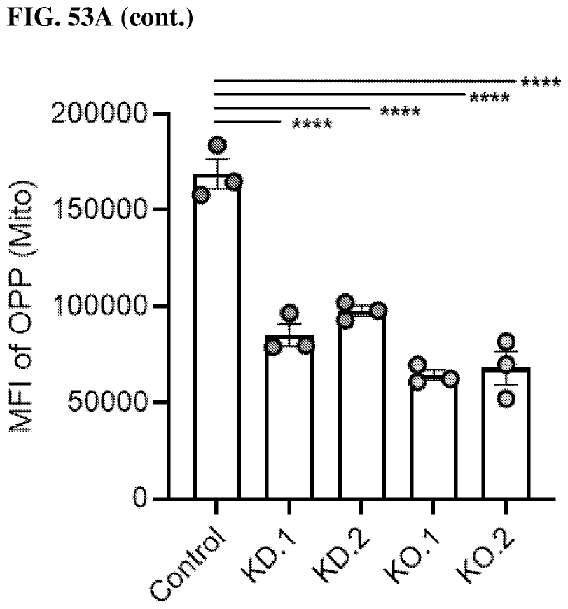

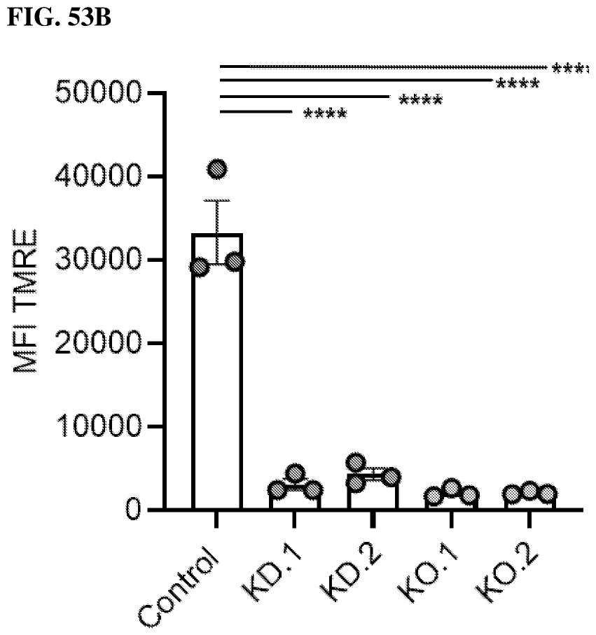

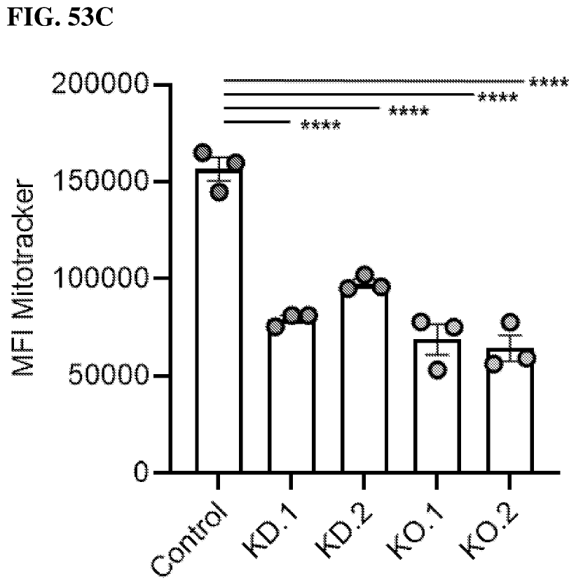

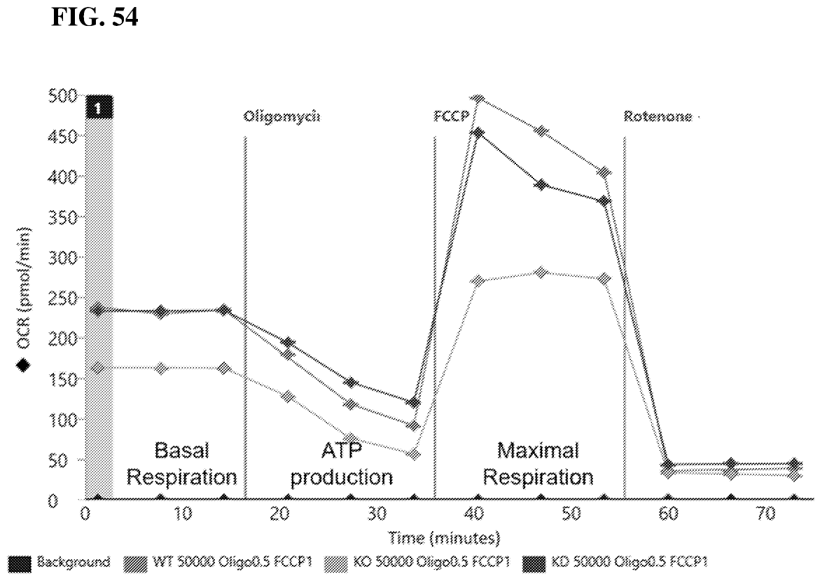

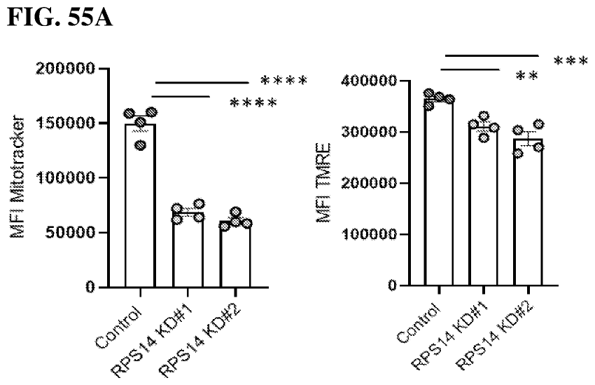

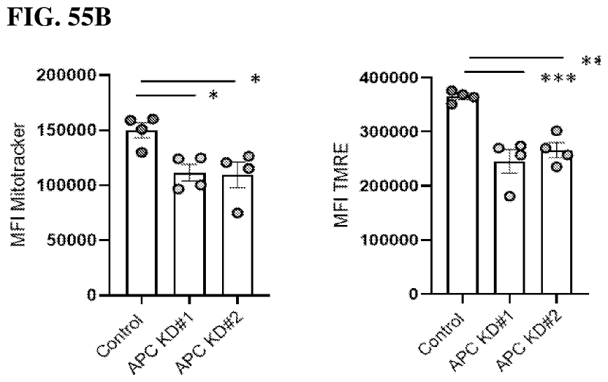

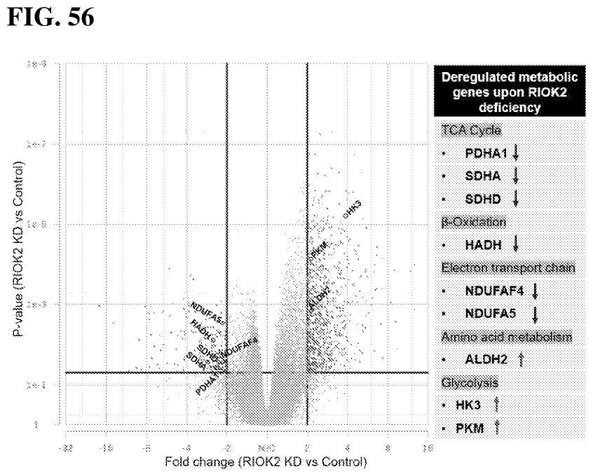

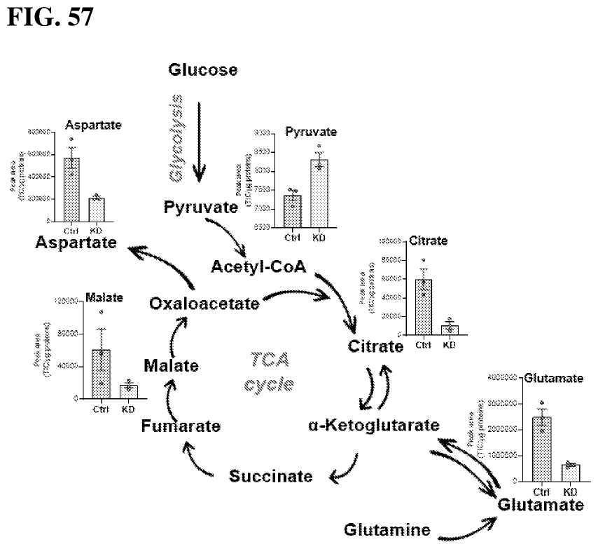

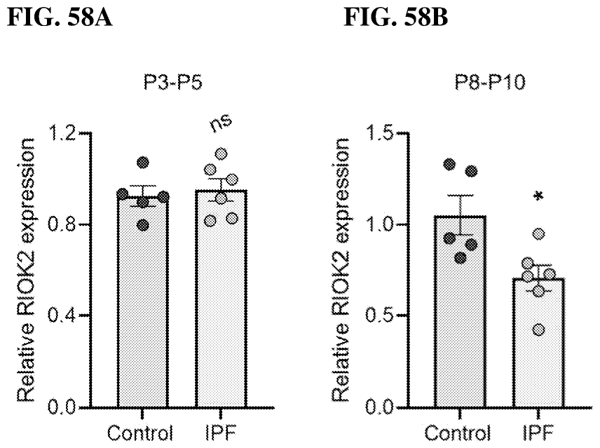

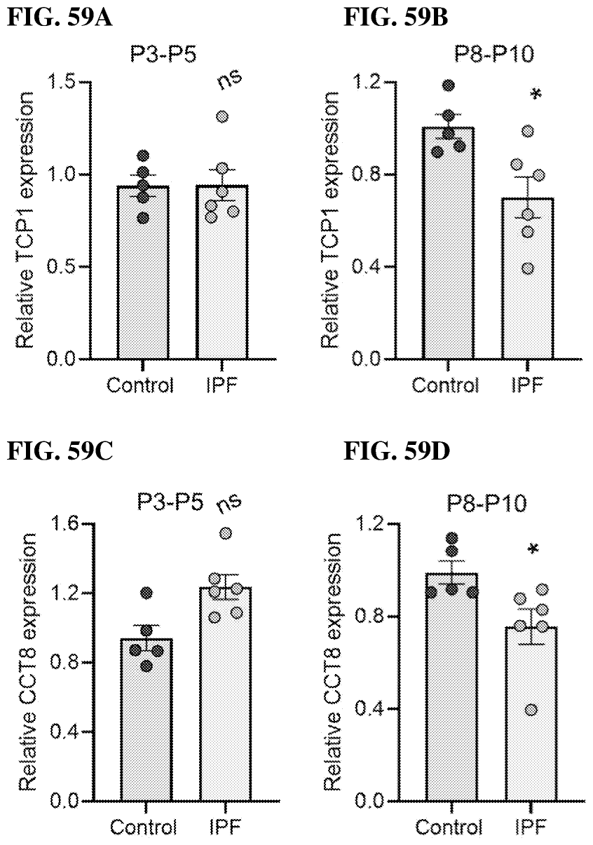

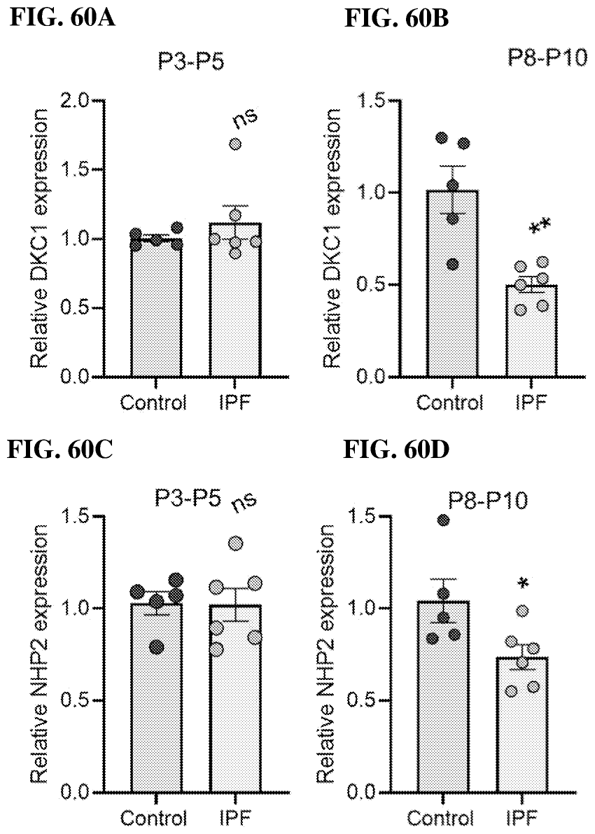

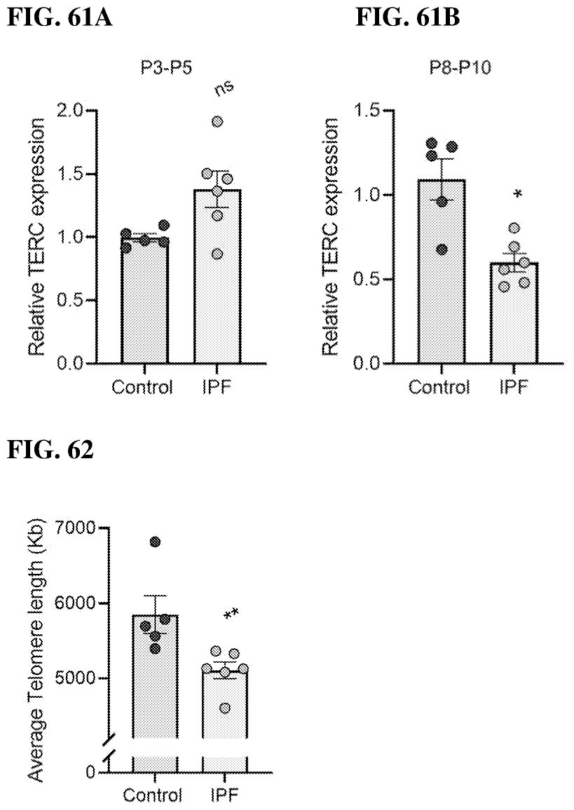

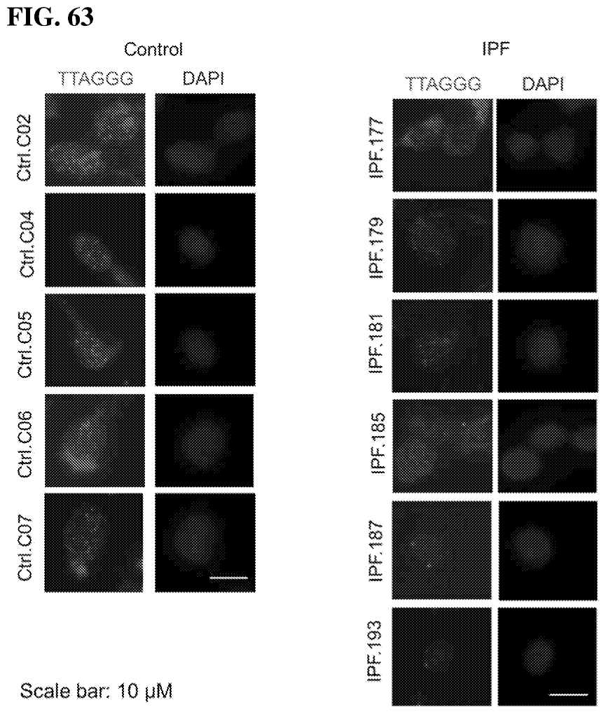

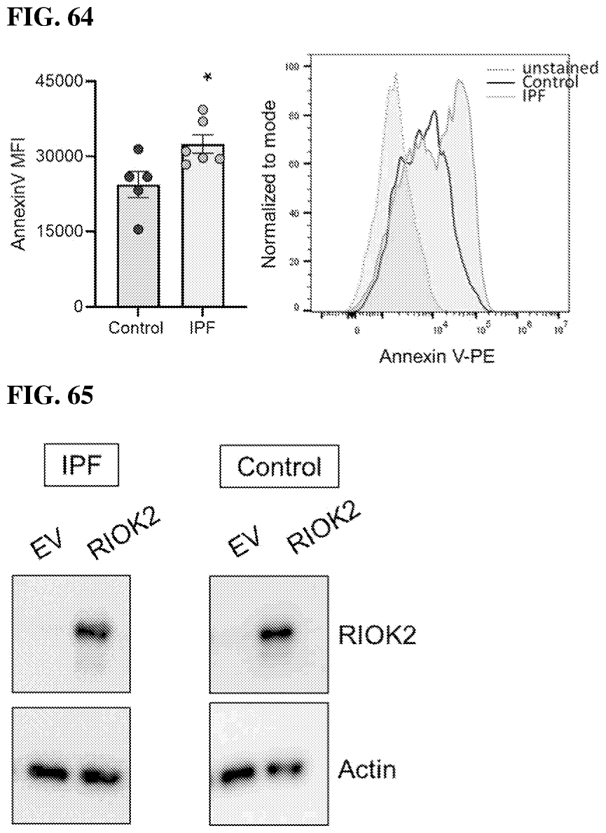

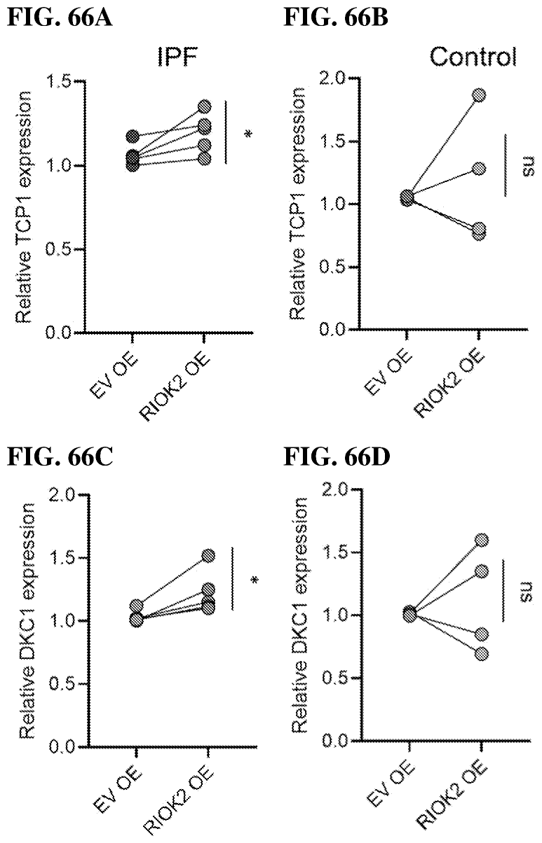

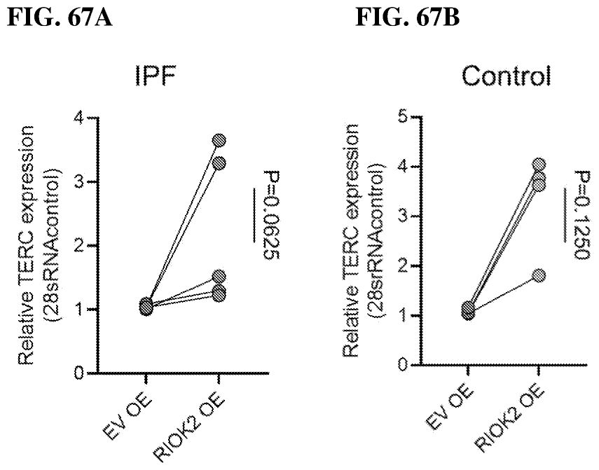

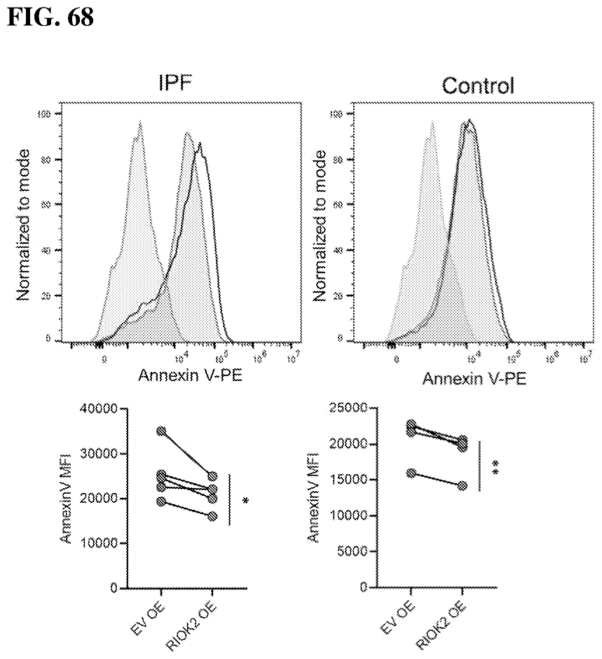

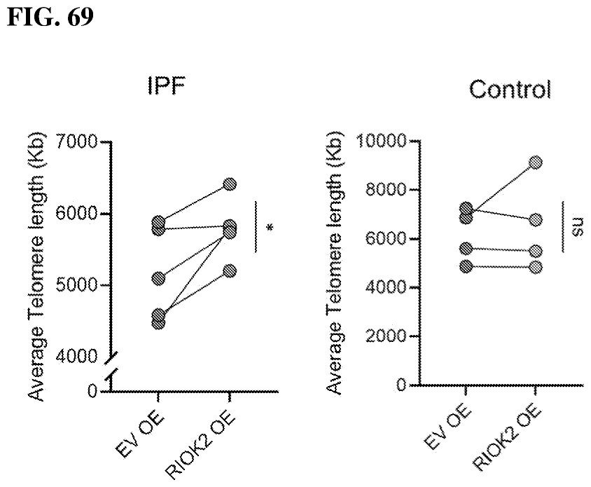

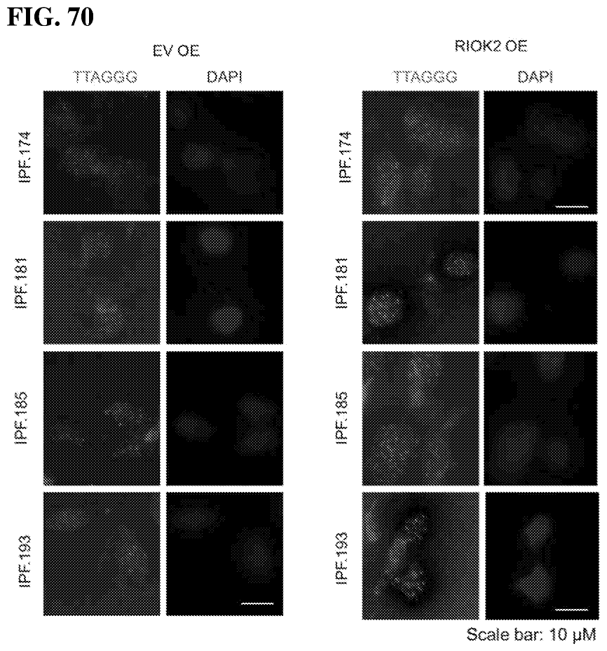

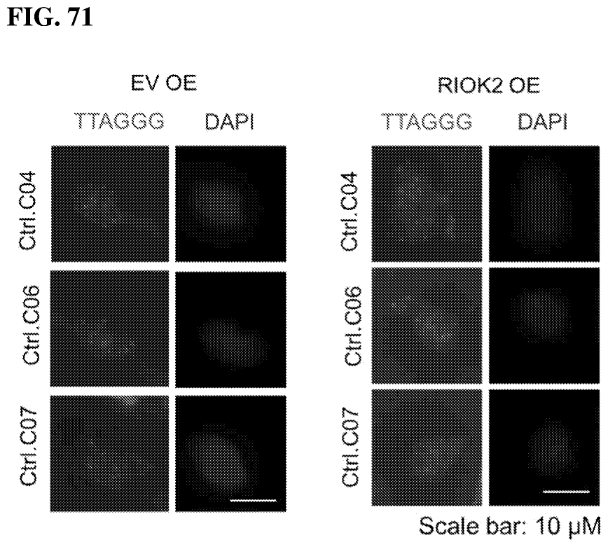

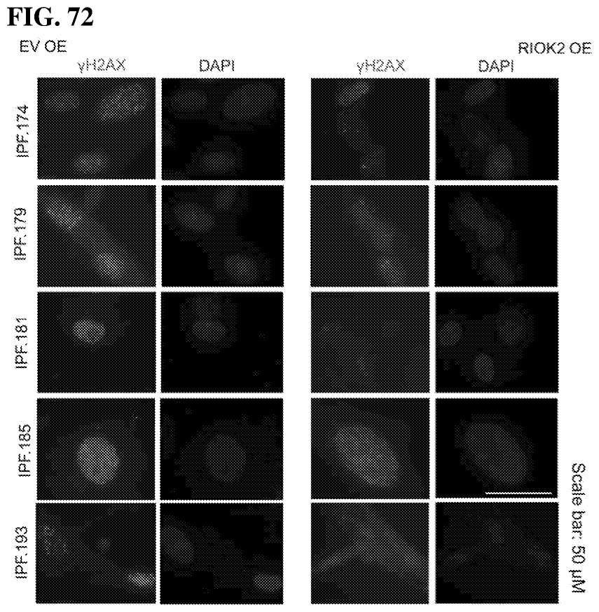

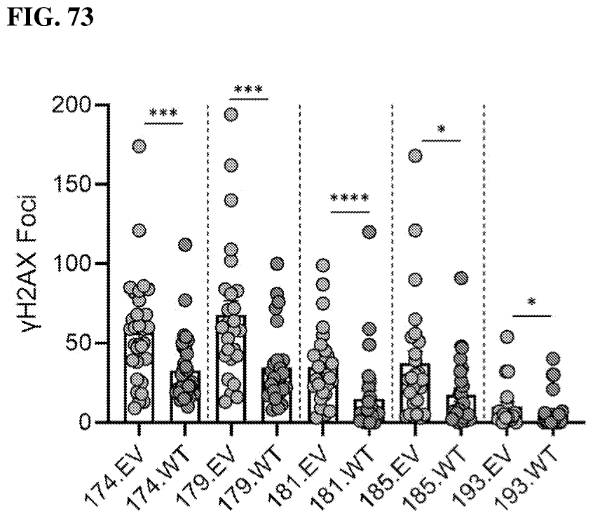

FIG. 51 shows the presence of de novo nucleotide binding motif specific for RIOK2 at the promoter regions of its putative mitochondrial target genes: MRPS5 (SEQ ID NO: 96), MRPS2 (SEQ ID NO: 97), MRPL4 (SEQ ID NO: 98), and MRPL40 (SEQ ID NO: 99).

FIG. 52 shows chromosome view plots depicting reduced chromatin accessibility at the promoter regions of MRPS2, MRPS5, MRPL4, MRPL40 in control vs. RIOK2 Knockout (KO) HSPCs.

FIG. 53A-FIG. 53C show loss of RIOK2 impedes mitochondrial biogenesis. FIG. 53A shows OPP incorporation depicting nascent protein synthesis in the presence (mitochondrial translation) and absence (cytoplasmic translation) of cycloheximide upon knockdown (KD) and knockout (KO) of RIOK2. FIG. 53B shows TMRE incorporation representing reduced dose-dependent mitochondrial membrane potential upon KD and KO of RIOK2. FIG. 53C shows the mitotracker incorporation showing decreased mitochondrial mass after KD and KO of RIOK2. ****p<0.0001, ANOVA. Data represented as mean±s.e.m.

FIG. 54 shows loss of RIOK2 impedes oxygen consumption rates (OCR) in viable RIOK2 KD and RIOK2 KO cells as compared to WT control cells, showing defective mitochondrial oxidative phosphorylation upon RIOK2 deficiency.

FIG. 55A-FIG. 55B show a deficiency of genes known to be involved in MDS results in compromised mitochondrial mass and membrane potential that are indicative of mitochondrial dysfunction. FIG. 53A shows RPS14 and FIG. 53B shows APC.

FIG. 56 shows RNA-sequencing analysis in RIOK2 knockdown (KD) vs control primary human HSPCs. The adjoining table shows differentially expressed metabolic genes upon deficiency of RIOK2. TCA: Tricarboxylic acid cycle.

FIG. 57 shows altered metabolite profiles assessed by targeted metabolomics of TCA cycle intermediates in control (Ctrl) vs RIOK2 knockdown (KD) HSPCs.

FIG. 58A-FIG. 58B show mRNA expression of RIOK2 is decreased in IPF patient-derived fibroblasts. FIG. 58A shows quantification of mRNA expression of RIOK2 in lung fibroblasts derived from 5 healthy controls vs. 6 IPF patients in early passage (labeled P3-P5). FIG. 58B shows quantification of the mRNA expression of RIOK2 in lung fibroblasts derived from 5 healthy controls vs. 6 IPF patients in late passage (labeled P8-P10). Two-tailed Student's t test in FIG. 58A and FIG. 58B. * p<0.05. Data represented as mean±SEM.

All comparisons are done w.r.t. control.

FIG. 59A-FIG. 59D show mRNA expression of TRiC complex subunits is decreased in IPF patient-derived fibroblasts. FIG. 59A and FIG. 59B show quantification of mRNA expression of TCP1 and CCT8 in lung fibroblasts derived from 5 healthy controls vs. 6 IPF patients in early passage (labeled P3-P5). FIG. 59C and FIG. 59D show quantification of mRNA expression of TCP1 and CCT8 in lung fibroblasts derived from 5 healthy controls vs 6 IPF patients in late passage (labeled P8-P10). Two-tailed Student's t test in FIG. 59A-FIG. 59D. * p<0.05. Data represented as mean±SEM. All comparisons are done w.r.t. control.

FIG. 60A-FIG. 60D show mRNA expression of Dyskerin complex subunits is decreased in IPF patient-derived fibroblasts. FIG. 60A and FIG. 60B show quantification of mRNA expression of DKC1 and NHP2 in lung fibroblasts derived from 5 healthy controls vs. 6 IPF patients in early passage (labeled P3-P5). FIG. 60C and FIG. 60D show quantification of the mRNA expression of DKC1 and NHP2 in lung fibroblasts derived from 5 healthy controls vs 6 IPF patients in late passage (labeled P8-P10). Two-tailed Student's t test in FIG. 60A-FIG. 60D. * p<0.05, ** p<0.01. Data represented as mean±SEM. All comparisons are done w.r.t. control.

FIG. 61A-FIG. 61B show TERC expression is decreased in IPF patient-derived fibroblasts. FIG. 61A show quantification of the RNA expression of TERC in lung fibroblasts derived from 5 healthy controls vs. 6 IPF patients in early passage (labeled P3-P5). FIG. 61B show quantification of the RNA expression of TERC in lung fibroblasts derived from 5 healthy controls vs. 6 IPF patients in late passage (labeled P8-P10). Two-tailed Student's t test in FIG. 61A and FIG. 61B. * p<0.05. Data represented as mean±SEM. All comparisons are done w.r.t. control.

FIG. 62 shows telomere length is decreased in IPF patient-derived fibroblasts.

Quantification of telomere lengths (from genomic DNA) was performed via qPCR technique in lung fibroblasts derived from 5 healthy controls vs. 6 IPF patients. Two-tailed Student's t test. ** p<0.01. Data represented as mean±SEM. All comparisons are done w.r.t. control.

FIG. 63 shows telomere length is decreased in IPF patient-derived fibroblasts. This figure shows fluorescence in-situ hybridization of telomeric DNA (telomeric puncta) in lung fibroblasts derived from 5 healthy controls and 6 IPF patients via in situ hybridization.

FIG. 64 shows apoptosis is increased in IPF patient-derived fibroblasts. Flow-plot and quantification of apoptosis was performed via Annexin V staining in lung fibroblasts derived from 5 healthy controls and 6 IPF patients. Two-tailed Student's t test. * p<0.05. Data represented as mean±SEM. All comparisons are done w.r.t. control.

FIG. 65 shows ectopic expression of RIOK2 in control and IPF patient-derived fibroblasts. Immunoblots show ectopic expression of RIOK2 (via lentiviral transduction) in lung fibroblasts derived from healthy control and IPF patient. EV stands for empty vector.

FIG. 66A-FIG. 66D show mRNA expression of TRiC and Dyskerin complex subunits is enhanced in IPF patient-derived fibroblasts upon ectopic expression of RIOK2. FIG. 66A-FIG. 66B show quantification of the mRNA expression of TCP1 and DKC1 in lung fibroblasts derived from 5 IPF patients with or without ectopic expression of RIOK2. FIG. 66C-FIG. 66D show quantification of the mRNA expression of TCP1 and DKC1 in lung fibroblasts derived from 4 healthy controls with or without ectopic expression of RIOK2. Two-tailed paired Student's t test in FIG. 66A- FIG. 66D. * p<0.05. Data represented as mean±SEM. All comparisons are done w.r.t. control. EV=empty vector, OE=overexpression.

FIG. 67A-FIG. 67B show RNA expression of TERC is mildly enhanced in IPF patient-derived fibroblasts upon ectopic expression of RIOK2. FIG. 67A-FIG. 67B show quantification of the RNA expression of TERC in lung fibroblasts derived from 5 IPF patients and 4 healthy (control) individuals with or without ectopic expression of RIOK2. Two-tailed paired Student's t test in FIG. 67A-FIG. 67B. Data represented as mean±SEM. All comparisons are done w.r.t. control. EV=empty vector, OE=overexpression.

FIG. 68 shows apoptosis is reduced in IPF patient-derived fibroblasts upon ectopic expression of RIOK2. Flow-plot and quantification of apoptosis was performed via Annexin V staining in lung fibroblasts derived from 4 healthy controls and 5 IPF patients upon ectopic expression of RIOK2. Two-tailed paired Student's t test. * p<0.05, ** p<0.01. Data represented as mean±SEM. All comparisons are done w.r.t. control. EV=empty vector, OE=overexpression.

FIG. 69 shows telomere length is increased in IPF patient-derived fibroblasts upon ectopic expression of RIOK2. Quantification of telomere lengths (from genomic DNA) via qPCR technique was performed in lung fibroblasts derived from 4 healthy controls and 5 IPF patients upon ectopic expression of RIOK2. Two-tailed paired Student's t test. * p<0.05.

Data represented as mean±SEM. All comparisons are done w.r.t. control. EV=empty vector, OE=overexpression.

FIG. 70 shows telomere length is increased in IPF patient-derived fibroblasts upon ectopic expression of RIOK2. Fluorescence in-situ hybridization of telomeric DNA (telomeric puncta) was performed in lung fibroblasts derived from 4 IPF patients upon ectopic expression of RIOK2.

FIG. 71 shows telomere length is not altered in healthy control-derived fibroblasts upon ectopic expression of RIOK2. This figure shows fluorescence in-situ hybridization of telomeric DNA (telomeric puncta) in lung fibroblasts derived from 3 healthy controls upon ectopic expression of RIOK2.

FIG. 72 shows ectopic expression of RIOK2 reduces DNA damage responses in IPF patient-derived fibroblasts. Immunofluorescence staining of y-H2AX foci (puncta-like staining shows DNA damage foci) was performed in lung fibroblasts derived from 5 IPF patients with or without ectopic expression of RIOK2.

FIG. 73 shows ectopic expression of RIOK2 reduces DNA damage responses in IPF patient-derived fibroblasts. Quantification of y-H2AX foci (puncta-like staining shows DNA damage foci) was performed in lung fibroblasts derived from 5 IPF patients with or without ectopic expression of RIOK2.

### DETAILED DESCRIPTION OF THE INVENTION

It has been determined herein that stabilization and/or an increase in the copy number, expression level, and/or activity of RIOK2 can prevent and/or treat a red blood cell disorder such as anemia (e.g., anemia associated with MDS or AML), a metabolic disorder, and/or aging and/or telomere shortening. It has also been determined herein that a decrease in the copy number, expression level, and/or activity of RIOK2 gene can treat or prevent polycythemia vera. Accordingly, the modulation of the level of RIOK2 provides an important treatment strategy for patients afflicted with aging, or another disease or disorder disclosed herein. Furthermore, the levels, expression of, or activity of, RIOK2 can serve as an indication for various diagnostic and prognostic methods described herein. It should be appreciated that, as used herein, reference to a “biomarker” includes RIOK2 peptides and nucleic acid sequences (Table 1), as well as the sequences listed in Table 2.

### I. DEFINITIONS

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid (e.g., any biomarker nucleic acid disclosed in Table 1), e.g., increased or decreased expression level in a biological sample, as compared to the expression level or copy number of the biomarker nucleic acid in a control sample. The term “altered amount” of a biomarker also includes an increased or decreased protein level of a biomarker protein in a sample, e.g., a biological sample, as compared to the corresponding protein level in a normal, control sample. Furthermore, an altered amount of a biomarker protein may be determined by detecting posttranslational modification such as glycosylation, ubiquitylation, phosphorylation, and/or proteolytic cleavage of the marker, which may affect the expression or activity of the biomarker protein (e.g., a biomarker nucleic acid disclosed in Table 1).

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or more than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, immune response (e.g., differentiation of a dendritic cell, T cell exhaustion, phagocytosis, etc.), cytotoxicity, cell growth, and the like.

The term “altered level of expression” of a biomarker refers to an expression level or copy number of the biomarker in a test sample, e.g., a sample derived from a patient suffering from aging or any disease or disorder disclosed herein, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a biological sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” refer to antigen-binding portions adaptable to be expressed within cells as “intracellular antibodies” or intrabodies (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., myelomas like multiple myeloma, Waldenstram's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), myeloma, multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue or cells isolated from a subject, such as a normal patient or the patient afflicted with a disease or disorder disclosed herein or a subject afflicted with aging, cultured primary cells/tissues isolated from a subject such as a normal subject or the afflicted patient, adjacent normal cells/tissues obtained from the same organ or body location of the afflicted patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care therapy). It will be understood by those of skill in the art that such control samples and reference standard expression product levels can be used in combination as controls in the methods of the present invention. In one embodiment, the control may comprise normal or non-diseased cell/tissue sample. In another preferred embodiment, the control may comprise an expression level for a set of patients, such as a set of afflicted patients, or for a set of afflicted patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another embodiment, the control may also comprise a measured value for example, average level of expression of a particular gene in a population compared to the level of expression of a housekeeping gene in the same population. Such a population may comprise normal subjects, afflicted patients who have not undergone any treatment (i.e., treatment naive), or afflicted patients undergoing standard of care therapy. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control.

In particularly preferred embodiments, the control comprises a control sample which is of the same lineage and/or type as the test sample. In another embodiment, the control may comprise expression product levels grouped as percentiles within or based on a set of patient samples. In one embodiment a control expression product level is established wherein higher or lower levels of expression product relative to, for instance, a particular percentile, are used as the basis for predicting outcome. In another preferred embodiment, a control expression product level is established using expression product levels from afflicted control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product.

Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with aging or a disease or disorder disclosed herein.

The term “determining a suitable treatment regimen for the subject” is taken to mean the determination of a treatment regimen (i.e., a single therapy or a combination of different therapies that are used for the prevention and/or treatment of aging, or a disease or disorder disclosed herein, in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. Expression data and gene expression levels can be stored on computer readable media, e.g., the computer readable medium used in conjunction with a microarray or chip reading device. Such expression data can be manipulated to generate expression signatures. “Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “erythroid progenitor cell” refers to the hematopoietic stem cell-derived progenitor cell that gives rise to erythrocytes (red blood cells) after terminal differentiation.

The term “RIOK2,” also known as Serine/Threonine-Protein Kinase RI02, refers to a serine/threonine-protein kinase involved in the final steps of cytoplasmic maturation of the 40S ribosomal subunit. It is involved in export of the 40S pre-ribosome particles (pre-40S) from the nucleus to the cytoplasm. Its kinase activity is required for the release of NOB1, PNO1 and LTV1 from the late pre-40S and the processing of 18S-E pre-rRNA to the mature 18S rRNA (PubMed:19564402). It regulates the timing of the metaphase-anaphase transition during mitotic progression, and its phosphorylation, most likely by PLK1, regulates this function.

The term “RIOK2” is intended to include fragments, variants (e.g., allelic variants) and derivatives thereof. The nucleic acid and amino acid sequences of a representative human RIOK2 is available to the public at the GenBank database (Gene ID 55781) and is shown in Table 1. The nucleic acids encoding RIOK2 and its alternatively spliced variants have been annotated in multiple NCBI Reference Sequences: NM_018343.3, NM_001159749.2, and XM_017009628.1.

Nucleic acid and polypeptide sequences of RIOK2 orthologs in organisms other than humans are well known and include, for example, chimpanzee RIOK2 (XM_009449398.3), dog RIOK2 (XM_038660744.1, XM_038660743.1, XM_038660742.1, XM_038660741.1, XM_038660740.1, XM_022416693.2, XM_022416692.2, XM_536291.6, XM_022416691.2, and XM_005618064.3), mouse RIOK2 (NM_025934.2), and Frog RIOK2 (NM_001016682.2), zebrafish RIOK2 (AY398407.1).

The term “RIOK2 activity” includes the ability of a RIOK2 polypeptide (and its fragments, domains, and/or motifs thereof, discussed herein) to bind DNA and/or modify its function.

The term “RIOK2-regulated pathway(s)” includes pathways in which the levels of RIOK2 (and its fragments, domains, and/or motifs thereof, discussed herein) affect TRiC and Dyskerin complex gene expression.

An agent that stabilizes and/or increases the copy number, expression level, amount, and/or activity of RIOK2 includes any natural or non-natural agent prepared, synthesized, manufactured, and/or purified by human that is capable of increasing, improving, and/or enhancing the ability of a RIOK2 polypeptide (and its fragments, domains, and/or motifs thereof, discussed herein). In some embodiments, the agent is a nucleic acid encoding RIOK2 or a fragment thereof (e.g., a biologically active fragment thereof). Importantly, the nucleic acid or a fragment thereof may encode a wild-type protein, or may comprise one or more mutations that enhances its activity. The mutation may be deletion, substitution, addition, or other chemical modifications. Such mutation may lack a negative regulatory domain, or confer increased interaction with one of its substrates. The nucleic acid may be transiently expressed in the target cell, or it may be integrated into the target cell genome.

Such nucleic acid may be DNA, RNA, or cDNA, and may be delivered by a viral particle (e.g., lentiviral particle or adeno-associated viral particle). In some embodiments, the agent may modulate the interaction between RIOK2 or a fragment thereof and one or more of its substrates and/or interacting partners. In some embodiments, the agent may decrease or reduce the turnover rate. In some embodiments, the agent may increase the stability of the mRNA and/or protein of RIOK2. In some embodiments, the agent comprises an internally cross linked peptide (e.g., a stapled peptide), a small molecule compound, a peptide, a polypeptide, an aptamer, an antibody or antigen-binding fragment thereof, an intrabody or antigen-binding fragment thereof, and/or a nucleic acid. An antibody or intrabody (including biparatopic or bispecific) or antigen-binding fragment thereof may stabilize or increase the interaction between RIOK2 and a substrate/interacting partner, thereby increasing its activity.

Purified RIOK2 proteins are commercially available (Cat. #TP760270) from Origene (Rockville, MD). Similarly, gene clones or ORF clones for human RIOK2 are commercially available (e.g., cat #RC201484) from Origene (Rockville, MD). In addition, gene clones or a fragment thereof in an expression vector or a viral vector (e.g., lentivirus, adenovirus, AAV (single-stranded AAV, self-complementary AAV), MMLV retrovirus, MSCV retrovirus, etc.) are commercially available from vendors such as Origene (Rockville, MD). Furthermore, anti-RIOK2 antibodies for various methods described herein are commercially available (cat. #TA505140, CF505140, and TA505177) from Origene (Rockville, MD).

An agent that decreases the copy number, expression level, amount, and/or activity of RIOK2 includes any natural or non-natural agent prepared, synthesized, manufactured, and/or purified by humans that is capable of reducing, inhibiting, blocking, preventing, and/or that inhibits a RIOK2 signaling pathway, including inhibition of a RIOK2 polypeptide (and its fragments, domains, and/or motifs thereof, discussed herein) directly. In one embodiment, such inhibitors may reduce or inhibit the binding/interaction between RIOK2 and its substrates or other binding partners. In another embodiment, such inhibitors may reduce or inhibit an upstream and/or downstream member of the RIOK2 signaling pathway. In still another embodiment, such inhibitors may increase or promote the turnover rate, reduce or inhibit the expression and/or the stability (e.g., the half-life) of RIOK2, resulting in at least a decrease in RIOK2 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to RIOK2 or also inhibit at least one RIOK2 signaling pathway member. RNA interference agents for RIOK2 polypeptides are well-known and commercially available (e.g., shRNA (Cat. TF320713, TL320713, TL320713V etc.) products, siRNA products (Cat. SR310938, SR417120, etc.), shRNA products (Cat. #sc-91773, shRNA Plasmids (sc-152973, etc.) and human or mouse gene knockout kit via CRISPR (Cat. c-405172, sc-426346) from Santa Cruz Biotechnology (Dallas, Texas).

The term “Dyskerin,” also known as dyskeratosis congenita 1, refers to a protein involved in telomerase stabilization and maintenance, as well as recognition of snoRNAs containing H/ACA sequences which provides stability during biogenesis and assembly into H/ACA small nucleolar RNA ribonucleoproteins (snoRNPs). This gene is highly conserved and widely expressed, and may play additional roles in nucleo-cytoplasmic shuttling, DNA damage response, and cell adhesion. Mutations have been associated with X-linked dyskeratosis congenita. Alternative splicing results in multiple transcript variants. Dyskerin is a protein involved in the formation of small nucleolar and small Cajal body ribonucleoproteins. These complexes participate in RNA pseudouridylation and are also components of the telomerase complex required for telomere elongation. Dyskerin is part of the Dyskerin complex, which is a key component of the telomerase enzyme. The Dyskerin complex is a 4-member group constituting Dyskerin (encoded by DKC1), NHP2, NOP10 and GAR1. Therefore, as used herein, when measuring the level or amount of Dyskerin, the method may comprise measuring any one of the Dysterin subunits, which includes any protein encoded by DKC1, NHP2, NOP10, and/or GAR1.

As used herein, a “red blood cell disorder” includes anemia, such as an anemia is selected from the group consisting of macrocytic anemia, anemia associated with inflammation such as chronic kidney disease (CKD) and other inflammatory diseases such as autoimmune disorders (e.g., rheumatoid arthritis and systemic lupus erythematosus), anemia caused by insufficiency of serine/threonine-protein kinase RIOK2, anemia caused by one or more mutations and/or deletions in human chromosome 5 or in an ortholog thereof, stress-induced anemia, aplastic anemia, Diamond Blackfan anemia, and Schwachman-Diamond syndrome. The anemia may be an anemia associated with a cancer, optionally wherein the cancer is a hematologic malignancy (e.g., myelodysplastic syndromes (MDS) or acute myeloid leukemia (AML)). In some embodiments, the red blood cell disorder is a disorder that is associated with increased megakaryopoiesis and/or myelopoiesis, such as myelodysplatic syndromes. The anemia may be an anemia associated with a bone marrow failure disorder.

The term “Dyskerin” is intended to include fragments, variants (e.g., allelic variants) and derivatives thereof. The nucleic acid and amino acid sequences of a representative human Dyskerin is available to the public at the GenBank database (CAB51168.1) and is shown in Table 2.

The term “TRiC,” also known as eukaryotic chaperonin TRiC (T-complex protein-1 Ring Complex, also known as CCT: Chaperonin Containing T-complex protein-1) plays an important role in ensuring efficient folding of nascent or stress-denatured proteins. TRiC interacts with approximately 10% of the entire proteome and its function is absolutely essential for viability of the cell. The cellular accumulation of misfolded protein has been associated with several human diseases, including Alzheimer's disease, Huntington's disease, and cancer. The nucleic acid and amino acid sequences of a representative TRiC is shown in Table 2. “Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold.

They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

The term “inhibit” includes to reduce, decrease, limitation, or blockage, of, for example a particular action, function, or interaction. In some embodiments, aging or a disease or disorder disclosed herein is “inhibited” if at least one symptom of the cancer is alleviated, terminated, slowed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized.

The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The terms “metabolic disorder” and “obesity related disorders” are used interchangeably herein and include a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can be characterized by a misregulation (e.g., downregulation or upregulation) of expression, structure, and/or expression of one or more biomarkers (including fragments thereof) and/or assays listed in Tables 1 and 2. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin response). Mitochondriopathies are also characterized as metabolic disorders. Mitochondrial defects have also been reported in subjects with MDS.

Examples of metabolic disorders include obesity, including insulin resistant obesity, diabetes, noninsulin dependent diabetes mellitus (NIDDM or Type H diabetes), insulin dependent diabetes mellitus (IDDM or Type I diabetes), type II diabetes, insulin resistance such as impaired glucose tolerance, glucose intolerance, atherosclerosis, atheromatous disease, heart disease, hypertension, stroke, Syndrome X, hyperphagia, endocrine abnormalities, triglyceride storage disease, Bardet-Biedl syndrome, Lawrence-Moon syndrome, Prader-Labhart-Willi syndrome, Werner's syndrome, dysfunctions associated with lipid biosynthesis, lipid transport, triglyceride levels, plasma levels, and plasma cholesterol, dyslipidemias associated with hyperlipidemia, elevated free fatty acids, hypercholesterolemia, hypertriglyceridemia, elevated low density lipoprotein-(LDL)-cholesterol, elevated very low density lipoprotein-(VLDL)-cholesterol, elevated intermediate density lipoprotein-(IDL)-cholesterol, or reduced high density lipoprotein-(HDL)-cholesterol.

A metabolic disorder (e.g., diabetes and/or obesity) is “inhibited” if at least one symptom of the metabolic disorder (e.g., diabetes and/or obesity) is alleviated, terminated, slowed, or prevented. As used herein, a metabolic disorder (e.g., diabetes and/or obesity) is also “inhibited” if recurrence or metastasis of the metabolic disorder (e.g., diabetes and/or obesity) is reduced, slowed, delayed, or prevented.

In addition, metabolic disorders are associated with one or more discrete phenotypes.

For example, body mass index (BMI) of a subject is defined as the weight in kilograms divided by the square of the height in meters, such that BMI has units of kg/m². Overweight is defined as the condition wherein the individual has a BMI greater than or 25 kg/m² and less than 30 kg/m². In some embodiments, obesity is defined as the condition wherein the individual has a BMI equal to or greater than 30 kg/m². In another aspect, the term obesity is used to mean visceral obesity which can be defined in some embodiments as a waist-to-hip ratio of 1.0 in men and 0.8 in women, which, in another aspect defines the risk for insulin resistance and the development of pre-diabetes. Euglycemia is defined as the condition in which a subject has a fasting blood glucose concentration within the normal range, greater than 70 mg/dl (3.89 mmol/L) and less than 110 mg/dl (6.11 mmol/L). The word fasting has the usual meaning as a medical term. Impaired glucose tolerance (IGT), is defined as the condition in which a subject has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). The term impaired glucose tolerance is also intended to apply to the condition of impaired fasting glucose. Hyperinsulinemia is defined as the condition in which a subject with insulin resistance, with or without euglycemia, in which the fasting or postprandial serum or plasma insulin concentration is elevated above that of normal, lean individuals without insulin resistance, having a waist-to-hip ration <1.0 (for men) or <0.8 (for women). The terms “diabetes”, “prediabetes”, and “insulin-sensitizing”, “insulin resistance-improving” or “insulin resistance-lowering” (used interchangeably herein) have been described herein.

As used herein, “metabolic syndrome” refers to a condition present when more than one of these factors are present in a single individual. The factors include: central obesity (disproportionate fat tissue in and around the abdomen), atherogenic dyslipidemia (these include a family of blood fat disorders including, e.g., high triglycerides, low HDL cholesterol, and high LDL cholesterol that can foster plaque buildups in the vascular system, including artery walls), high blood pressure (130/85 mmHg or higher), insulin resistance or glucose intolerance (the inability to properly use insulin or blood sugar), a chronic prothrombotic state (e.g., characterized by high fibrinogen or plasminogen activator inhibitor [−1]levels in the blood), and a chronic proinflammatory state (e.g., characterized by higher than normal levels of high-sensitivity C-reactive protein in the blood). In some embodiments, the “Third Report of the National Cholesterol Education Program (NCEP) Expert Panel on Detection, Evaluation, and Treatment of High Blood Cholesterol in Adults (Adult Treatment Panel III)” may be used in the diagnosis of a metabolic disorder. Under the NCEP criteria, metabolic syndrome can be clinically identified by the presence of three or more of the following components in a single subject: (1) central obesity, as measured by waist circumference (women with a waist circumference greater than 35 inches; for men greater than 40 inches); (2) fasting blood triglycerides greater than or equal to 150 mg/dL; (3) blood HDL cholesterol (for women less than 50 mg/dL, for men less than 40 mg/dL); (4) blood pressure greater than or equal to 130/85 mmHg; and (5) fasting glucose greater than or equal to 110 mg/dL. Other features such as insulin resistance (e.g., increased fasting blood insulin), prothrombotic state or proinflammatory state are not generally required for clinical diagnosis, though they are certainly also indicative of metabolic syndrome and follow-up studies on these attributes can be used to further confirm diagnosis of metabolic syndrome.

For example, insulin resistance, even in the absence of the NCEP criteria, is often indicative of metabolic syndrome.

As used herein, the term “mitochondriopathy” refers to any disease or disorder associated with one or more mitochondrial defects. They may be the result of either de novo or inherited mutations in nuclear or mitochondrial DNA located genes, or due to exogenous factors. These disorders are usually exhibit a chronic, slowly progressive course and present with multiorgan involvement with varying onset between birth and late adulthood. Additional information regarding mitochondriopathies can be found at Finsterer J. Mitochondriopathies. Eur J Neurol. 2004 March;11(3):163-86. doi: 10.1046/j.1351-5101.2003.00728.x. PMID: 15009163.

As used herein, myelodysplastic syndromes (MDS), include, but are not limited to, a heterogeneous group of myeloid neoplasms, which are characterized in common by manifestations of bone marrow failure with abnormal cell morphology and, in some cases, a propensity to acute myeloid leukemia (AML). Telomere shortening may be associated with an MDS disclosed herein.

The “normal” level of expression of a biomarker is the level of expression of the biomarker in cells of a subject, e.g., a human patient, not afflicted with aging or a disease or disorder disclosed herein. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) may be a biomarker amount and/or activity measurement(s) used to, by way of example only, evaluate a subject that may be selected for a particular treatment. A pre-determined biomarker amount and/or activity measurement(s) may be determined in populations of patients with or without a disease or disorder disclosed herein. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or under expression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with aging or a disease or disorder disclosed herein; (3) its absolute or relatively modulated presence or absence in clinical subset of afflicted patients (e.g., those responding to a particular inhibitor/immunotherapy combination therapy or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of a disease or the likelihood of recovery from the disease. In some embodiments, the use of statistical algorithms provides a prognosis of a disease in an individual.

The terms “response” or “responsiveness” refers to an anti-disease response. The terms can also refer to an improved prognosis or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of a nucleic acid expression” includes any decrease in expression or protein activity or level of the nucleic acid or protein encoded by the nucleic acid. The decrease may be of at least 30%, 40%, 50%, 60%, 70%, 80%, 90%, 95% or 99% or more as compared to the expression of a nucleic acid or the activity or level of the protein encoded by the nucleic acid which has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample. “Short interfering RNA” (siRNA), also referred to herein as “small interfering RNA” is defined as an agent which functions to inhibit expression of a nucleic acid, e.g., by RNAi.

An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr;9(4):493-501 incorporated by reference herein).

RNA interfering agents, e.g., siRNA molecules, may be administered to an aging patient or a patient having or at risk for having a disease or disorder disclosed herein, to inhibit expression of a biomarker gene which is overexpressed in disease or disorder and thereby treat, prevent, or inhibit the disease or disorder in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10-⁷ M, such as approximately less than 10-8 M, 10-9 M or 10-¹⁰ M or even lower when determined by surface plasmon resonance (SPR) technology in a BIACORE®assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a disease or disorder disclosed herein or an aging patient.

The term “subject” is interchangeable with “patient.” The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival. The length of said survival may be calculated by reference to a defined start point (e.g. time of diagnosis or start of treatment) and end point (e.g. death, recurrence or metastasis). In addition, criteria for efficacy of treatment can be expanded to include response to a known therapy, probability of survival, probability of metastasis within a given time period, and probability of tumor recurrence.

The term “synergistic effect” refers to the combined effect of two or more agents (e.g., an agent described herein) can be greater than the sum of the separate effects of the one agents/therapy alone.

As used herein, “telomere shortening” refers to any decrease in length of telomeres in an individual. Telomeres, the specific DNA-protein structures found at both ends of each chromosome, protect genome from nucleolytic degradation, unnecessary recombination, repair, and interchromosomal fusion. Telomeres therefore play a vital role in preserving the information in our genome. As a normal cellular process, a small portion of telomeric DNA is lost with each cell division. When telomere length reaches a critical limit, the cell undergoes senescence and/or apoptosis. Telomere length may therefore serve as a biological clock to determine the lifespan of a cell and an organism. The term telomere shortening can refer to the normal or typical process of aging (e.g., such as the shortening of telomeres during DNA replication), as well as any pathology that results in a decrease of telomere length. Replicative senescence, apoptosis, and cell cycle arrest at S or G2/M phase are markers of telomere shortening in cells. In addition, telomere shortening can be measured in change of base pairs (bp) within an individual or subject. Therefore, as a non-limiting example, telomere shortening may be measured as a decrease of at least 25 bp, at least 50 bp, at least 75 bp, at least 100 bp, at over a period of time. Such a decrease can represent a normal or average decrease in telomere length of the individual as compared to a population average or compared to a measurement of telomere length in the individual earlier in time.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀(i.e., the concentration which achieves half-maximal cytotoxic or cytostatic effect on diseased or afflicted cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, the disease or afflicted cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Table 1) are well-known in the art and readily available on publicly available databases, such as the National Center for Biotechnology Information (NCBI). For example, exemplary nucleic acid and amino acid sequences derived from publicly available sequence databases are provided below.

TABLE 1
Serine/threonine-protein kinase RIO2, such as from a mammal (e.g., a rodent or
human)

SEQ ID NO: 12 Human serine/threonine-protein kinase RIO2 isoform 1 (RIOK2)
Amino Acid Sequence (NP 060813.2)

| | |
|---|---|
| 1 | mgkvnvaklr ymsrddfrvl tavemgmknh eivpgslias iaslkhggen kvlrelvkhk |
| 61 | liawertktv qgyrltnagy dylalktlss rqvvesvgnq mgvgkesdiy ivaneegqqf |
| 121 | alklhrlgrt sfrnlknkrd yhkhrhnvsw lylsrlsamk efaymkalye rkfpvpkpid |
| 181 | ynrhavvmel ingyplcqih hvedpasvyd eamelivkla nhglihgdfn efnlildesd |
| 241 | hitmidfpqm vstshpnaew yfdrdvkcik dffmkrfsye selfptfkdi rredtldvev |
| 301 | sasgytkemq addellhplg pddknietke gsefsfsdge vaekaevygs eneserncle |
| 361 | esegcycrss gdpeqikeds lseesadars femtefnqal eeikgqvven nsvtefseek |
| 421 | nrtenynrqd gqrvqggvpa gsdeyedecp hlialsslnr efrpfrdeen vgamnqyrtr |
| 481 | tlsitssgsa vscstippel vkqkvkrqlt kqqksavrrr lqkgeanift kqrrenmqni |
| 541 | kssleaasfw ge |

SEQ ID NO: 13 Human serine/threonine-protein kinase RIO2 isoform 2 (RIOK2)
Amino Acid Sequence (NP_001153221.1)

| | |
|---|---|
| 1 | mgkvnvaklr ymsrddfrvl tavemgmknh eivpgslias iaslkhggen kvlrelvkhk |
| 61 | liawertktv qgyrltnagy dylalktlss rqvvesvgnq mgvgkesdiy ivaneegqqf |
| 121 | alklhrlgrt sfrnlknkrd yhkhrhnvsw lylsrlsamk efaymkalye rkfpvpkpid |
| 181 | ynrhavvmel ingyplcqih hvedpasvyd eamelivkla nhglihgdfn efnlildesd |
| 241 | hitmidfpqm vstshpnaew yfdrdvkcik dffmkrfsye selfptfkdi rredtldvev |
| 301 | sasgytkemq addellhplg pddknietke gsefsfsdge vaekaevygs eneserncle |
| 361 | esegcycrss gdpeqikeds lseesadars femtefnqal eeikgqvven nsvtefseek |
| 421 | nrtenynrqd gqrvqggvpa gsdeyedecp hlialsslnr efrpfryrll siaf |

SEQ ID NO: 14 Human RIO kinase 2 (RIOK2), transcript variant 2, mRNA
(NM_001159749.2)

| | |
|---|---|
| 1 | gccttacgct ttgcggctgt cgtcggagag gcatctgggt tcggactggg gccgccatgg |
| 61 | ggaaagtgaa tgtggccaag ttgcgttaca tgagccgaga tgacttcagg gtcttgaccg |
| 121 | cggttgaaat gggcatgaag aaccatgaaa ttgttcccgg cagtttgatt gcttctatag |
| 181 | ccagccttaa acatggtggc tgtaataaag ttttaagaga attagtgaaa cataaactca |
| 241 | tagcttggga gcgtaccaaa actgtccagg gctatcggtt gacaaatgca ggatatgatt |
| 301 | acctagcttt gaaaacactt tcttctaggc aagtagttga gtctgttgga aaccagatgg |
| 361 | gtgttggcaa agaatcagat atttacattg ttgcaaatga agaaggacaa caatttgcat |
| 421 | taaagcttca cagactagga agaacctcgt ttcgaaattt gaaaaacaaa cgcgattatc |
| 481 | ataaacatag gcacaatgtg tcatggctat atttatctcg tctctctgcc atgaaggaat |
| 541 | ttgcctatat gaaggcattg tatgagagga aatttccagt tccaaagcca attgattaca |
| 601 | atcgtcatgc agtggtcatg gaactcataa atggttatcc actatgtcag atacaccatg |
| 661 | ttgaagatcc tgcatcagta tatgatgaag ctatggaact aattgtcaaa cttgcaaatc |
| 721 | atgggctgat tcatggagat tttaatgaat ttaatctcat tttggatgaa agtgaccata |
| 781 | tcaccatgat tgattttcca cagatggttt caacttctca tcccaatgct gagtggtatt |
| 841 | ttgacagaga tgttaaatgc attaaagatt tctttatgaa acgtttcagc tacgaaagtg |
| 901 | agctttttcc aacttttaag gatatcagga gagaagacac tcttgatgtg gaggtttctg |
| 961 | ccagtggcta cacaaaggaa atgcaggcag atgatgaact gcttcatcca ttaggtccag |
| 1021 | atgataaaaa tattgaaaca aaagagggat ctgaattctc attttcagat ggagaagtgg |
| 1081 | cagaaaaagc agaggtttac gggtcagaaa atgaaagtga acggaactgt ctagaagaat |
| 1141 | cagagggctg ctattgcaga tcatctggag accctgaaca aataaaggaa gacagtttat |
| 1201 | cagaagagag tgctgatgca cggagttttg aaatgactga attcaatcaa gctttagaag |
| 1261 | aaataaaagg gcaggttgtt gaaaacaact ctgtaactga attttctgag gagaaaaaca |
| 1321 | gaactgaaaa ttacaacagg caagatggtc agagagttca aggaggagtc cctgctggct |
| 1381 | ctgacgagta tgaagatgaa tgccctcatc taattgcctt gtcgtcatta aatagagaat |
| 1441 | tcaggccttt caggtataga cttctgtcga ttgcttttta actttagtct tgagactgat |
| 1501 | actttgttag attctagtaa aagaatgttt ttttcaaatg atgtgacaaa attgtattct |
| 1561 | gccaccttga tgtataaaat gaaagccaaa taattttttg ttgttaaaaa ctcaaaatag |
| 1621 | cttccttttc aattgacctg attctaggca gcctattgac taaatttcaa gtaattctgc |
| 1681 | cttaaaatac aaactgatgg ctgggcacag tggctcgctc atgcctgtaa tctcagcact |
| 1741 | tctgggaggc caaggcaggc aaatcacctg aggtcaggag tttgagacca gccaggccaa |
| 1801 | catggcaaaa ccctgtctct actaaaaata taaaaattaa ccgggcatgg tggcctgtgc |
| 1861 | ttgtaatccc agctacttgg gagactgagg caggagaatc acttgaacct gggaggcaga |
| 1921 | ggttgcagta agccaaagat cgtaccactg cactccagcc tgggtgacga gcaaaaaact |
| 1981 | ccgtctcaaa aatatatata tatatacaaa ctgatgattt taaaattatt gcaggtctct |
| 2041 | ggatacattg atcaggtttt agatggatga gttattcctc atatattttc ctaattacac |
| 2101 | atctgatgtg tgtttgagtt atgtaaaggc atgtaaatat taaaacatgc aataaatgta |
| 2161 | ctttctatat gaaa |

SEQ ID NO: 15 Homo sapiens RIO kinase 2 (RIOK2), transcript variant 1, mRNA
(NM_018343.3)

| | |
|---|---|
| 1 | gccttacgct ttgcggctgt cgtcggagag gcatctgggt tcggactggg gccgccatgg |
| 61 | ggaaagtgaa tgtggccaag ttgcgttaca tgagccgaga tgacttcagg gtcttgaccg |
| 121 | cggttgaaat gggcatgaag aaccatgaaa ttgttcccgg cagtttgatt gcttctatag |
| 181 | ccagccttaa acatggtggc tgtaataaag ttttaagaga attagtgaaa cataaactca |
| 241 | tagcttggga gcgtaccaaa actgtccagg gctatcggtt gacaaatgca ggatatgatt |
| 301 | acctagcttt gaaaacactt tcttctaggc aagtagttga gtctgttgga aaccagatgg |
| 361 | gtgttggcaa agaatcagat atttacattg ttgcaaatga agaaggacaa caatttgcat |
| 421 | taaagcttca cagactagga agaacctcgt ttcgaaattt gaaaaacaaa cgcgattatc |
| 481 | ataaacatag gcacaatgtg tcatggctat atttatctcg tctctctgcc atgaaggaat |
| 541 | ttgcctatat gaaggcattg tatgagagga aatttccagt tccaaagcca attgattaca |
| 601 | atcgtcatgc agtggtcatg gaactcataa atggttatcc actatgtcag atacaccatg |
| 661 | ttgaagatcc tgcatcagta tatgatgaag ctatggaact aattgtcaaa cttgcaaatc |
| 721 | atgggctgat tcatggagat tttaatgaat ttaatctcat tttggatgaa agtgaccata |
| 781 | tcaccatgat tgattttcca cagatggttt caacttctca tcccaatgct gagtggtatt |
| 841 | ttgacagaga tgttaaatgc attaaagatt tctttatgaa acgtttcagc tacgaaagtg |
| 901 | agctttttcc aacttttaag gatatcagga gagaagacac tcttgatgtg gaggtttctg |
| 961 | ccagtggcta cacaaaggaa atgcaggcag atgatgaact gcttcatcca ttaggtccag |
| 1021 | atgataaaaa tattgaaaca aaagagggat ctgaattctc attttcagat ggagaagtgg |
| 1081 | cagaaaaagc agaggtttac gggtcagaaa atgaaagtga acggaactgt ctagaagaat |
| 1141 | cagagggctg ctattgcaga tcatctggag accctgaaca aataaaggaa gacagtttat |
| 1201 | cagaagagag tgctgatgca cggagttttg aaatgactga attcaatcaa gctttagaag |
| 1261 | aaataaaagg gcaggttgtt gaaaacaact ctgtaactga attttctgag gagaaaaaca |
| 1321 | gaactgaaaa ttacaacagg caagatggtc agagagttca aggaggagtc cctgctggct |
| 1381 | ctgacgagta tgaagatgaa tgccctcatc taattgcctt gtcgtcatta aatagagaat |
| 1441 | tcaggccttt cagagatgaa gaaaatgtgg gagctatgaa tcagtataga acaagaactc |
| 1501 | tgagtatcac ttcttcaggc agtgctgtaa gctgttcaac aattcctcca gaactggtga |
| 1561 | aacagaaggt gaaacgtcag ttgacaaaac agcaaaaatc agctgtcaga cgtcgattgc |
| 1621 | agaaaggaga agcaaatata tttaccaagc aacgtaggga aaacatgcaa aatatcaaat |
| 1681 | caagtttgga agcagccagc ttttggggag aataatatat ttaggatctt ggatatgttt |
| 1741 | aatatatttt ttaaagttac tgtaattcct ttttgagccc tcatttgtct tttttgagcc |
| 1801 | aaggctatca tatattaata aataaaccct ctttcatcta taaatttggt cattcagtat |
| 1861 | gtgtcttgca aagaataatt acctgccaaa gcagtccatt tttttcaaag taatttgggt |
| 1921 | aactacagtt acgtaacttt attagagaca tttgcatatt tttaaaaatc taaataacat |
| 1981 | ttttgttgtt ttctgtttgt tgaggatgat atgctacaat gcacataaag caattagaac |
| 2041 | aatgtttagc acattcttaa gcattcaata aaacatggct attagaatca taatgattct |
| 2101 | gaaattctgc tgacaatttg gttgcatttt atttggcaac tggaggactt cactgtctta |
| 2161 | tttttcttta gaagtaaaga taagcctttg gaagtaaaga taagccagtg atgcacaatt |
| 2221 | tgcttaatta attgattaat aatctgtttt gtgccactta cattggtaat gtggtcatga |
| 2281 | gaatggaaac ttacttgaga tagtctacca caatttttac agttggcagc ttgtaagcag |
| 2341 | gcctgagtag cagaagagtt aggggttggg gaaggtggag tagaatgagg aatgaaatgt |
| 2401 | tctttctcat atgcttatta tataagtggg tccggaacat gttctcacct caaggttaac |
| 2461 | aaatacctaa tgcaggaaag aaaatacata gccatggtcc cttcctcaag gactttataa |
| 2521 | ttcagtgaga tgtaaagatg agtgcttaag tgtatgcacc tgattattgt ctcttcattg |
| 2581 | ccgcataagt cggaaaaggg gatcacttgc agcgaaaaat ggccactaga tggcaatatc |
| 2641 | accccatagt caccaaacag gattacttgc atccctaatt atgttctacc agagggaata |
| 2701 | tagtacagac ttttccaaga ttgataaaga cagcagattc cctttcacct ttgccaaatt |
| 2761 | aactctacta gctatttgca agacctgtaa ttatctaata aattggctta aatatataga |
| 2821 | agctagaaaa tgccaattcc tcttccctca aaagttacct gacattcaaa tatttaaata |
| 2881 | cccttgcttt gcattttaga tatatctttg aattcaggta ttcattttgt ataatctctc |
| 2941 | aacttttaag catttttata atgtaaagtt gaaaacagct taatagaacc acagaatgtt |
| 3001 | tattttggag tcattttctt atattcagag ctatgaaagc aaattgggaa taaagcctta |
| 3061 | tcctgctcag ctttacctag ttgactcagt tgacttttga gtatctacta cataaaaagc |
| 3121 | ccattactat taagaaactc aagacaaaca tgatttttac ctttaatagc ctaagatttc |
| 3181 | ttgtgggcca aggaattaat tattccaatc agaaatggag gccttacctc atttgggcaa |
| 3241 | agattagtgc tagttattga agatttacta ataaatgatc tgttaaggaa tttagttttt |
| 3301 | tttggatatg ttgttttggt tgttgaaaac taaggtataa ttataatagt tattttttga |
| 3361 | agatatgttt ggctagccat caattagaag ttagagagtt tgtcctaaca gataagctat |
| 3421 | aaccctcaca ttttaaaaca tgcatagtga acaatgaaca aaaacaaatt caaaatttta |
| 3481 | ctaatgtaaa gctgtatatt gtgattgtta aaacactttt ttttgcctag gagttccaga |
| 3541 | tttaaatcct ggttctacac ttcccaaacc tcaggtacct tctgtggcct ctctaacctc |
| 3601 | aattttttta cttgtaaaat ggggacattt aatagtatac ctcagtgtcg tgaagactaa |
| 3661 | atgaagtacc acttgtaaag agtttattac agtgcctgac atatactgta tggttagtac |
| 3721 | tgctaaaatg acttcttaag gttaccaaaa ggaaaattcc acaagctgac cacctttcct |
| 3781 | attgtgatgg tcactggtat ttcagctatg atcctttgtt gtgtatttat ccagactgaa |
| 3841 | agatgttttt ctagtaacca tcttgcatcg tcttagtctc cccattaaat aaacttttcc |
| 3901 | tacaaacta |

SEQ ID NO: 16 Mouse serine/threonine-protein kinase RIO2 Amino Acid
Sequence (NP_080210.1)

| | |
|---|---|
| 1 | mgkvnvaklr ymsrddfrvl tavemgmknh eivpcslias iaslkhggen kilrelvkhk |
| 61 | liawertktv qgyrltnagy dylalktlss rqvvesvgnq mgvgkesdiy ivaneagqql |
| 121 | alklhrlgrt sfrnlknkrd yhkhrhnvsw lylsrlsamk efaymkalye rkfpvpkpid |
| 181 | ynrhavimel ingyplcqih hvedpasvyd eamelivklg nhglihgdfn efnlmldkdd |
| 241 | hitmidfpqm vstshpnaew yfdrdvkcir effmkrfsye selyptfsdi rkedsldvev |
| 301 | sasgytkemq addellhpvg pddkitetee dsdftfsdee mlekakvwrs elekeadpad |
| 361 | esggswccss tdskqikdgg lpeesahvss fevtalsqav eemerqvlph rsvtefsees |
| 421 | rrtendgqpg qrspagsedc ddepphlial ssvnrefrpf rdeesmssvt rhrtrtlsvt |
| 481 | sagsalscst ippelvkqkv krqltrqqka aarrrlqkge anvftkqrre nmqnikssle |
| 541 | aasfwgd |

SEQ ID NO: 17 Mouse Mus musculus RIO kinase 2 (Riok2), mRNA Sequence
(NM_025934.2)

| | |
|---|---|
| 1 | gtggcatact atgtaaaaca tttgcaactt gccaaatcag tcaaacgtaa attatttatt |
| 61 | taggggctga aaaatcactt ctgcctgatt tttagctaca aaaacgagaa aaaaatagca |
| 121 | tttttctact gcttcttgag cacgtgttct tacctagcga taagcacctt aaatacaggg |
| 181 | cgcgtgcgcg ggcaggtcgc cgccgaaagc ctgcgagatt acccagcaac tttgcgtctt |
| 241 | cccagagagc gtccgtggtg gaactgggca agcatgggga aggtgaatgt agccaagctt |
| 301 | aggtacatga gccgcgatga cttcagagtt ctgactgcgg ttgaaatggg catgaaaaac |
| 361 | catgagattg ttccctgcag tttgattgct tctatagcca gcctgaaaca tggtggctgt |
| 421 | aataagattc taagagaatt ggtgaagcat aaacttatag cgtgggaacg tactaaaact |
| 481 | gtgcagggct atcggttgac aaatgctggc tatgattacc tagctctgaa aacgctgtct |
| 541 | tctagacagg tagttgaatc tgttggaaac cagatgggtg tgggcaaaga gtcagacatt |
| 601 | tacattgttg ccaatgaagc cgggcagcag ctcgcactga agctccacag actcggaaga |
| 661 | acttcctttc gaaatctgaa aaacaagcgc gattaccaca agcacaggca caatgtttct |
| 721 | tggctttact tatctcggct ctcggccatg aaggaatttg cctatatgaa ggcattatat |
| 781 | gagaggaaat ttccagttcc aaagccaatt gattataatc gccatgcagt gatcatggag |
| 841 | cttattaatg gttatcccct atgtcagata catcatgttg aagaccctgc atcagtatat |
| 901 | gatgaagcta tggaactgat agtcaaactg ggaaaccatg gactgattca tggagatttc |
| 961 | aatgaattca atctcatgtt ggacaaagat gaccacatca ccatgattga ttttccgcag |
| 1021 | atggtttcca catcccaccc aaatgctgaa tggtattttg acagagatgt taaatgcatt |
| 1081 | cgagaattct tcatgaaacg ttttagctat gaaagtgagc tctacccaac cttcagtgat |
| 1141 | atcaggaagg aggattctct tgatgttgag gtctctgcca gtggttacac aaaggaaatg |
| 1201 | caagcagatg atgaactgct acatccagta ggcccagatg ataaaattac tgaaacagaa |
| 1261 | gaggactctg acttcacgtt ttctgatgaa gaaatgttag aaaaagccaa agtgtggagg |
| 1321 | tcagaattgg aaaaggaagc agacccagca gatgaatcag gtggatcctg gtgctgctcg |
| 1381 | tctacagaca gcaaacaaat aaaggatggc ggcttaccag aggagagtgc tcatgtgtcc |
| 1441 | agtttcgaag tgacggcatt aagccaagct gtagaagaga tggaaaggca ggttcttccc |
| 1501 | cacaggtcgg ttactgagtt ctctgaggag agcaggagaa ctgaaaatga tgggcagcca |
| 1561 | ggtcagagaa gccctgctgg ctctgaggac tgtgacgatg aaccccccca tctgattgcc |
| 1621 | ttgtcatcag tgaacaggga gttcaggcct ttcagagacg aagagagtat gagtagtgtt |
| 1681 | actcggcata gaacaagaac attgagtgtt acgtctgcgg gcagtgccct aagctgttca |
| 1741 | acaatccctc cagaactggt gaagcagaag gtgaagcgtc agctgacaag gcagcagaag |
| 1801 | gcggctgcca gacgccggct gcagaaagga gaagccaatg tgtttaccaa gcagcggagg |
| 1861 | gaaaacatgc agaatattaa atcgagtttg gaagcagcca gcttttgggg agactaatag |
| 1921 | atttctgata tgtattattt ttttagatgc tataatttta tttttagcca gtatattggt |
| 1981 | cttttttgga ccaaagccag taaattatgg aaataaagcc tctttagact cagagcttcc |
| 2041 | aaggtctgat taatagtaaa tctgccaagc actctgtttc ttaaaatttt tcaggtaact |
| 2101 | aatttaaata taaaataacg tgtgaaaaaa acttcattgg cagtaattgc aaaacatgga |
| 2161 | catcattttt atttcactgt gatgttcaga ctctgcctta aacaggtaga aaaatgactt |
| 2221 | gcctcagatt ctggatttac atgtaataaa taatagaaca acttaatgca atctgtagtt |
| 2281 | ctctcttttc tccttcagtc ggtcacactt gggcttttac tacattggtg agttggcttt |
| 2341 | aggatcacct gagacagtca gtacacagtt atttgaccat tggagtgaaa ctgatcgtct |
| 2401 | tctgtttctt tagacttctg gagtggagat gaatcagtaa tgggtagacg agcagcagcg |
| 2461 | ggtcttttgg tgccgttctc attggccctg tggtcgtggg aaaaggaagt ttaggttcct |
| 2521 | accagtattt ttgcagttgt cagctttccc caggtttcag cagaagtggg gggagggagg |
| 2581 | gagggaggga gggagggagg gagagagaga gagagagaga gagagagaga gagagagaga |
| 2641 | gagagagaga gaatgcatga ctccaggatg tgttcctacc tcagtgttaa caagtctcta |
| 2701 | gaacaacact ctggtgtgat tcatccctca agcgcattct aattcagtca gtggaagtga |
| 2761 | gtgtaagtat acaagcactg tcccttcagc ttccctgagc agatctcgaa tagaagatcg |
| 2821 | tcttcagcaa gaagtgacca ccagatggca gtgtttgcca gcagtgacat gataaaaaca |
| 2881 | ccagcttccc ttttacctta gcaaaattca ttctgactat ttgtaactga tcactatcta |
| 2941 | agcgcattta aatgtttgga tatatgaata tgtcaagtgc agtttctttg aaaattagac |
| 3001 | tgatttttgg aagtgttgat atagccctgt tttaaaatgt agtttaagct cagatactca |
| 3061 | ttttttatac tctgctaaaa gtgtttctat aacataaact gatgttgaag atagtttaat |
| 3121 | aagaccacag aatgtttatt ttgtaatacg tttttcttat gtccaaagca gattgggaat |
| 3181 | aaaagtgttt gacctcaaaa a |

SEQ ID NO: 18 Rat serine/threonine-protein kinase RIO2 Amino Acid Sequence
(NP_001009687.1)

| | |
|---|---|
| 1 | mgkvnvaklr ymsrddfrvl tavemgmknh eivpcslvas iaslkhggen kilrelvkhk |
| 61 | liawertktv qgyrltnagy dylalktlss rqvvdsvgnq igvgkesdiy ivaneegqqf |
| 121 | alklhrlgrt sfrnlknkrd yhkhrhnvsw iylsrlsamk efaymkalhe rkfpvpkpvd |
| 181 | ynrhavimel ingyplcqih hvedpasvyd eamelivklg nhglihgdfn efnlmldkdd |
| 241 | hitmidfpqm vstshpnaew yfdrdvkcir efflkrfnye selyptfsdi rredsldvev |
| 301 | sasgytkemq addellhpvg pddkitetee esdlslsdee mlerakvqrs elenepnpad |
| 361 | esgssccfss adskqmkedg lpeesadvss fevtklhqav eevgrqvlth ksvaevsees |
| 421 | trteddprhd gqpgqsspag caegedelpd lialssinke frpfrdedsv ssvtqhrtrt |
| 481 | lsvtstgsvl scstippelv kqkvkrqltr qqkaaarrrl qkgeaniftk qrrenmqnik |
| 541 | ssleadsfwg d |

SEQ ID NO: 19 Rat RIO kinase 2 (Riok2), cDNA (NM_001009687.1)

| | |
|---|---|
| 1 | gctgggcaag catggggaag gtgaatgtgg ccaagctaag gtacatgagt cgagatgact |
| 61 | tcagagttct gactgcggtt gaaatgggca tgaaaaacca tgagatcgtt ccctgcagtt |
| 121 | tggttgcttc tatagccagc ctgaaacacg gtggctgtaa taaaatctta agagaattgg |
| 181 | tgaagcataa acttatagca tgggaacgta ctaaaactgt ccagggctat cggttgacaa |
| 241 | atgctggcta tgattaccta gctttgaaaa ccctgtcttc cagacaggta gttgattctg |
| 301 | ttggaaacca gataggtgtc ggcaaagagt cagatattta cattgttgca aatgaagaag |
| 361 | ggcagcagtt tgcactgaag ctgcaccgac tggggagaac ctcctttcga aatctgaaaa |
| 421 | acaagcgtga ttaccataag cacagacata atgtttcttg gatttattta tctcgtctct |
| 481 | ctgccatgaa ggaatttgcc tatatgaagg cattacatga gcggaaattc ccagttccaa |
| 541 | agccagttga ctacaatcgc cacgcagtga tcatggagct tatcaatggc taccccctgt |
| 601 | gtcagataca tcatgttgaa gaccctgcat cagtgtatga cgaagctatg gaactgatag |
| 661 | tcaaactggg aaaccatggg ctgattcatg gagatttcaa tgaattcaac ctcatgttgg |
| 721 | acaaagatga ccacatcacc atgattgatt ttccacagat ggtttccact tcccacccga |
| 781 | acgctgaatg gtattttgac agagatgtta aatgcattcg agagttcttc ctgaaacgtt |
| 841 | ttaactatga aagtgagctc tacccaacct tcagtgatat caggagagag gattctctcg |
| 901 | atgttgaggt ctctgccagt ggttacacga aggaaatgca ggcagatgat gaactgctac |
| 961 | atccagtagg tccggatgat aaaattactg aaacagaaga ggaatctgac ttgtcactct |
| 1021 | ctgatgaaga aatgttagaa agagccaaag ttcagaggtc agaactggaa aatgaaccaa |
| 1081 | acccagcgga tgaatcaggt agctcctgtt gcttctcatc tgcagacagc aaacagatga |
| 1141 | aggaagacgg cttacctgag gagagtgctg atgtgtccag ttttgaagta actaagttac |
| 1201 | atcaagctgt agaagaagtg ggaaggcagg ttcttaccca caagtctgtt gctgaggttt |
| 1261 | ctgaggagag cactagaact gaggatgatc ccaggcatga tggccagccg ggtcagagca |
| 1321 | gccctgctgg ctgtgcagag ggggaggatg aactccccga tctcattgcc ctgtcatcat |
| 1381 | tgaacaagga gttcaggcct ttcagagatg aagacagtgt gagcagtgtt actcagcata |
| 1441 | gaacaagaac cttgagtgtt acgtctaccg gcagtgtcct gagctgctcc acaatccctc |
| 1501 | cagaactggt gaagcagaag gtgaagcgtc agctgacgag gcagcagaag gcagctgcca |
| 1561 | gacgccggct gcagaaagga gaagccaata tatttaccaa gcagcggagg gaaaacatgc |
| 1621 | agaatatcaa atcaagtttg gaagcagaca gcttttgggg agactaataa atttttgatc |
| 1681 | tgtatatttt tttagatact gtaattctat ttttagccag tatattggtc tttttaagac |
| 1741 | caaagccaat atttatggaa attatggaaa taaaccctta ttagactcaa aaaaaaaaaa |
| 1801 | aaaaaaaaaa aaaa |

TABLE 2
T-complex protein 1 subunit, such as from a mammal (e.g., a or human)

SEQ ID NO: 20 Human T-complex protein 1 subunit delta isoform a Amino Acid
Sequence (NP_006421.2)

| | |
|---|---|
| 1 | mpenvaprsg atagaaggrg kgayqdrdkp aqirfsnisa akavadairt slgpkgmdkm |
| 61 | iqdgkgdvti tndgatilkq mqvlhpaarm lvelskaqdi eagdgttsvv iiagslldsc |
| 121 | tkllqkgihp tiisesfqka lekgieiltd msrpvelsdr etllnsatts lnskvvsqys |
| 181 | sllspmsvna vmkvidpata tsvdlrdiki vkklggtidd celveglvlt qkvsnsgitr |
| 241 | vekakigliq fclsapktdm dnqivvsdya qmdrvlreer ayilnlvkqi kktgcnvlli |
| 301 | qksilrdals dlalhflnkm kimvikdier ediefickti gtkpvahidq ftadmlgsae |
| 361 | laeevnings gkllkitgca spgktvtivv rgsnklviee aersihdalc virclvkkra |
| 421 | liagggapei elalrlteys rtlsgmesyc vrafadamev ipstlaenag lnpistvtel |
| 481 | rnrhaqgekt aginvrkggi snileelvvq pllvsvsalt latetvrsil kiddvvntr |

SEQ ID NO: 21 Human T-complex protein 1 subunit delta isoform b Amino Acid
Sequence (NP_001243650.1)

| | |
|---|---|
| 1 | mpenvaprsg atagaaggrg kgayqdrdkp aqirfsnisa akavadairt slgpkgmdkm |
| 61 | lvelskaqdi eagdgttsvv iiagslldsc tkllqkgihp tiisesfqka lekgieiltd |
| 121 | msrpvelsdr etllnsatts lnskvvsqys sllspmsvna vmkvidpata tsvdlrdiki |
| 181 | vkklggtidd celveglvlt qkvsnsgitr vekakigliq fclsapktdm dnqivvsdya |
| 241 | qmdrvlreer ayilnlvkqi kktgcnvlli qksilrdals dlalhflnkm kimvikdier |
| 301 | ediefickti gtkpvahidq ftadmlgsae laeevnings gkllkitgca spgktvtivv |
| 361 | rgsnklviee aersihdalc virclvkkra liagggapei elalrlteys rtlsgmesyc |
| 421 | vrafadamev ipstlaenag lnpistvtel rnrhaqgekt aginvrkggi snileelvvq |
| 481 | pllvsvsalt latetvrsil kiddvvntr |

SEQ ID NO: 22 Human chaperonin containing TCP1 subunit 4 (CCT4),
transcript variant 1, mRNA, (NM 006430.4)

| | |
|---|---|
| 1 | cttctccgcc tccgcctcct cccgacgccg gcgccgcttt ctggaaggtt cgtgaaggca |
| 61 | gtgagggctt accgttatta cactgcggcc ggccagaatc cgggtccatc cgtccttccc |
| 121 | gagccaaccc agacacagcg gagtttgcca tgcccgagaa tgtggcaccc cggagcgggg |
| 181 | cgactgccgg ggctgccggc ggccgcggga aaggcgccta tcaggaccgc gacaagccag |
| 241 | cccagatccg cttcagcaac atttccgccg ccaaagcggt tgctgatgct attagaacaa |
| 301 | gccttggacc aaaaggaatg gataaaatga ttcaagatgg aaaaggtgat gtaaccatta |
| 361 | caaatgatgg tgctaccatt ctgaaacaaa tgcaagtatt acatccagca gccagaatgc |
| 421 | tggtggagct gtctaaggct caagatatag aagcaggaga tggcaccaca tcagtagtca |
| 481 | tcattgctgg ctccctctta gattcttgta ccaagcttct tcagaaaggg attcatccaa |
| 541 | ccatcatttc tgagtcattc cagaaggccc tggaaaaggg cattgaaatc ttgactgaca |
| 601 | tgtctcgacc tgtggaactg agtgacagag aaactttgtt aaatagtgca accacttcac |
| 661 | tgaactcaaa ggtggtttct cagtattcaa gtctgctttc tccaatgagt gtaaatgcag |
| 721 | tgatgaaagt gattgaccca gccacagcca ccagtgtaga tcttagagat attaaaatag |
| 781 | ttaagaagct tggtgggaca attgatgact gtgagttggt ggaagggctg gttctcaccc |
| 841 | aaaaagtgtc aaattctggc ataaccagag ttgaaaaggc caagattggg cttattcagt |
| 901 | tttgcttatc tgctcccaaa acagacatgg ataatcaaat agtggtttct gactatgccc |
| 961 | agatggaccg agtgctgcga gaagagagag cctatatttt aaatttagtg aagcaaatta |
| 1021 | aaaaaacagg atgtaatgtc cttctcatac agaaatctat tctaagagat gctcttagtg |
| 1081 | atcttgcatt acactttctg aataaaatga agatcatggt gattaaggat attgaaagag |
| 1141 | aagacattga attcatttgt aagacaattg gaaccaagcc agttgctcat attgaccaat |
| 1201 | ttactgctga catgctgggt tctgctgagt tagctgagga ggtcaattta aatggttctg |
| 1261 | gcaaactgct caagattaca ggctgtgcca gccctggaaa aacagttaca attgttgttc |
| 1321 | gtggttctaa caaactggtg attgaagaag ctgagcgctc cattcatgat gccctatgtg |
| 1381 | ttattcgttg tttagtgaag aagagggctc ttattgcagg aggtggtgct ccagaaatag |
| 1441 | agttggccct acgattaact gaatattcac gaacactgag tggtatggaa tcctactgcg |
| 1501 | ttcgtgcttt tgcagatgct atggaggtca ttccatctac actagctgaa aatgccggcc |
| 1561 | tgaatcccat ttctacagta acagaactaa gaaaccggca tgcccaggga gaaaaaactg |
| 1621 | caggcattaa tgtccgaaag ggtggtattt ccaacatttt ggaggaactg gttgtccagc |
| 1681 | ctctgttggt atcagtcagt gctctgactc ttgcaactga aactgttcgg agcattctga |
| 1741 | aaatagatga tgtggtaaac actcgataat ctggataact gactagcacc attatgatca |
| 1801 | ccagtattgt ggctggaatg gaagaagatc accttggtgt tccttgtttg gaagattatt |
| 1861 | tcctctgaat ttctgggctt ggtcttccag ttggcatttg cctgaagttg tattgaaaca |
| 1921 | atttaatgaa aatattaaat atttggtttc aaaaggcaga tttatcttct cccaacattc |
| 1981 | tgttatttct gatacttttg aaaaactaat aaaaactaat aaaagaagcg taaaaagtga |
| 2041 | gtttacatgt tgaggaaaaa aatggcccaa tatgctcatc actgataaat gctccctggc |
| 2101 | cttaaaaact accaacatat aatatatatg ctgtcttaaa agttaatgat ccaagtggca |
| 2161 | cctctctgaa cctactttgg cttgggaggc tgcccagtta aaacaaaaat aagttaatgg |
| 2221 | tacagaaaga gaatgaaaaa tgaaagcctc cttttatcct atcatcctaa ttccttttcc |
| 2281 | cagtaataat gactgctgtg ttggattcct tctgcaaata aaagtgtata catatatgta |
| 2341 | gcaaatctta cttaaacaaa agggtttctt aactaa |

SEQ ID NO: 23 Human chaperonin containing TCP1 subunit 4 (CCT4), transcript
variant 2, mRNA (NM_001256721.1)

| | |
|---|---|
| 1 | ggcaaggaag gcccccttct ccgcctccgc ctcctcccga cgccggcgcc gctttctgga |
| 61 | aggttcgtga aggcagtgag ggcttaccgt tattacactg cggccggcca gaatccgggt |
| 121 | ccatccgtcc ttcccgagcc aacccagaca cagcggagtt tgccatgccc gagaatgtgg |
| 181 | caccccggag cggggcgact gccggggctg ccggcggccg cgggaaaggc gcctatcagg |
| 241 | accgcgacaa gccagcccag atccgcttca gcaacatttc cgccgccaaa gcggttgctg |
| 301 | atgctattag aacaagcctt ggaccaaaag gaatggataa aatgctggtg gagctgtcta |
| 361 | aggctcaaga tatagaagca ggagatggca ccacatcagt agtcatcatt gctggctccc |
| 421 | tcttagattc ttgtaccaag cttcttcaga aagggattca tccaaccatc atttctgagt |
| 481 | cattccagaa ggccctggaa aagggcattg aaatcttgac tgacatgtct cgacctgtgg |
| 541 | aactgagtga cagagaaact ttgttaaata gtgcaaccac ttcactgaac tcaaaggtgg |
| 601 | tttctcagta ttcaagtctg ctttctccaa tgagtgtaaa tgcagtgatg aaagtgattg |
| 661 | acccagccac agccaccagt gtagatctta gagatattaa aatagttaag aagcttggtg |
| 721 | ggacaattga tgactgtgag ttggtggaag ggctggttct cacccaaaaa gtgtcaaatt |
| 781 | ctggcataac cagagttgaa aaggccaaga ttgggcttat tcagttttgc ttatctgctc |
| 841 | ccaaaacaga catggataat caaatagtgg tttctgacta tgcccagatg gaccgagtgc |
| 901 | tgcgagaaga gagagcctat attttaaatt tagtgaagca aattaaaaaa acaggatgta |
| 961 | atgtccttct catacagaaa tctattctaa gagatgctct tagtgatctt gcattacact |
| 1021 | ttctgaataa aatgaagatc atggtgatta aggatattga aagagaagac attgaattca |
| 1081 | tttgtaagac aattggaacc aagccagttg ctcatattga ccaatttact gctgacatgc |
| 1141 | tgggttctgc tgagttagct gaggaggtca atttaaatgg ttctggcaaa ctgctcaaga |
| 1201 | ttacaggctg tgccagccct ggaaaaacag ttacaattgt tgttcgtggt tctaacaaac |
| 1261 | tggtgattga agaagctgag cgctccattc atgatgccct atgtgttatt cgttgtttag |
| 1321 | tgaagaagag ggctcttatt gcaggaggtg gtgctccaga aatagagttg gccctacgat |
| 1381 | taactgaata ttcacgaaca ctgagtggta tggaatccta ctgcgttcgt gcttttgcag |
| 1441 | atgctatgga ggtcattcca tctacactag ctgaaaatgc cggcctgaat cccatttcta |
| 1501 | cagtaacaga actaagaaac cggcatgccc agggagaaaa aactgcaggc attaatgtcc |
| 1561 | gaaagggtgg tatttccaac attttggagg aactggttgt ccagcctctg ttggtatcag |
| 1621 | tcagtgctct gactcttgca actgaaactg ttcggagcat tctgaaaata gatgatgtgg |
| 1681 | taaacactcg ataatctgga taactgacta gcaccattat gatcaccagt attgtggctg |
| 1741 | gaatggaaga agatcacctt ggtgttcctt gtttggaaga ttatttcctc tgaatttctg |
| 1801 | ggcttggtct tccagttggc atttgcctga agttgtattg aaacaattta atgaaaatat |
| 1861 | taaatatttg gtttcaaaag gcagatttat cttctcccaa cattctgtta tttctgatac |
| 1921 | ttttgaaaaa ctaataaaaa ctaataaaag aagcgtaaaa agtgagttta catgttgagg |
| 1981 | aaaaaaatgg cccaatatgc tcatcactga taaatgctcc ctggccttaa aaactaccaa |
| 2041 | catataatat atatgctgtc ttaaaagtta atgatccaag tggcacctct ctgaacctac |
| 2101 | tttggcttgg gaggctgccc agttaaaaca aaaataagtt aatggtacag aaagagaatg |
| 2161 | aaaaatgaaa gcctcctttt atcctatcat cctaattcct tttcccagta ataatgactg |
| 2221 | ctgtgttgga ttccttctgc aaataaaagt gtatacataa aa |

dkc1 (DYSKERIN) dyskeratosis congenita 1, dyskerin, such as from a mammal
(e.g., a rodent or human)
SEQ ID NO: 24 Human dkc1 (DYSKERIN) dyskeratosis congenita 1,
dyskerin Amino Acid Sequence (CAB51168.1)

| | |
|---|---|
| 1 | madaeviilp kkhkkkkerk slpeedvaei qhaeeflikp eskvakldts qwplllknfd |
| 61 | klnvrtthyt placgsnplk reigdyirtg finldkpsnp sshevvawir rilrvektgh |
| 121 | sgtldpkvtg clivcierat rlvksqqsag keyvgivrlh naieggtqls raletltgal |
| 181 | forppliaav krqlrvrtiy eskmieydpe rilgifwvsc eagtyirtlc vhlglllgvg |
| 241 | gqmqelrrvr sgvmsekdhm vtmhdvldaq wlydnhkdes ylrrvvyple klltshkrlv |
| 301 | mkdsavnaic ygakimlpgv lryedgievn qeivvittkg eaicmaialm ttavistcdh |
| 361 | givakikrvi merdtyprkw glgpkasqkk lmikqglldk hgkptdstpa twkqeyvdys |
| 421 | esakkevvae vvkapqvvae aaktakvsgm lssvwnrkre sesesdetpp aapqlikkek |
| 481 | kkskkdkkak aglesgaepg dgdsdttkkk kkkkkakeve lvse |

SEQ ID NO: 25 Mouse dkc1 (DYSKERIN) dyskeratosis congenita 1, dyskerin
Amino Acid Sequence (CAC04528.1)

| | |
|---|---|
| 1 | madaevitfp kkhkkkkdrk plqeddvaei qhaeeflikp eskvaqldts qwplllknfd |
| 61 | klnvrtahyt plpcgsnplk reigdyirtg finldkpsnp sshevvawir rilrvektgh |
| 121 | sgtldpkvtg clivcierat rlvksqqsag keyvgivrlh naieggtqls raletltgal |
| 181 | fqrppliaav krqlrvrtiy eskmieydpe rilgifwvsc eagtyirtlc vhlcfvlgsg |
| 241 | comqelrrvr sgvmsekdhm vtmhdvldaq wlydnhkdes ylrrvvyple klltshkrlv |
| 301 | mkdsavnaic ygakimlpgl lryedgievn qeivvittkg eaicmaialm ttavistcdh |
| 361 | givakikrvi merdtyprkw glgpkasqkk mmikqglldk hgkptdntpa twkqdyidys |
| 421 | dsgkntlvte avqapqlaae avnvikrkrd sesesdetpt vpqlkekkkk kdkkpktvle |
| 481 | sggetgdgdn dttkkkkkkk vkvveemse |

SEQ ID NO: 26 Human dkc1 (DYSKERIN) dyskeratosis congenita 1, dyskerin
exons 1-11 (AJ010395.1)

| | |
|---|---|
| 1 | gagcagcgcg gcctgacggg accaaggcgg cgggagtctg cggtcgttcc ctcggctgtg |
| 61 | gaccgggcgg cacgcacgcg gtgcagggta acatggcgga tgcggaaggt aagggctgca |
| 121 | ggcttccggg ccgtgctaac tccgggcgac tcggggaacg ggggtggggg gatggtatcg |
| 181 | gggcccgcgc acgcctccct ctggttcccg ccggcgtgtc ggcctcttca ccgcccggcc |
| 241 | ttaagccggc cttgtctctc ggccctggct ccgtggcaag ggcgcgagag tgggagcccc |
| 301 | tcgcggtgat ttggcatgat ggggctcggc cctctcacct ccctggggtc cgggtcgccg |
| 361 | tcgaaagaca tctgccgtgc tgcccacccc gcggtggcgt gggacgtggc agtgtctgtg |
| 421 | ctgctgccag cccggacgcc gccgttacag ctccatgcga ctaggggagc aagggtagtg |
| 481 | gaaacgggcc gagaagaaaa ggagcggagg gaccccggag cagggataga gctccgagtt |
| 541 | gcaagaaagt tctagaggcc acaagcccgc ggggacagtc gagtggtcgc ttatggaact |
| 601 | ttccggcccc ctgcgtgagc gcttctgtgg gatgtttaag ctctgccata gccacgtggt |
| 661 | gaaccggaag ggaagccgac acgtgcagta gcctacagtc agttacccag ccatgggacc |
| 721 | catacatttg gccttacaca ccttgttttc tcgcttacct acggatatac gttctaagaa |
| 781 | atgggtcgtt aggcgatttc atcattgtgt gaacatcaga gggctcttac acaaacctag |
| 841 | gtggcatagg ctactacaca cctaggctgt atatggtgta gcgtatggct cctaggcttc |
| 901 | aaacctggac agcatggtat tgtgcggaat gctgtaggca gttgggacac agcgtcgaga |
| 961 | gtttgtgtac ttaaacatgt ctaagcagaa aaaggacagt aaaaatatag cacagggccg |
| 1021 | ggcgcggtgg ctcaggcctg tgatcccagc ccaggctggc ggatcacctg cggtcaggag |
| 1081 | ttcaagacca gcctggccaa catggtgaaa ccccgtctct actaaaaata caaaaaattt |
| 1141 | aggtgggcgt ggtggtatac gcctgtaatc ccagctactc gggaggctga ggcaggagaa |
| 1201 | ttgcttgaac ctggagaagg aggttgcagt gagccgagtt tctgccactg cactccagcc |
| 1261 | tggggaaaga gtgtgaccgt ctcaaaaaaa aaaaattata tatatataaa tatgtgtgtg |
| 1321 | tatatatgta tattatatat gtatgtatta tatgtatgta tattatatat atatagtaca |
| 1381 | caaggttata agtggtgcac ccgaagagga gggcacttac catgaatgga gcttgctgag |
| 1441 | ctggaagttg ccctggttga gtcggtgact gaatgtgaag gcctcagaca ttacactact |
| 1501 | ggagacttta taatctgtac acctaggcta cactaaattt ataaaaatat atttttcttc |
| 1561 | agaataaatc aaccttagct tactgtaact ttataaactt tttacttcga ctgttgtaat |
| 1621 | aatacttagc ttaaaacaaa cacattattt agctgtacgt aaatattttc tctgcttatc |
| 1681 | tccttattta taagttttct attttaaaat tttttaattt ttttcttaaa aaattttttt |
| 1741 | ttgttaacta agacacaaac acagcacaca cagtagcctt gacctacaca gggtcaggat |
| 1801 | catcaagaca tcactaggag ataggaattt ttcagctctg ttataatctt atgggggcca |
| 1861 | ccatcacata cgcagtctgt acttgcaaaa cattatgtgg catgtgactg taattgtaga |
| 1921 | cctaaaatcc atttcctacc tgccctaatt tcggtcttta ttttaaaccc ttgtttcatt |
| 1981 | tattcttggg gaaaattccc aaatcctgtg tttgtttttt tagtaattat tttgccaaag |
| 2041 | aaacataaga agaaaaagga gcggaagtca ttgccagaag aagatgtagc cgtgagtagt |
| 2101 | aatgtgtttg acttcacttt gactaaaaag aaagtttact taaataacaa actaaaggaa |
| 2161 | agaagtgttt ttaaaggttt ccaaggaatg ggccagcatt ggtagtcatt gtgtagattc |
| 2221 | gaaaaagcac tgaaatgaca cagccccaga cagtccaagt gtccttcagg catagcttac |
| 2281 | taaatggtgc tggccttacc aactgctgtc tctgaatttg tcttccttat gccacccaca |
| 2341 | gacccgccat ccccctttct ctttaaatgt ttggacccat cttagtgaca tttcacttcc |
| 2401 | cctctgtact cctggttcat ttcttctctc tctttccctt ggaggtccca gtgaaaaggc |
| 2461 | ataggaagcc ttgttttctg tttaaaaggc atacatttcc atggcagtaa tggaattgtt |
| 2521 | caaaatcggg tgggaagttt aataggttat atttctgtcc tgtcgaagga aatacaacac |
| 2581 | gctgaagaat ttcttatcaa acctgaatcc aaagttgcta agttggacac gtctcagtgg |
| 2641 | ccccttttgc taaaggtatg tggttaaaat cgtgcatcag atgaatgcct gcttttacgt |
| 2701 | tatttccaag tgttaaacaa ttgattgaga atgcttactg ctggcatcct tgactttggg |
| 2761 | aagtgcccac tgatacagtt taaagcagga ttccttaagg tatctgaaat gagaaccaag |
| 2821 | tttttaaaaa attggcagtg cactcaacac tgttgtagaa cacatgtccc cacaatgtta |
| 2881 | aattgctgag tttgtcaaca gttctcagtt tttgtactta gtccatcttg cagacctatg |
| 2941 | ccagtttgtg ggccacatag tggtactgac tcttttttta catgtgctca cgacatggta |
| 3001 | ttttgttttg tgttgtgtcg ttgctttgta gaattttgat aagctgaatg taaggacaac |
| 3061 | acactataca cctcttgcat gtggttcaaa tcctctgaag agagagattg gggactatat |
| 3121 | caggtaagtg ttgggaggag gcactgtgca aacttacttt cttttgaaaa tgctttcaca |
| 3181 | tcagctattc aggaaggcag tggcatgcca gaatagcttt gtcttgaata tataacctgt |
| 3241 | acccgcagtg aattattttt ctttttaact tttcagattt gttgtttcac tggagcatta |
| 3301 | agagtgggtg atacattaat tttttttttt tccattccag gacaggtttc attaatcttg |
| 3361 | acaagccctc taacccctct tcccatgagg tggtagcctg gattcgacgg atacttcggg |
| 3421 | tggagaagac agggcacagt ggtactctgg atcccaaggt gactggttgt ttaatcgtgt |
| 3481 | gcatagaacg agccactcgc ttggtgaagt cacaacagag tgcaggtatg tgggagaggg |
| 3541 | agggaaggac tggctagagt gaaaagcttt ctgtatttcc cttctatgtt ttgtctgctg |
| 3601 | gaaactattt cttcattaag aaaaaaaaaa atcctaaagc aaggatattg ttttggtttc |
| 3661 | tgtgtacgtt ctgtcccctg ccagatcttg gtggtccgcg ctttttggaa ttccacatac |
| 3721 | taagcatgta ttcatcaagc tgactggatc catttgtccg ggttcagctc tcagcttgtc |
| 3781 | actccaggct tctcactctg tgctaccgct tctgcctaaa gtatccttcg ttcattttta |
| 3841 | tctgagaaaa gcctgcttat cttgcaaggc tttttccacg cagcttctac actgagtact |
| 3901 | cccaactgtc cctgttagta cattgaattc ccagtgagaa agtgtcttca ttctttccca |
| 3961 | catcatcacc atgaaatctc tcttaatgca aattttatag aaacacaagg tcaaactgcc |
| 4021 | gcttttcctg ctgtgcaaaa tgggagtgac tgagcatata agaaatatgg aaaacagaaa |
| 4081 | actcagtggc cacacctgac tactcttttg tcatttttca ggcaaagagt atgtggggat |
| 4141 | tgtccggctg cacaatgcta ttgaaggggg gacccagctt tctagggtaa gtctgcaatt |
| 4201 | gtagggagga caccctttgt tgacttggat ctctgagccc ctaaacatct ggagatgggt |
| 4261 | tagctgtcct gactgactcc ttttgtcccc ttttcagtcg gctgctgcag ccagcctgga |
| 4321 | ccagtgactg cacatgtaca ttctgatgag gtgtttgttt tcatttgtgt ttgttcttag |
| 4381 | gccctagaaa ctctgacagg tgccttattc cagcgacccc cacttattgc tgcagtaaag |
| 4441 | aggcagctcc gagtgaggac catctacgag agcaaaatga ttgaatacga tcctgaaaga |
| 4501 | agattaggtg agtcattagg cctgtgagtg ggtatgaaca cattcttcag gattctgttc |
| 4561 | tcttattaaa agtagatgcc cttgaagttg aagactttct aaaacctgcc ctacagctgg |
| 4621 | gatattgtct gtgggcgata gtttttgtta tcttttgttg aaaaagtgaa cagtgcctag |
| 4681 | taaagtgaga tggcttttac accttttatg atgactgctt ctgctggagt tgaaatgttt |
| 4741 | tcattctgca tttgtgtagt tcggtgcttt gttcaaaggt aagtgttttc accaaagtat |
| 4801 | gttttgcatg tgttttttat ttgctgcaat tttgactgct tagaagtttc tgttgtcaga |
| 4861 | agtaaattta agacaatgcc aattttcctc ccatccatga aacataccat gctgaatata |
| 4921 | cctgcaactt aagtgttaaa aattctgcta atatcagaat tacagatctt tttttttttt |
| 4981 | tttttttttt tgagacggag tctcgctctg tcgcccaggc tggagtgcgg tggcatgatc |
| 5041 | tcggctcact gcaaactctg tctcccgggt tcacaccatt ttcctgcctc agcctcccaa |
| 5101 | gtagctggga ctataggcgc ccgccatcat gcccggctaa ttttttgtac atttagtaga |
| 5161 | gacggggttt caccatgtta gccaggatgg tcttgacctc ctgacgtcgt gatccacccg |
| 5221 | cctctgcctc ccaaagtgct ggaattacag gcgtgagcca ccgtgcctgg ccagaattac |
| 5281 | agatctttac agcaaatgct tatattttaa gaagtgtgtc tgtcacctct agtcttggtg |
| 5341 | gctcagatga aggataactg catttctcaa ccagtgatgt atttacagta ggtgctgctt |
| 5401 | ttcttttgtg tgtgtgtatc ttaggaatct tttgggtgag ttgtgaggct ggcacctaca |
| 5461 | ttcggacatt atgtgtgcac cttggtttgt tattgggagt tggtggtcag atgcaggagc |
| 5521 | ttcggagggt tcgttctgga gtcatgagtg aaaaggtatg tgttacgggg ctagaagttt |
| 5581 | tagagctggt tcttaccatc tgccagcctt tgatggcact ccacttgctt catgtcgtgg |
| 5641 | gaaagatgct ttccaaagtg ttgagttcag tccagggcag cttccctgtt ctgttaatta |
| 5701 | aactttggga cattaaaatg ggctaaggga gatgattggg tagaaagtat tattctattc |
| 5761 | atttgcctcc cagcctacaa aaatgcctgc ttggggtcta atacttcaac ggttaaagat |
| 5821 | gcctggaaga gggcgcggtg gctcacgcct gtaatcccag cactttggga ggccgaggcg |
| 5881 | ggtggatcat gaggtcagga gatcgagacc atcctggcta acaaggtgaa accccgtctc |
| 5941 | tactaaaaat acaaaaaaaa ttagccgggc gcggtggcgg gcgcctgtag tcccagctac |
| 6001 | tcgggaggct gaggcaggag aatggcgtga acccgggaag cggagcttgc agtgagccga |
| 6061 | gattgcgcca ctgcagtccg cagtcccacc tgggcgacag agcgagactc cgtctcaaaa |
| 6121 | aaaaaaaaaa aaaaaaaaaa agatgcctgg aagagccagg actgaagagg ctgaaaacat |
| 6181 | aatgagcttg gactagtaaa caagtgacat tcagttttag gaagtttggg gtctgatggg |
| 6241 | ctgagataca aataaactga attattttca tacatggctg cttttcagga ccacatggtg |
| 6301 | acaatgcatg atgtgcttga tgctcagtgg ctgtatgata accacaagga tgagagttac |
| 6361 | ctgcggcgag ttgtttaccc tttggaaaag ctgttgacat ctcataaacg gctggttatg |
| 6421 | aaagacagtg cagtaagttc cgggttagga gtttatattt ggaaagaacg tactccaaag |
| 6481 | atgacgcttg ctctttttat tgttttcctt gggttttatt ttattttttt caagtccaaa |
| 6541 | ccaagtatat taatttaaag taaaagttta atggtaggca tacttaaaaa taaggcacat |
| 6601 | taggccaggc acagtggctg atgcctataa tcctagcact ttggcaggct gaggcaagag |
| 6661 | gatcacttga gtccaggaag gagtttgaga ccagcttgag cccaggagat caaggctgtg |
| 6721 | agctgtgttt ttatgatgat cagagagttg gccttttata aattaaaaca ttgtcttttt |
| 6781 | ctctaacttg agggataaaa atttctggat atttaaaatg tttgtagttg gtaattattt |
| 6841 | atattcaaat cttgtataga tgttcacatt ttttgtatgt gtataggctg taataaatac |
| 6901 | tgaaagatgg caccataata gttgaaaaaa ctcagtcaca aaacagtgga caacataaat |
| 6961 | gtgatgtcct ctccttatca aaccattggc ttctgggtga atgtaaaggg aggggagaat |
| 7021 | gtgatcaaaa gaggtctagg tgagtggaga tggtatgagg cagatgggtg cattgggtac |
| 7081 | cattgaacct ctggagaggt agccttaact tgtacccact gacctataaa ttctgcacca |
| 7141 | agacttttgt gtgattggtt caacagggct cagtaatctt gttctcaagt tctttaagag |
| 7201 | cctgttattg cctggatctc cagtaaatct gttctttctc ctcataagga ggagagagga |
| 7261 | gtaattaagg aatgccaagg ctatttttat gtttcaaatg aaaatatgga ccacgtgcgg |
| 7321 | tggctcacac ccataatccc agcgctttgg gaggccgggc caggcaaatt gcttgagctc |
| 7381 | aggagtttga gaccagcctg ggcaatatgg tgaaaccctg tctctattaa aaatacaaaa |
| 7441 | attagctggg cgtggtggca cgtgcctgta atcccagctc cttgggagac tgagaatcac |
| 7501 | ttgaacttgg gagatggagg ttgcagtgag ccgagaatgt gccactgcac tccaccctgg |
| 7561 | acaacagagg gaggttctgc ctgaaaatta aaaaaaaaaa aaaaaaaaag aaaatacttg |
| 7621 | tgagtctcta gaacctgaaa acctgaaagt cttctttact acttttgagt cgtgtgtaat |
| 7681 | gtgcggagag tggtacctat agcacagtga ggtggctctt ctgagtacca ctcatgcccc |
| 7741 | ttgcagctag tgggctataa gtgtcatccc tgtttcctgg tgtcaggccc cactcccttg |
| 7801 | ttgtcctcct ttactctggt gtgggagtgg ggtggagggc agcagctggg ccccttacac |
| 7861 | ttggtgccat ctctgcaggt aaatgccatc tgctatgggg ccaagattat gcttccaggt |
| 7921 | gttcttcgat atgaggacgg cattgaggtc aatcaggaga ttgtggttat caccaccaaa |
| 7981 | ggagaagcaa tctgcatggg taagagggga tgtttatttt ttaaattcta aatgtttgtg |
| 8041 | gttacatact aggtgtatat atttatgggg ttcatgagat gttttgatac aggcatgcaa |
| 8101 | tacataataa tcacatcatg gagaatgggg catccatctt aagcatttat cctttgtgtt |
| 8161 | acaaacaatc ctattttact gttagttatt tatcacctta gttattttaa aatatacact |
| 8221 | taagttatta ttgattatag tcaccccgtt gtgctattaa atagtctcac tcttcttgtt |
| 8281 | cattcttttg gttttttggt ggtggtgttt tttttttttt tttttttttg agacagtctt |
| 8341 | gctcttttca cccagactgc agtgcagaga cgtgacctcg gctcactgca acttctgctt |
| 8401 | cccaggttca agcgattctc gtgcctcagc cttccgagta gctaggatta caggtgcaca |
| 8461 | ccatgacacc tggctaattt tttgtatttt tagtagagac agggttttgt catgttggtc |
| 8521 | aggctggtct cgaacttctg acctcaggtg atctgccctc ccaaagtgct gggattacag |
| 8581 | gtgtgagcca ccatgccggg cctgattttt tttttttttg tacccattaa ccatccctac |
| 8641 | ctccctgcca gccccccact accctcccag tctctggtaa ccattctttt actcttctct |
| 8701 | gtccatgagt tcaattgttt cgattcttag ctcccacaaa taagtgagaa catgcaatgt |
| 8761 | ttgtctttct gtgactggcc gattacactt aatataacga tcttcagttc catccatgtt |
| 8821 | gttggaaatg acaggatctc attctttttt aaggctgaat agtaatccat cgtgtatatg |
| 8881 | taccacattt tctttatcca tttatctgtc gatgggcact tcggttgctt ccaaatcttg |
| 8941 | gctactgtga tctctttgat aacactgatt tcctttattt ggggtatata cccagcagtg |
| 9001 | ggattgctgg atcctatggt agctcaattt agtttttcga ggaacctcca aactgttctt |
| 9061 | catagtggtt atactaattt acattcccat caacaatgta caaaggttcc cttttctcca |
| 9121 | catccttgcc agcacttgtc attgcctgcc ttttggatat aaaccatttt aactggagtg |
| 9181 | aaatgatatt tcattgtagt tttgatttgc atttctctcg tgatcagcat tgagcacttt |
| 9241 | ttcatatgcc tgtttgccat ttatttgtct tcttttgaga agtgtctgtt caaatctttt |
| 9301 | gcctgttttt tgatcagatt attagatttt ttttcctata gtgctgagtt ccttatatat |
| 9361 | tctggttatt aatcccttgt cagatgggta gtttgcggat attttctccc attcttatgc |
| 9421 | atatggatat ctagttttcc cagcaccatt tattgaagag actggttttt acccagtgta |
| 9481 | tgttcttggc acctttgtca aaaatgagtt aaatgtaggt gtgtgcattt gcttctgggt |
| 9541 | tccttattct gttccattgg tttatgtgcc tgtttttatg ccagtaccat actgttttgg |
| 9601 | atactgtagc tctatactat aatttgaagt caggtaatgt gattcctcca ggtttgttct |
| 9661 | ttttgcttag gatagctttg gctattttga gtcttttgtg gttccatgta aagtaccagt |
| 9721 | gttttttaac ttgtgaaagc ttagctactg ttggattgtc tcttaagact ttaaatgctc |
| 9781 | tgcgcaccct cttagtgaat gaaccttaga gctattcatt taatgggtct aatttgtacc |
| 9841 | agaagtaagc atgtcatcag gattccttag ctctgtgagc aggcaaggca gccacggctt |
| 9901 | atggacagca gcagaaaagg aatccttaaa acttcaaagg ttctgtggcc ccaaagggag |
| 9961 | tgtgcatgcc gttcttatag acgtcttgag ctgcaagcct gttatgtgta tcgttaggtg |
| 10021 | ctttggttag gtgtttttta acttcagaag tttaacgtca tttcaaaggc ctccttctca |
| 10081 | taatggagta tttcctccat atgcacatcc tgagcaaagg taaccccaat taaagtagta |
| 10141 | gctcagagtt ttaaaagttt taaagtggca tacaacagta gaattggggc tcatcaatta |
| 10201 | tcaattcttt cacccttcaa ataattcttt tctttattca atgcctgtag ctattgcatt |
| 10261 | aatgaccaca gcggtcatct ctacctgcga ccatggtata gtagccaaga tcaagagagt |
| 10321 | gatcatggag agagacactt accctcggaa gtggggttta ggtccaaagg taagtggtac |
| 10381 | tgggttgcgt gccctgccag gttctttttt tttttttttt ttttttaaat ttattttttt |
| 10441 | attgataatt tttgggtgtt tctcacagag ggggatttgg cagggtcata ggacaatagt |
| 10501 | ggagggaagg tcagcagata aacaagtgaa caaaggtctc tggttttcct aggcagagga |
| 10561 | ccctgcggcc ttccgcagtg tttgtgtccc tgggtacttg agattaggga gtggtgatga |
| 10621 | ctcttaacga gcatgctgcc ttcaagcatc tgtttaacaa agcacatctt gcaccgccct |
| 10681 | taatccattt aaccctgagt ggacacagca catgtttcag agagcacagg gttgggggta |
| 10741 | aggtcacaga tcaacaggat cccaaggcag aagaattttt cttagtacag aacaaaatga |
| 10801 | aaagtctccc atgtctactt ctttccacac agacacggca accatccgat ttctcaatct |
| 10861 | tttccccacc tttcccccgc tttctattcc acaaaaccag cattgacacc attctaaa |

SEQ ID NO: 100 Mouse dkc1 (DYSKERIN) dyskeratosis congenita 1, dyskerin,
exon 1 and join CDS (AJ250973.1)

| | |
|---|---|
| 1 | agccaatggg attgagtaga cagtccgcgg gttcgtagag attcctgccc ccgtaccccg |
| 61 | tcctgtcctc ctcctcctcc tectcttctc tacgtccctt ttttatatca gcaagctagg |
| 121 | ggcggtgcaa cggtgggcgg gtcctaatga cgtgcccgga cgggtctcac accataggag |
| 181 | tctacagttc ttagtttgtg gcggcttggg cgggacgcac gcggtgcagt tcaagatggc |
| 241 | ggatgcagaa ggtaagaggt tctggtggca gtgccatgag acctgtgggg cctcctacac |
| 301 | gacccggagc tagttatagt gagattgcat cgtgacccgc ggttccccgt gttcccgctc |
| 361 | gcagtttggc tgtctcacca ctcggctctg gctctacctg tgaatgcacg cattc |

* Included in Table 1 and 2 are RNA nucleic acid molecules (e.g., thymidines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.

* Included in Table 1 and 2 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.

TABLE 3
List of wild-type RIOK2 and RIOK2 loss of function variants

Wild-type RIOK2

| | |
|---|---|
| 1 | MGKVNVAKLR YMSRDDFRVL TAVEMGMKNH EIVPGSLIAS IASLKHGGCN KVLRELVKHK |
| 61 | LIAWERTKTV QGYRLTNAGY DYLALKTLSS RQVVESVGNQ MGVGKESDIY IVANEEGQQF |
| 121 | ALKLHRLGRT SFRNLKNKRD YHKHRHNVSW LYLSRLSAMK EFAYMKALYE RKFPVPKPID |
| 181 | YNRHAVVMEL INGYPLCQIH HVEDPASVYD EAMELIVKLA NHGLIHGDFN EFNLILDESD |
| 241 | HITMIDFPQM VSTSHPNAEW YFDRDVKCIK DFFMKRFSYE SELFPTFKDI RREDTLDVEV |
| 301 | SASGYTKEMQ ADDELLHPLG PDDKNIETKE GSEFSFSDGE VAEKAEVYGS ENESERNCLE |
| 361 | ESEGCYCRSS GDPEQIKEDS LSEESADARS FEMTEFNQAL EEIKGQVVEN NSVTEFSEEK |
| 421 | NRTENYNRQD GQRVQGGVPA GSDEYEDECP HLIALSSLNR EFRPFRDEEN VGAMNQYRTR |
| 481 | TLSITSSGSA VSCSTIPPEL VKQKVKRQLT KQQKSAVRRR LQKGEANIFT KQRRENMQNI |
| 541 | KSSLEAASFW GE (552 aas) (SEQ ID NO: 27) |

Mutation 1: Frameshift Deletion in exon 8

| | |
|---|---|
| 1 | MGKVNVAKLR YMSRDDFRVL TAVEMGMKNH EIVPGSLIAS IASLKHGGCN KVLRELVKHK |
| 61 | LIAWERTKTV QGYRLTNAGY DYLALKTLSS RQVVESVGNQ MGVGKESDIY IVANEEGQQF |
| 121 | ALKLHRLGRT SFRNLKNKRD YHKHRHNVSW LYLSRLSAMK EFAYMKALYE RKFPVPKPID |
| 181 | YNRHAVVMEL INGYPLCQIH HVEDPASVYD EAMELIVKLA NHGLIHGDFN EFNLILDESD |
| 241 | HITMIDFPQM VSTSHPNAEW YFDRDVKCIK DFFMKRFSYE SELFPTFKDI RREDTLDVEV |
| 301 | SASGYTKEMQ ADDELLHPLG PDDKNIETKE GSEFSFSDGE VAEKAEVYGS ENESERNCLE |
| 361 | ESEGCYCRSS GDPEQIKEDS LSEESADARS FEMTEFNQAL EEIKGQVVEN NSVTEFSEEK |
| 421 | NRTENYNRQD GQRVQGGVPA GSDEYEDECP H STOP (451 aas) (SEQ ID NO: 28) |

Mutation 2: Frameshift Addition in exon 3

| | |
|---|---|
| 1 | MGKVNVAKLR YMSRDDFRVL TAVEMGMKNH EIVPGSLIAS IASLKHGGCN KVLRELVKHK |
| 61 | LIAWERTKTV QGYRLTNAGY DYLALKTL FF STOP (90 aas) (SEQ ID NO: 29) |

Mutation 3: Frameshift Deletion in exon 3

| | |
|---|---|
| 1 | MGKVNVAKLR YMSRDDFRVL TAVEMGMKNH EIVPGSLIAS IASLKHGGCN KVLRELVKHK |
| 61 | LIAWERTKTV QGYRLTNAGY DYLALK HFLLGK STOP (92 aas) (SEQ ID NO: 30) |

Mutation 4: Frameshift Deletion in exon 2

| | |
|---|---|
| 1 | MGKVNVAKLR YMSRDDFRVL TAVEMGMKNH EIVPGSLIAS IASLKHGGCN SFKRISET |
| | STOP (58 aas) (SEQ ID NO: 31) |

Mutation 5: Substitution at splice junction exon 2-intron 2

| | |
|---|---|
| 1 | MGKVNVAKLR YMSRDDFRVL TAVEMGMKNH EIVPGSLIAS IASLKHGGCN KVLRELVKHK |
| 61 | LIAWERTK TKYFEAPFVIFQVIGFHQSKCSKSQEVEVARLVKH STOP (103 aas) (SEQ |
| | ID NO: 32) |

### II. Subjects

In some embodiments, the subject is a mammal (e.g., mouse, rat, primate, non-human mammal, domestic animal, such as a dog, cat, cow, horse, and the like), and is preferably a human. In other embodiments, the subject is an animal model of a red blood cell disorder. In some embodiments, the subject is not limited to animals or humans with genetic mutation in RIOK2 or other ribosomal or non-ribosomal protein mutations. For example, the subject may be any aging subject or any subject afflicted by a disorder associated with aging, a hematologic disorder disclosed herein or telomere shortening. The subject may be any subject afflicted by polycythemia vera, a metabolic disorder, MDS or anemia (e.g., anemia associated with a cancer disclosed herein). The subject may have a loss of function mutation in RIOK2. The subject may have a frameshift mutation (e.g., a frameshift deletion mutation or a frameshift addition mutation) that results in a partial or total loss of function in RIOK2.

The subject may have a nucleic acid substitution or deletion mutation that results in a partial or total loss of function in RIOK2. The subject may be any subject comprising a RIOK2 variant listed in Table 3.

In addition, cells can be used according to the methods described herein, whether in vitro, ex vivo, or in vivo, such as cells from such subjects. In some embodiments, the cells are a collection of erythroid progenitors and/or erythroid progenitors defined according to developmental stage (e.g., I, II, III, and IV, and combinations thereof).

In some embodiments encompassed by the methods of the present invention, the subject has not undergone treatment, such as with an erythropoiesis-stimulating agent, an agent to treat anemia, an agent to treat a metabolic disorder, or an anti-aging agent. In other embodiments, the subject has undergone treatment, such as with an erythropoiesis-stimulating agent, an agent to treat anemia, an agent to treat a metabolic disorder, or an anti-aging agent.

The methods and compositions encompassed by the present invention can be used across many different red blood cell disorders in subjects such as those described herein. The red blood cell disorders that can be treated with the disclosed methods include myelodysplastic syndromes (MDS) and anemias, such as, without limitation, anemia caused by insufficiency of serine/threonine-protein kinase RIOK2, anemia caused by mutations or deletions on human chromosome 5, macrocytic anemia, anemia associated with inflammatory disorders, such as rheumatoid arthritis or systemic lupus erythematosus, anemia associated with chronic kidney disease (CKD), stress-induced anemia, aplastic anemia, Diamond Blackfan anemia, and Shwachman-Diamond syndrome. Similarly, the methods and compositions encompassed by the present invention can be used across bone marrow failure syndromes since it has been determined herein that RIOK2 regulates blood cell development and agonists of RIOK2 activity reverse anemia associated with bone marrow failure syndromes, such as aplastic anemia, Diamond Blackfan anemia, amegakaryocytic thrombocytopenia (Amega), dyskeratosis congenita (DC), fanconi anemia (FA), Pearson syndrome, severe congenital neutropenia (SCN), Shwachman-Diamond syndrome (SDS), thrombocytopenia absent radii (TAR) and others. The methods and compositions encompassed by the present invention also can be used across many different disease or disorders associated with aging and/or telomere shortening in subjects such as those described herein. Also, the methods and compositions encompassed by the present invention can be used across many metabolic disease and disorders in subjects such as those described herein. Finally, the methods and compositions encompassed by the present invention can be used in subjects with polycythemia vera.

The ordinarily skilled artisan will appreciate from the results of a wide variety of experimental models described herein that the methods encompassed by the present invention apply generally to a subject having a disease or disorder described herein, and are not limited to individuals having particular genetic mutations. In some particular embodiments, subjects have an loss of function mutation affected the function of RIOK2.

The methods encompassed by the present invention can be used to stratify subjects and/or determine responsiveness of subjects described herein to RIOK2 modulation.

### III. Sample Collection, Preparation and Separation

In some embodiments, biomarker amount and/or activity measurement(s) in a sample from a subject is compared to a predetermined control (standard) sample. The sample from the subject is typically from a diseased tissue. The control sample can be from the same subject or from a different subject. The control sample is typically a normal, non-diseased sample. However, in some embodiments, such as for staging of disease or for evaluating the efficacy of treatment, the control sample can be from a diseased tissue. In some embodiments, the control sample comprises two wild-type copies of RIOK2. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment (e.g., based on the number of genomic mutations and/or the number of genomic mutations causing non-functional proteins), evaluate a response to an agent described herein, and/or evaluate a response to agent described herein. A pre-determined biomarker amount and/or activity measurement(s) may be determined in populations of patients with or without a disease or disorder disclosed herein or an aging patient. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual.

Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of therapy. Post-treatment biomarker measurement can be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment can comprise therapy, such as a therapeutic regimen comprising one or more agents that alter the copy number, expression level, and/or activity of RIOK2.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

a. Polypeptides

An aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly, such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

b. Stapled Peptides

As disclosed herein, the polypeptide agent may comprise an internally cross-linked peptide, including an internally cross-linked peptide that stabilizes or activates a biomarker listed herein or one that blocks or inhibits a biomarker listed herein. The present disclosure provides structurally stabilized peptides related to RIOK2 comprising at least two modified amino acids joined by an internal (intramolecular) cross-link (or staple). Stabilized peptides as described herein include stapled peptides as well as peptides containing multiple staples (e.g., at least one staple, at least two staples, at least three staples, at least four staples, or at least five staples).

The present disclosure provides structurally-stabilized peptides comprising at least two modified amino acids joined by an internal (intramolecular) cross-link (or staple), wherein the at least two amino acids are separated by, e.g., 2, 3, or 6 amino acids. Stabilized peptides herein include stapled peptides, including peptides having, e.g., 1, 2, 3, 4, 5, or more staples and/or stitched peptides.

A compound herein can exhibit helical stability by the maintenance of u-helical structure by a compound of the invention as measured by circular dichroism or NMR. For example, in some aspects, the compound exhibits at least a 1.25, 1.5, 1.75 or 2-fold increase in u-helicity as determined by circular dichroism compared to a corresponding un-cross-linked peptide. In some aspects, the compound can exhibit about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% helicity.

Amino acids are the building blocks of the peptides herein. The term “amino acid” may refers to a molecule containing both an amino group and a carboxyl group as well as a side chain. Amino acids suitable for inclusion in the peptides disclosed herein include, without limitation, natural alpha-amino acids such as D- and L-isomers of the 20 common naturally occurring alpha-amino acids found in peptides (e.g., Ala (A), Arg (R), Asn (N), Cys (C), Asp (D), Gln (Q), Glu (E), Gly (G), His (H), Ile (I), Leu (L), Lys (K), Met (M), Phe (F), Pro (P), Ser (S), Thr (T), Trp (W), Tyr (Y), and Val (V), unnatural alpha-amino acids (including, but not limited to α,α-disubstituted and N-alkylated amino acids), natural beta-amino acids (e.g., beta-alanine), and unnatural beta-amino acids. Amino acids used in the construction of peptides of the present invention can be prepared by organic synthesis, or obtained by other routes, such as, for example, degradation of or isolation from a natural source.

There are many known unnatural amino acids any of which may be included in the peptides of the present invention. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-i-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta-and/para-substituted phenylalanines (e.g., substituted with —C(═O)C6H5; —CF3; —CN; -halo; —NO2; CH3), disubstituted phenylalanines, substituted tyrosines (e.g., further substituted with —C═O)C6H5; —CF3; —CN; -halo; —NO2; CH3), and statine. Additionally, amino acids can be derivatized to include amino acid residues that are hydroxylated, phosphorylated, sulfonated, acylated, and glycosylated, to name a few.

A “peptide” or “polypeptide” comprises a polymer of amino acid residues linked together by peptide (amide) bonds. The terms, as used herein, refer to proteins, polypeptides, and peptides of any size, structure, or function. Typically, a peptide or polypeptide will be at least three amino acids long. A peptide or polypeptide may refer to an individual protein or a collection of proteins. In some instances, peptides can include only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. “Dipeptide” refers to two covalently linked amino acids.

In some aspects, the present disclosure provides internally cross-linked peptides comprising the amino acid sequence: Set forth in SEQ ID NO: 5 and 11, wherein: wherein 8 is R5-octenyl alanine and X is S5-pentenyl alanine. The side chains of two amino acids may be separated by two, three, or six amino acids are replaced by an internal staple; the side chains of four amino acids are replaced by two internal staples, or the side chains of five amino acids are replaced by the combination of an internal staple and an internal stitch.

In some instances, the internal staples and/or the internal stitch comprises at least two internal staples (replacing the side chains of 4 amino acids, i.e., each staple is between two amino acids separated by 3 amino acids). In some instances, the internal staples and/or the internal stitch comprises a combination of at least one internal staple and an internal stitch. In some instances, the internal stitch replaces the side chain of a first amino acid and a second and a third amino acid thereby cross-linking the first amino acid (which lies between the second and third amino acids) to the second and third amino acid via an internal cross-link, wherein the first and second amino acid are separated by two, three, or six amino acids, the first and the third amino acids are separated by two, three, or six amino acids, and the second and third amino acids are distinct amino acids. In some aspects, the internal stitch replacing the side chains of the three amino acids cross-links a pair of amino acids separated by two, three, or six amino acids. In some aspects, the side chains of the four amino acids of the internally cross-linked polypeptides of the disclosure are replaced by two distinct internal staples. In some aspects, a first of the two distinct internal staples cross-links a first pair of amino acids separated by two, three, or six amino acids, and a second of the at least two distinct internal staples cross-links a second pair of amino acids separated by two, three, or six amino acids.

In some instances, peptides can include (e.g., comprise, consist essentially of, or consist of) at least 5, at least 6, at least 7, at least 8, at least 9, at least 10, at least 11, or more contiguous amino acids of a sequence selected from:

(SEQ ID NO: 5)
GSLIASIAS
(SEQ ID NO: 11)
SNKVLRELVKH.

The peptides can be modified by an internal cross-link. In each case, an example of a cross-linked variant is included (X indicates an amino acid whose side chain has been replaced by an internal staple). Also provided are internally cross-linked polypeptides that include (i) the amino acid sequence of GSLIASIAS (SEQ ID NO: 5) or (ii) a sequence containing between one to six amino acid substitutions in the sequence of SEQ ID NO: 5.

Also provided are internally cross-linked polypeptides that include (i) the amino acid sequence of SNKVLRELVKH (SEQ ID NO: 11) or (ii) a sequence containing between one to seven amino acid substitutions in the sequence of SEQ ID NO: 11.

In some embodiments of any of the internally cross-linked polypeptides described herein, the internal staples and/or the internal stitch replacing the side chains of the three amino acids comprises at least two internal staples. The internally cross-linked peptide may comprise all-hydrocarbon staples, such as i, i+4 or i, i+7 staples.

Exemplary peptides that can be used to generate the internally cross-linked polypeptides described herein can include a sequence (e.g., at least 10, 11, 12, 13, 14, 15, 16, 17, 18, 19, 20, 21, 22, 23, 24, 25, 26, 27, 28, 29, or 30 amino acids, or between 10-30, 15-24, 15-25, 17-25, or 15-22 amino acids) that is at least 70% (e.g., at least 75%, 80%, 85%, 90%, 95%, 96%, 97%, 98%, 99%, or 100%) identical to a DNA binding domain or a transrepressor domain of RIOK2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared.

When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total #of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natd. Acad. Sci. U.S.A. 87:2264-2268, modified as in Karlin and Altschul (1993) Proc. Natd. Acad. Sci. U.S.A. 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See the following address on the World Wide Web:.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natd. Acad. Sci. U.S.A. 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide of the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. U.S.A. 89:7811-7815; Delgrave et al., 1993, Protein Engineering 6(3):327-331).

An isolated polypeptide or a fragment thereof (or a nucleic acid encoding such a polypeptide) corresponding to one or more biomarkers encompassed by the present disclosure, including the biomarkers listed in Table 1 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein). While there are commercially available antibodies against RIOK2, generation of an antibody as described herein may provide additional detection tools for the diagnostic and prognostic methods described herein. Furthermore, generation of an antibody or bispecific antibody/intrabody (e.g., those stabilizing the interaction between the biomarker and its substrate to increase the activity of the biomarker) may be useful for the methods described herein.

c. Antibodies

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more biomarkers encompassed by the present disclosure, including the biomarkers listed in Table 1, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, MD. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody encompassed by the present disclosure are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. U.S.A. 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. U.S.A. 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an introbody, to bind a desired target, such as one or more biomarkers listed in Table 1, and/or a binding partner thereof, either alone or in combination with an immunotherapy, such as the one or more biomarkers, the binding partners/substrates of such biomarkers, or an immunotherapy effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially introbodies, that retain at least one functional property of the antibodies of the present invention, such as binding to one or more biomarkers listed in Table 1, the binding partners/substrates of such one or more biomarkers, and/or an immune checkpoint. Another functional property includes inhibiting binding of the original known, non-human or human antibodies in a competition ELISA assay.

Antibodies, immunoglobulins, and polypeptides encompassed by the present disclosure can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present invention can be conjugated to a radioisotope, e.g., radioactive iodine, to generate cytotoxic radiopharmaceuticals for treating a related disorder.

Conjugated antibodies, in addition to therapeutic utility, can be useful for diagnostically or prognostically to monitor polypeptide levels in tissue as part of a clinical testing procedure, e.g., to determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, 0-galactosidase, or acetylcholinesterase; examples of suitable prosthetic group complexes include a flag tag, a myc tag, an hemagglutinin (HA) tag, streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody, as well as indirect labeling of the antibody by reactivity with a detectable substance.

The antibody conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. U.S.A., 83:1453, and Staerz and Bevan (1986) Immunol. Today 7:241). Bispecific antibodies are also described in U.S. Pat. No. 5,959,084. Fragments of bispecific antibodies are described in U.S. Patent 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present disclosure, including one or more biomarkers listed in Table 1, or a fragment thereof. In one embodiment, the bispecific antibody could specifically bind to both a polypeptide or a fragment thereof and its natural binding partner(s) or a fragment(s) thereof.

Techniques for modulating antibodies, such as humanization, conjugation, recombinant techniques, and the like are well-known in the art.

In another aspect of this invention, peptides or peptide mimetics can be used to antagonize the activity of one or more biomarkers encompassed by the present disclosure, including one or more biomarkers listed in Table 1, or a fragment(s) thereof. In one embodiment, variants of one or more biomarkers listed in Table 1 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. U.S.A. 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers encompassed by the present disclosure, including one or more biomarkers listed in Table 1, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences described herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments encompassed by the present disclosure. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides described herein can be used therapeutically to treat disease, e.g., by altering costimulation in a patient.

Peptidomimetics (Fauchere (1986) Adv. Drug Res. 15:29; Veber and Freidinger (1985) TINS p.392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2—CH2—, —CH═CH— (cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2—); Spatola, A. F. et al. (1986) Life Sci. 38:1243-1249 (—CH2—S); Hann, M. M. (1982) J. Chem. Soc. Perkin Trans. 1. 307-314 (—CH—CH—, cis and trans); Almquist, R. G. et al. (190) J. Med. Chem. 23:1392-1398 (—COCH2—); Jennings-White, C. et al. (1982) Tetrahedron Lett. 23:2533 (—COCH2—); Szelke, M. et al. European Appln. EP 45665 (1982) CA: 97:39405 (1982)(—CH(OH)CH2—); Holladay, M. W. et al. (1983) Tetrahedron Lett. (1983) 24:4401-4404 (—C(OH)CH2—); and Hruby, V. J. (1982) Life Sci. (1982) 31:189-199 (—CH2—S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present disclosure are small molecules which can modulate (either enhance or inhibit) interactions, e.g., between biomarkers described herein or listed in Table 1 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. U.S.A. 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP 409), plasmids (Cull et al. (1992) Proc. Natl. Acad. Sci. U.S.A. 89:1865-1869) or on phage (Scott and Smith (1990) Science 249:386-390); (Devlin (1990) Science 249:404-406); (Cwirla et al. (1990) Proc. Natl. Acad. Sci. U.S.A. 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Chimeric or fusion proteins can be prepared for one or more biomarkers listed in Table 1, and/or agents for the immunotherapies described herein. As used herein, a “chimeric protein” or “fusion protein” comprises one or more biomarkers encompassed by the present disclosure, including one or more biomarkers listed in Table 1, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers encompassed by the present disclosure, including one or more biomarkers listed in Table 1, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ4 domain (e.g., the hinge, CH2 and CH3 regions of human IgCγ1, or human IgC γ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein encompassed by the present disclosure is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

The fusion proteins encompassed by the present disclosure can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

d. Nucleic Acids

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, NY, 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6X sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention.

Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an u-anomeric nucleic acid molecule. An u-anomeric nucleic acid molecule forms specific double-stranded hybrids with complementary RNA in which, contrary to the usual a-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742).

Alternatively, an mRNA encoding a polypeptide of the present invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N. Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. U.S.A. 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. U.S.A. 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. U.S.A. 84:648-652; PCT Publication No.

WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers encompassed by the present disclosure, including one or more biomarkers listed in Table 1, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21,20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA. In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences encompassed by the present disclosure may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Table 1). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21,20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. WO88/09810) or the blood-brain barrier (see, e.g., PCT Publication No. WO89/10134), hybridization-triggered cleavage agents. (See, e.g., Krol et al. (1988) BioTech. 6:958-976) or intercalating agents. (See, e.g., Zon (1988) Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are a-anomeric oligonucleotides. An u-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells, or piwiRNAs. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nat. Biotechnol. 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET I1d (Studier et al., p. 60-89, In Gene Expression Technology: Methods in Enzymology vol. 185, Academic Press, San Diego, CA, 1991). Target biomarker nucleic acid expression from the pTrc vector relies on host RNA polymerase transcription from a hybrid trp-lac fusion promoter. Target biomarker nucleic acid expression from the pET I1d vector relies on transcription from a T7 gn10-lac fusion promoter mediated by a co-expressed viral RNA polymerase (T7 gnl). This viral polymerase is supplied by host strains BL21 (DE3) or HMS174(DE3) from a resident prophage harboring a T7 gnl gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, CA, 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSecl (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, CA), and pPicZ (Invitrogen Corp, San Diego, CA).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. U.S.A. 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the a-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes.

The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995).

Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the a- and 0-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. U.S.A. 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. U.S.A. 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed.

Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J.Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 2, 3, 4, 5, 6, 7, 8, 9, or 10 is predictive of better outcome of a treatment with an agent discribed herein.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number.

In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. U.S.A. 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci U.S.A. 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan™ and SYBR™ green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. U.S.A. 86: 1173), self-sustained sequence replication (Guatelli, et al. (1990) Proc. Nat. Acad. Sci. U.S.A. 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, NY).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS U.S.A. 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. U.S.A., 87, 1874 (1990)); and transcription amplification (see, e.g., Kwoh et al., Proc. Natl. Acad. Sci. U.S.A. 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, 32P and 35S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample. c. Methods for Detection of Biomarker Protein Expression The activity or level of a biomarker protein can be detected and/or quantified by detecting or quantifying the expressed polypeptide. The polypeptide can be detected and quantified by any of a number of means well-known to those of skill in the art. In some embodiments, aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response to an agent. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as 1251 or 35S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected.

Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including 1251, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (125I, 1211), carbon (14C), sulphur (35S), tritium (3H), indium (1121n), and technetium (99mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10-6M, 10-7M, 10-8M, 10-9M, 10-10M, 10-11M, 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule for the treatment, diagnostic, and prognostic methods described herein.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. U.S.A. 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. U.S.A. 74:560 or Sanger (1977) Proc. Natl. Acad Sci. U.S.A. 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. U.S.A. 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci U.S.A. 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. U.S.A. 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a new restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci U.S.A. 88:189). In such cases, ligation will occur only if there is a perfect match at the 3′ end of the 5′ sequence making it possible to detect the presence of a known mutation at a specific site by looking for the presence or absence of amplification.

### VI. Further Uses and Methods Encompassed by the Present Invention

The methods and compositions described herein can be used in a variety of screening, diagnostic, and prognostic applications in addition to therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell-based assays and animal model assays. In one embodiment, the assays provide a method for identifying whether an agent is useful for treating a disease or disorder disclosed herein, such as anemia (e.g., an anemia disclosed herein), a metabolic disorder, polythycemia vera, and/or aging and/or a disease or disorder associated with telomere shortening.

In one embodiment, the present invention relates to assays for screening test agents which bind to, or modulate the biological activity of, at least one biomarker described herein (e.g., in the Tables, Figures, Examples, or otherwise in the specification). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate the activity of the biomarker, such as by measuring direct binding of substrates or by measuring indirect parameters as described below, and optionally further determining the effect on treating a disease or disorder disclosed herein, such as a disease or disorder disclosed herein, such as anemia, a metabolic disorder, polythycemia vera, and/or aging and/or a disease or disorder associated with telomere shortening.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemission or by scintillation counting.

Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further encompasses novel agents identified by the above-described screening assays. Accordingly, it is within the scope of the present invention to further use an agent identified as described herein, such as in an appropriate animal model.

For example, an agent identified as described herein can be used in an animal model to determine the efficacy, toxicity, or side effects of treatment with such an agent.

Alternatively, an agent, such as an antibody, identified as described herein can be used in an animal model to determine the mechanism of action of such an agent.

b. Diagnostic and Predictive Medicine

The present invention also pertains to the field of predictive medicine in which diagnostic assays, prognostic assays, and monitoring clinical trials are used for prognostic (predictive) purposes to thereby stratify subject populations and/or treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an an aging individual or an individual afflicted with a disease or disorder disclosed herein is likely to respond to biomarker inhibitor treatments. Such assays can be used for prognostic or predictive purpose alone or can be coupled with a therapeutic intervention to thereby prophylactically treat an individual prior to the onset or after recurrence of a disorder characterized by or associated with biomarker polypeptide, nucleic acid expression or activity. The ordinarily skilled artisan will appreciate that any method can use one or more (e.g., combinations) of biomarkers described herein, such as those in the Tables, Figures, Examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The ordinarily skilled artisan will also appreciate that, in certain embodiments, the methods of the present invention may implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from tissue of interest.

In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods encompassed by the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the tissue of a subject not afflicted with a disease or disorder disclosed herein, and/or profiles generated from population-based distributions of informative loci of interest in relevant populations of the same species can be stored and later compared to that of a sample derived from tissue of instructed, such as tissue suspected of being relevant to a disease or disorder disclosed herein of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Prognostic assay methods are also provided that may be used to identify subjects having or at risk of developing a disease or disorder disclosed herein that is likely or unlikely to be responsive to a modulator of RIOK2. Assays described herein, such as the preceding diagnostic assays or the following assays, can be utilized to identify a subject having or at risk of developing a disorder associated with a dysregulation of the amount or activity of at least one biomarker described herein, such as a disease or disorder disclosed herein. Alternatively, the prognostic assays can be utilized to identify a subject having or at risk for developing a disorder associated with a dysregulation of at least one biomarker described herein. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

The present invention provides, in part, methods, systems, and code for accurately classifying whether a biological sample is associated with a disease or disorder disclosed herein that is likely to respond to a modulator of RIOK2. In some embodiments, the present invention is useful for classifying a sample (e.g., from a subject) as associated with or at risk for responding to or not responding to RIOK2 modulation using a statistical algorithm and/or empirical data (e.g., the amount or activity of a biomarker described herein, such as in the Tables, Figures, Examples, and otherwise described in the specification).

An exemplary method for detecting the amount or activity of a biomarker described herein, and thus useful for classifying whether a sample (e.g., a sample from a subject having a disease or disorder disclosed herein or an in vitro model of such a disease or disorder) is likely or unlikely to respond to RIOK2 modulation involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely responder or non-responder with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of ordinary skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used.

In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., a hematologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have an a disease or disorder disclosed herein of interest or a sample that is susceptible to biomarker inhibitor treatment), a biological sample from the subject during remission, or a biological sample timepoint during treatment for the condition.

c. Clinical Efficacy

Similarly, clinical efficacy can be measured by any method known in the art. For example, the benefit from a therapy with an agent that modulates a biomarker disclosed herein (e.g., RIOK2), alone or in combination with a another agent, or an erythropoiesis-stimulating agent (e.g., erythropoietin, epoetin alfa, epoetin beta, epoetin omega, epoetin zeta, darbepoetin alfa, IL-9), relates to an increase in the level of healthy red blood cells so that adequate oxygen can be carried to the tissues of the subject. As another example, the benefit from a therapy disclosed herein can relate to the level of red blood cells in the blood (e.g., hematocrit) or the level of hemoglobin in the blood, both of which can be measured as part of a routine complete blood count.

The benefit from a therapy with an agent that modulates a biomarker disclosed herein (e.g., RIOK2), alone or in combination with another agent, for the treatment of aging or telomere shortening may relate to a slowing of telomere shortening or a lack of change in telomere length.

The benefit from a therapy with an agent that modulates a biomarker disclosed herein (e.g., RIOK2), alone or in combination with another agent, for the treatment of metabolic disorder may relate to restoration of glucose homeostasis. As another example, the benefit from a therapy disclosed herein may relate to weight loss.

The benefit from a therapy with an agent that modulates a biomarker disclosed herein (e.g., RIOK2), alone or in combination with an additional agent, for the treatment of polycythemia vera may relate to inhibiting or blocking excessive red blood cell formation.

The benefit from using agents encompassed by the present invention can be determined by measuring the level of cytotoxicity in a biological material. The benefit from using agents encompassed by the present invention can be assessed by measuring transcription profiles, viability curves, microscopic images, biosynthetic activity levels, redox levels, and the like. The benefit from using agents encompassed by the present invention can also be determined by measuring the presence and severity of side effects from the treatment such as autoimmune or allergic sequelae.

In some embodiments, clinical efficacy of the therapeutic treatments described herein can be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular therapeutic regimen is at least 25%, 30%, 35%, 40%, 45%, 50%, 55%, 60%, 65%, 70%, 75%, 80%, 85%, or more.

For example, in order to determine appropriate threshold values, a particular therapeutic regimen can be administered to a population of subjects and the outcome can be correlated to biomarker measurements detailed previously that were determined prior to administration of any therapy. The outcome measurement can be pathologic response to therapy. Alternatively, outcome measures, such as overall survival and disease-free survival can be monitored over a period of time for subjects following therapies for whom biomarker measurement values are known as detailed previously. In certain embodiments, the same doses of therapy agents, if any, are administered to each subject. In related embodiments, the doses administered are standard doses known in the art for those agents used in therapies. The period of time for which subjects are monitored can vary. For example, subjects can be monitored for at least 2, 4, 6, 8, 10, 12, 14, 16, 18, 20, 25, 30, 35, 40, 45, 50, 55, or 60 months.

### VII. Administration of Agents

The agents encompassed by the present invention (e.g., agents that modulate RIOK2) are administered to subjects in a biologically compatible form suitable for pharmaceutical administration in vivo, to enhance their effects. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition encompassed by the present invention is defined as an amount effective, at dosages and for periods of time necessary, to achieve the desired result. For example, a therapeutically active amount of an agent can vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Agents encompassed by the present invention can be administered either alone or in combination with an additional therapy. In the combination therapy, an agent encompassed by the present invention and another agent, such as lenalidomide, azacitidine, decitabine, or an erythropoiesis-stimulating agent (e.g., erythropoietin, epoetin alfa, epoetin beta, epoetin omega, epoetin zeta, darbepoetin alfa), a hypomethylating agent, or an anti-aging agent, can be delivered to the same or different cells and can be delivered at the same or different times. The agents encompassed by the present invention can be incorporated into pharmaceutical compositions suitable for administration. Such compositions can comprise one or more agents or one or more molecules that result in the production of such one or more agents and a pharmaceutically acceptable carrier.

The therapeutic agents described herein can be administered using a mode or route of administration that delivers them to the particular locations in the body where RIOK2 expression can be modulated.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which can inactivate the compound. For example, for administration of agents, by other than parenteral administration, it can be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions encompassed by the present invention can be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intra-vaginally or intra-rectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods encompassed by the present invention can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like.

Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations can conveniently be presented in unit dosage form and can be prepared by any methods well-known in the art of pharmacy.

The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound can also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered peptide or peptidomimetic moistened with an inert liquid diluent.

Tablets, and other solid dosage forms, such as dragees, capsules, pills and granules, can optionally be scored or prepared with coatings and shells, such as enteric coatings and other coatings well-known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions, which can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration can be presented as a suppository, which can be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component can be mixed under sterile conditions with a pharmaceutically-acceptable carrier, and with any preservatives, buffers, or propellants which can be required.

The ointments, pastes, creams and gels can contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agents disclosed herein can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions encompassed by the present invention suitable for parenteral administration comprise one or more therapeutic agents in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain antioxidants, buffers, bacteriostats, solutes which render the formulation isotonic with the blood of the intended recipient or suspending or thickening agents.

Examples of suitable aqueous and nonaqueous carriers which can be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in pharmaceutical compositions encompassed by the present invention can be determined by the methods encompassed by the present invention to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

In one embodiment, an agent encompassed by the present invention is an antibody.

As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors can influence the dosage required to effectively treat a subject, including but not limited to the severity of the disease or disorder, previous treatments, the general health and/or age of the subject, and other diseases present.

Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment can increase or decrease over the course of a particular treatment. Changes in dosage can result from the results of diagnostic assays.

### EXAMPLES

### Example 1: Materials and Methods for Example 2

### Primary and Secondary Cell Culture

CD34+ primary human hematopoietic stem and progenitor cells (HSPCs) were obtained from Fred-Hutchinson Cancer Research center, Seattle, USA. The cells were thawed and washed with lx PBS constituting 1% human AB serum, followed by revival in expansion medium- StemSpan SFEM II medium supplemented with 1X CC100 (STEMCELL Technologies), 1% penicillin-streptomycin (P/S), 1% Glutamine and 10 ng/ml TPO. The cells were subjected to differentiation medium containing IMDM reconstituted with 3% human AB serum, 2% human AB plasma, 1% P/S, 0.06% heparin solution (STEMCELL Technologies), 1 ng/ml IL-3 (Peprotech, Inc.), 10 ng/ml SCF (Peprotech, Inc.), 200 μg/ml holo-transferrin (Sigma-Aldrich) and 3U/ml erythropoietin (DFCI pharmacy). For selective differentiation of HSPCs into myeloid and megakaryocytic lineages, HSPCs were cultured in SFEM II media supplemented with myeloid expansion supplement II 100X (#02694) or megakaryocyte expansion supplement 100X (#02696) (STEMCELL Technologies), respectively. Cells were cultured at a density of 0.1-0.01×10⁶ cells/ml and incubated at 37° C. with 5% CO₂. Media were changed every alternate day, as required. These protocols are previously described (Khajuria, R. K. et al. Ribosome Levels Selectively Regulate Translation and Lineage Commitment in Human Hematopoiesis. Cell 173, 90-103 e119 (2018)).

TF-1 human erythroblast cells were purchased from ATCC (ATCC® CRL-2003TM) and maintained in RPMI-1640 medium (ATCC® 30-2001TM) containing 10% fetal bovine serum (FBS), 2 ng/ml GM-CSF and 1% P/S. For differentiation, the cells were cultured in RPMI-1640 medium containing 10% FBS, 5 U/ml erythropoietin and 1% P/S. K562 human erythroid cells were purchased from ATCC (ATCC® CCL-243™) and maintained in IMDM medium (ATCC® 30-2005TM) containing 10% FBS and 1% P/S. For differentiation, the cells were cultured in IMDM medium containing 10% FBS, 40 μM hemin and 1% P/S. HEK293FT cells were purchased from ATCC and maintained in DMEM medium supplemented with 10% FBS and 1% P/S.

### Generation of Lentiviral Vectors and Infection

The shRNA constructs targeting human RIOK2 (shRIOK2-1 and shRIOK2-2) and scrambled shRNA were obtained from the Mission shRNA collection (SHCLNG-NM_018343, Sigma-Aldrich). RIOK2 was cloned in the pHAGE-MCS-IRES-ZsGreen lentiviral vector with an N-terminal Hemagglutinin (HA) tag. DNA binding mutant (DBM), transactivation domain deletion 1 and 2 (ΔTAD1 and ΔTAD2), transrepressor domain deleted (ATRD) and kinase-dead (K123A) mutants were generated by site-directed mutagenesis. N-terminal extension (NTE) construct was generated by cloning the first 92 amino acids of RIOK2 with an N-terminal HA-tag.

For lentiviral production, HEK293FT cells were transiently transfected with pVSV-G, pDelta8.9 and RIOK2 vectors using X-tremeGENE™ HP DNA transfection reagent as per the manufacturer's protocol. Viral supernatant was collected 48 hours post transfection. Primary HSPCs or secondary cells were spinfected at a density of 0.1-0.2×10⁶ cells per well in 6-well plates with 8 μg/ml polybrene (Millipore) at 3392 xg for 1.5 h at 32° C. and left O/N. The medium was changed the next morning. Lentiviral transduction efficiency reached 60-75% for primary human HSPCs and >95% for secondary cells after 48 hours of infection. The positively transduced cells (ZsGreen+) were FAC-sorted for further analysis.

### CRISPR/Cas9 Gene Editing

Primary human HSPCs and human erythroblast cells (TF-1) were electroporated using Lonza-Amaxa 4D nucleofector unit as per the manufacturer's protocols. For ribonucleoprotein (RNP) formation, tracer RNAs and crRNAs (IDT) were incubated at 95° C. for 5 mins at equal molar ratios and mixed with equal molar concentration of Cas9 peptide (IDT). This was followed by incubation at 37° C. for 15 minutes and the RNP mix was kept at 4° C. until use within the next 2 hours. Cells were electroporated at a density of 0.1-0.2×10⁶ cells per well of a 16-well electroporation-strip. Immediately after electroporation, fresh medium was added to the cells and transferred to 37° C. Genome editing efficiency was analyzed 48-72 h after electroporation by nucleotide sequencing, quantitative RT-PCR, and immublotting. The crRNAs used for knockdown (KD) of RIOK2:

(SEQ ID NO: 33)
GAACGGCGGGTTTCTTACCG
and
(SEQ ID NO: 34)
CATTTGTCAACCGATAGCCC,
crRNAs used for knockout (KO) of RIOK2:
(SEQ ID NO: 35)
TGACTTCAGGGTCTTGACCG
and
(SEQ ID NO: 36)
TGATTACAATCGTCATGCAG.
crRNA against LacZ (Control):
(SEQ ID NO: 37)
TTCTCCGCGGGAACAAACGG.

### Luciferase Reporter Assay

For transactivation assays, the promoter regions of RIOK2, GATA1, GATA2, SPIl, RUNX3 and KLF1 (500-750 bps upstream of the ATG start codon) were cloned in pGL3.1 basic vector. Luminescence intensities were normalized using co-transfection of Renilla expression vector in HEK293 cells using Lipofectamine 3000 reagent. Dual luciferase assays (Promega) were performed as per manufacturer's instructions. Luminescence intensities were captured using EnVision™ Multimode plate reader (Perkin Elmer).

Quantitative RT-PCR RNA was isolated from cells using the RNeasy Plus Micro kit (Qiagen) using manufacturer's protocols. Genomic DNA (gDNA) was removed using gDNA eliminator spin columns, followed by isolation of total RNA using phenol-free RNeasy MinElute spin columns. Reverse transcription was performed using qScript cDNA Synthesis kit (QuantaBio). Quantitative real-time polymerase chain reaction (RT-PCR) was performed using Quantstudio6™ RT-PCR system (Applied Biosciences) and PerfeCTa SYBR Green FastMix™ Reaction Mixes (QuantaBio). Comparative C_{T} method has been used for all quantifications using corresponding 13-Actin mRNA levels for normalization. The primers used for RT-PCR are listed in Table 4 below.

TABLE 4
Primers for quantitative RT-PCR

| Primer | | |
|---|---|---|
| Name | Sequence | SEQ ID NO: |
| RIOK2- | 5′-GCT ATC GGT TGA CAA | 38 |
| forward | ATG CAG G-3′ | |
| primer | | |
| RIOK2- | 5′-GAA ACG AGG TTC TTC | 39 |
| reverse | CTA GTC TG-3′ | |
| primer | | |
| GATA1- | 5′-TGC GGC CTC TAT CAC | 40 |
| forward | AAG ATG-3′ | |
| primer | | |
| GATA1- | 5′-CTG CCC GTT TAC TGA | 41 |
| reverse | CAA TCA-3′ | |
| primer | | |
| GATA2- | 5′-ACT GAC GGA GAG | 42 |
| forward | CAT GAA GAT-3′ | |
| primer | | |
| GATA2- | 5′-CCG GCA CAT AGG | 43 |
| reverse | AGG GGT A-3′ | |
| primer | | |
| SPI1- | 5′-GTG CCC TAT GAC ACG | 44 |
| forward | GAT CTA-3′ | |
| primer | | |
| SPI1- | 5′-AGT CCC AGT AAT GGT | 45 |
| reverse | CGC TAT-3′ | |
| primer | | |
| RUNX3- | 5′-AGG CAA TGA CGA | 46 |
| forward | GAA CTA CTC C-3′ | |
| primer | | |
| RUNX3- | 5′-CGA AGG TCG TTG | 47 |
| reverse | AAC CTG G-3′ | |
| primer | | |
| KLF1- | 5′-GGT TGC GGC AAG | 48 |
| forward | AGC TAC A-3′ | |
| primer | | |
| KLF1- | 5′-GTC AGA GCG CGA | 49 |
| reverse | AAA AGC AC-3′ | |
| primer | | |
| HBA1- | 5′-CAA CTT CAA GCT | 50 |
| forward | AAG CCA CTG C-3′ | |
| primer | | |
| HBA1- | 5′-CGG TGC TCA CAG | 51 |
| reverse | AAG CCA G-3′ | |
| primer | | |
| ITGB3- | 5′-CAT GAA GGA TGA | 52 |
| forward | TCT GTG GAG C-3′ | |
| primer | | |
| ITGB3- | 5′-AAT CCG CAG GTT ACT | 53 |
| reverse | GGT GAG-3′ | |
| primer | | |
| ITGAM- | 5′-ACT TGC AGT GAG | 54 |
| forward | AAC ACG TAT G-3′ | |
| primer | | |
| ITGAM- | 5′-TCA TCC GCC GAA AGT | 55 |
| reverse | CAT GTG-3′ | |
| primer | | |
| HBB- | 5′-CTG AGG AGA AGT | 56 |
| forward | CTG CCG TTA-3′ | |
| primer | | |
| HBB- | 5′-AGC ATC AGG AGT | 57 |
| reverse | GGA CAG AT-3′ | |
| primer | | |
| FECH- | 5′-GGA GAT GTT CAC | 58 |
| forward | GAC TTC CTT C-3′ | |
| primer | | |
| FECH- | 5′-GAA TGG TGC CAG CTT | 59 |
| reverse | ATT CTG A-3′ | |
| primer | | |
| KEL- | 5′-CGG GTG CTG ACA | 60 |
| forward | GCT ATC C-3′ | |
| primer | | |
| KEL- | 5′-ACA CAC AGA TGT CTC | 61 |
| reverse | ACA GGG-3′ | |
| primer | | |
| SPTA1- | 5′-GCG ACA TCA GTG | 62 |
| forward | GAG CTA GG-3′ | |
| primer | | |
| SPTA1- | 5′-CGG CAC ACT CAT TGG | 63 |
| reverse | CAT ATT G-3′ | |
| primer | | |
| TFRC- | 5′-GGC TAC TTG GGC TAT | 64 |
| forward | TGT AAA GG-3′ | |
| primer | | |
| TFRC- | 5′-CAG TTT CTC CGA CAA | 65 |
| reverse | CTT TCT CT-3′ | |
| primer | | |
| ACTIN- | 5′-GCG AGA AGA TGA | 66 |
| forward | CCC AGA TC-3′ | |
| primer | | |
| ACTIN- | 5′-CCA GTG GTA CGG | 67 |
| reverse | CCA GAG G-3′ | |
| primer | | |

### Immunoblotting

Cells were washed with ice cold 1x PBS, then resuspended in RIPA lysis buffer (Life Technologies) supplemented with 1x complete protease inhibitor cocktail and 1x phosphatase inhibitor cocktail (Thermo Scientific). Lysis was carried out on a rocker at 4° C. for 15-30 minutes followed by removal of cellular debris by centrifugation at 14,000×g for 20 minutes. The supernatant was mixed with Laemmli buffer and incubated at 95° C. for 10 minutes. Equal amounts of proteins were resolved by SDS-PAGE. The proteins were then transferred onto PVDF membranes (Thermo Fisher Scientific) followed by blocking and probing with the primary antibodies diluted in fresh blocking buffer at 4° C. overnight (O/N): HA rabbit monoclonal antibody at 1:1000 (C29F4, 3724S, Cell Signaling Technology), beta-Actin rabbit polyclonal antibody at 1:1000 (3967S, Cell Signaling Technology), RIOK2 mouse monoclonal antibody at 1:1000 (OTI3E11, TA505140, Origene). After O/N incubation, membranes were washed four times with PBST buffer for 5 minutes each on a rocker at 25° C., followed by incubation with HRP-linked anti-mouse IgG (7076S, Cell Signaling Technology) or HRP-linked anti-rabbit IgG (7076S, Cell Signaling Technology) at 1:3000 dilution in fresh blocking buffer for 1 h at 25° C. After incubation with secondary antibodies, membranes were washed four times with PBST buffer for 5 minutes each on a rocker at 25° C., followed by incubation with Pierce Western blotting substrates (Thermo Scientific) mixed at 1:1 ratio for 2-5 mins at 25° C. The protein bands were then visualized using the ChemiDoc Touch Imaging system (Bio-Rad).

### Immunoprecipitation and Mass Spectrometry

For collection of cellular lysates, lysis was similarly performed as described above. 5% of the supernatant was saved as Input, and pre-washed monoclonal anti-HA antibody tagged-agarose beads slurry (A2095, Sigma Aldrich) was added to the rest of the supernatant and incubated at 4° C. on a rocker O/N. The beads were washed with RIPA lysis buffer, resuspended in 2× laemmli buffer, boiled at 95° C. for 10 minutes, and immediately loaded for SDS-PAGE separation. Electrophoresis was performed until the proteins ran 1/6^{th} of the entire gel size. The gel was Coomassie stained for 1 h at 25° C., de-stained at 4° C. O/N, and were cut with sterile blades, washed with MS-grade acetonitrile solution and submitted for Mass Spectrometry analysis at BIDMC Mass Spectrometry Core. For elution of mutant RIOK2 proteins, whole cell lysate collection and washing of beads post immunoprecipitation were done in RIPA lysis buffer containing 500 mM NaCl concentration. After washing, the beads were incubated in elution buffer (0.1M glycine solution, pH 2.0) supplemented with HA-peptides (Millipore Sigma, 12149) at 0.2 μg/μl for 30 minutes with gentle flicking of tubes every 10 minutes. The beads were then centrifuged at 2500×g to collect the eluted proteins in the supernatant.

Gel separated protein samples were reduced with 55 mM DTT, alkylated with 10 mM iodoacetamide (Sigma-Aldrich), and digested O/N with TPCK modified trypsin/LysC (Promega) at pH=8.3. Peptides were extracted, dried out in a SpeedVac, resuspended in 10 μL of 1% Acetonitrile/98.9% Water, 0.1% Formic acid. 3 μL of the digested protein samples were analyzed in positive ion mode via microcapillary tandem mass spectrometry (LC-MS/MS) using a high resolution hybrid QExactive HF Orbitrap Mass Spectrometer (Thermo Fisher Scientific) via HCD with data-dependent analysis (DDA) with 1 MS1 scan followed by 8 MS2 scans per cycle (Top 8). Peptides were delivered and separated using an EASY-nLCII nanoflow HPLC (Thermo Fisher Scientific) at 300 nL/min using self-packed 15 cm length x 75 μm i.d. Cis fritted microcapillary columns. Solvent gradient conditions were 120 minutes from 3% B buffer to 38% B (B buffer: 100% acetonitrile; A buffer: 0.9% acetonitrile/0.1% formic acid/99.0% water). MS/MS spectra were analyzed using Mascot Version 2.6 (Matrix Science) by searching the reversed and concatenated human protein database (at the world wide web at ebi.ac.uk/uniprot/database/download.html) with a parent ion tolerance of 18 ppm and fragment ion tolerance of 0.05 Da. Carbamidomethylation of cysteine (+57.0293 Da) was specified as a fixed modification and oxidation of Methionine (+15.9949 Da), deamidation of Asparagine/Glutamine (+0.984 Da) as variable modifications. Results were imported into Scaffold Q+S 4.11 software (Proteome Software, Inc.) with a peptide threshold of ˜75%, protein threshold of 95%, resulting in a peptide false discovery rate (FDR) of ˜1%. Known contaminants such as keratins, caseins, trypsin and BSA were removed from the analysis.

The immunoprecipitated proteins yielded quantitative spectral counts in mass spectrometry which reflect their binding intensities. The spectral counts of individual proteins were normalized using spectral counts of respective immunoprecipitated HA-tagged versions of RIOK2 and the fold changes with respect to wild-type (WT) RIOK2 were calculated.

### Fluorescence-Activated Cell Sorting (FACS) Analysis

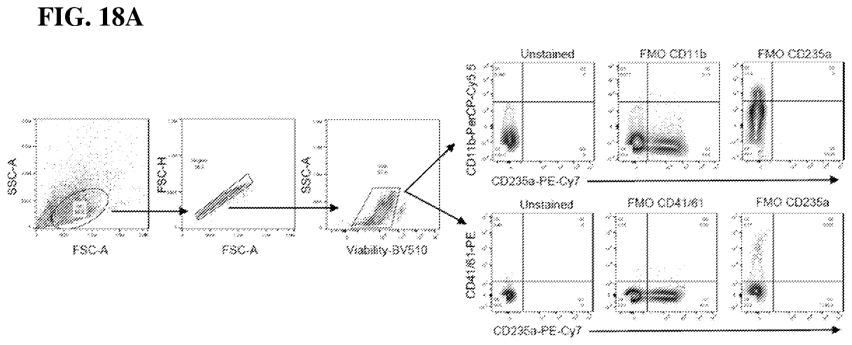

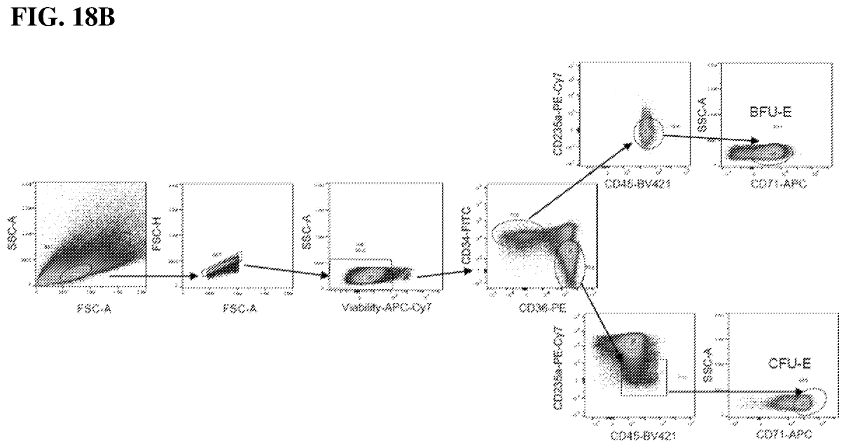

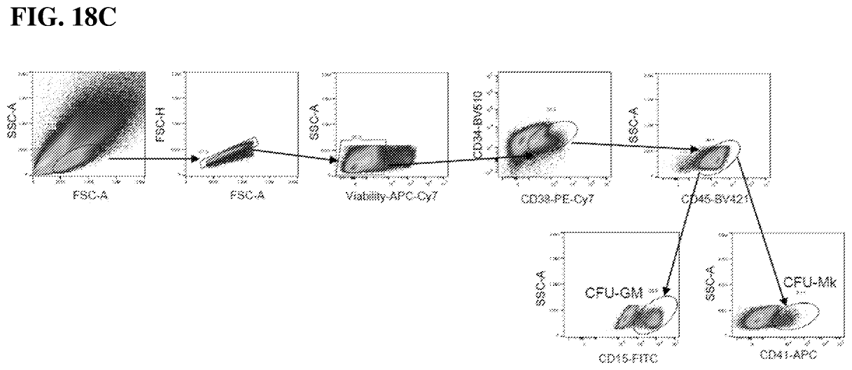

For analysis of cellular surface marker expressions via flow cytometry, in vitro cultured cells were washed with PBS and incubated with fixable viability dyes (Tonbo Biosciences) at 1:800 dilution in PBS for 20 mins at 25° C. in dark. The cells were washed with staining buffer and labelled with fluorochrome-conjugated antibodies diluted (1:100/200) in staining buffer for 30 minutes at 4° C.: FITC-CD34 (343604, BioLegend), BV421-CD38 (356617, BioLegend)/PE-Cγ7-CD38 (356608, BioLegend), APC-CD71 (OKT9, eBioscience), PE-Cγ7-CD235 (306620, BioLegend), PE-CD41/CD61 (359806, BioLegend), PerCP-Cγ5.5-CD11b (301328, 101228, 393106, BioLegend), AF700-CD42b (303928, BioLegend), APC-CD135 (313307, BioLegend), BV510-Human Lineage (348807, BioLegend), PE-CD68 (333807, BioLegend), BV421-CD45RA (304129, BioLegend), BV421-CD45 (304031, 368521, BioLegend), BV785-CD45 (368527, BioLegend), PerCP-Cγ5.5-CD123 (306015, BioLegend), AF700-CD117 (313245, Biolegend), PE-CD36 (336205, BioLegend), BV510-CD34 (343527, BioLegend), FITC-CD15 (301904, BioLegend), APC-CD41 (343709, BioLegend), PE-CD14 (325605, BioLegend), APC-CD13 (301706, BioLegend) For intracellular staining, cells were labelled for surface markers as mentioned above, followed by fixation and permeabilization using Foxp3/Transcription factor staining kit (Thermo Fisher Scientific). PE-conjugated GATA1 rabbit monoclonal antibodies (13353S, Cell Signaling), PE-conjugated HA-tag rabbit monoclonal antibodies, or PE-conjugated control rabbit IgG isotype (5742, Cell Signaling) were used at 1:100 dilution. All FACS analyses were performed using CytoFLEX Flow Cytometer (Beckman Coulter). Data were analyzed using FlowJo 10.0.7 and plotted using GraphPad Prism. The gating strategies are shown in FIG. 18.

### Methylcellulose Assay

Primary human HSPCs were washed with 1× PBS and mixed with semi-solid methylcellulose medium (H4034, StemCell Technologies) by brief vortexing. Cells were plated at a density of 1000 per well in a 6-well plate followed by incubation in a humidified chamber at 37° C. for 14 days. Imaging of the colonies was performed using EVOS M5000 Imaging system (ThermoFisher Scientific). The colony-forming cells were then collected by triturating the wells using staining buffer and multi-color flow cytometry was performed as described above and in (Li, J. et al. Isolation and transcriptome analyses of human erythroid progenitors: BFU-E and CFU-E. Blood 124, 3636-3645 (2014); Kaufman, D.S., Hanson, E. T., Lewis, R. L., Auerbach, R. & Thomson, J.A. Hematopoietic colony-forming cells derived from human embryonic stem cells. Proc Natl Acad Sci USA 98, 10716-10721 (2001)). The gating strategies are shown in FIG. 18. The colonies were scored using STEMvision automated CFU counter. The STEMvision Acquisition, Analyzer and colony marker software programs were used to acquire images, analyze colonies and mark them, respectively.

### Quantitative Proteomic Analysis

Quantitative proteomic analysis of primary Human HSPCs was performed as described (Myers, S. A. et al. Streamlined Protocol for Deep Proteomic Profiling of FAC-sorted Cells and Its Application to Freshly Isolated Murine Immune Cells. Mol Cell Proteomics 18, 995-1009 (2019)). Lysis of cells was done by adding 10 μL of 8 M urea, 10 mM TCEP and 10 mM iodoacetamide in 50 mM ammonium bicarbonate (ABC) solution to 1×10⁶ cells followed by incubation at 25° C. for 30 min in the dark. Urea was diluted to 1.5 M with ABC followed by trypsin digestion at 37° C. O/N. The lysate digest was spun directly onto a C18 Stage tip at 3500 × g through the glass mesh.

On-column TMT Labeling: For resin conditioning, 50 μL methanol (MeOH) was added followed by 50 μL 50% acetonitrile (ACN)/0.1% FA and equilibration was done using 75 μL 0.1% FA twice. After centrifugation of the entire digest at 3500 × g for loading, 1 μL of TMT reagent in 100% ACN was added to 100 μL freshly made HEPES (pH 8) and passed over the C18 resin at 350 xg. After washing with 75 μL 0.1% FA twice, peptide elution was performed with 50 μL 50% ACN/0.1% FA followed by a second elution with 50% ACN/20 mM ammonium formate, pH 10. Measurement of absorbance at 280 nm was used for estimating peptide concentrations followed by analysis of labelling efficiency of the elution. The samples were then mixed before Stage tip-based fractionation and analysis.

Stage tip bSDB Fractionation: -20 μg total peptides were loaded using 200 μL pipette tips packed with two punches of sulfonated divinylbenzene (SDB-RPS, Empore) with a 16-gauge needle. Then 25 μL 20 mM NH4HCO2, pH 10, was used to perform a pH switch. This was regarded as part of fraction one. This was followed by step fractionation using 5, 7.5, 10, 12.5, 15, 17.5, 20, 25, 42, and 50% ACN concentrations with each fraction collected in individual autosampler vials. They were then dried via vacuum centrifugation and stored at −80° C. until further analysis.

### Data Acquisition

On-line chromatography was performed by an Easy-nLC 1200 (Thermo Fisher) at a flow rate of 200 nl/min. Peptide separation was performed using 75 μm i.d. PicoFrit (New Objective, Woburn, MA) columns packed with 1.9 μm AQ-C18 material (Dr. Maisch, Germany) up to 20 cm at 50° C. LC gradient went from 6% B at 1 min to 30% B in 85 mins, then an increase to 60% B by minute 94 and 90% by min 95, and finally to 50% B until the end of the run that lasted 110 minutes. Thermo Scientific Lumos Tribrid was used to perform mass spectrometry. A precursor scanning was done from 350 to 1800 m/z at 60,000 resolution. Then the topmost intense multiply charged precursors within a 2 second window were selected for higher energy collisional dissociation (HCD) at 50,000 resolution. Threshold for precursor isolation width was set to 0.7 m/z and the maximum MS2 injection time was set at 110 milliseconds for an automatic gain control of 6e4. Dynamic exclusion was set at 45 seconds and only charge states of two to six were selected for MS2. For data acquisition run, half of each fraction was injected.

### Data Processing

Uniprot Human database (12/28/2017) containing common laboratory contaminants was used to search all data with Spectrum Mill (Agilent). For the search, variable modifications of N- terminal protein acetylation, oxidation of methionine, TMT-1Iplex labels and a fixed modification of carbamidomethylation of cysteine were used. The following cut offs were employed for search: enzyme specificity was set to trypsin, a maximum of three missed cleavages was used, maximum precursor-ion charge state was six, and the MS1 and MS2 mass tolerance were set to 20 ppm. Using a reverse, decoy database, FDRs for peptides and proteins were found to be less than 1%. Identification with at least two distinct peptides and a Spectrum Mill score protein level score - 20 were set as cut offs for reporting of proteins.

The Spectrum Mill protein/peptide summary module was used to correct the TMT11 reporter ion intensities in each MS/MS spectrum for isotopic impurities. The afRICA correction method was used which implemented determinant calculations according to Cramer's Rule and general correction factors obtained from the reagent manufacturer's certificate of analysis (CoA). Median was normalized, and median absolute deviation-scale data set was subjected to a two-sample moderated T-test. Then Benjamini-Hochberg Procedure was implemented to correct for multiple hypothesis testing. An arbitrary cutoff at adj. p val <0.05 was drawn for differentially abundant proteins.

### Bulk RNA-sequencing

RNA library preparations, sequencing reactions and initial bioinformatic analysis were conducted at GENEWIZ, LLC. (South Plainfield, NJ, USA) as follows: Library Preparation with polyA selection and HiSeq Sequencing: RNA samples received were quantified using Qubit 2.0 Fluorometer (Life Technologies, Carlsbad, CA, USA) and RNA integrity was checked using Agilent TapeStation 4200 (Agilent Technologies, Palo Alto, CA, USA). RNA sequencing libraries were prepared using the NEBNext Ultra RNA Library Prep Kit for Illumina following manufacturer's instructions (NEB, Ipswich, MA, USA). Briefly, mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA). The sequencing libraries were clustered on 1 lane of a flowcell. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

### Data Analysis

The sequence reads were trimmed to remove possible adapter sequences and nucleotides with poor quality using Trimmomatic v.0.36. The trimmed reads were mapped to the Homo sapiens reference genome available on ENSEMBL using the STAR aligner v.2.5.2b to generate BAM files. Unique gene hit counts were calculated by using feature Counts from the Subread package v.1.5.2. Only unique reads that fell within exon regions were counted. Gene set enrichment analysis (GSEA) was performed using Broad Institute's GSEA Software. Sets of erythroid, megakaryocytic and myeloid-lineage genes and genes involved in ribosome biogenesis were derived by accounting for the differentially regulated genes that passed the cut off of adjusted p value <0.05 in control vs RIOK2-depleted HSPCs. Additionally, canonical pathways such as KEGG, REACTOME and BIOCARTA were consulted.

Assay of Transposase accessible Chromatin with high-throughput Sequencing (ATAC-Seq)

ATAC-seq on snap-frozen control and RIOK2-depleted HSPCs was performed at Center for Functional Cancer Epigenetics (CFSE) at DFCI. Briefly, after high throughput Illumina sequencing of pair-ended reads, the raw reads were mapped to the reference genome (hg19) using alignment software (conFigureyaml). After alignment, the mapped reads were then normalized (by down-sampling) to 4 million reads. The uniquely mapped reads were then piled-up. Nd is defined as the number of unique region locations. N1 is defined as the number of unique region locations with only one read. The PBC (a common method to determine sample complexity) is then simply calculated as N1/Nd. ATAC-seq samples with a good PBC score >=0.90 were analyzed. Peaks were called using the MACS peak calling software. Chromatin accessibility was analyzed using IGV software.

### Chromatin Immunoprecipitation (ChIP)

Chromatin immunoprecipitation was performed using EZChIP™ kit (Millipore Sigma) according to the manufacturer's instructions. 2-4 μg of primary antibodies were used-(GATA-1 (D24E4) XP Rabbit monoclonal antibody (4589S, Cell Signaling); GATA-1 rat monoclonal antibody (N6) (SC-265, Santa Cruz Biotechnology); HA rabbit monoclonal antibody (C29F4, 3724S, Cell Signaling Technology), RIOK2 mouse monoclonal antibody (OTI3E11, TA505140, Origene); RIOK2 rabbit polyclonal antibody (NBP130098, Thermo Fisher Scientific)) or control IgGs. The immunoprecipitated DNA was purified using Spin columns (Millipore Sigma/Qiagen) and used for quantitative RT-PCR using Quantstudio6 (Applied Biosciences).

### ChIP Sequencing

ChIP-Seq Library Preparation, HiSeq Sequencing and initial bioinformatic analysis were conducted at GENEWIZ, LLC. (South Plainfield, NJ, USA) as follows: ChIP DNA samples were quantified using Qubit 2.0 Fluorometer (Life Technologies, Carlsbad, CA, USA) and the DNA integrity was checked with 4200 TapeStation (Agilent Technologies, Palo Alto, CA, USA). NEB NextUltra DNA Library Preparation kit was used following the manufacturer's recommendations (Illumina, San Diego, CA, USA). Briefly, the ChIP DNA was end repaired and adapters were ligated after adenylation of the 3′ends. Adapter-ligated DNA was size selected, followed by clean up, and limited cycle PCR enrichment. The ChIP library was validated using Agilent TapeStation and quantified using Qubit 2.0 Fluorometer as well as real time PCR (Applied Biosystems, Carlsbad, CA, USA). The sequencing libraries were multiplexed and clustered on one lane of a flowcell. After clustering, the flowcell was loaded on the Illumina HiSeq instrument according to manufacturer's instructions (Illumina, San Diego, CA, USA). Sequencing was performed using a 2×150 Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mis-match was allowed for index sequence identification.

Bioinformatics Analysis workflow: Sequence reads were trimmed to remove possible adapter sequences and nucleotides with poor quality at 3′ end (error rate >0.01) using CLC Genomics Server 9.0. Trimmed data was then aligned to reference genome for human GRCh38. During the mapping, only specific alignment was allowed. The TSS plots and chromosome view plots were generated using Partek Flow software. The HOMER algorithm was used to curate de novo nucleotide binding motifs of RIOK2 across the entire human genome.

### Electrophoretic Mobility Shift Assay (EMSA)

Recombinant human GST-tagged RIOK2 (rhRIOK2, Abcam) was incubated with duplex DNA (Table 5) at 5:1 molar ratio in a low-salt buffer for 20-30 minutes at 4° C. and 2% agarose gel was cast and cooled to 4° C. The DNA-protein mixture was supplemented with 0.22p filtered 50% glycerol solution to facilitate loading. The gel was run for 1.5 hours at 4° C., followed by incubation in ethidium bromide solution for 30 minutes and visualized using ChemiDoc Touch Imaging system (Bio-Rad). ImageJ was used to quantify band shifts.

### Nascent Protein Synthesis In Vitro

Measurement of nascent protein synthesis in vitro was done using Click-iT™ Plus OPP Alexa Fluor™ 647 Protein Synthesis Assay kit (Thermo Fisher Scientific) using manufacturer's instructions. Briefly, cells in culture were supplemented with Click-iT™ OPP (o-propargyl puromycin) at a final working concentration of 20 μM and incubated for 30 minutes at 37° C. The cells were washed with PBS, stained with fixable viability dye, fixed and permeabilized followed by incubation with Click-iT™ reaction buffer containing Alexa Fluor™ 647 picolyl azide and incubated at 25° C. for 30 minutes. The cells were washed and analyzed via flow cytometry.

### Structural Modelling of RIOK2

The crystal structure of residues 2-301 of human RIOK2 (PDB code 6hk6)(Wang, J., Varin, T., Vieth, M. & Elkins, J. M. Crystal structure of human RIOK2 bound to a specific inhibitor. Open Biol 9, 190037 (2019)), comprising the N-terminal winged helix-turn-helix (wHTH) domain (residues 2-92) and the kinase domain (residues 93-289) was docked onto B-form DNA by aligning the wHTH domain with the crystal structure of the DNA binding domain of the diphtheria toxin repressor bound to its DNA operator sequence (PDB code lf5t)(Chen, C. S., White, A., Love, J., Murphy, J. R. & Ringe, D. Methyl groups of thymine bases are important for nucleic acid recognition by DtxR. Biochemistry 39, 10397-10407 (2000)). Modeling was done using Modeller software solely to give a crude impression of the relative positions of the various sub-domains of the N-terminal part of the protein when RIOK2 binds to DNA, because the structure of most of the C-terminal half of the protein is unknown.

### Statistical Tests

Data are presented as mean±SEM. Unpaired two-tailed Student's t-test was used for comparing two groups. Analysis of variance (ANOVA) with Tukey's/Dunnett's correction or Kruskal-Wallis test with Dunn's correction was used for comparisons amongst multiple groups, wherever applicable. No statistical methods were used to pre-determine sample sizes, but these sample sizes are similar to those reported in previous publication (Gutierrez, L. et al. Ablation of Gatal in adult mice results in aplastic crisis, revealing its essential role in steady-state and stress erythropoiesis. Blood 111, 4375-4385 (2008)). Data distribution was assumed to be normal, but this was not formally tested. Data points were not excluded from the analysis. Microsoft Excel was used to list all numerical values and GraphPad Prism v8.0/9.0 (GraphPad Software Inc., San Diego, CA) was used to perform statistical analyses. Sample size was not predetermined.

### List of all RIOK2 Mutants

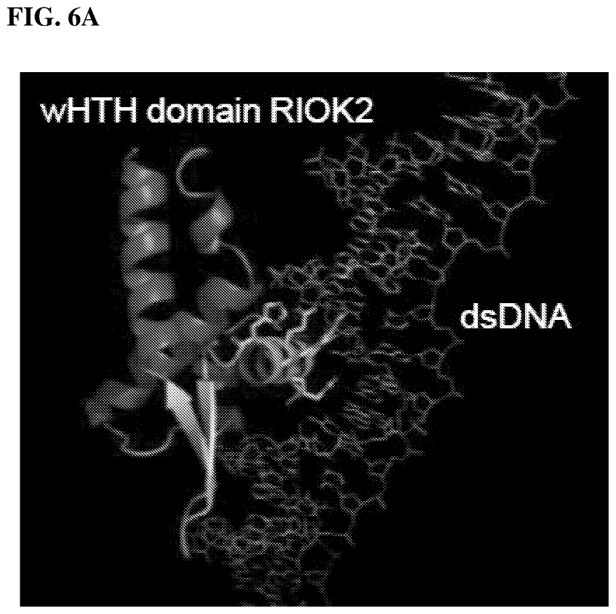

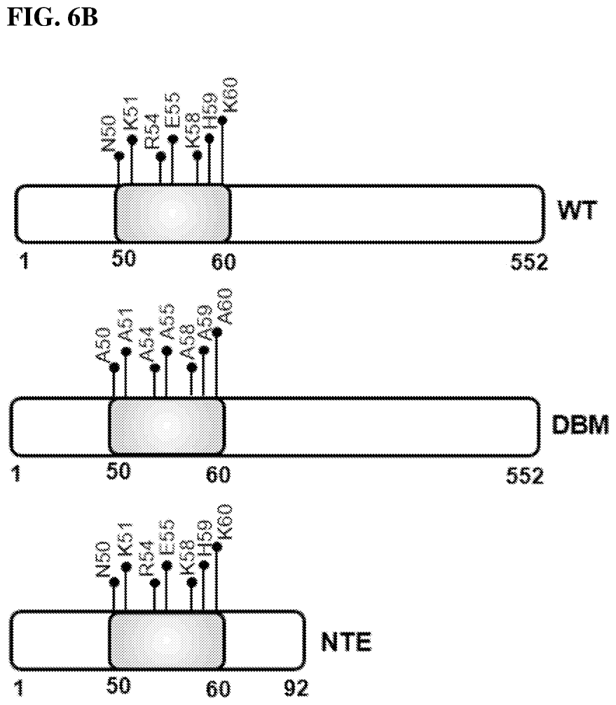

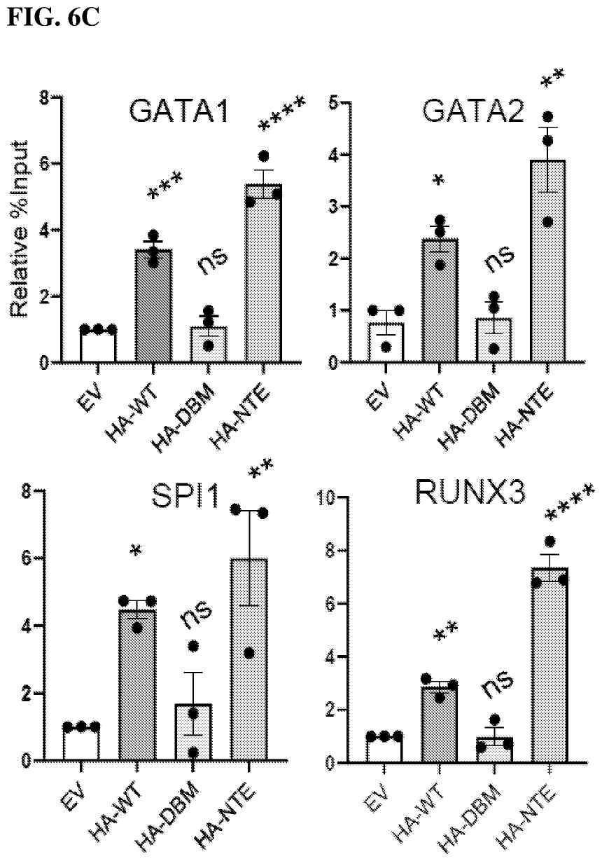

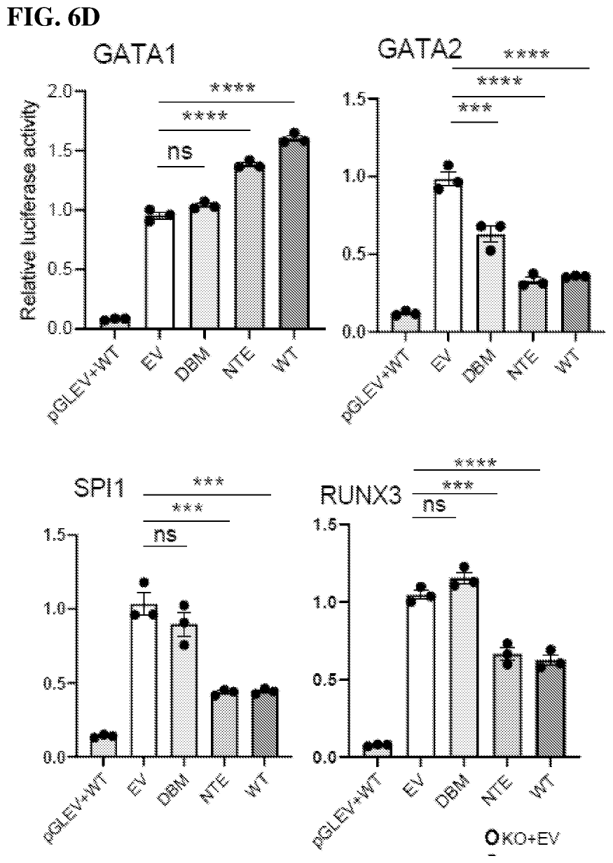

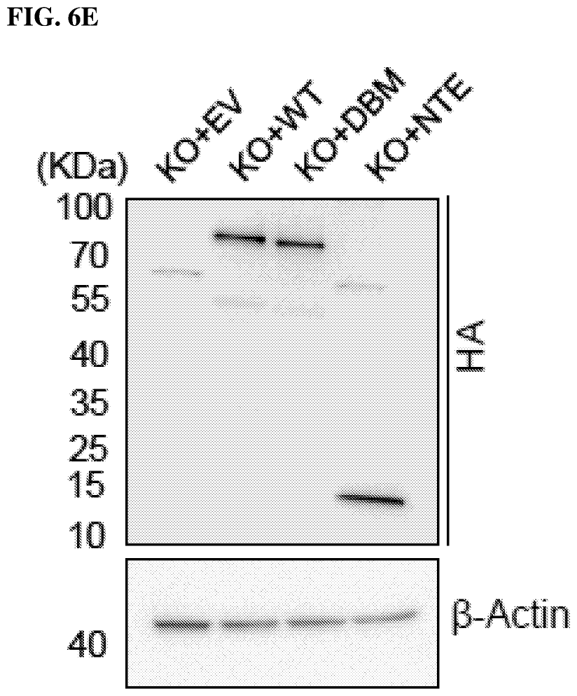

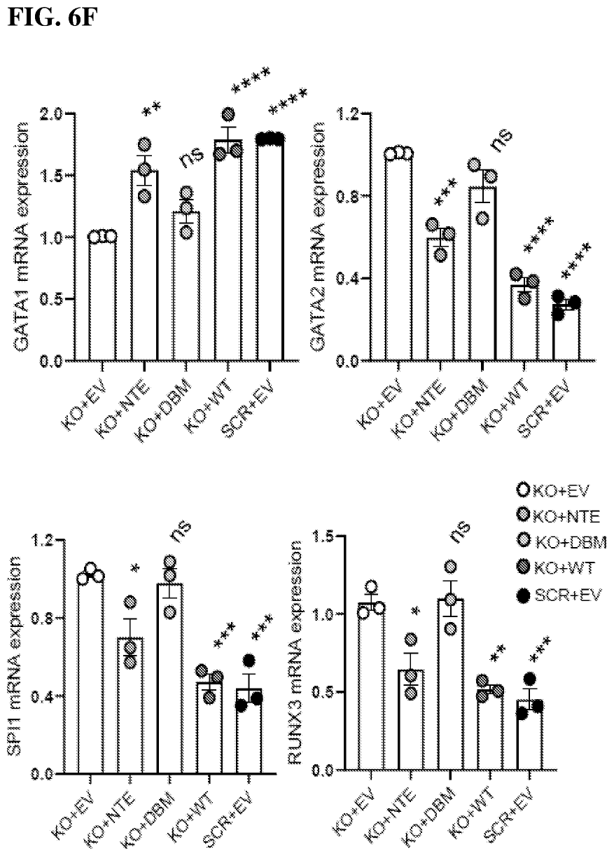

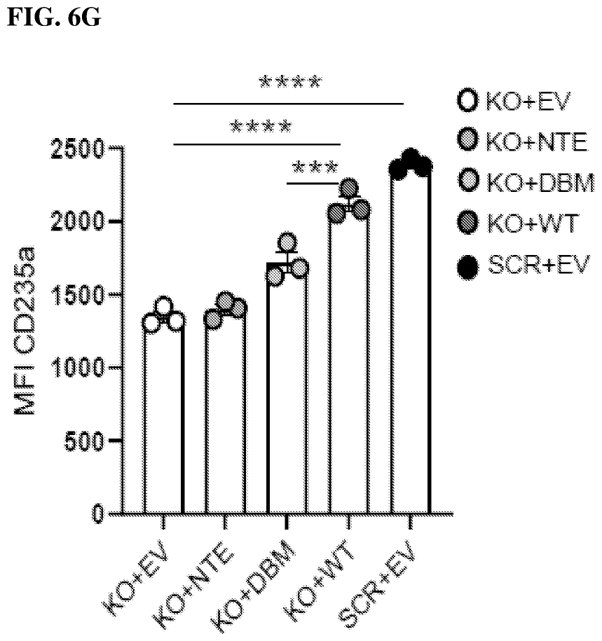

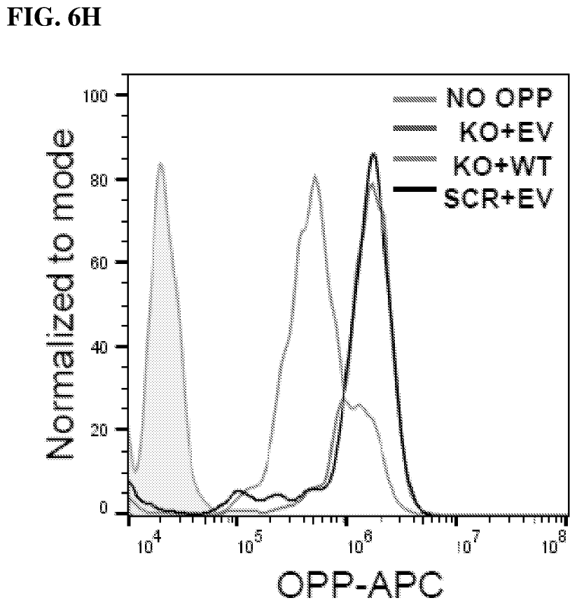

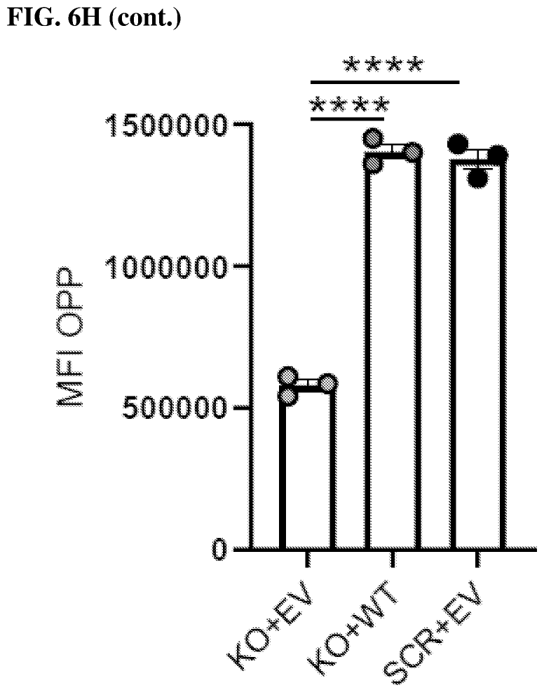

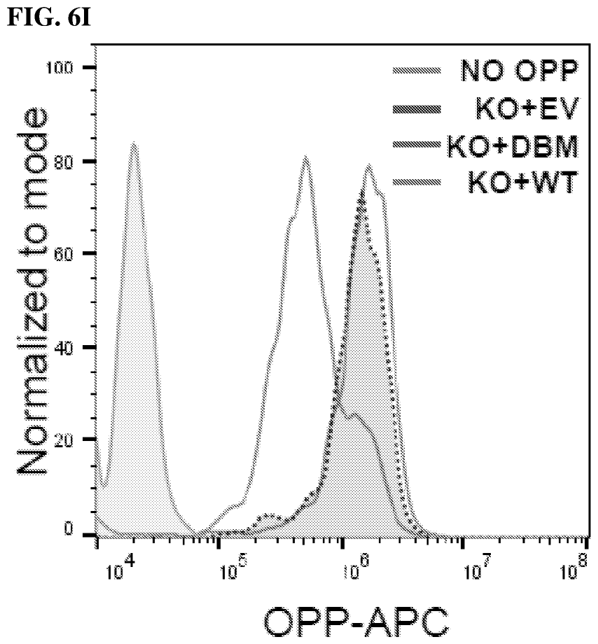

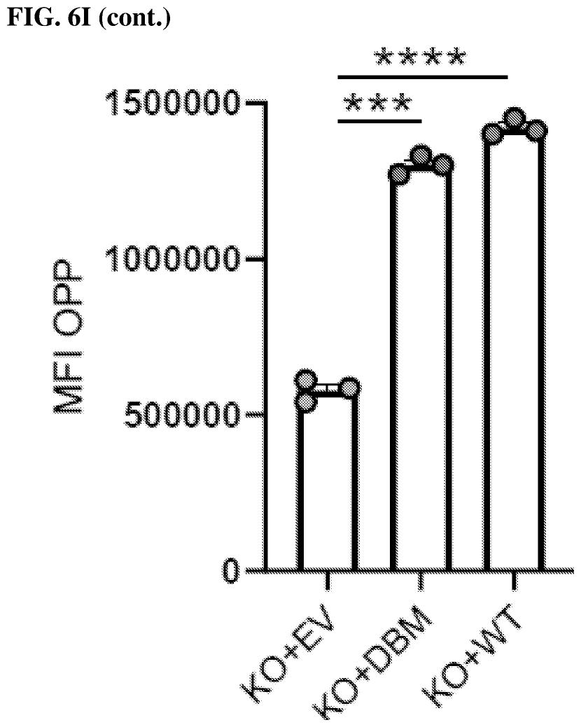

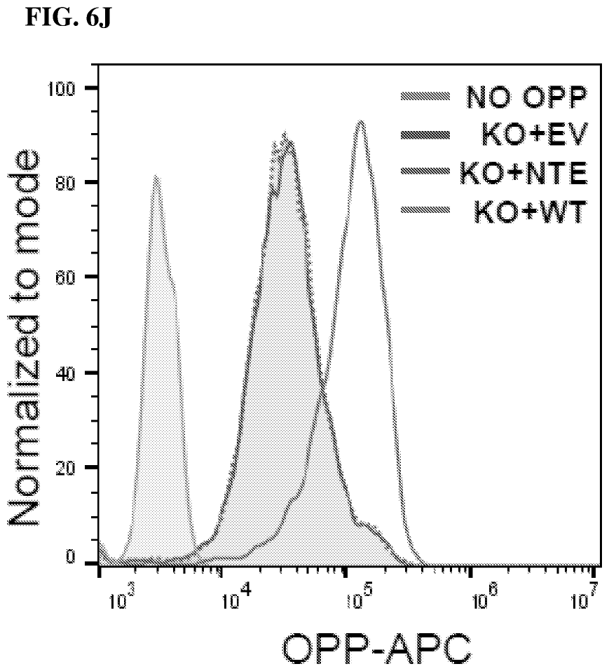

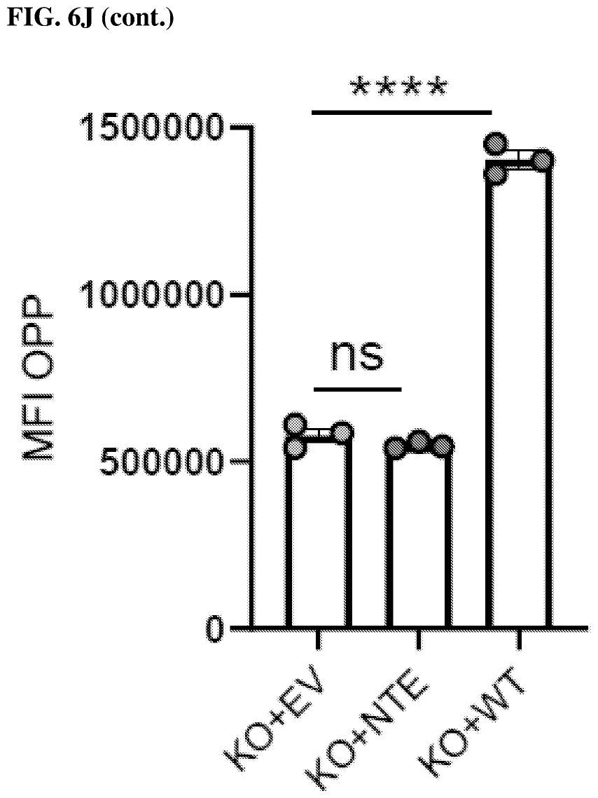

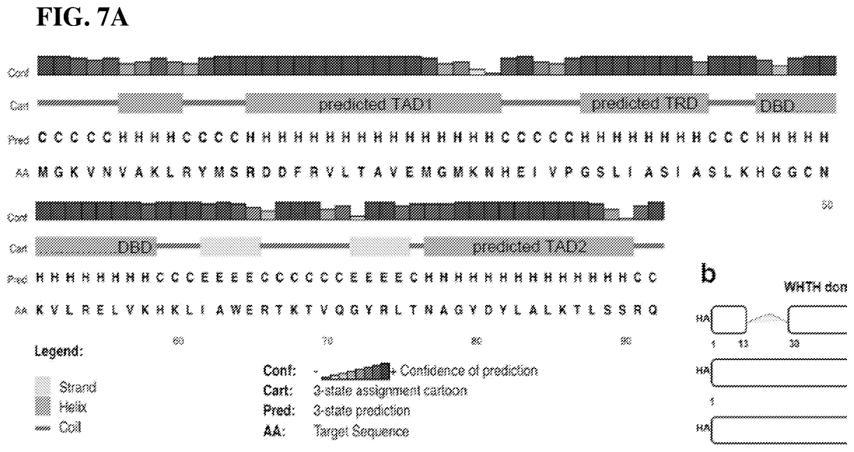

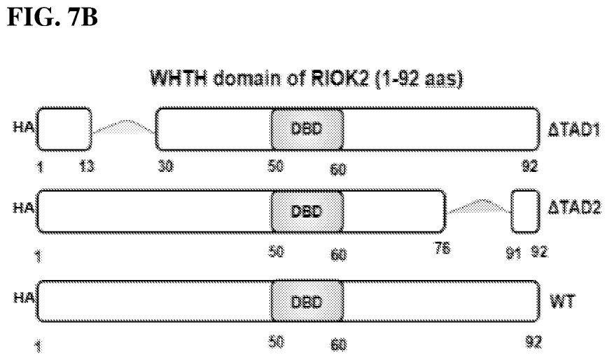

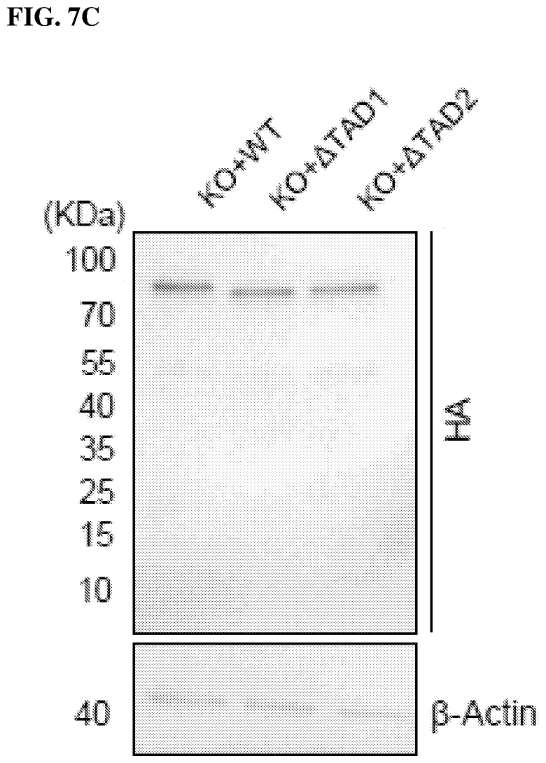

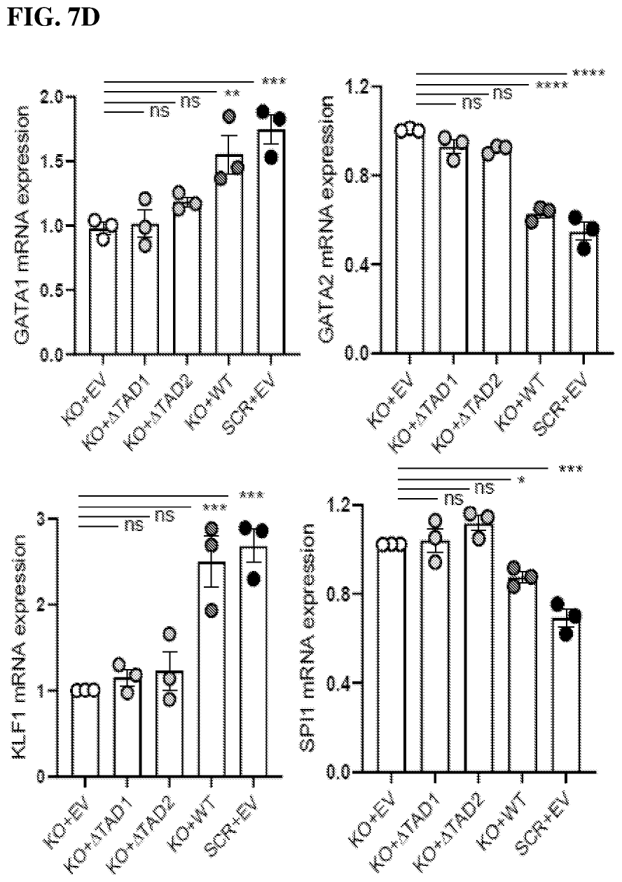

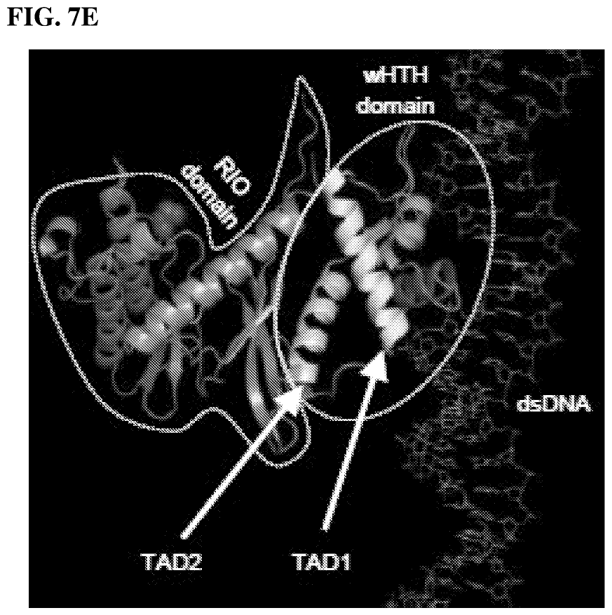

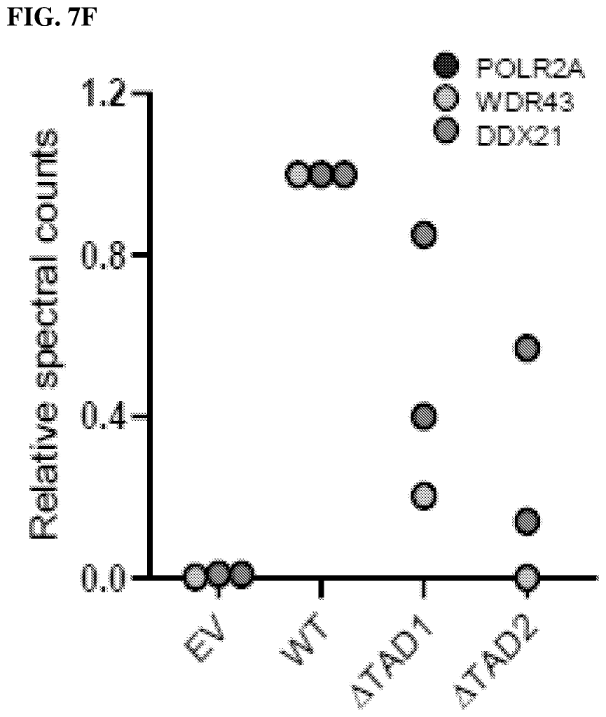

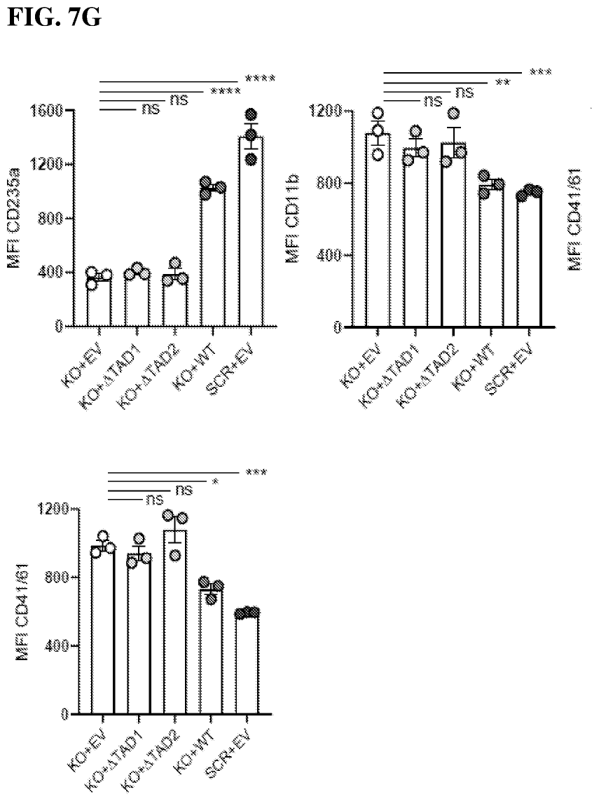

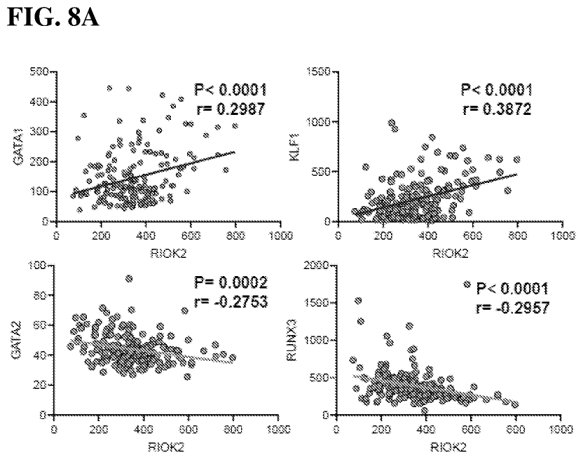

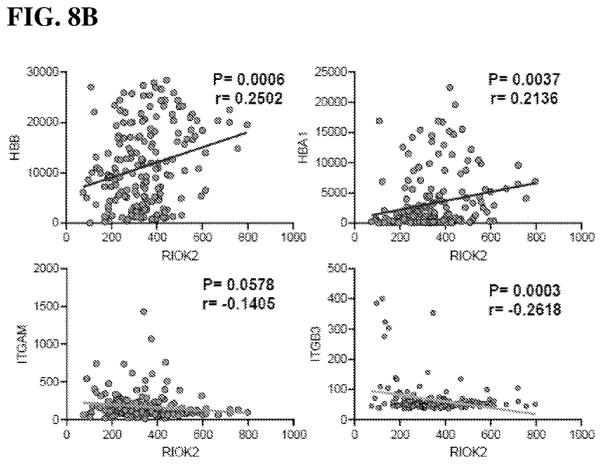

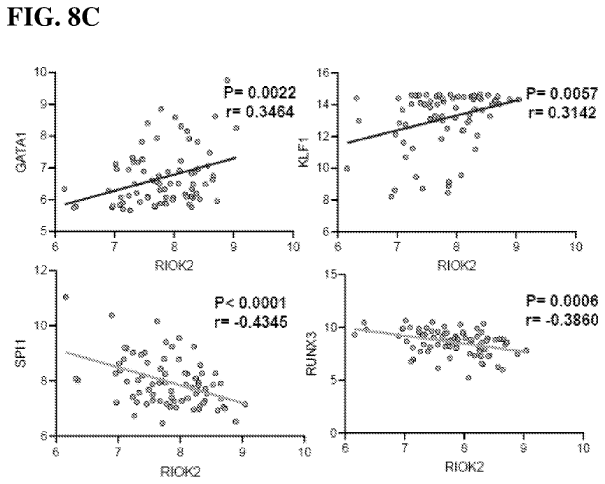

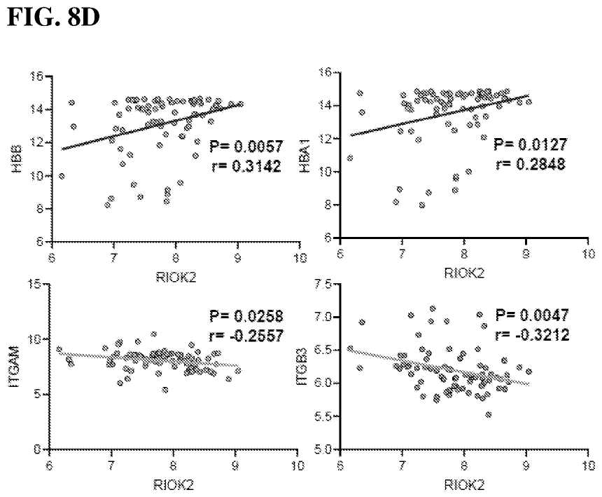

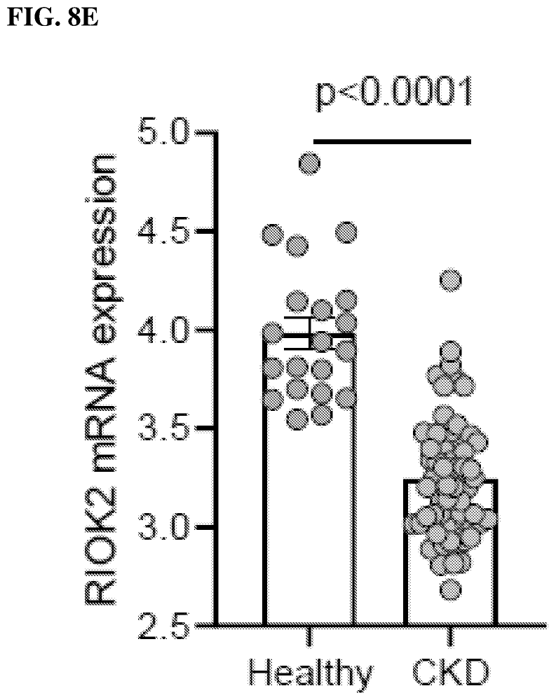

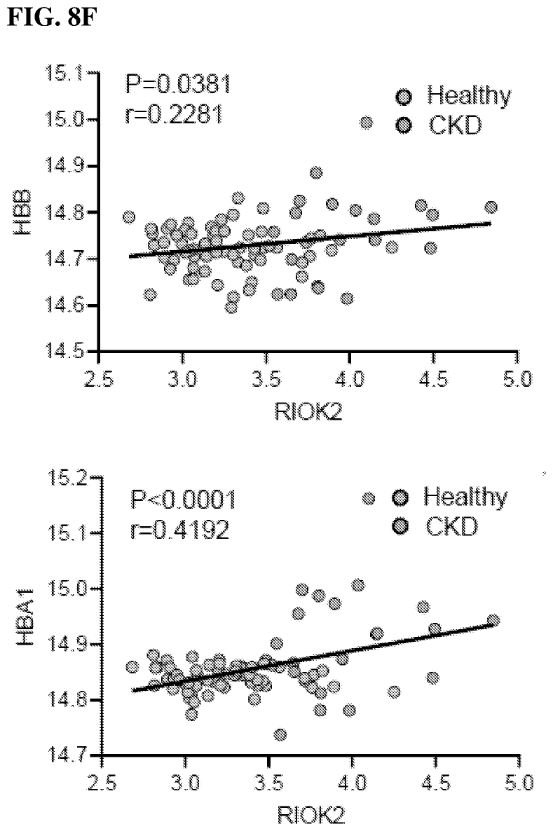

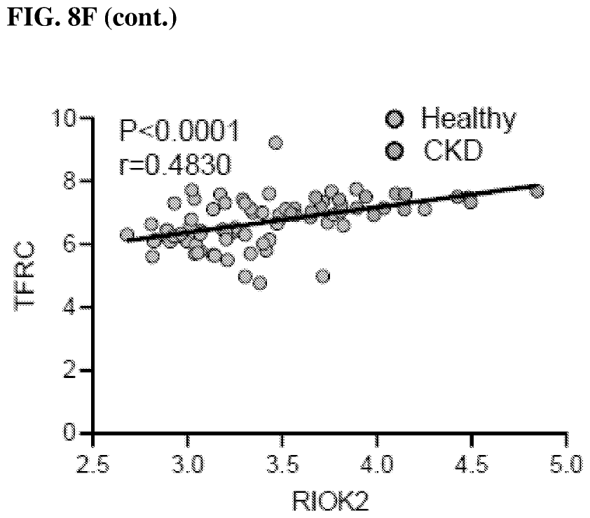

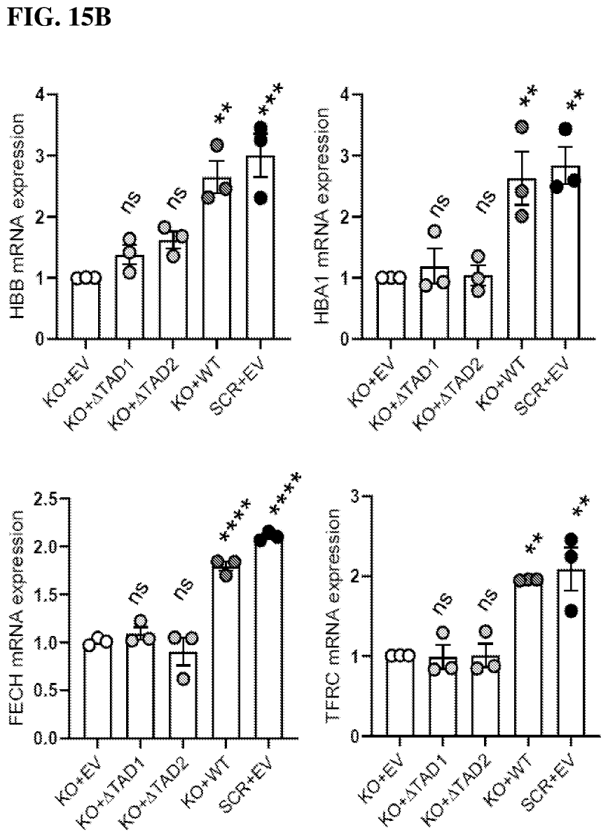

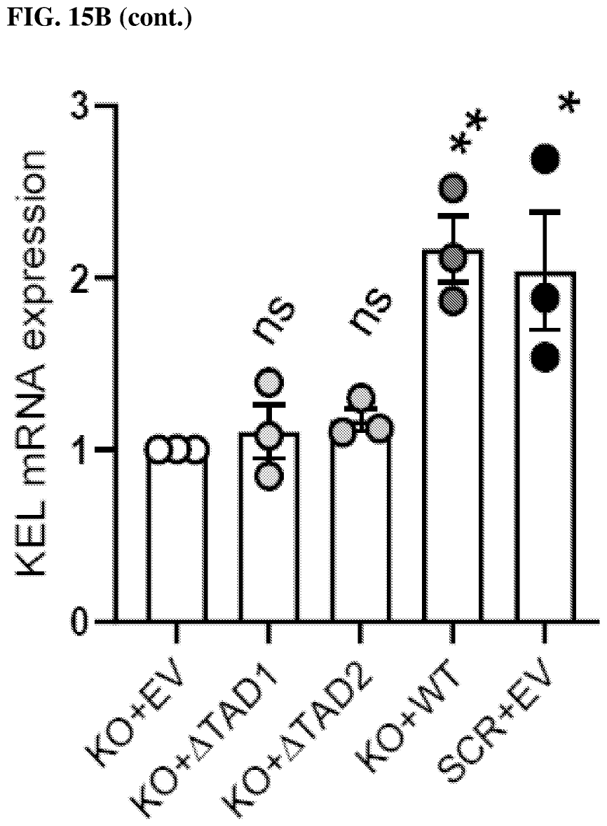

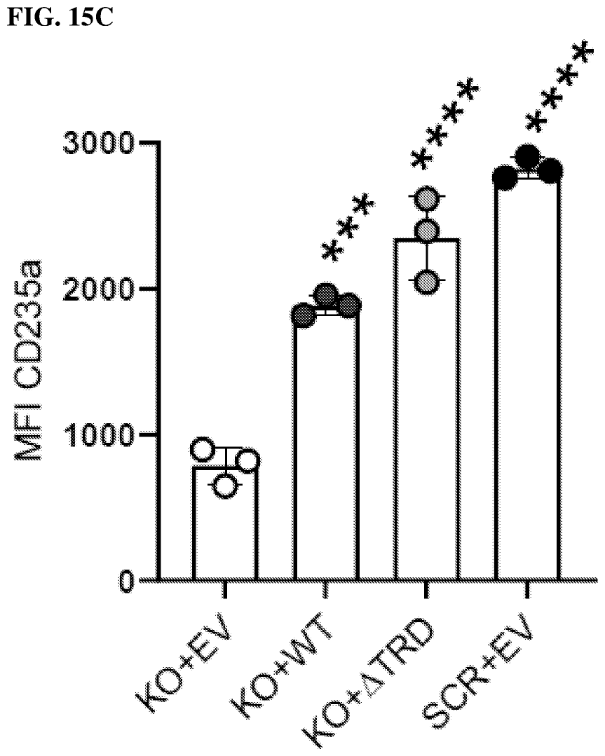

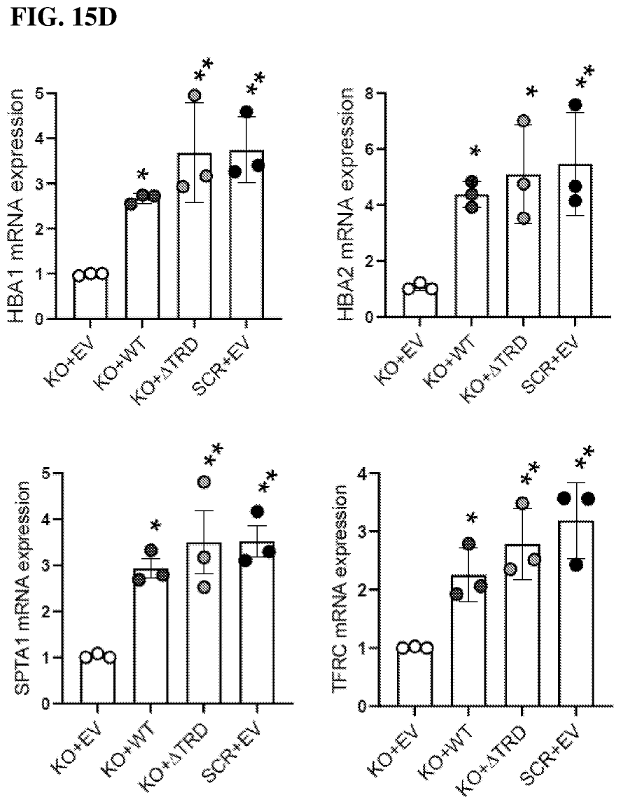

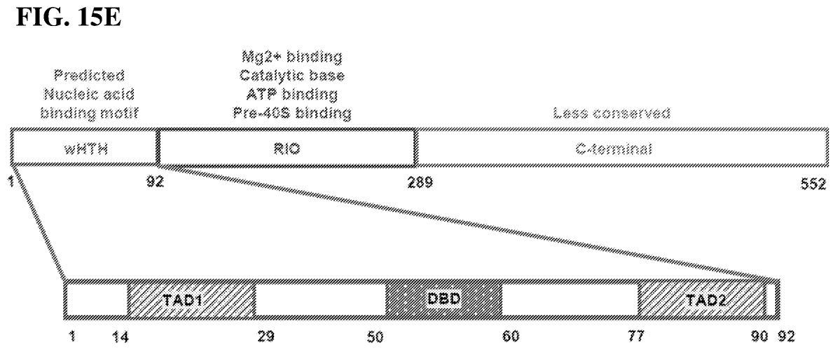

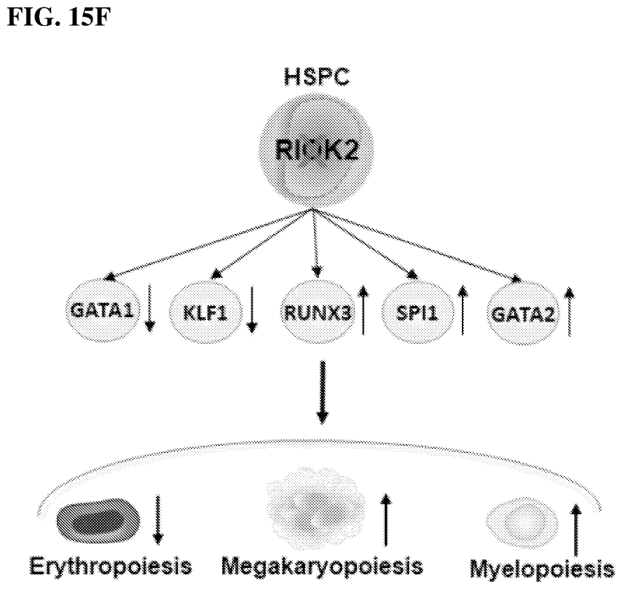

1. DNA-binding mutant (DBM): Seven evolutionarily conserved residues in the N-terminal wHTH domain of RIOK2: Asn50 Lys51 Arg54 Glu55 Lys58 His59 Lys60 have been mutated to Alanine (FIG. 6B). This mutant displays defective binding to DNA (FIG. 6C) and impaired hematopoietic differentiation when expressed in an endogenous RIOK2-Knockout (KO) setting (FIGS. 6F and 6G).
2. Transactivation domain deleted 1 (ΔTAD1): N-terminal HA-tagged mutant with deletion in helices forming the wHTH domain of RIOK2 (FIGS. 7A and 7B). Deletion of amino acids 14-29 created ΔTAD1 mutant of RIOK2 which does not influence RIOK2's DNA-binding affinity (FIG. 15A), but significantly impairs hematopoietic differentiation when expressed in an endogenous RIOK2-KO setting (FIGS. 7D and 7G).
3. Transactivation domain deleted 2 (ΔTAD2): N-terminal HA-tagged mutant with deletion in helices forming the wHTH domain of RIOK2 (FIGS. 7A and 7B). Deletion of amino acids 77-90 created ΔTAD2 mutant of RIOK2 which does not influence RIOK2's DNA-binding affinity (FIG. 15A), but significantly impairs hematopoietic differentiation when expressed in an endogenous RIOK2-KO setting (FIGS. 7D and 7G).
4. Transrepressor domain deleted (ATRD): N-terminal HA-tagged mutant by deleting residues 35-43, comprising another helix in the wHTH domain of RIOK2 (FIG. 7A). This mutant displayed more potent erythroid differentiation than wild-type (WT) RIOK2 in the KO-setting, indicating that this helix functions as a transrepressor domain (TRD) of RIOK2 (FIGS. 7C and 7D).
5. N-terminal extension (NTE): A lentiviral vector expressing only the N-terminal extension i.e. 1-92 residues of RIOK2, which forms its winged helix-turn-helix (wHTH) domain (FIG. 6B). This mutant can efficiently bind with DNA (FIG. 6C) but has negligible impact on reversing the defective erythroid differentiation in RIOK2-KO primary human stem cells (FIG. 6G) because it lacks the RIO domain which is responsible for cytoplasmic translation (FIG. 6J).
6. The kinase-dead mutant of RIOK2 K123A created by cloning in a lentiviral vector (FIG. 8) has been described in Cerca et al., Nat. Struc. Biol. 2012.

### Example 2: Identification of RIOK2 as a Master Regulator of Human Blood Cell Development

Anemia is a hallmark of a plethora of hematologic disorders associated with aging, chronic diseases such as renal failure and inflammation, bone marrow failure and myeloid neoplasms (Palapar, L. et al. Anaemia and physical and mental health in the very old: An individual participant data meta-analysis of four longitudinal studies of ageing. Age Ageing 50, 113-119 (2021); Lopes, M. B. et al. A real-world longitudinal study of anemia management in non-dialysis-dependent chronic kidney disease patients: a multinational analysis of CKDopps. Sci Rep 11, 1784 (2021); Becktell, K. et al. Aplastic Anemia & MDS International Foundation (AA & MDSIF): Bone Marrow Failure Disease Scientific Symposium 2018. Leuk Res 80, 19-25 (2019)). Aberrant red blood cell differentiation (erythropoiesis) underlies anemias and can be accompanied by myeloid proliferation. For example, myelodysplastic syndromes (MDS), a heterogeneous group of clonal hematologic disorders, are classically characterized by anemia and myeloproliferation (Saygin, C. & Carraway, H.E. Current and emerging strategies for management of myelodysplastic syndromes. Blood Rev, 100791 (2020). The average survival time following diagnosis of MDS is 3 years owing to few treatment options and roughly 20-30% of MDS patients progress to acute myeloid leukemia (Hong, S. et al. Survival following relapse after allogeneic hematopoietic cell transplantation for acute leukemia and myelodysplastic syndromes in the contemporary era. Hematol Oncol Stem Cell Ther S1658-3876(20) 30178-3 (2020); Garcia-Manero, G., Chien, K. S. & Montalban-Bravo, G. Myelodysplastic syndromes: 2021 update on diagnosis, risk stratification and management. Am JHematol 95, 1399-1420 (2020)). Hence, the substantial risks of allogeneic bone marrow transplants in elderly patients, together with a dearth of effective FDA-approved drugs make it imperative to revisit the origins of blood cell differentiation (hematopoietic) defects underlying anemia and myeloproliferation to identify new druggable targets (Feld, J., Navada, S.C. & Silverman, L.R. Myelo-deception: Luspatercept & TGF-Beta ligand traps in myeloid diseases & anemia. Leuk Res 97, 106430 (2020); Bhatt, V.R. & Steensma, D.P. Hematopoietic Cell Transplantation for Myelodysplastic Syndromes. J Oncol Pract 12, 786-792 (2016)).

RIOK2 (right open reading frame kinase 2) is an atypical serine threonine kinase that plays important roles in the final maturation steps of the pre-40S ribosomal complex to facilitate cytoplasmic translation (Ferreira-Cerca, S. et al. ATPase-dependent role of the atypical kinase Rio2 on the evolving pre-40S ribosomal subunit. Nat Struct Mol Biol 19, 1316-1323 (2012); Zemp, I. et al. Distinct cytoplasmic maturation steps of 40S ribosomal subunit precursors require hRio2. J Cell Biol 185, 1167-1180 (2009); Ameismeier, M., Cheng, J., Berninghausen, O. & Beckmann, R. Visualizing late states of human 40S ribosomal subunit maturation. Nature 558, 249-253 (2018)). A recent study revealed that hematopoietic cell-specific heterozygous deletion of Riok2 leads to anemia and myeloproliferation in mice (Raundhal, M. et al. Blockade of IL-22 signaling reverses erythroid dysfunction in stress-induced anemias. Nat Immunol 22(4):520-529 (2021)), reminiscent of Myelodysplastic Syndrome (MDS)-associated phenotypes in patients (Pellagatti, A. & Boultwood, J. The molecular pathogenesis of the myelodysplastic syndromes. Eur J Haematol 95, 3-15 (2015)). It was also noted that reduced RIOK2 mRNA levels in patients with the del(5q) subtype of MDS as compared to healthy individuals (Pellagatti, A. et al. Deregulated gene expression pathways in myelodysplastic syndrome hematopoietic stem cells. Leukemia 24, 756-764 (2010)). However, the mechanisms underlying RIOK2-driven hematopoietic differentiation remain largely unexplored. The presence of a putative DNA-binding winged helix-turn-helix (wHTH) domain next to the RIO (kinase) domain in RIOK2 was observed (Ferreira-Cerca, S. et al. ATPase-dependent role of the atypical kinase Rio2 on the evolving pre-40S ribosomal subunit. Nat Struct Mol Biol 19, 1316-1323 (2012); LaRonde-LeBlanc, N. & Wlodawer, A. A family portrait of the RIO kinases. J Biol Chem 280, 37297-37300 (2005)). Since wHTH domains form key structural motifs in both prokaryotic and eukaryotic bona fide transcription factors, the inventors asked whether this atypical kinase might also function as a transcription factor (TF) in hematopoietic differentiation to regulate lineage fate determination (Teichmann, M., Dumay-Odelot, H. & Fribourg, S. Structural and functional aspects of winged-helix domains at the core of transcription initiation complexes. Transcription 3, 2-7 (2012)). It was discovered that RIOK2 is a master transcriptional regulator of hematopoietic lineage commitment and that its ablation drives primary human stem and progenitor cells (HSPCs) towards MDS-associated hematopoietic differentiation defects. The transcriptomic profiling, structural modeling, ChIP sequencing, ATAC-sequencing and structure-function domain deletion mutants described herein revealed that RIOK2 regulates specific genetic programs in hematopoiesis via its previously unappreciated winged helix-turn-helix DNA-binding domain and two transactivation domains. Mechanistically, RIOK2 transcriptionally modifies the expression of key lineage-specific transcription factors, such as GATA1, GATA2, SPIl, RUNX3 and KLF1 to fine-tune lineage fate determination in primary human hematopoietic stem cells. It was further demonstrated that GATA1 and RIOK2 function in a positive feedback loop to drive erythroid differentiation. These discoveries thus present therapeutic opportunities to correct hematopoietic differentiation defects in a range of hematologic disorders. This is the first evidence of a protein that not only controls 40S ribosome biogenesis governing translation but also functions in the nucleus as a master transcription factor (TF) by regulating the expression of key transcription factors that determine hematopoietic stem cell fate.

### Results

### RIOK2 Regulates Differentiation of Hematopoietic Lineages

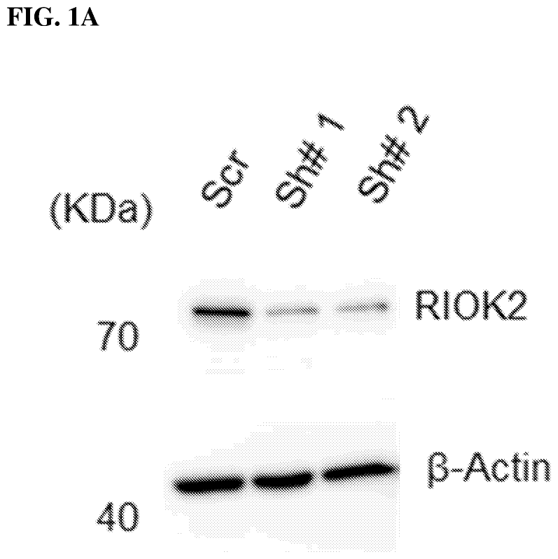

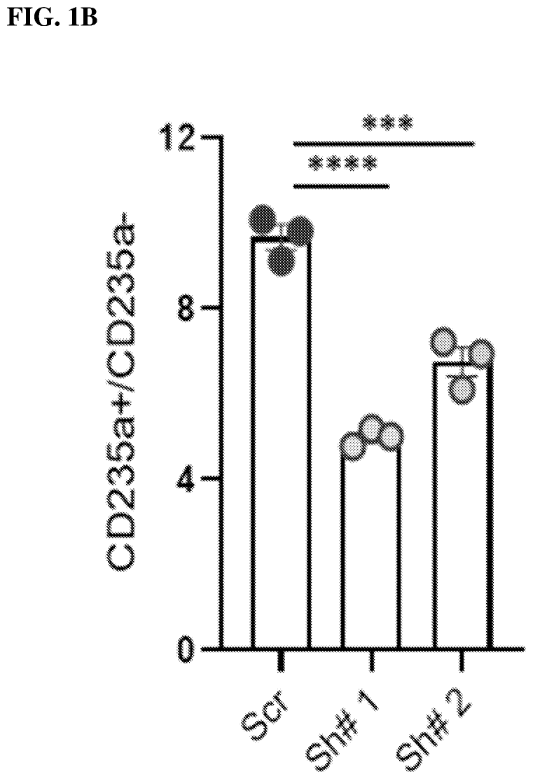

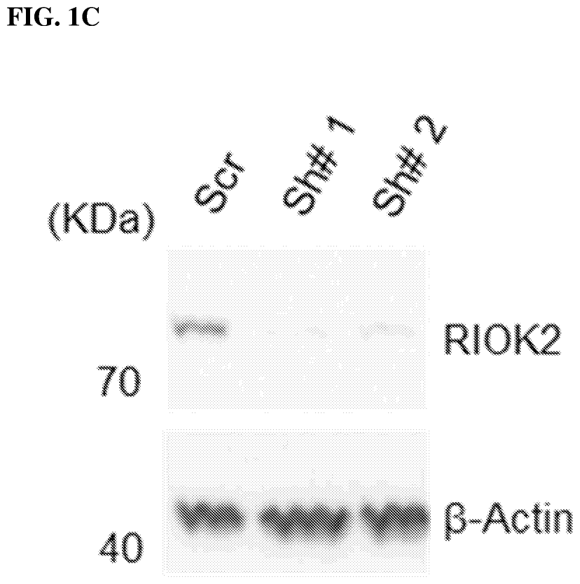

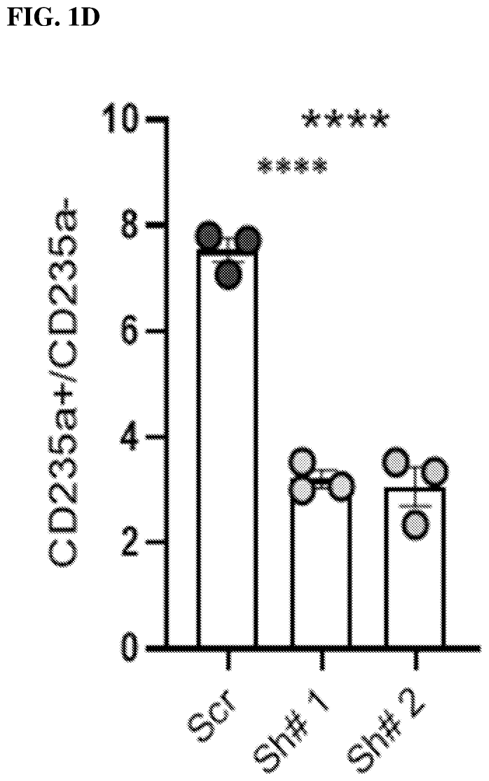

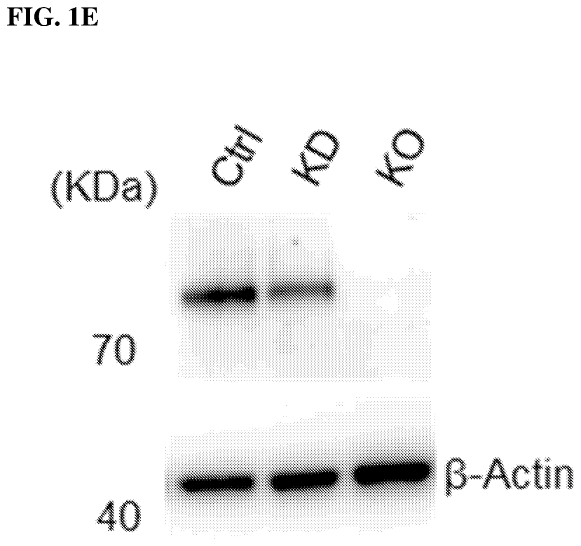

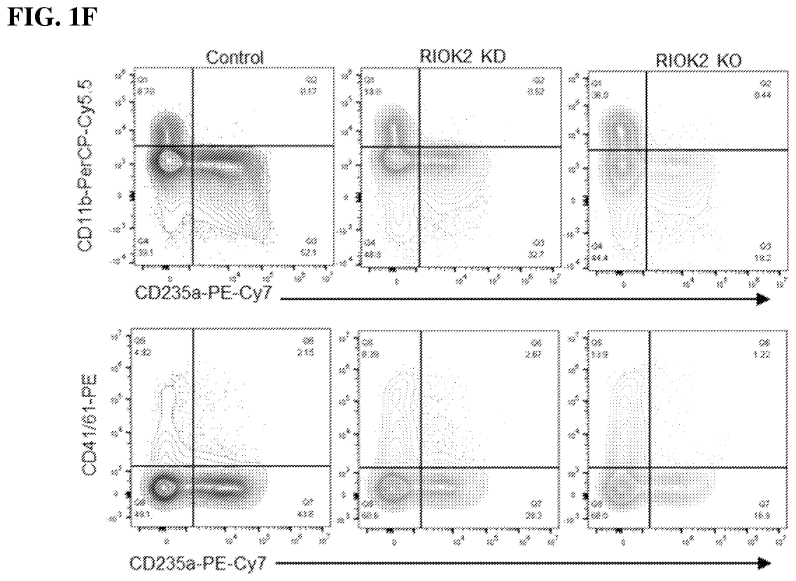

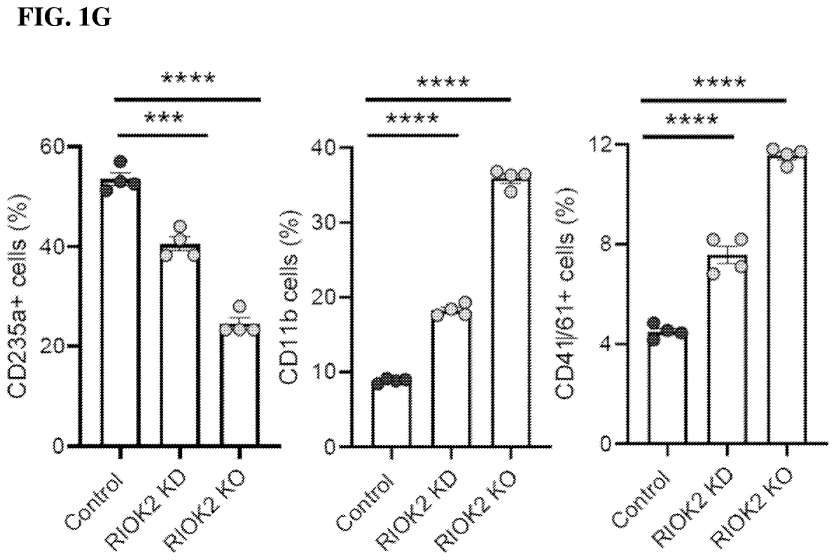

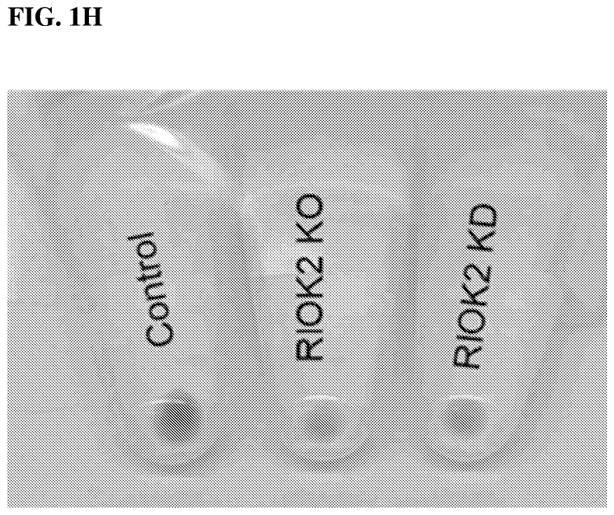

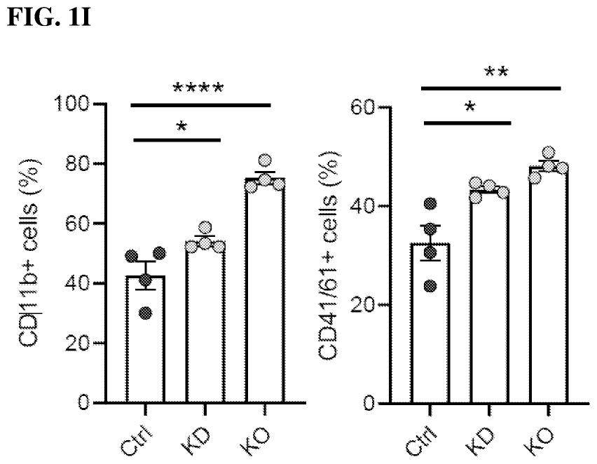

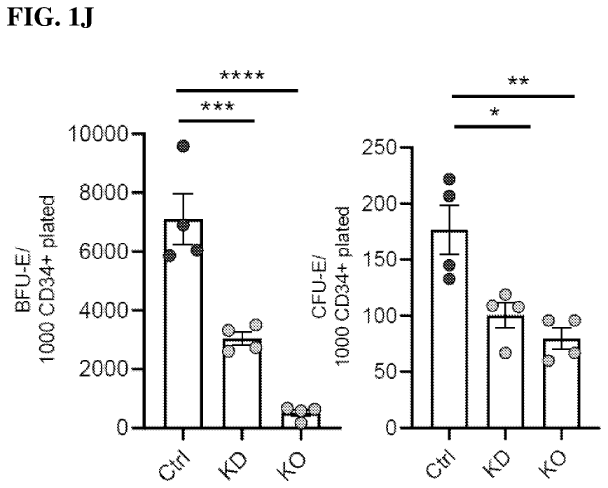

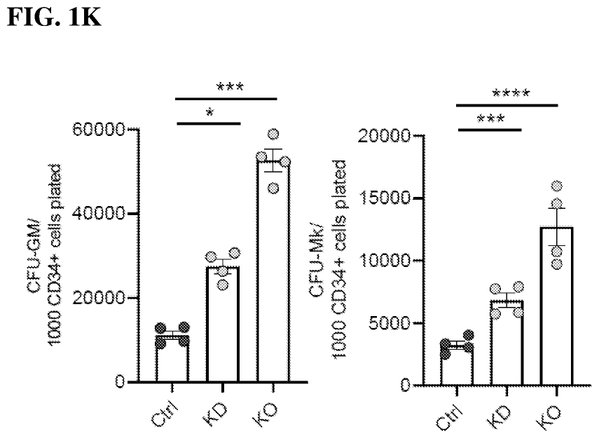

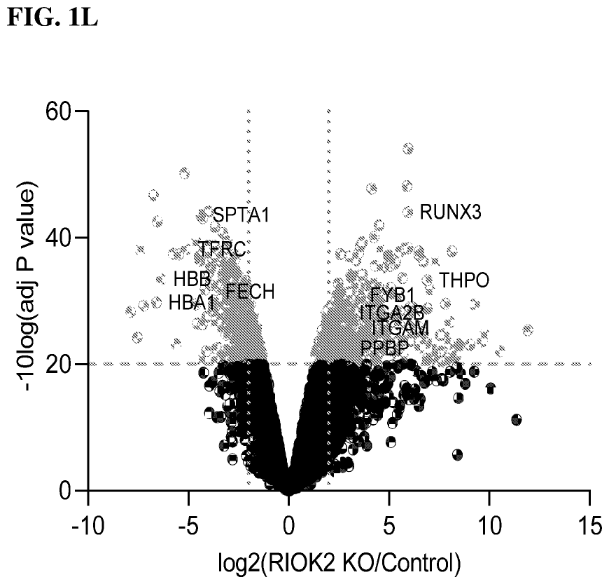

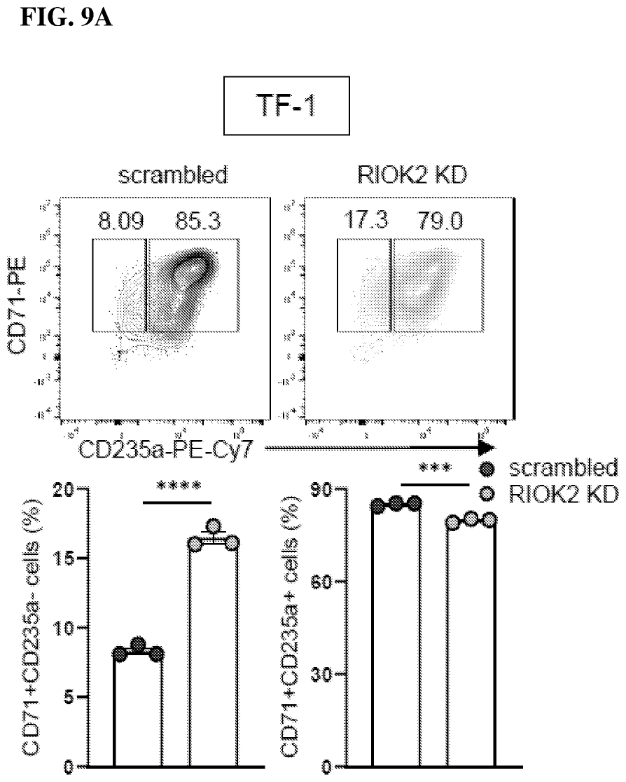

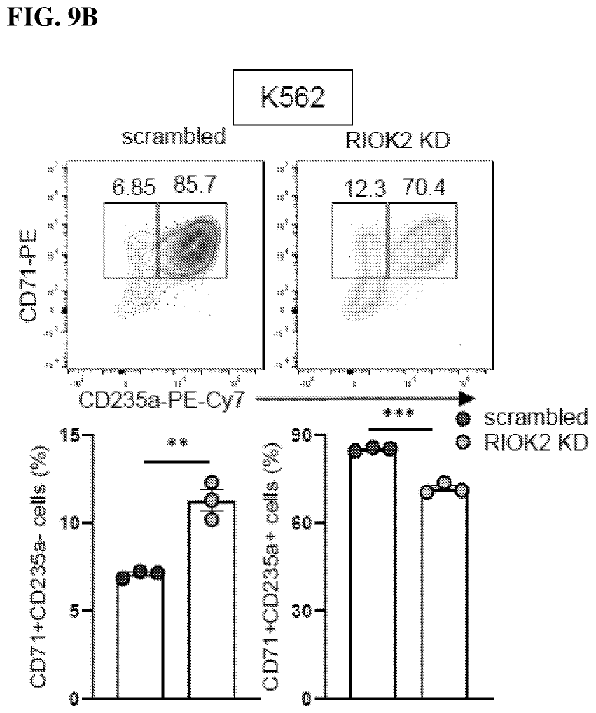

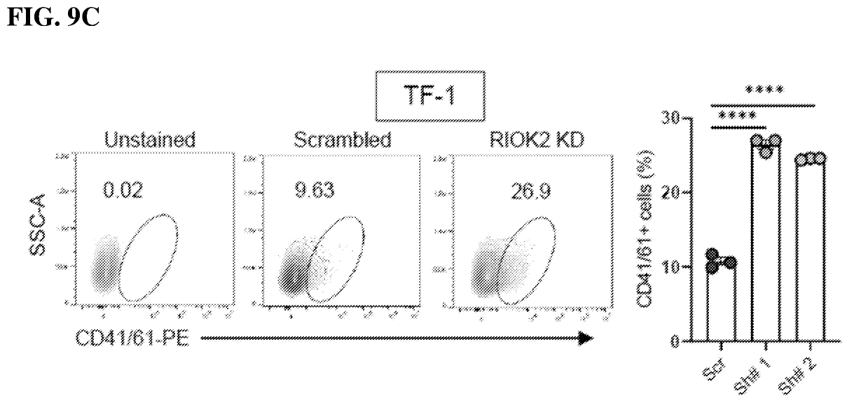

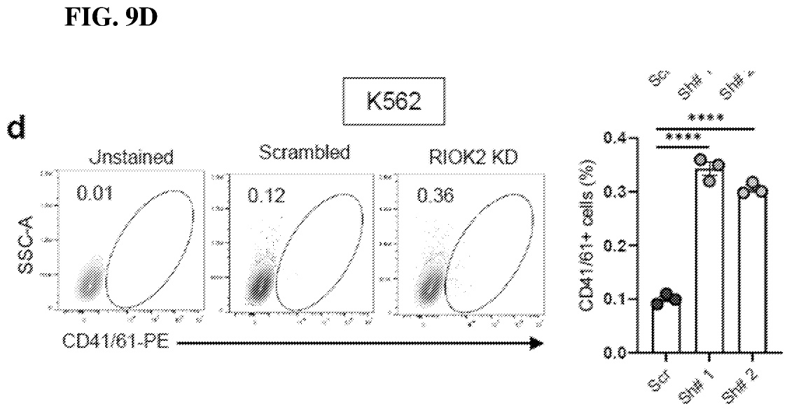

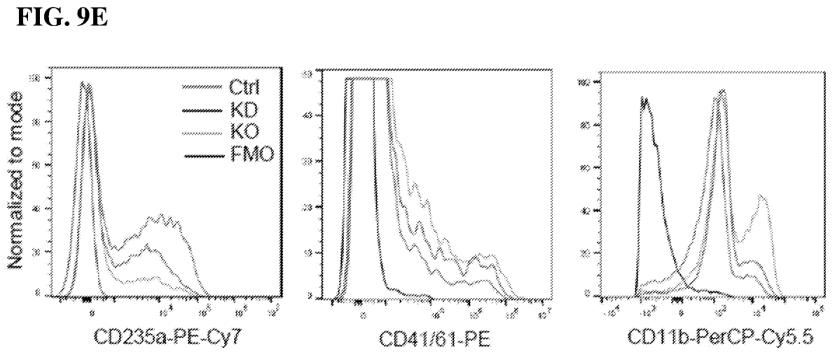

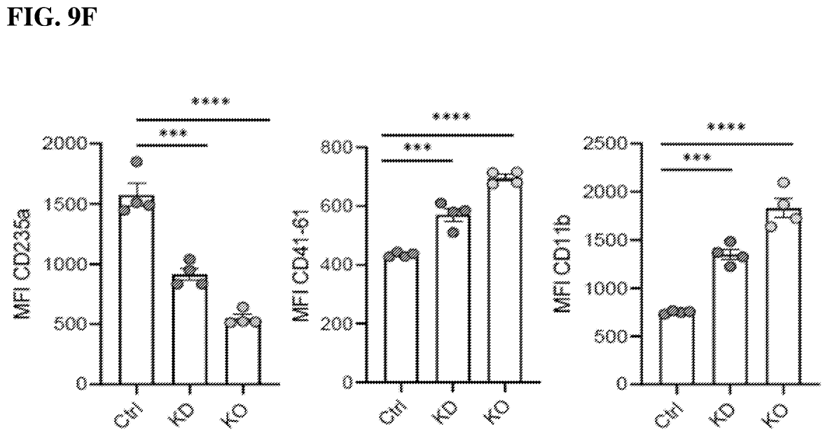

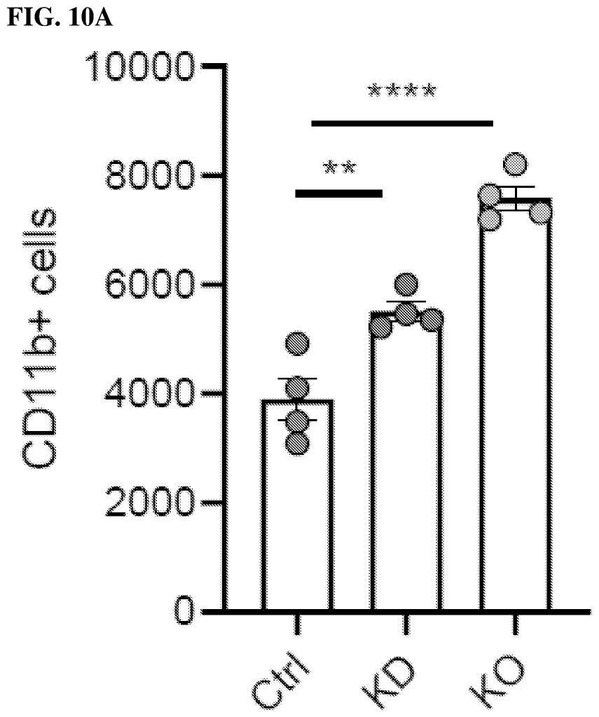

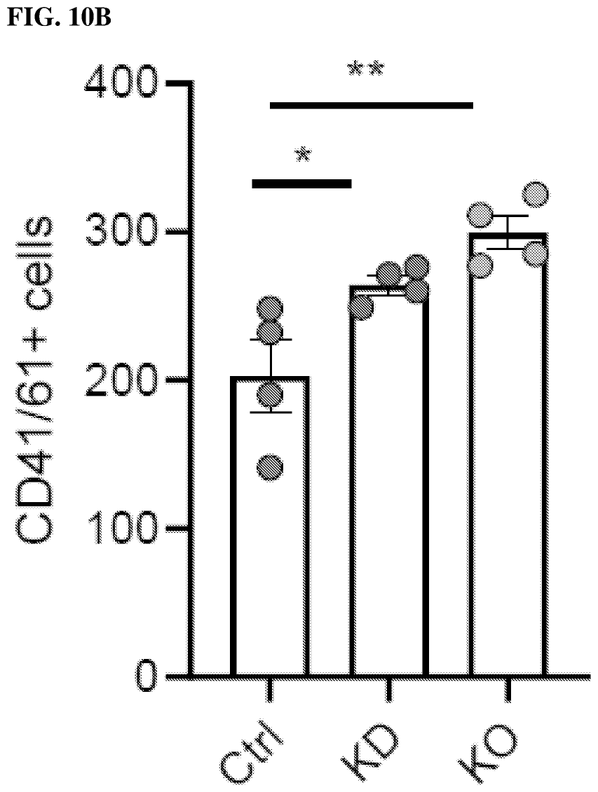

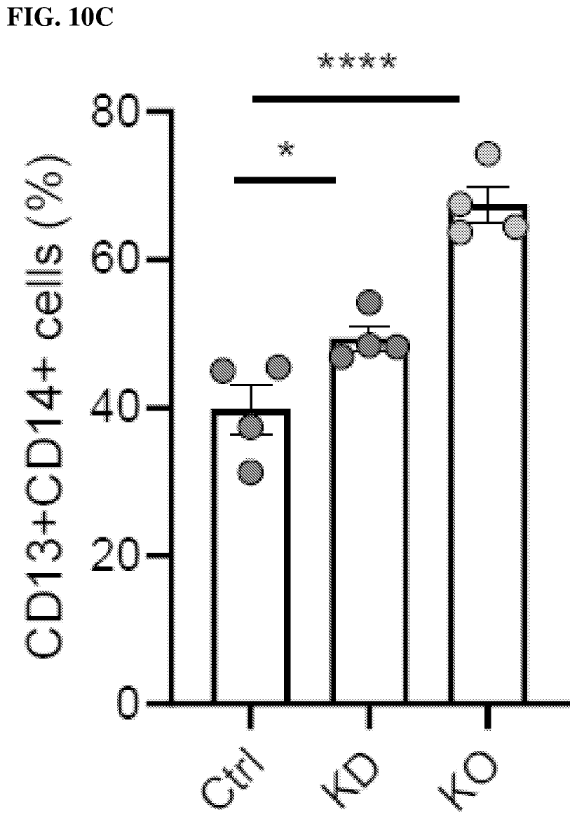

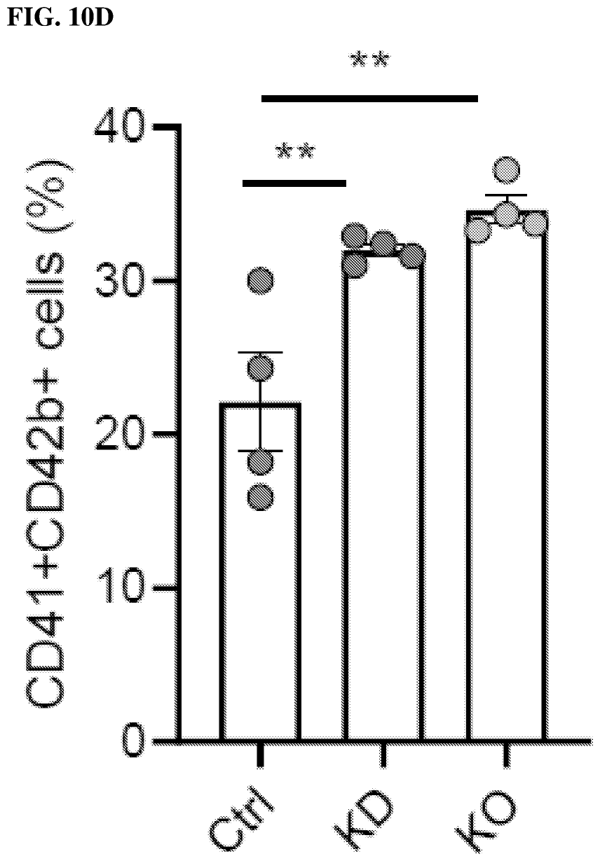

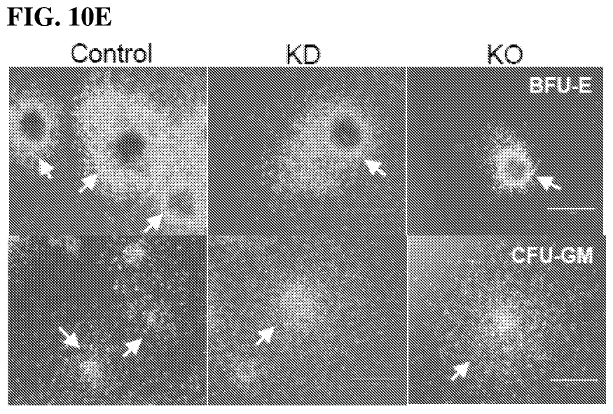

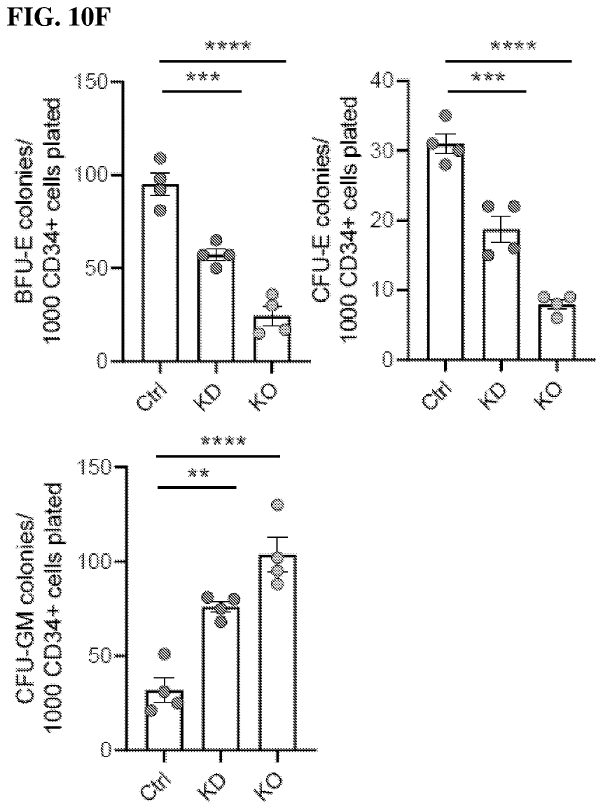

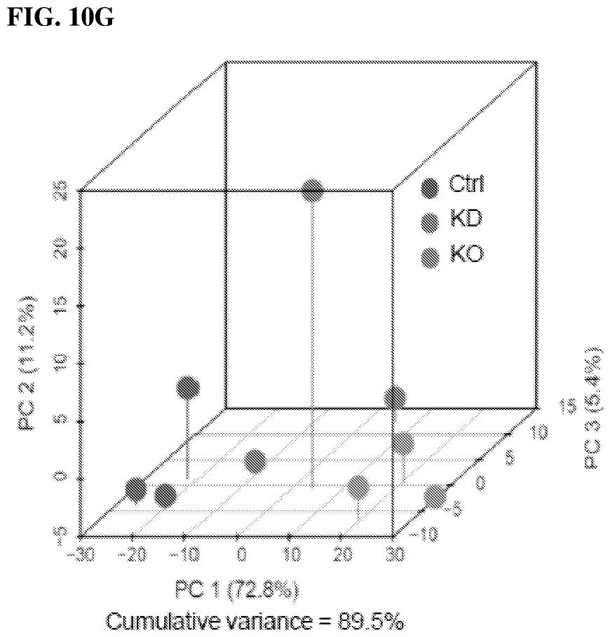

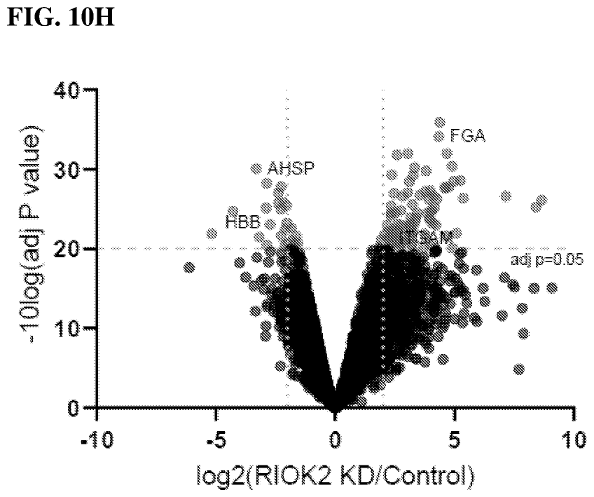

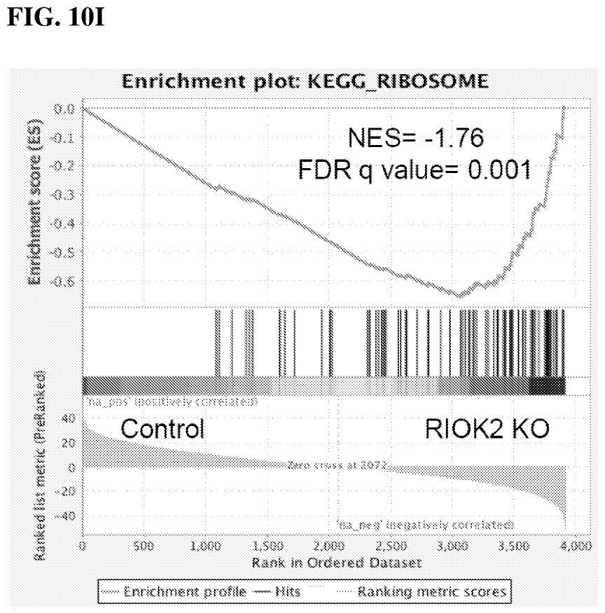

In agreement with a previous pre-clinical study in mouse models, it was observed that shRNA-mediated knockdown of RIOK2 led to a significant decline in erythroid differentiation in two different human erythroleukemia cell lines: TF-1 and K562 (FIGS. 1A-1D) (Raundhal, M. et al. Blockade of IL-22 signaling reverses erythroid dysfunction in stress-induced anemias. Nat Immunol 22(4):520-529 (2021)). Partial loss of RIOK2 modestly, but significantly, inhibited progression from the proerythroblast-like stage (CD71+CD235a-) to the more differentiated stage (CD71+CD235a+) in both cell lines (FIG. 9A-9B) (Nakahata, T. & Okumura, N. Cell surface antigen expression in human erythroid progenitors: erythroid and megakaryocytic markers. Leuk Lymphoma 13, 401-409 (1994)). Additionally, a concomitant skewing towards megakaryopoiesis in RIOK2-knockdown cells was observed (FIGS. 9C and 9D). To validate this observation and its correlation with the emergence of hematologic disorders, healthy donor-derived primary human hematopoietic stem and progenitor cells (HSPCs) were derived, which are the cells of origin for hematologic neoplasms in patients. Using CRISPR/Cas9 genome editing, RIOK2 knocked down (KD) or knocked out (KO) (gRNAs targeting LacZ used as control) in primary human HSPCs (FIG. 1E). Loss of RIOK2 in HSPCs not only blocked erythroid lineage commitment; it also increased megakaryopoiesis and myelopoiesis (FIGS. 1F, 1G and FIG. 9E, 9F, and 18). Depletion of RIOK2 led to substantial retention of HSPCs in the uncommitted stage (Q4) rather than passive progression to other lineages (FIG. 1F), suggesting that RIOK2 is actively involved in regulating early hematopoietic stem cell differentiation. Analysis of cell pellets in RIOK2-proficient vs deficient HSPCs underscored the significance of RIOK2 in driving erythroid differentiation (FIG. 1H). To further define the direct roles of RIOK2 in myelopoiesis and megakaryopoiesis, HSPCs were cultured in differentiation media that selectively promoted myeloid or megakaryocytic differentiation. Consistently, loss of RIOK2 markedly elevated myelopoiesis and megakaryopoiesis (FIG. 1I and FIG. 10A-10D). To investigate if RIOK2 functions further upstream in the differentiation pathway, colony forming unit (CFU) assays using primary human HSPCs was performed. Loss of RIOK2 significantly impaired BFU-E (blast forming unit-erythroid) and CFU-E (colony forming unit-erythroid) formation and concomitantly increased megakaryocytic (CFU-Mk) and granulocyte-monocyte (CFU-GM) progenitors (FIGS. 1J and 1K and FIG. 10). Quantitative mass spectrometry-based proteomics further validated that loss of RIOK2 impedes expression of erythroid lineage proteins (HBB, HBA1, SPTA1, TFRC, FECH), while promoting the expression of megakaryocytic and myeloid lineage-specific proteins (ITGA2B, ITGAM, THPO, PPHB, FYB1) (FIGS. 1L, 1M, 10G and 10H). Gene set enrichment analysis confirmed defects in ribosome biogenesis upon loss of RIOK2 (FIG. 10I). Given that reduced ribosome levels selectively inhibit erythroid differentiation, it is beleived that RIOK2's kinase activity in 40S ribosome biogenesis and translation is also required for erythroid differentiation (Khajuria, R. K. et al. Ribosome Levels Selectively Regulate Translation and Lineage Commitment in Human Hematopoiesis. Cell 173, 90-103 el19 (2018)). However, it seemed unlikely that the significant reduction of RIOK2 mRNA in del(5q) MDS could be explained by defective ribosome biogenesis (Raundhal, M. et al. Blockade of IL-22 signaling reverses erythroid dysfunction in stress-induced anemias. Nat Immunol 22(4):520-529 (2021); Pellagatti, A. et al. Deregulated gene expression pathways in myelodysplastic syndrome hematopoietic stem cells. Leukemia 24, 756-764 (2010)). One explanation for reduced RIOK2 mRNA levels, however, could be control of RIOK2 transcription by other factors.

### GATA1 and RIOK2 Form a Positive Feedback Loop

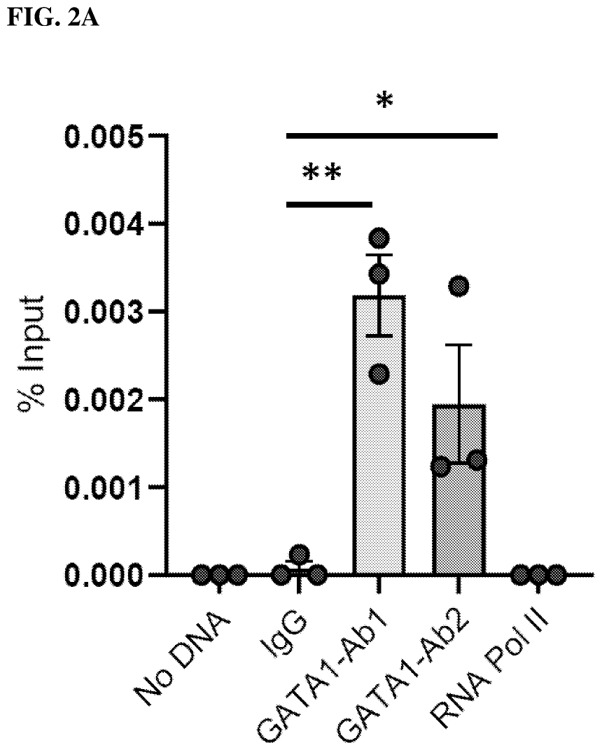

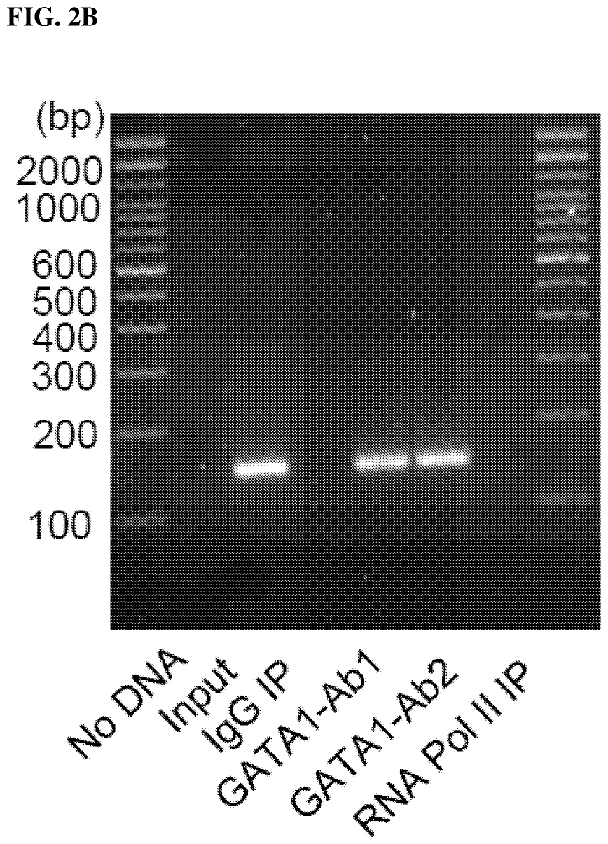

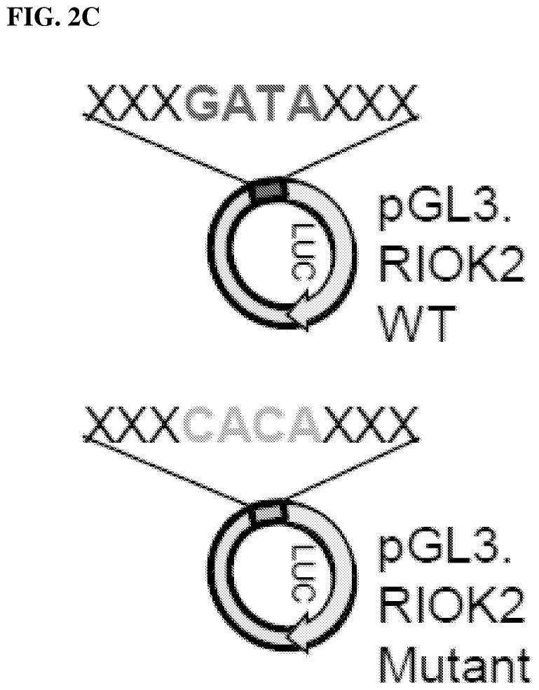

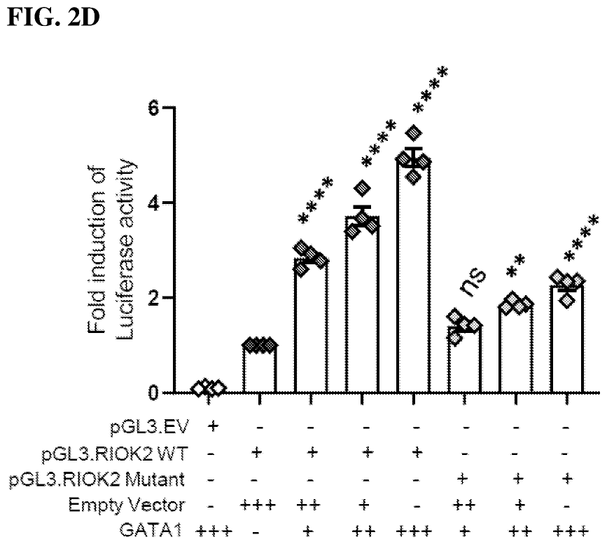

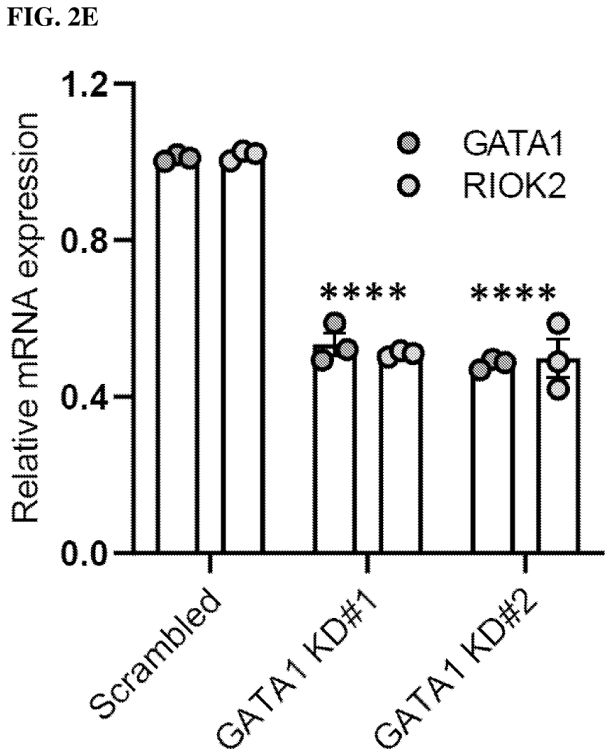

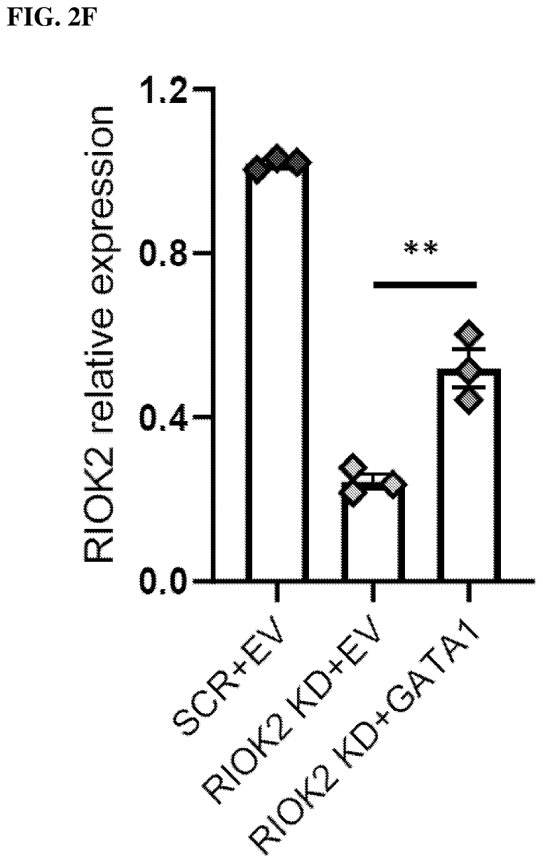

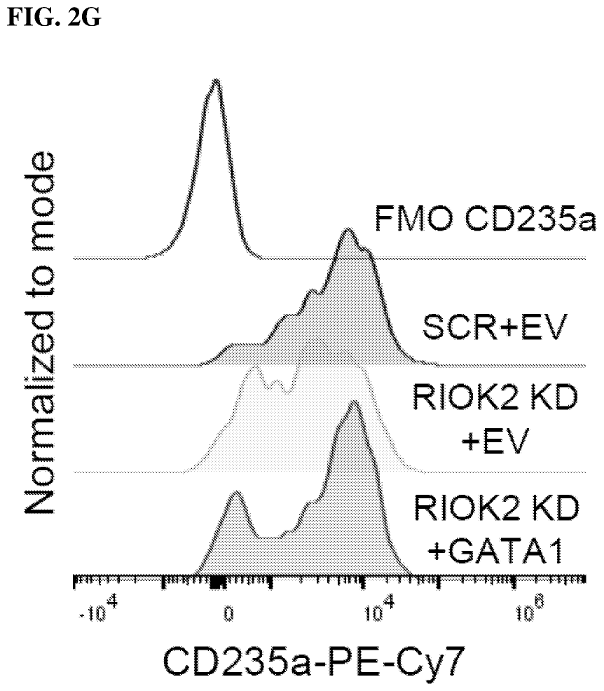

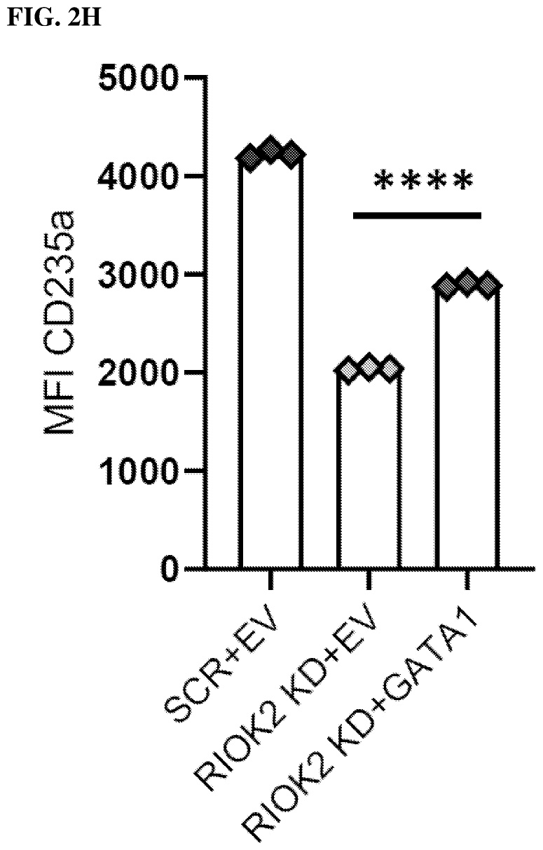

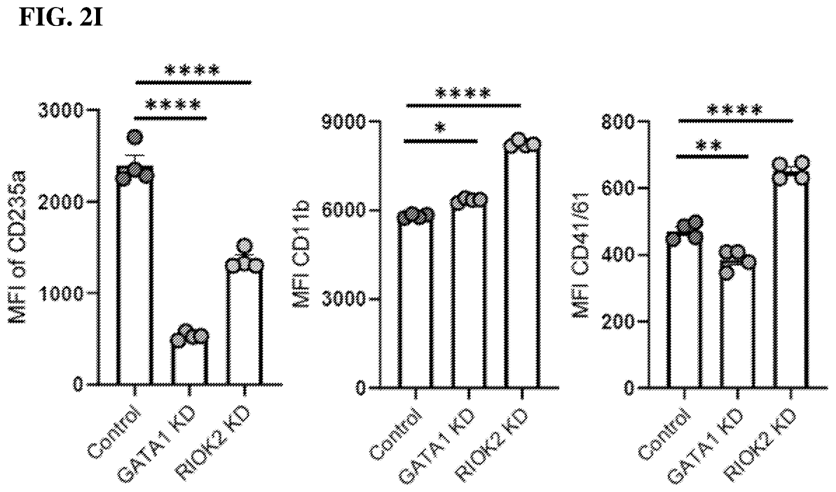

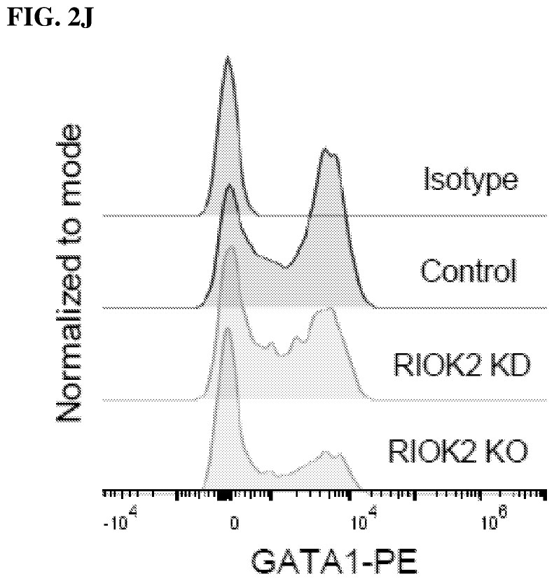

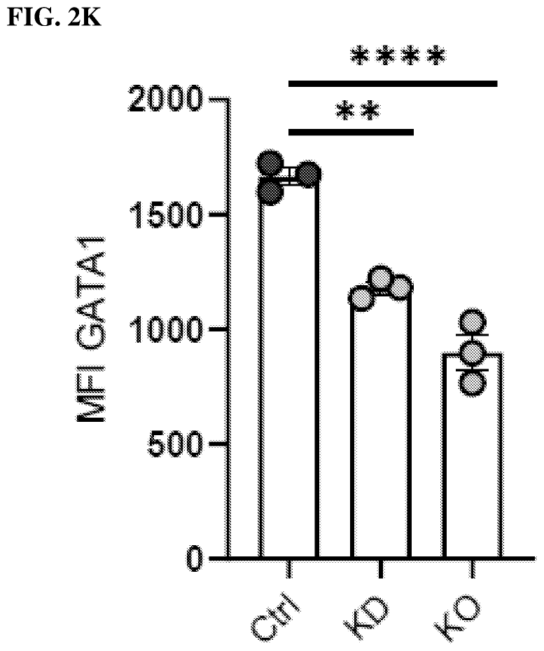

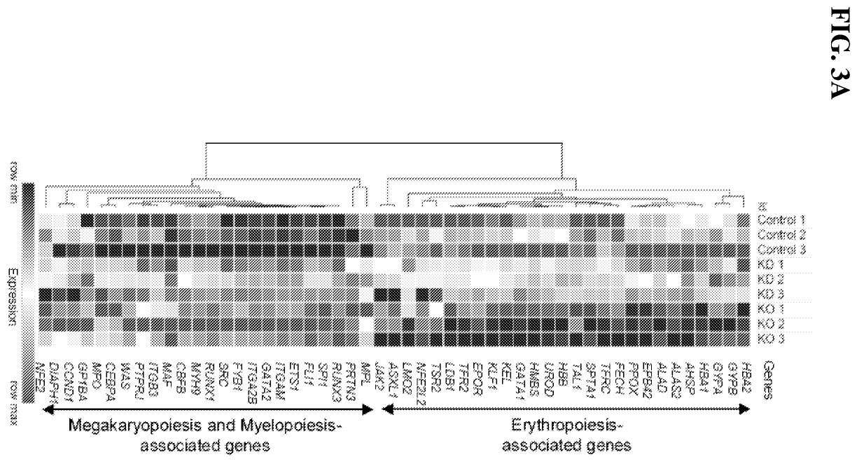

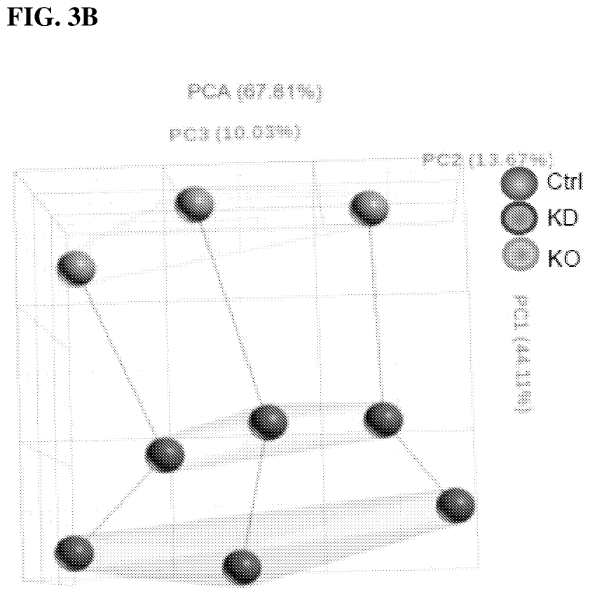

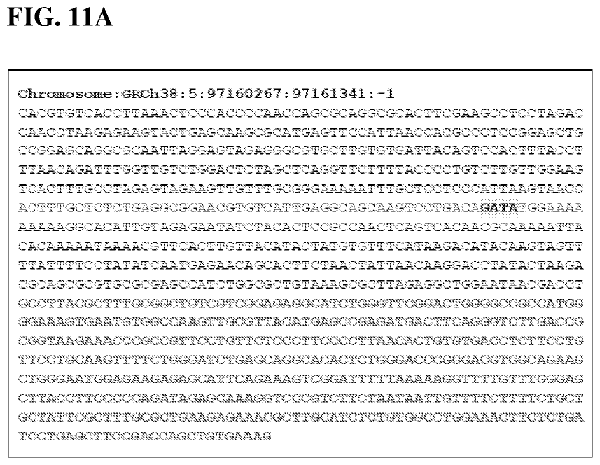

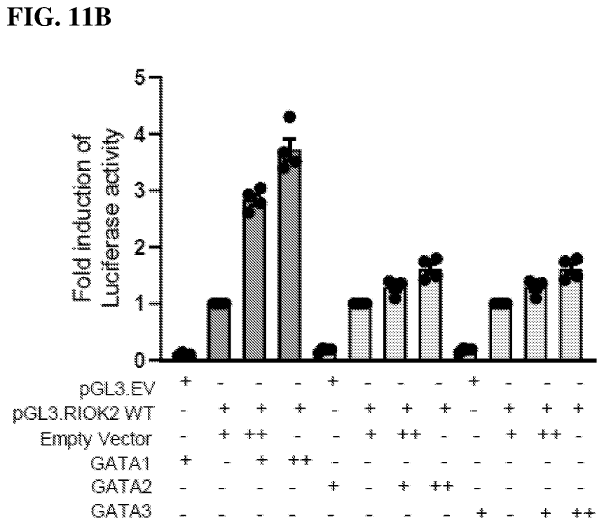

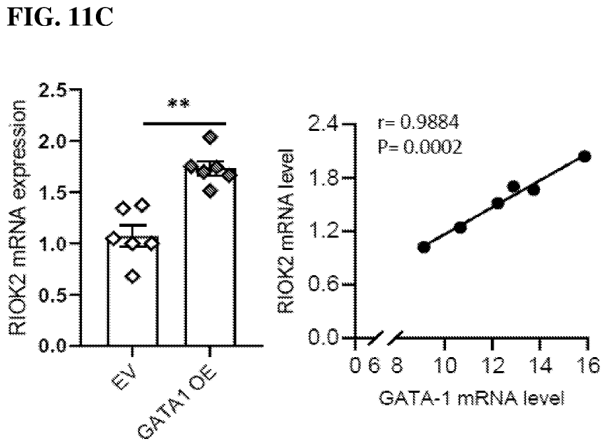

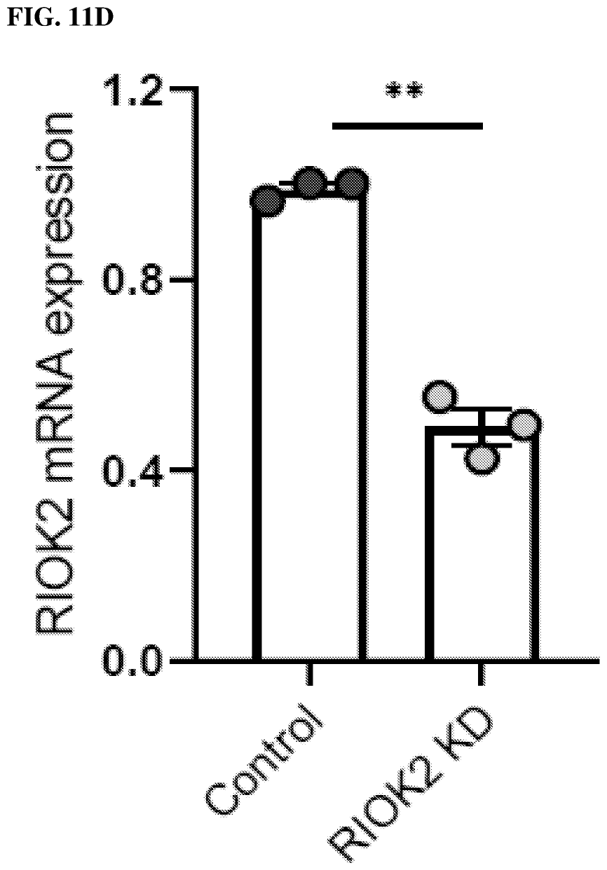

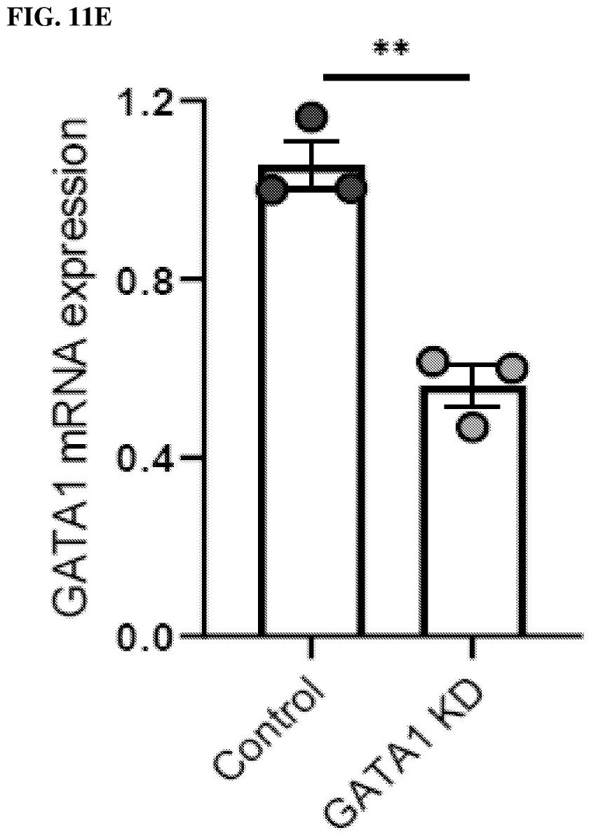

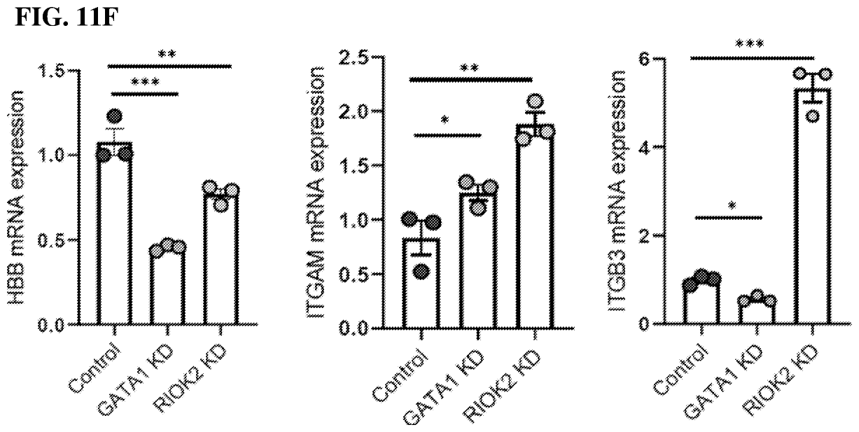

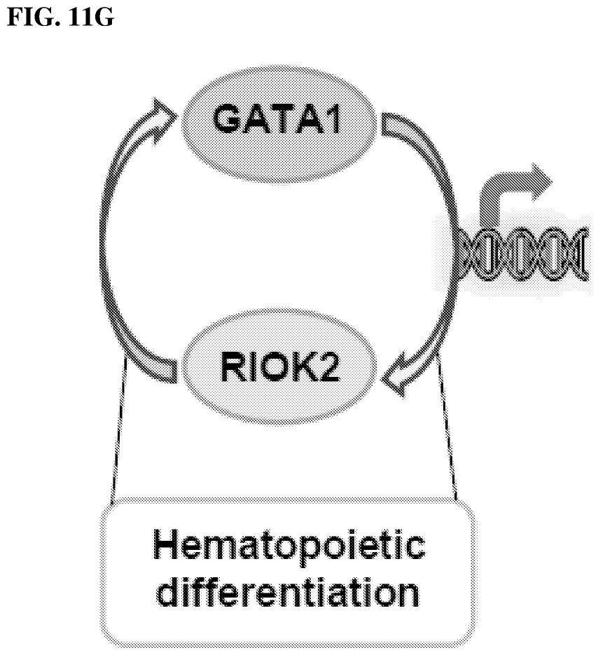

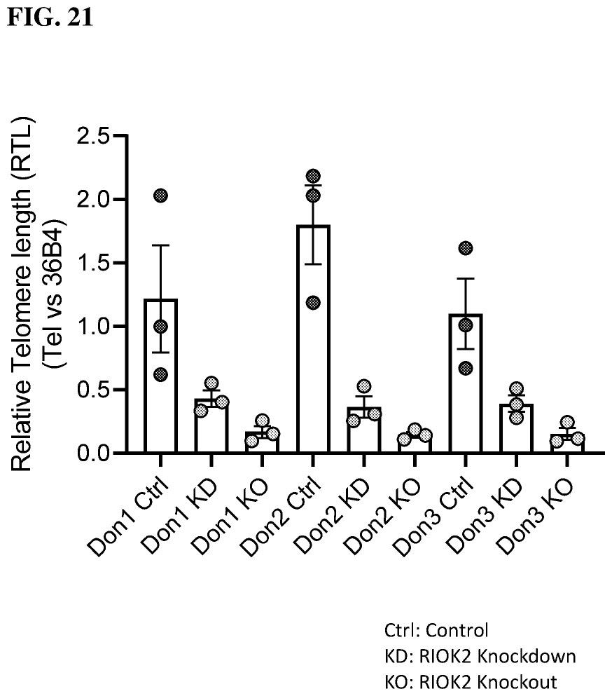

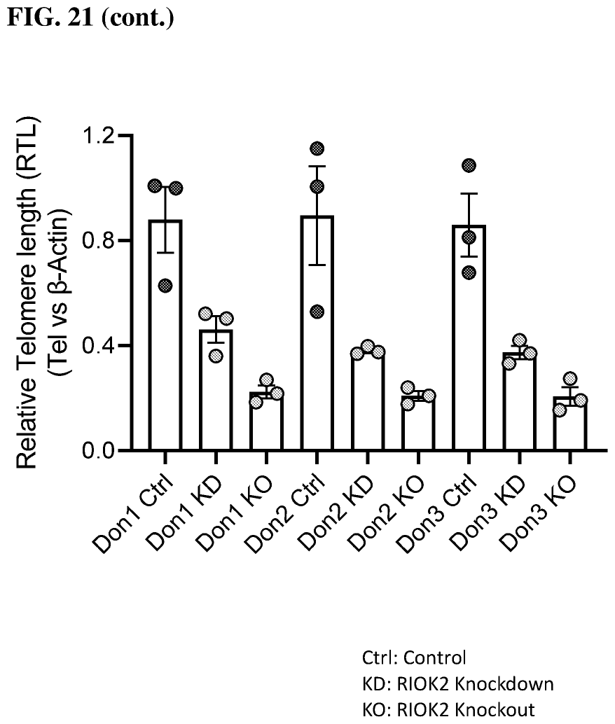

GATA1 is the most critical known transcriptional regulator of erythropoiesis. Given the integral roles of RIOK2 in hematopoietic differentiation, the inventors sought to identify whether RIOK2 itself was regulated by GATA1 in hematopoietic stem cells. Analysis of the promoter region of RIOK2 indeed revealed a GATA binding motif (FIG. 11A). Amongst GATA family members involved in hematopoiesis (GATA1, 2 and 3), only GATA1 robustly drove RIOK2 expression in luciferase reporter assays (FIG. 11B) Fujiwara, T. GATA Transcription Factors: Basic Principles and Related Human Disorders. Tohoku J Exp Med 242, 83-91 (2017)). Chromatin immunoprecipitation (ChIP) using two different monoclonal antibodies against GATA1 confirmed its binding to the RIOK2 promoter (FIGS. 2A and 2B). Mutation of the GATA binding site in the RIOK2 promoter significantly reduced GATA1-driven expression of RIOK2 (FIG. 2C and D). Additionally, a positive correlation between GATA1 and RIOK2 mRNA levels when GATA1 was either reduced or overexpressed was observed (FIG. 2E and FIG. 11C). Overexpression of GATA1 partially restored RIOK2 mRNA levels and erythroid differentiation in RIOK2 knockdown (KD) cells (FIG. 2F-2H), indicating that defects in erythroid differentiation due to loss of RIOK2 are only partially driven by GATA1. To address this, hematopoietic differentiation patterns were compared upon loss of GATA1 and RIOK2 in primary human HSPCs (FIGS. 11D and 11E). Although GATA1 was more potent than RIOK2 in driving erythroid differentiation, RIOK2 was much more potent than GATA1 in regulating myelopoiesis and megakaryopoiesis (FIG. 21 and FIG. 11F). Unexpectedly, it was also observed that decreased GATA1 protein expression upon gradual loss of RIOK2 (FIGS. 2J and 2K). These findings indicate a positive feedback loop between RIOK2 and GATA1 in driving hematopoietic differentiation, however it remained unclear how RIOK2 drove GATA1 expression (FIG. 11G). These data along with the previously reported dynamic localization of RIOK2 in the nucleus suggest that RIOK2's regulation of lineage commitment could not be wholly explained by its known functions in 40S ribosome biogenesis and cytoplasmic translation (Zemp, I. et al. Distinct cytoplasmic maturation steps of 40S ribosomal subunit precursors require hRio2. J Cell Biol 185, 1167-1180 (2009)).

### RIOK2 Regulates Key Hematopoietic Lineage-Specific TFs

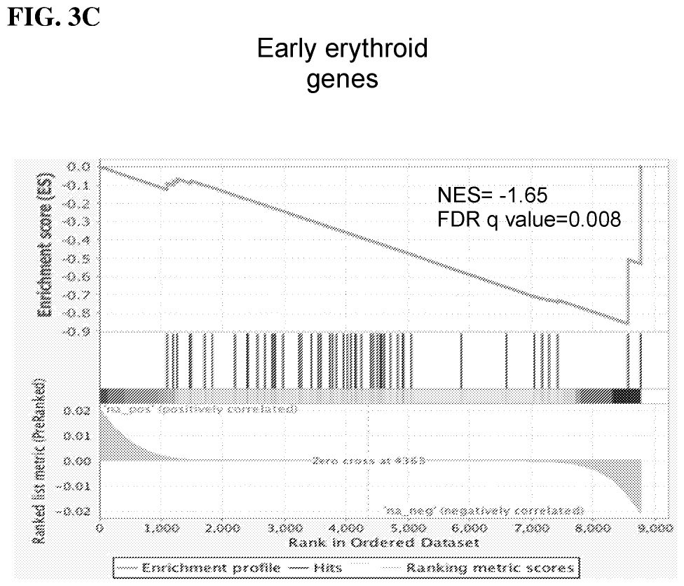

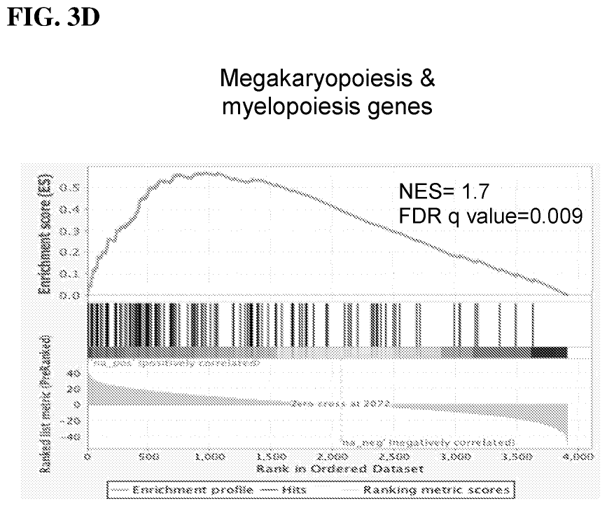

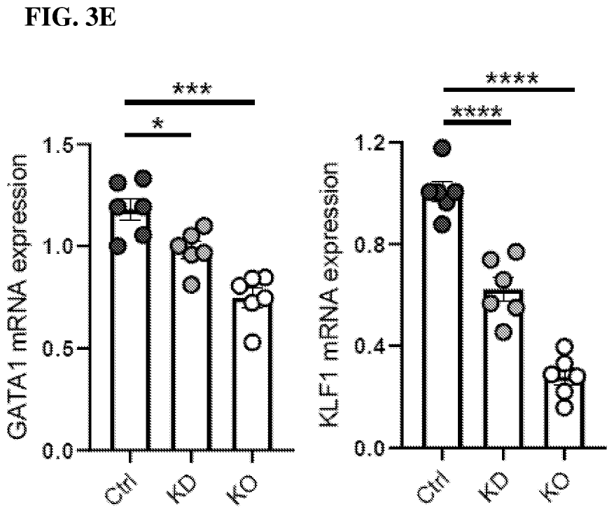

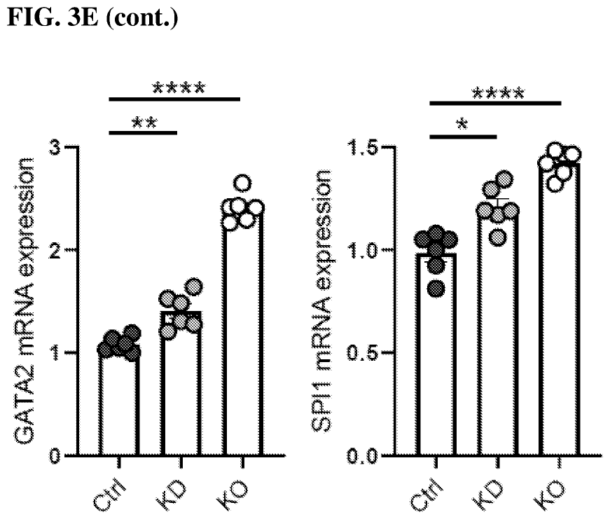

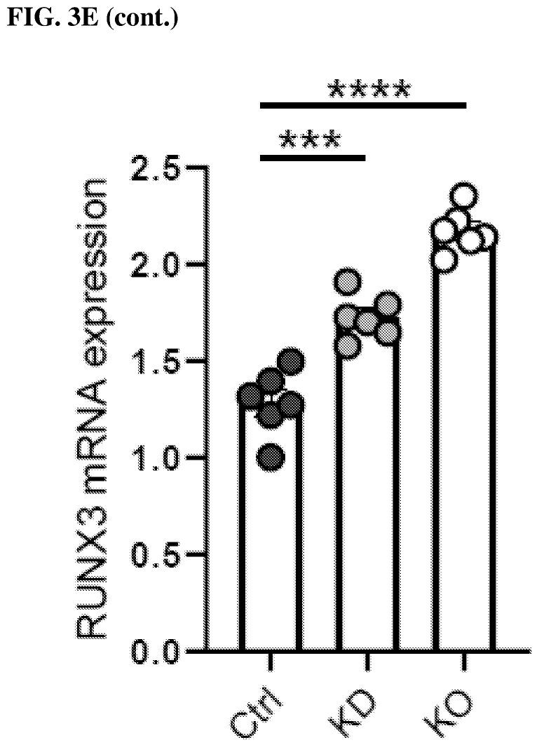

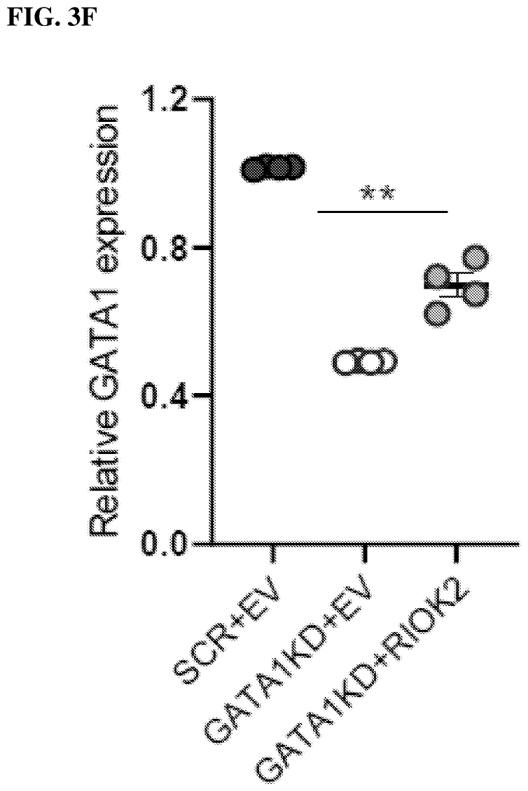

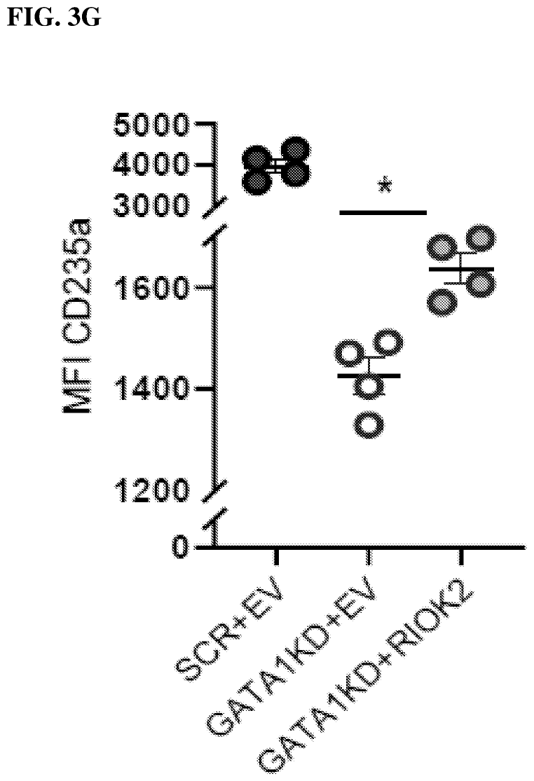

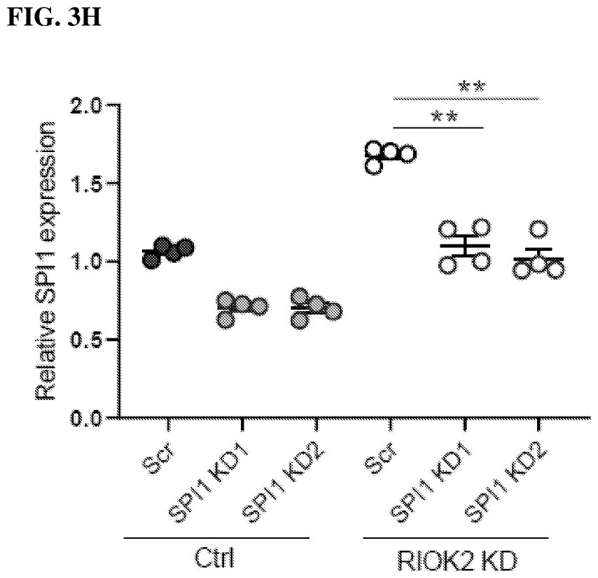

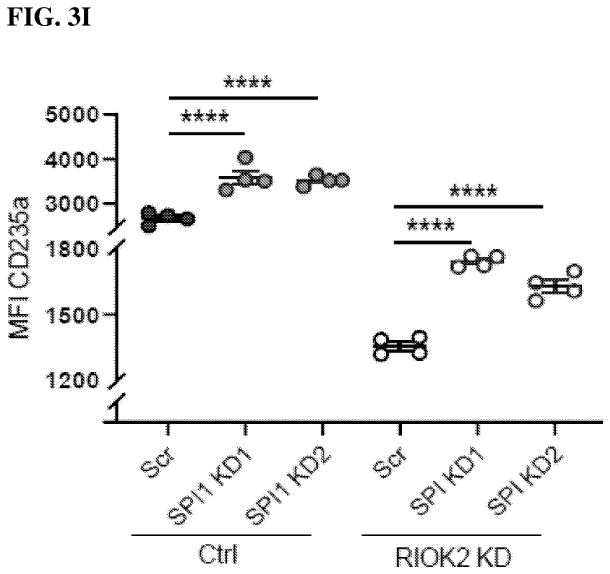

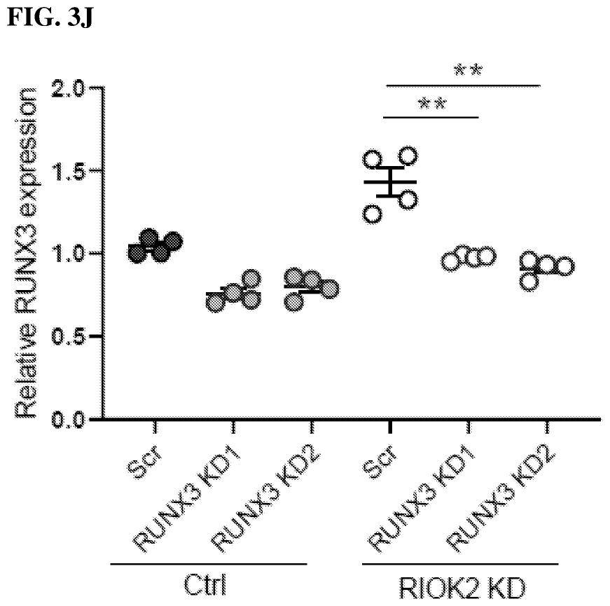

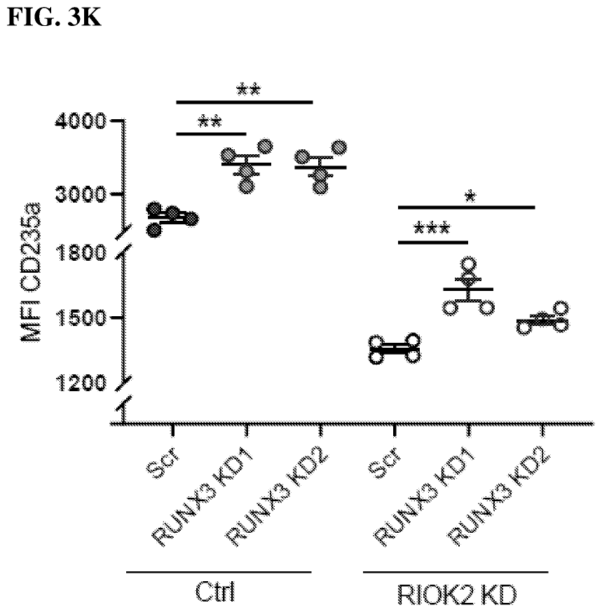

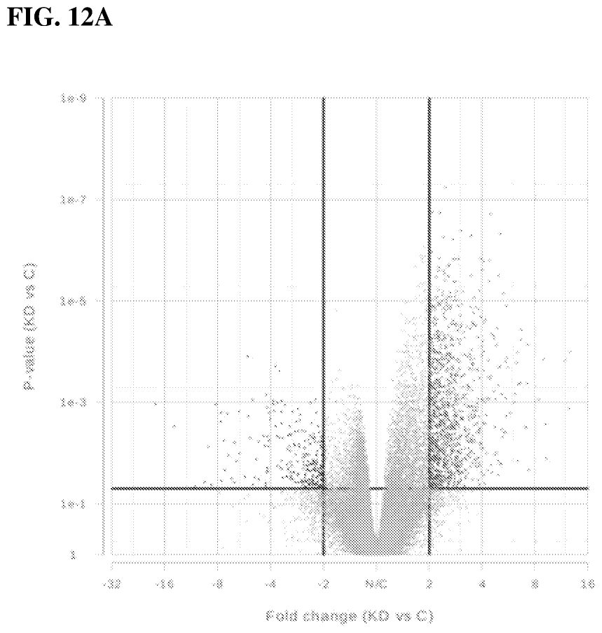

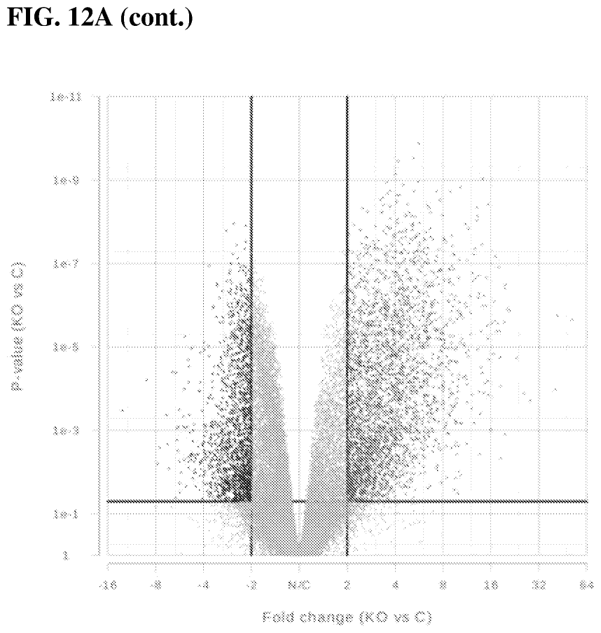

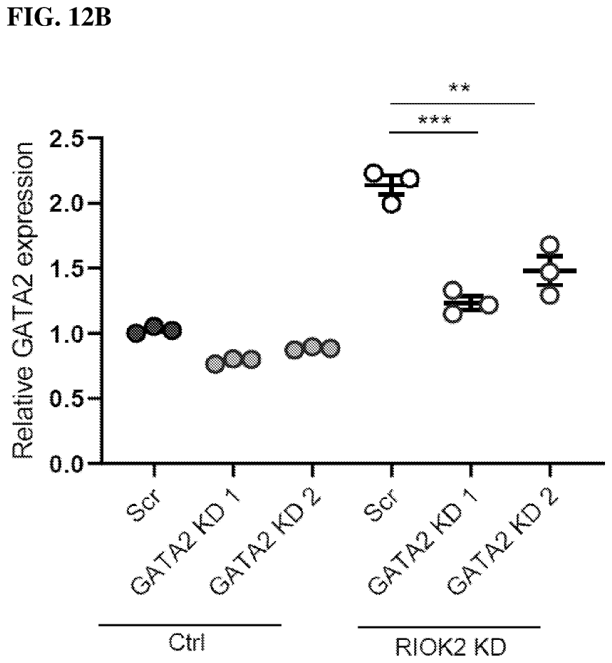

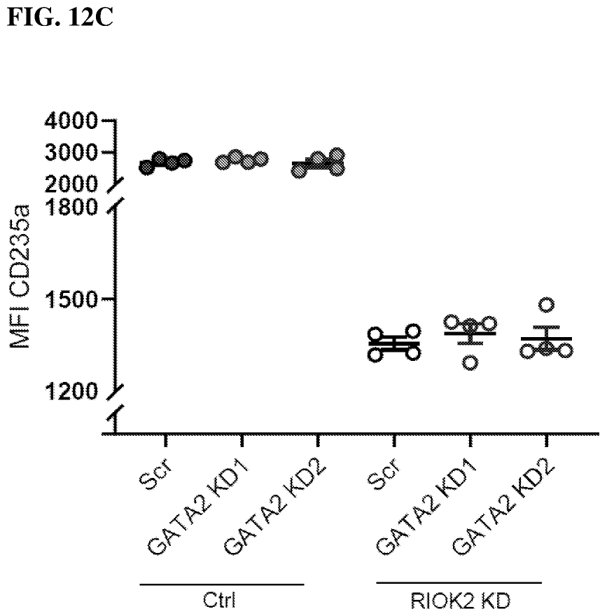

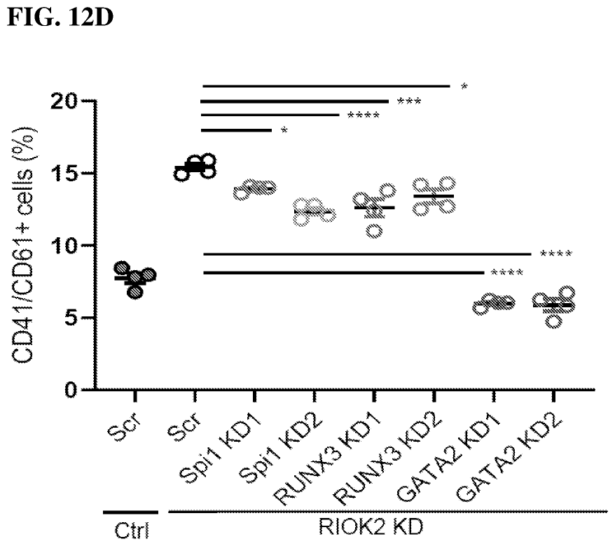

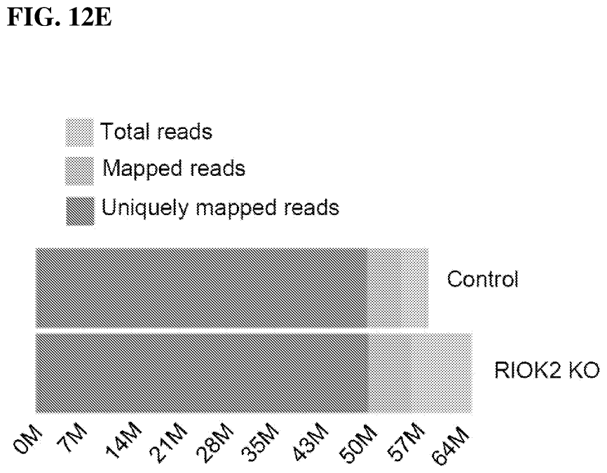

To gain deeper mechanistic insights into RIOK2's involvement in hematopoietic differentiation, bulk RNA sequencing was performed in RIOK2 knockdown (KD) and knockout (KO) primary human HSPCs (FIG. 3A, 3B). Consistent with full versus partial RIOK2 depletion, the KO vs control group generated more differentially-expressed genes than KD vs control group (FIG. 12A). Significantly altered genes overlapping in both KD and KO cells were pursued. Gene expression profiling clearly indicated a block in erythroid lineage commitment and a concomitant elevation in myelopoiesis and megakaryopoiesis-associated gene signatures with dose-dependent loss of RIOK2 (FIGS. 3A and 3B). Gene set enrichment analyses confirmed this observation (FIG. 3C, 3D).

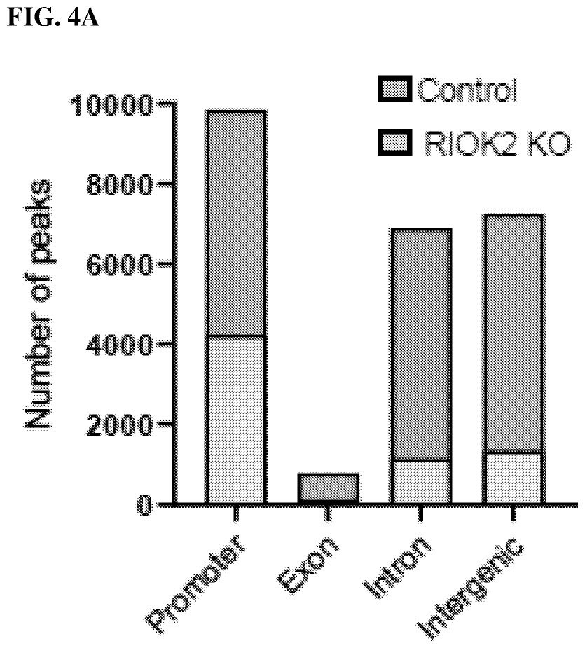

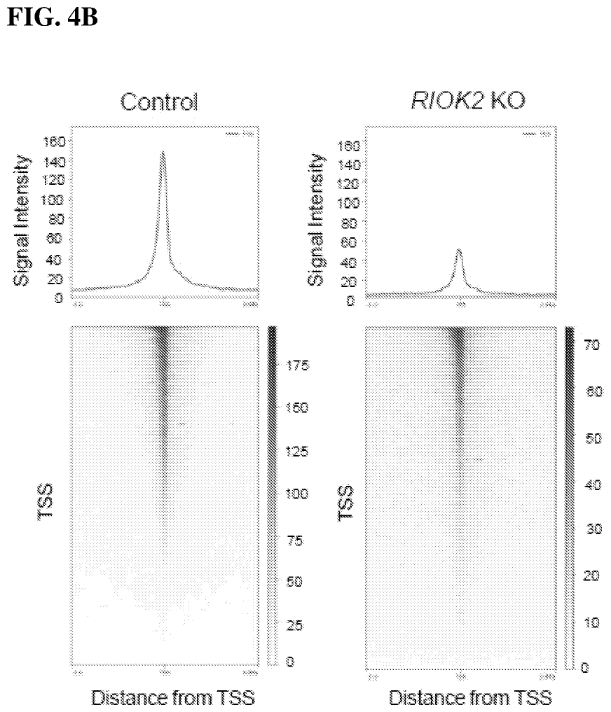

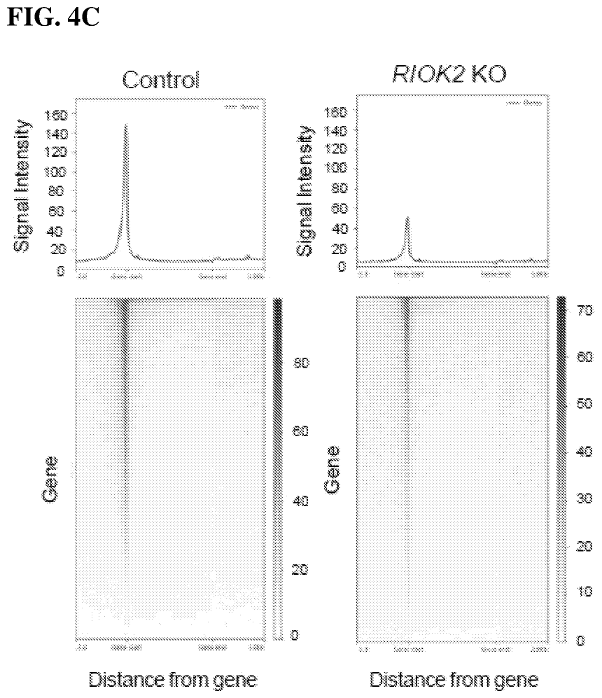

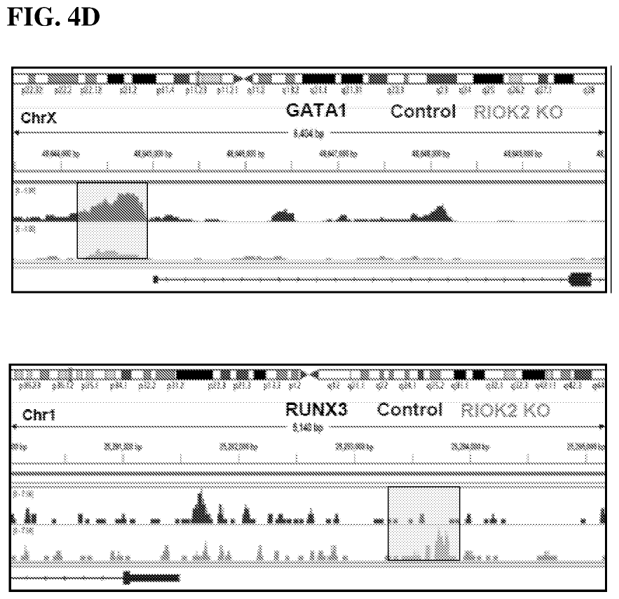

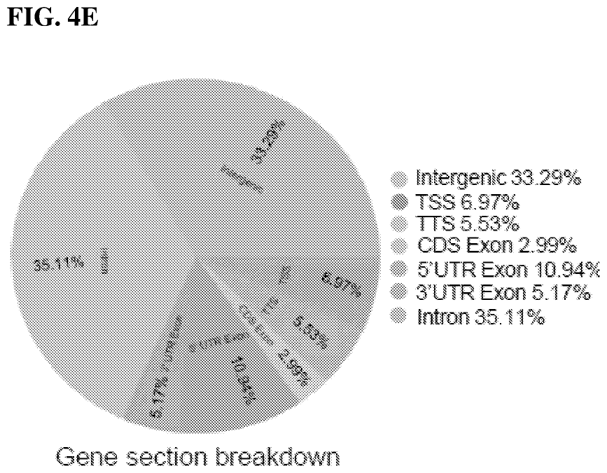

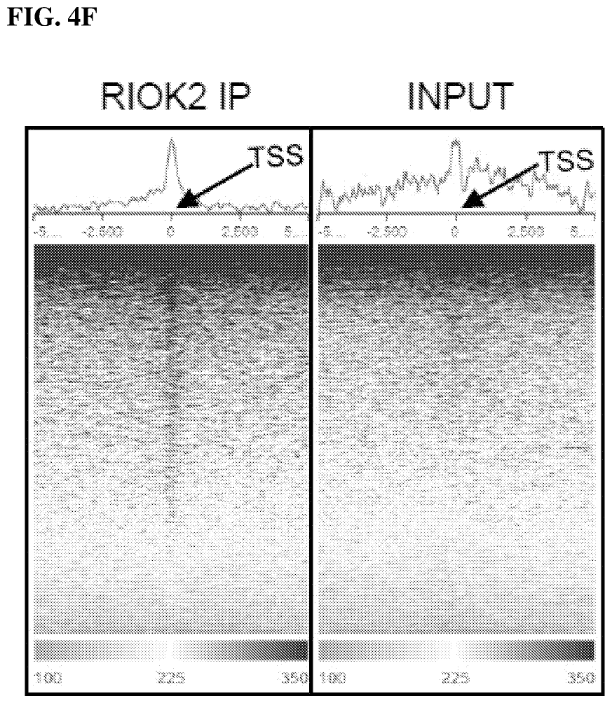

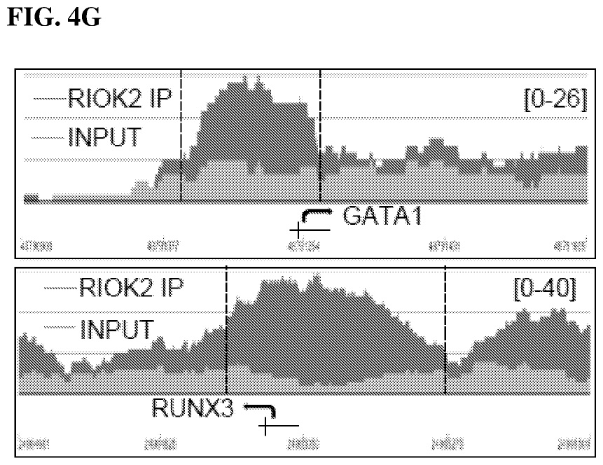

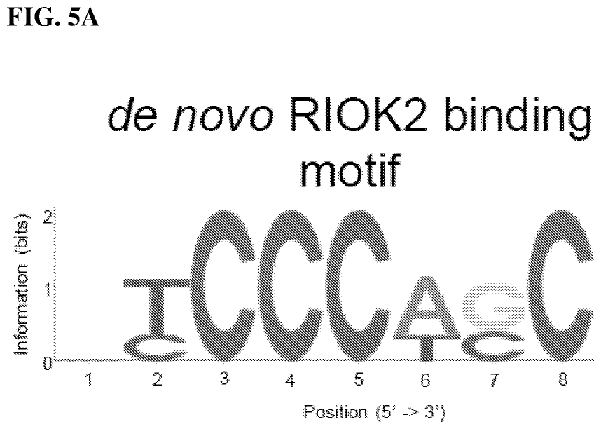

Notably, GATA1 mRNA levels were significantly downregulated upon RIOK2 depletion (FIGS. 3A and 3E), explaining the previously observed diminution in GATA1 protein levels in RIOK2-depleted HSPCs (FIGS. 2J and 2K). Remarkably, loss of RIOK2 also altered the expression profiles of additional key TFs that determine hematopoietic lineage commitment: specifically, GATA1 and KLF1 were downregulated; GATA2, SPI1 and RUNX3 were upregulated, amongst others (FIGS. 3A and 3E) (Ling, T. & Crispino, J.D. GATA1 mutations in red cell disorders. IUBMB Life 72, 106-118 (2020); Gnanapragasam, M. N. & Bieker, J.J. Orchestration of late events in erythropoiesis by KLF1/EKLF. Curr Opin Hematol 24, 183-190 (2017); Zhang, D. E. et al. Function of PU.1 (Spi-1), C/EBP, and AML1 in early myelopoiesis: regulation of multiple myeloid CSF receptor promoters. Curr Top Microbiol Immunol 211, 137-147 (1996); Yokomizo-Nakano, T. et al. Overexpression of RUNX3 Represses RUNX1 to Drive Transformation of Myelodysplastic Syndrome. Cancer Res 80, 2523-2536 (2020); Daw, S. & Law, S. The functional interplay of transcription factors and cell adhesion molecules in experimental myelodysplasia including hematopoietic stem progenitor compartment. Mol Cell Biochem 476(2):535-551 (2020)). It was next investigated if RIOK2-driven expression of the identified TFs promoted erythroid differentiation. Overexpression of RIOK2 partially but significantly increased GATA1 mRNA levels and erythroid differentiation in GATA1 KD cells (FIGS. 3F and 3G). Consistent with the established roles of key myeloid lineage TF SPIl (encoding PU.1) in blocking erythroid differentiation, it was noted that knockdown of SPIl partially rescued erythroid differentiation in RIOK2 KD cells (FIG. 3H, 3I). RUNX3 is another important hematopoietic TF whose elevated expression in MDS patients negatively correlates with median survival (Yokomizo-Nakano, T. et al. Overexpression of RUNX3 Represses RUNX1 to Drive Transformation of Myelodysplastic Syndrome. Cancer Res 80, 2523-2536 (2020)). An increased expression of RUNX3 upon RIOK2 deficiency (FIGS. 3A and 3E) was observed and noted as well that knockdown of RUNX3 partly rescued erythroid differentiation in RIOK2 KD cells (FIGS. 3J and 3K). However, ablation of GATA2, a key hematopoietic TF³¹, did not rescue erythroid differentiation in RIOK2 KD cells (Extended Data FIGS. 4B and 4C) (Shimizu, R. & Yamamoto, M. Quantitative and qualitative impairments in GATA2 and myeloid neoplasms. IUBMB Life 72, 142-150 (2020)). Consistent with the roles of GATA2 to promote megakaryopoiesis in the absence of GATA1, knockdown of GATA2 more robustly restored megakaryopoiesis to baseline levels in RIOK2 KD cells than suppression of SPIl or RUNX3 (FIG. 12D)(Huang, Z. et al. GATA-2 reinforces megakaryocyte development in the absence of GATA-1. Mol Cell Biol 29, 5168-5180 (2009)). This suggests that RIOK2 regulates the expression of multiple transcription factors critical for hematopoietic lineage commitment.

### RIOK2 Binds to Transcription Start Sites of its Target Genes

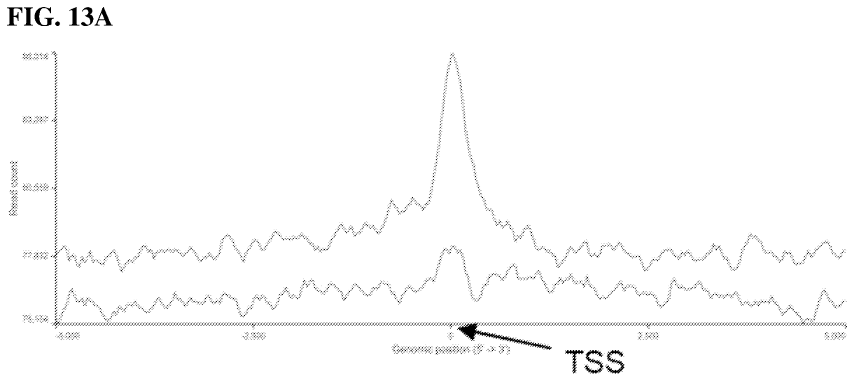

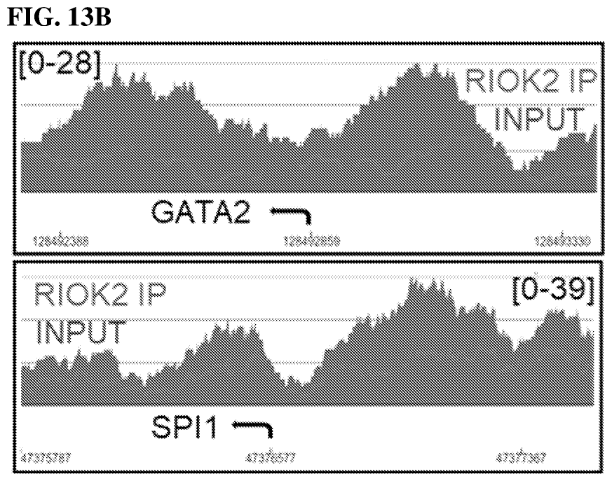

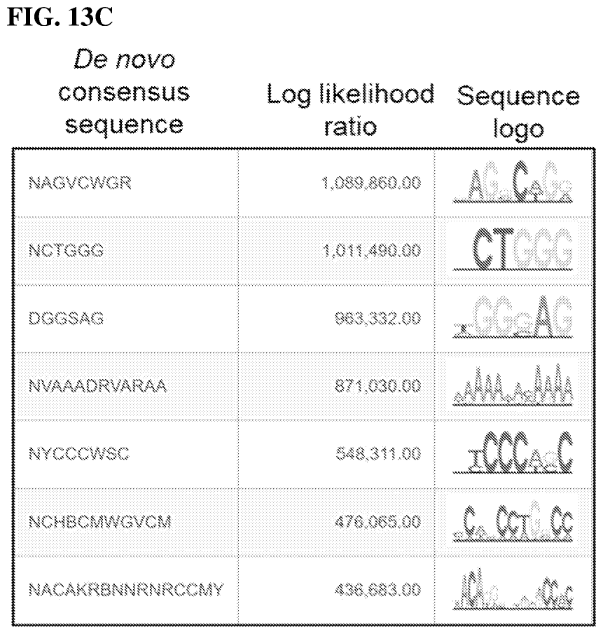

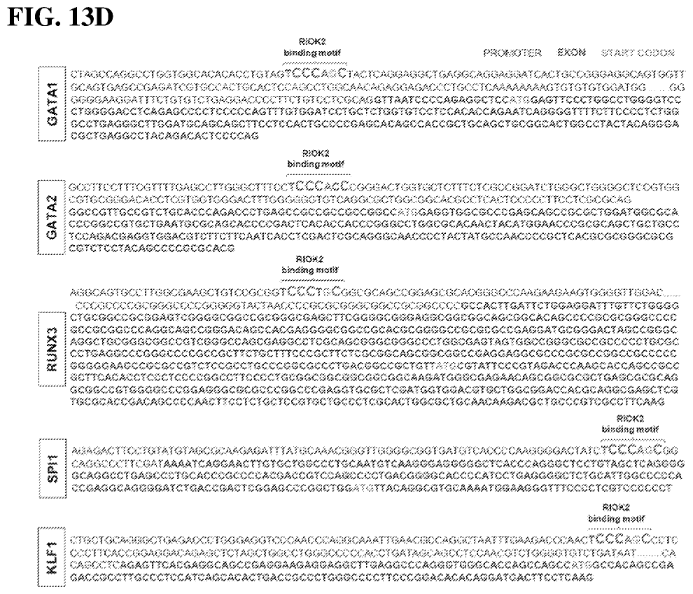

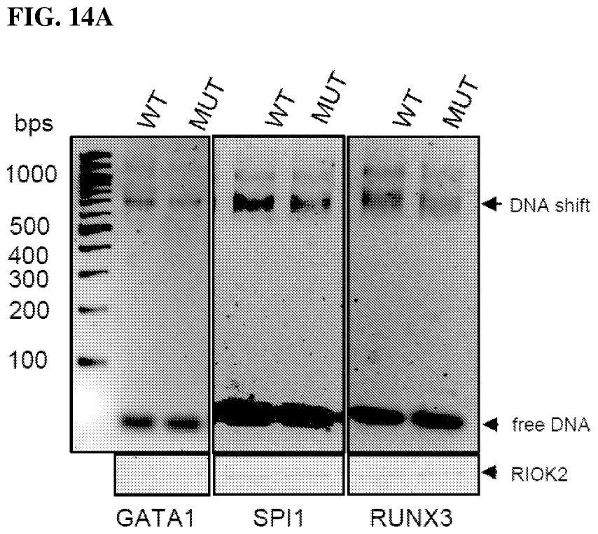

Given the notable alteration of transcriptomic profiles in RIOK2-depleted HSPCs as compared to control HSPCs (FIG. 3A), it was investigated whether loss of RIOK2 led to global changes in chromatin accessibility. To address this, an Assay of Transposase Accessible Chromatin with high-throughput Sequencing (ATAC-Seq) was proformed in differentiating RIOK2-proficient and deficient primary human HSPCs with comparable mapped reads (FIG. 12E). Indeed, loss of RIOK2 led to dramatic reduction in chromatin accessibility at the promoter, intronic and intergenic regions (FIG. 4A). Transcription start site (TSS) and gene plots further indicated an inhibition in chromatin accessibility at the promoter regions of genes in the absence of RIOK2 (FIGS. 4B and 4C; please note the maximum signal intensity of TSS plots in control HSPCs is 175 as opposed to only 70 in RIOK2-depleted HSPCs). Importantly, loss of RIOK2 led to differential chromatin accessibility at the promoter regions of its putative target genes, GATA1 and RUNX3 (FIG. 4D). These data indicated an integral role of RIOK2 in transcriptional regulation of genes. Examination of the amino acid sequence of RIOK2 revealed that it harbors a previously unexplored N-terminal winged helix-turn-helix (wHTH) domain, which is generally considered to be a structural motif involved in transcription (Ferreira-Cerca, S. et al. ATPase-dependent role of the atypical kinase Rio2 on the evolving pre-40S ribosomal subunit. Nat StructMol Biol 19, 1316-1323 (2012); Teichmann, M., Dumay-Odelot, H. & Fribourg, S. Structural and functional aspects of winged-helix domains at the core of transcription initiation complexes. Transcription 3, 2-7 (2012); Aravind, L., Anantharaman, V., Balaji, S., Babu, M. M. & Iyer, L. M. The many faces of the helix-turn-helix domain: transcription regulation and beyond. FEMS Microbiol Rev 29, 231-262 (2005)). Hence, it was asked whether RIOK2 functions as a TF to regulate hematopoietic differentiation. To address this, ChIP was preformed using monoclonal antibodies against RIOK2 followed by high-throughput DNA sequencing. Of note, occupancy of RIOK2 was observed at TSS and 5′UTR apart from intergenic and intronic regions (FIG. 4e), analogous to the genome-wide distribution patterns of bona fide TFs (Zhong, X. et al. HoxA9 transforms murine myeloid cells by a feedback loop driving expression of key oncogenes and cell cycle control genes. Blood Adv 2, 3137-3148 (2018); Xu, J. et al. Transcriptional silencing of {gamma}-globin by BCL11A involves long-range interactions and cooperation with SOX6. Genes Dev 24, 783-798 (2010); Chen, T. W. et al. ChIPseek, a web-based analysis tool for ChIP data. BMC Genomics 15, 539 (2014)). Notable enrichment of RIOK2 was observed around transcription start sites (FIG. 4F and FIG. 13A). Critically, occupancy of RIOK2 was observed at the promoter regions of its putative target genes (FIG. 4G and FIG. 13B).

### RIOK2 Binds to Specific De Novo Nucleotide Motifs

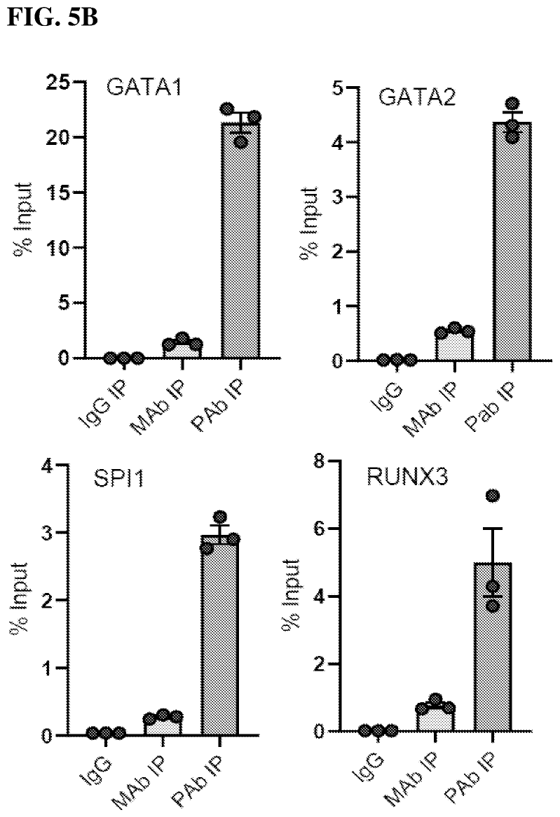

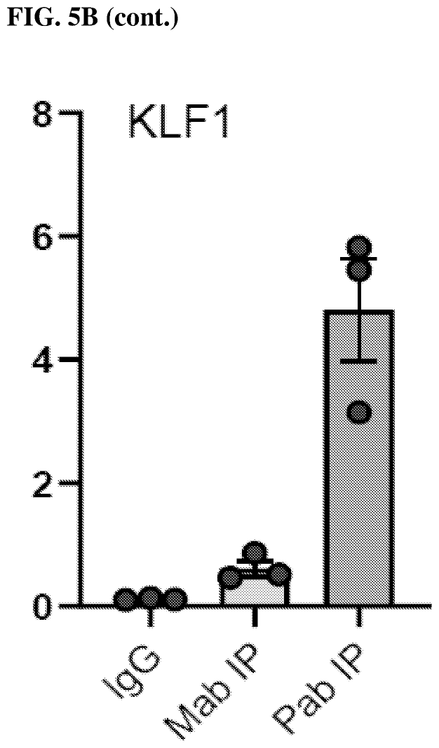

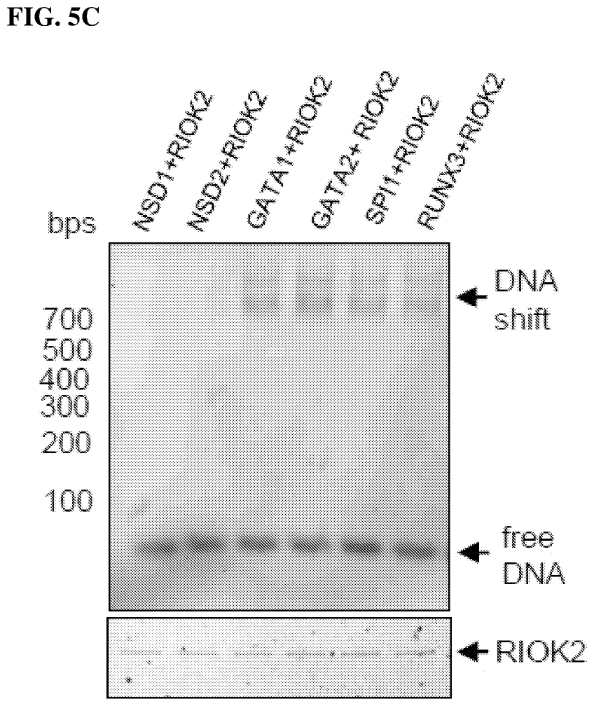

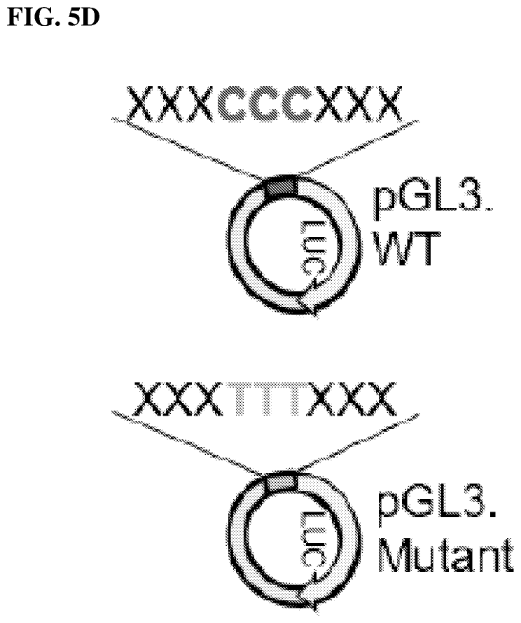

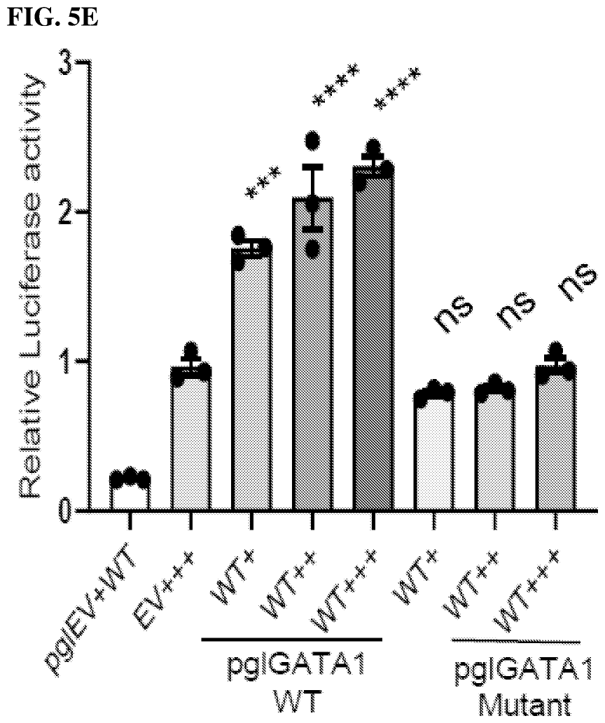

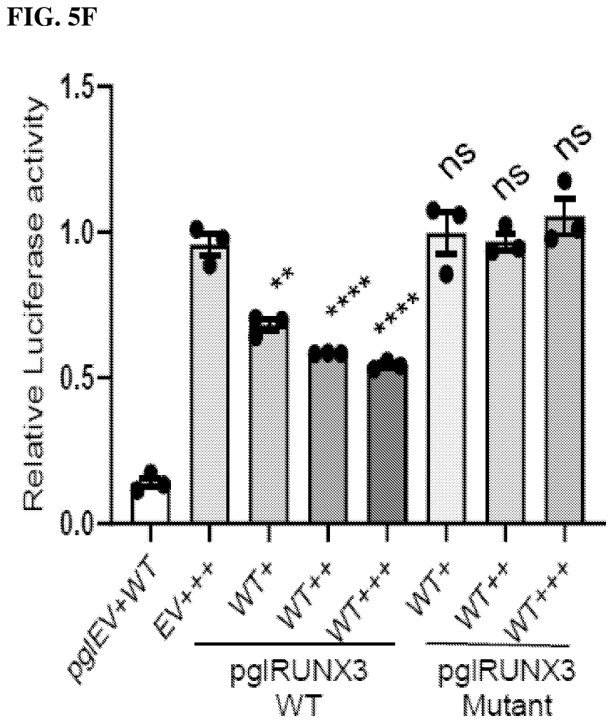

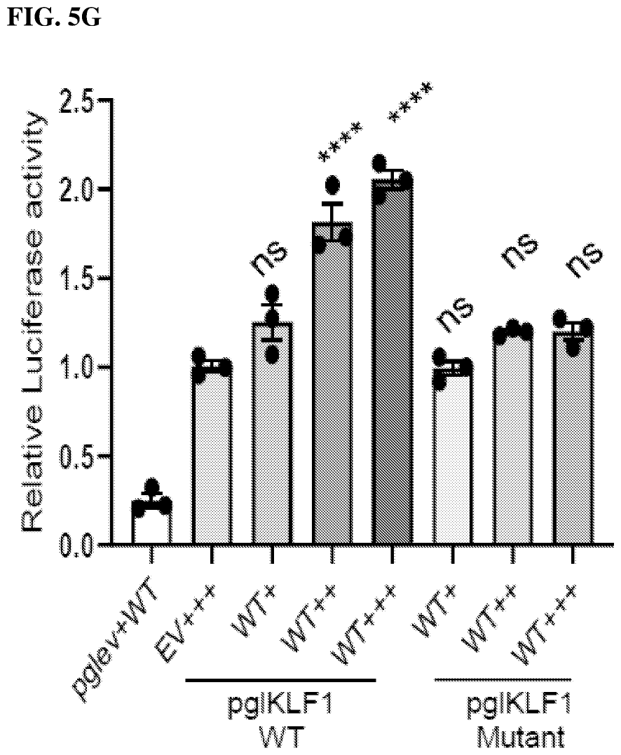

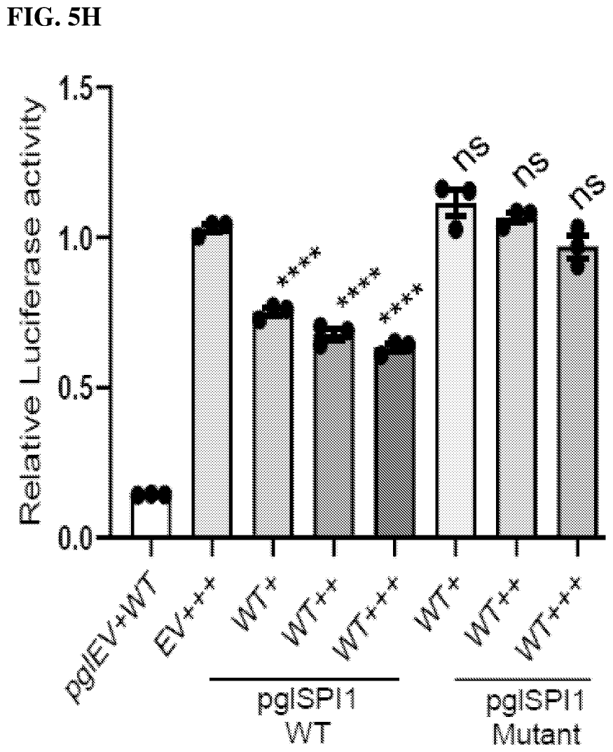

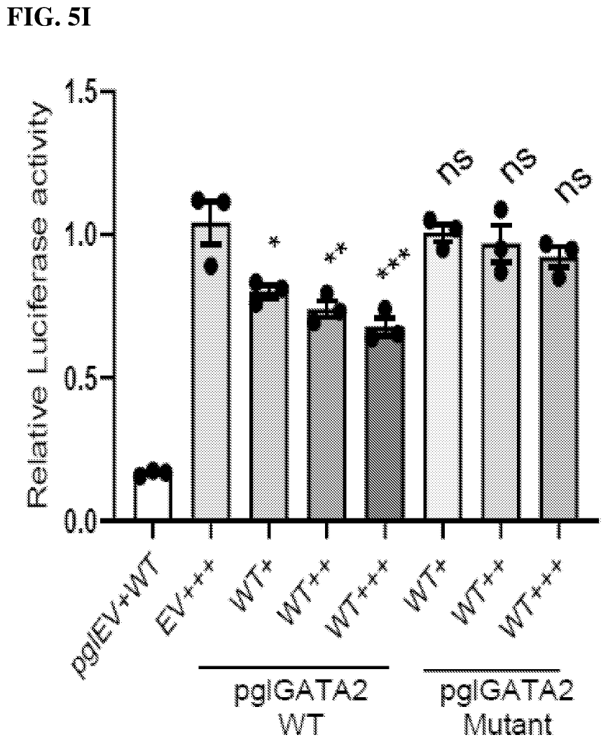

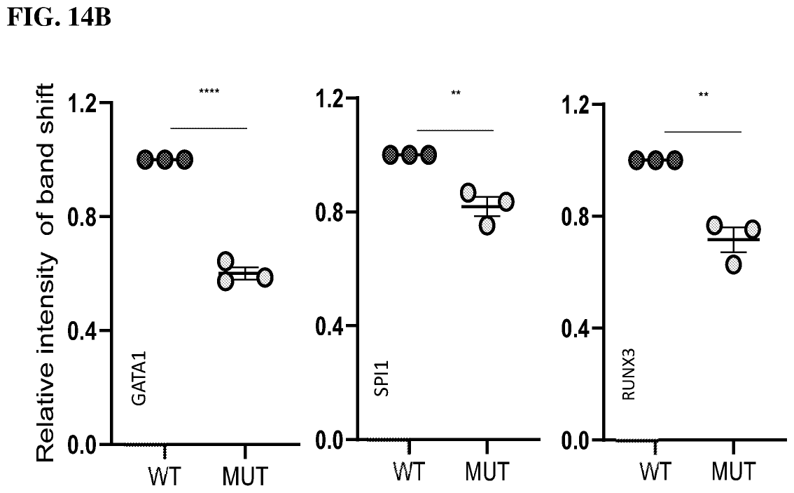

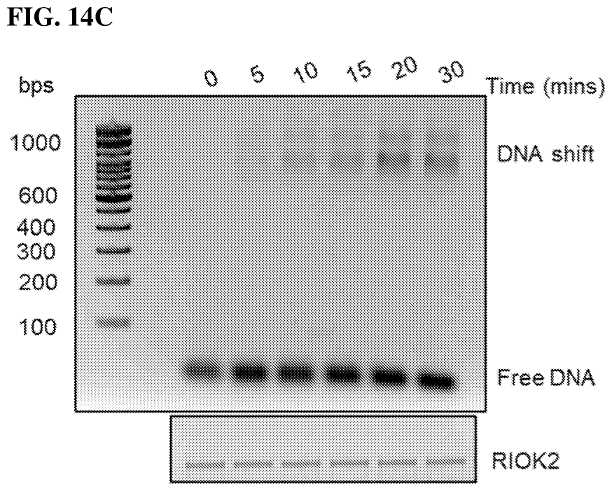

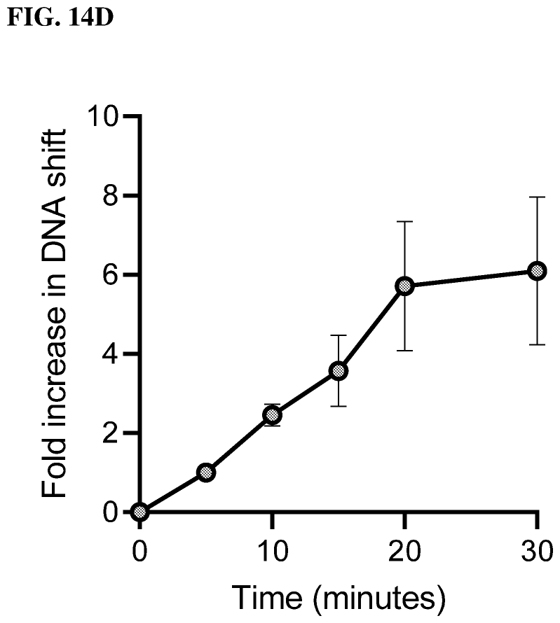

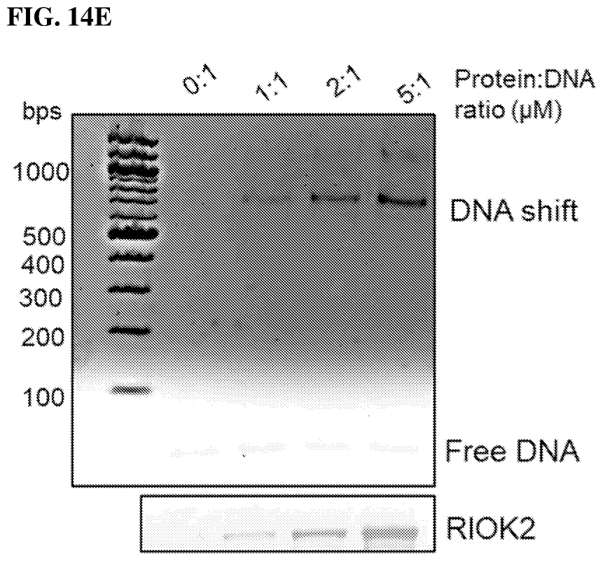

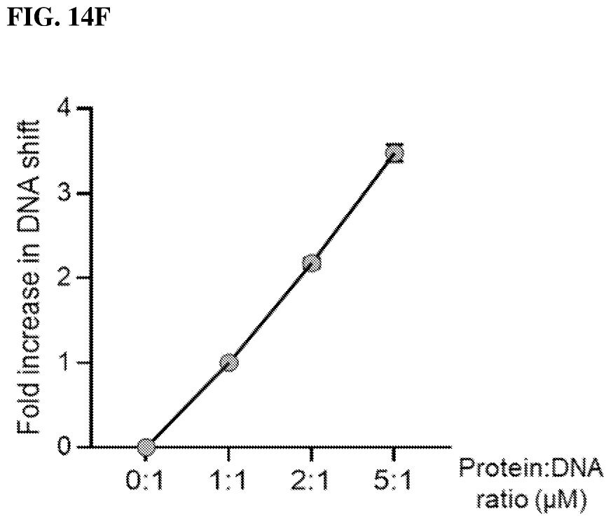

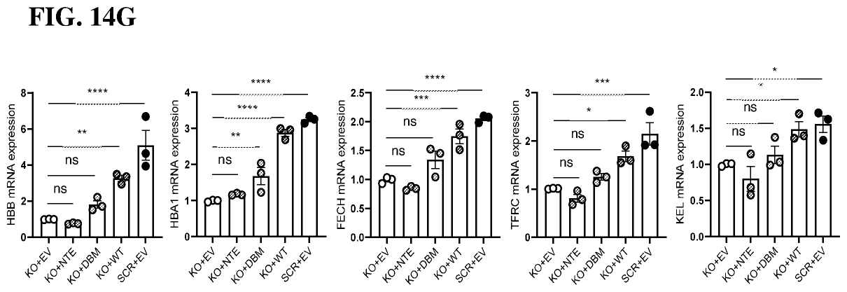

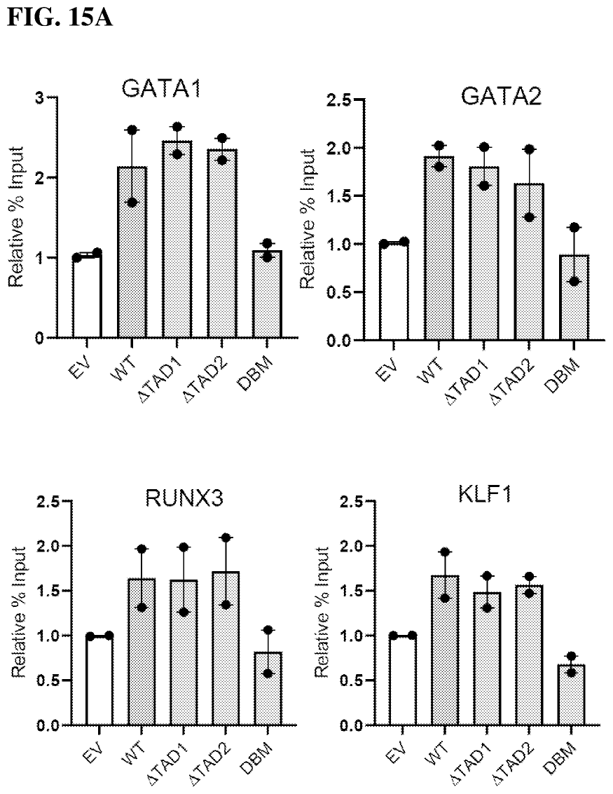

Next, de novo nucleotide binding motifs of RIOK2 across the entire human genome was curated (FIG. 13C) and identified a central cytosine-rich motif in the promoter regions of all TFs that were observed to be differentially regulated by RIOK2, including GATA1, GATA2, SPIl, RUNX3, and KLF1 (FIG. 5A and FIG. 13D). ChIP using both monoclonal and polyclonal antibodies against RIOK2 confirmed its binding to the promoter regions of target genes (FIG. 5B). Next, duplex DNAs were designed to encompass RIOK2-binding motifs in the promoter regions of GATA1 , GATA2, SPI1 and RUNX3 (Table 5). Electrophoretic mobility shift assay (EMSA) confirmed binding of RIOK2 protein to DNA in vitro (FIG. 5C). Mutation of 3 central cytosine nucleotides to thymine in the de novo motif was sufficient to partially, but significantly, attenuate RIOK2's DNA-binding affinity (FIGS. 14A and 14B). An increase in the DNA-binding affinity of RIOK2 was also noted over a time course (FIGS. 14C and 14D). Additionally, increasing protein concentrations of RIOK2 increased its DNA-binding ability in a dose-dependent manner (FIGS. 14E and 14F). Furthermore, luciferase reporter assays confirmed that RIOK2 activated GATA1 and KLF1 promoters while downregulating SPI1, RUNX3 and GATA2 promoter activities (FIG. 5D-5I). Mutation of the cytosine-rich motifs in the de novo nucleotide binding sequence markedly impaired RIOK2's transcriptional activity (FIG. 5D-5I). Hence, it was concluded that RIOK2 functions as a master TF via its ability to bind to a specific DNA motif and regulate the expression of several key hematopoietic transcription factors.

TABLE 5
Duplex DNA sequences for EMSA

| Duplex-strand name | Sequence | SEQ ID NO: |
|---|---|---|
| GATA1-WT-sense | 5′-ACA CAC CTG TAG TCC | 68 |
| strand | CAG CTA CTC AGG AGG-3′ | |
| GATA1-WT- | 5′-CCT CCT GAG TAG CTG | 69 |
| antisense strand | GGA CTA CAG GTG TGT-3′ | |
| GATA2-WT-sense | 5′-CTT GGG CTT TCC TCC | 70 |
| strand | CAC CCG GGA CTG GTG-3′ | |
| GATA2-WT- | 5′-CAC CAG TCC CGG GTG | 71 |
| antisense strand | GGA GGA AAG CCC AAG-3′ | |
| SPI1-WT-sense | 5′-AAG GGG ACT ATC TCC | 72 |
| strand | CAG CGG CAG GCC CTT-3′ | |
| SPI1-WT-antisense | 5′-AAG GGC CTG CCG CTG | 73 |
| strand | GGA GAT AGT CCC CTT-3′ | |
| RUNX3-WT-sense | 5′-CCG GGC GCC GCC CCC | 74 |
| strand | TGC GCC CTG AGG CCC-3′ | |
| RUNX3-WT- | 5′-GGG CCT CAG GGC GCA | 75 |
| antisense strand | GGG GGC GGC GCC CGG-3′ | |
| GATA1-Mutant- | 5′-ACA CAC CTG TAG TTT | 76 |
| sense strand | TAG CTA CTC AGG AGG-3′ | |
| GATA1-Mutant- | 5′-CCT CCT GAG TAG CTA | 77 |
| antisense strand | AAA CTA CAG GTG TGT-3′ | |
| SPI1-Mutant-sense | 5′-AAG GGG ACT ATC TTT | 78 |
| strand | TAG CGG CAG GCC CTT-3′ | |
| SPI1-Mutant- | 5′-AAG GGC CTG CCG CTA | 79 |
| antisense strand | AAA GAT AGT CCC CTT-3′ | |
| RUNX3-Mutant- | 5′-CCG GGC GCC GCC TTT | 80 |
| sense strand | TGC GCC CTG AGG CCC-3′ | |
| RUNX3-Mutant- | 5′-GGG CCT CAG GGC GCA | 81 |
| antisense strand | AAA GGC GGC GCC CGG-3′ | |

The DNA-binding domain of RIOK2 is critical in hematopoiesis Structural modeling of the N-terminal winged helix-turn-helix (wHTH) domain of RIOK2 predicted seven evolutionarily conserved residues: Asn50 Lys51 Arg54 Glu55 Lys58 His59 Lys60, that could associate with the B-form of double stranded DNA (FIG. 6A). A DNA binding mutant (DBM) of RIOK2 was generated by mutating all seven residues to alanine in a lentiviral vector expressing full length RIOK2 (1-552 residues) with an N-terminal Hemagglutinin (HA)-tag (FIG. 6B). A vector expressing only the N-terminal extension (NTE:1-92 residues) of RIOK2 was also generated, which forms its wHTH domain (FIG. 6B). ChIP revealed significantly compromised binding affinity of DBM RIOK2 to the target gene promoters (FIG. 6C). In contrast, NTE RIOK2 displayed more robust DNA binding as compared to wild-type RIOK2 (FIG. 6C), likely because its simpler conformation facilitated stronger interactions with DNA. While NTE RIOK2 could regulate the promoter activity of RIOK2's target genes, DBM RIOK2 displayed reduced transactivation (FIG. 6D). While DBM was unable to rescue altered mRNA levels of target genes in the RIOK2-KO setting (such as GATA1 ), NTE RIOK2 largely restored them to levels comparable with WT RIOK2 (FIG. 6E,6F). Consistently, DBM RIOK2 did not increase erythroid differentiation as compared to WT RIOK2, demonstrating the relevance of RIOK2's DNA binding activity in driving erythropoiesis (FIG. 6G and FIG. 14G). However, the NTE of RIOK2 also failed to restore impaired erythroid differentiation in RIOK2 KO cells (FIG. 6G and FIG. 14G). It was speculated that loss of the RIO (kinase) domain in NTE RIOK2 may have blocked cytoplasmic translation, thus compromising erythroid differentiation. While DBM displayed nascent protein synthesis comparable to WT RIOK2, expression of NTE RIOK2 failed to rescue protein translation in RIOK2 KO cells (FIG. 6H-6J). Together, these findings demonstrate that aberration in DNA-binding transcriptional function of RIOK2 is sufficient to impair hematopoiesis even when its translational activity is not affected, thus identifying RIOK2 as a master transcriptional regulator via the DNA binding properties of its wHTH domain.

### RIOK2 Encompasses Two Transactivation Domains

Eukaryotic transcription requires both DNA binding and transactivation domains, the latter of which recruit cofactors to initiate transcription. To identify the transactivation domains (TADs) of RIOK2, it was reasoned that a-helices are likely to facilitate binding with coactivators or corepressors, in part by the flexibility they can confer (Harish, B., Swapna, G. V., Kornhaber, G. J., Montelione, G. T. & Carey, J. Multiple helical conformations of the helix-turn-helix region revealed by NOE-restrained MD simulations of tryptophan aporepressor, TrpR. Proteins 85, 731-740 (2017)). Since winged helix domains mostly harbor TADs close to their DNA binding helices, N-terminal HA-tagged mutants with deletions in helices were generated by forming the wHTH domain of RIOK2 (FIGS. 7A and 7B)(Teichmann, M., Dumay-Odelot, H. & Fribourg, S. Structural and functional aspects of winged-helix domains at the core of transcription initiation complexes. Transcription 3, 2-7 (2012)). Deletion of amino acids 14-29 (ΔTAD1) and 77-90 (ΔTAD2) did not restore mRNA levels of target genes in the RIOK2 knockout cells (FIGS. 7C and 7D). ChIP confirmed comparable DNA-binding ability of ΔTAD1 and ΔTAD2 mutants to wild-type (WT) RIOK2 (FIG. 15A), indicating that the loss of transcriptional regulation in ΔTAD1 and ΔTAD2 did not stem from compromised DNA-binding. Examination of the crystal structure of residues 2-301 of RIOK2 further suggests that TAD1 (yellow) and TAD2 (pale grey) confer accessibility to the wHTH domain of RIOK2 to potentially bind coactivators or corepressors (FIG. 7E). Next, co-immunoprecipitation of ectopically expressed HA-tagged WT vs ΔTAD1 and ΔTAD2 RIOK2 using monoclonal anti-HA antibodies was proformed followed by mass spectrometry to analyze quantitative spectral counts of the immunoprecipitated proteins. Binding of RIOK2 with the core transcriptional assembly factors was observed at TSS, such as POLR2A, WDR43 and DDX21 (FIG. 7F) (Bi, X. et al. RNA Targets Ribogenesis Factor WDR43 to Chromatin for Transcription and Pluripotency Control. Mol Cell 75, 102-116 e109 (2019)). Loss of either TAD1 or TAD2 compromised these interactions (FIG. 7F), signifying that the TADs of RIOK2 facilitate its interaction with the core transcriptional complex at TSS. Furthermore, expression of ΔTAD1 or ΔTAD2 neither rescued erythroid differentiation nor restored megakaryocytic and myeloid differentiation to baseline levels in RIOK2-depleted HSPCs (FIG. 7G and FIG. 15B). In contrast, deletion of residues 35-43, comprising another helix in the wHTH domain, displayed more potent erythroid differentiation than WT RIOK2 in the KO-setting, indicating that this helix likely functions as a transrepressor domain (TRD) of RIOK2 (FIGS. 15c and 15D).

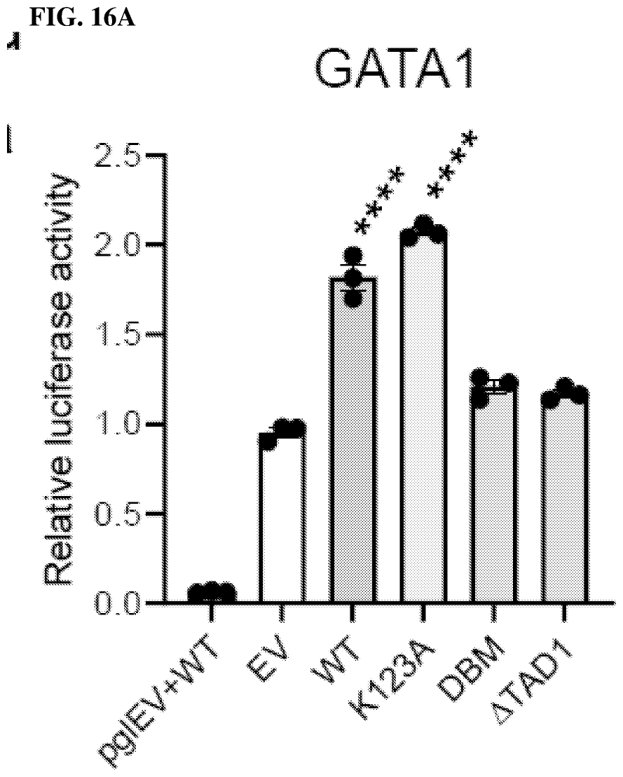

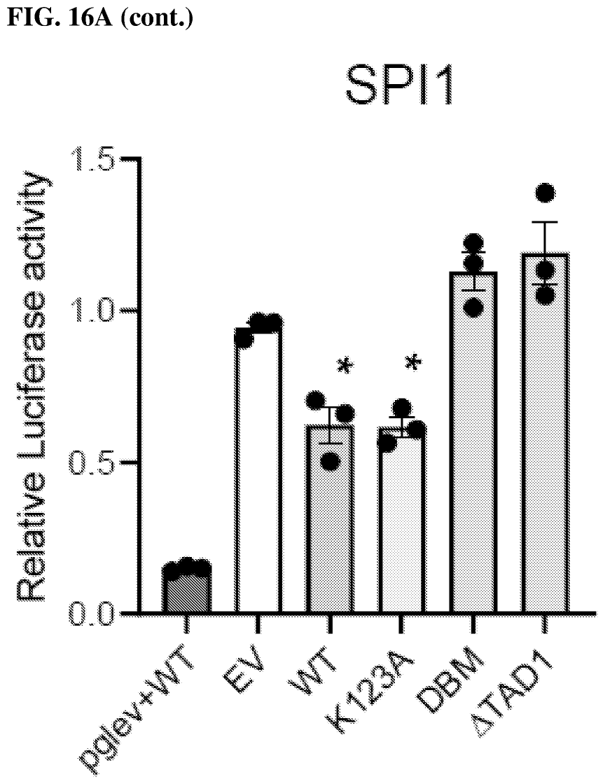

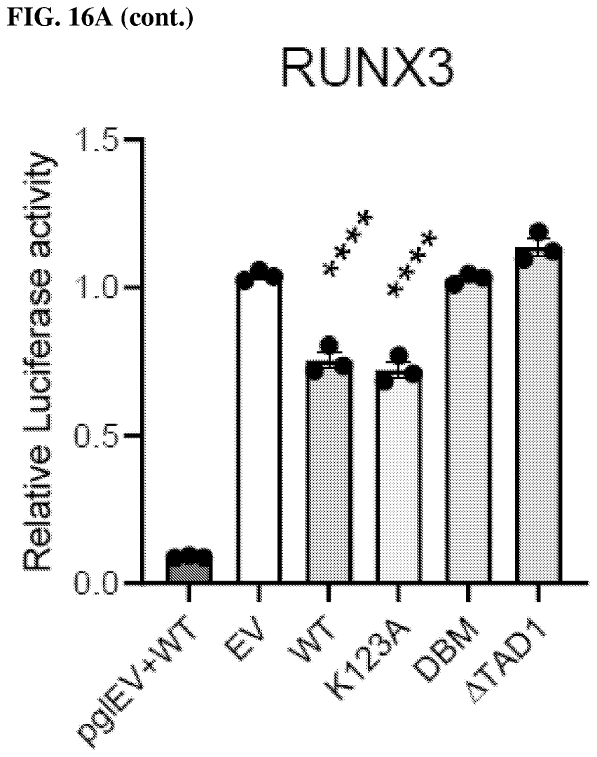

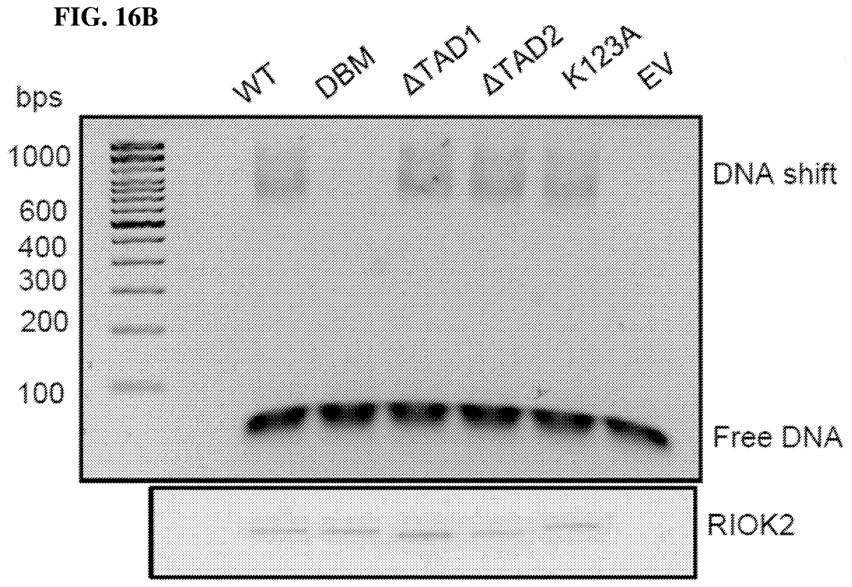

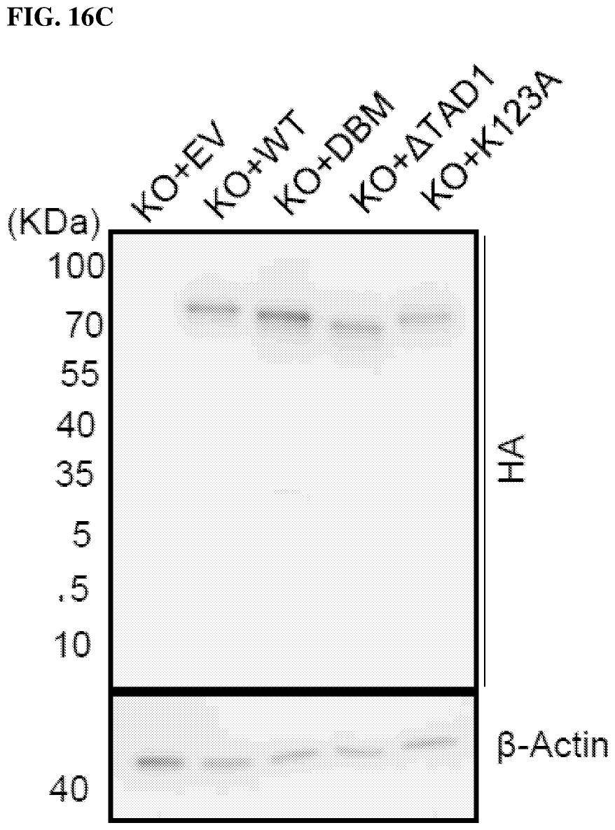

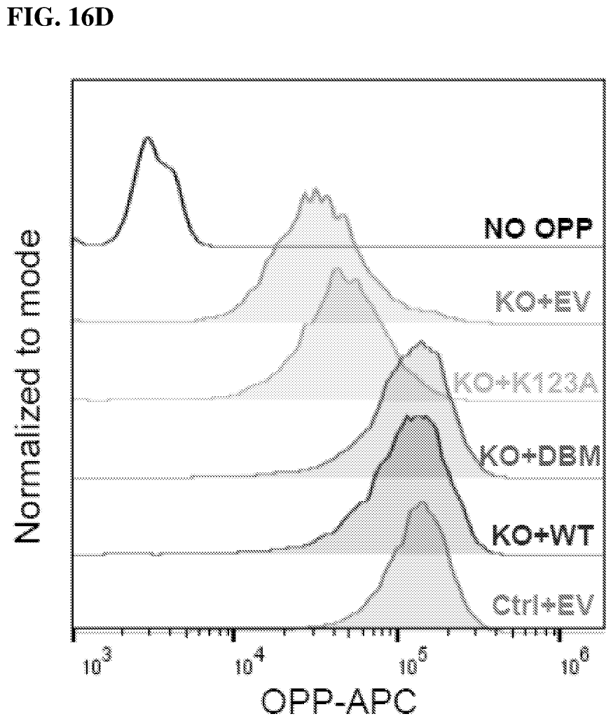

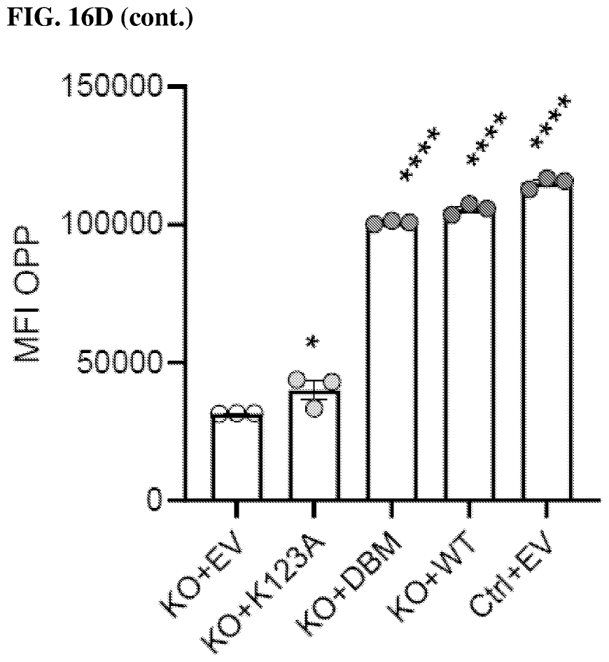

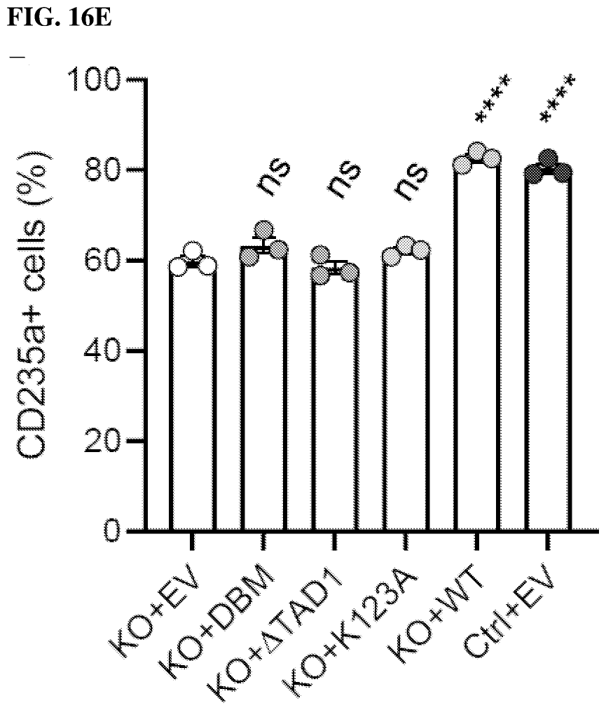

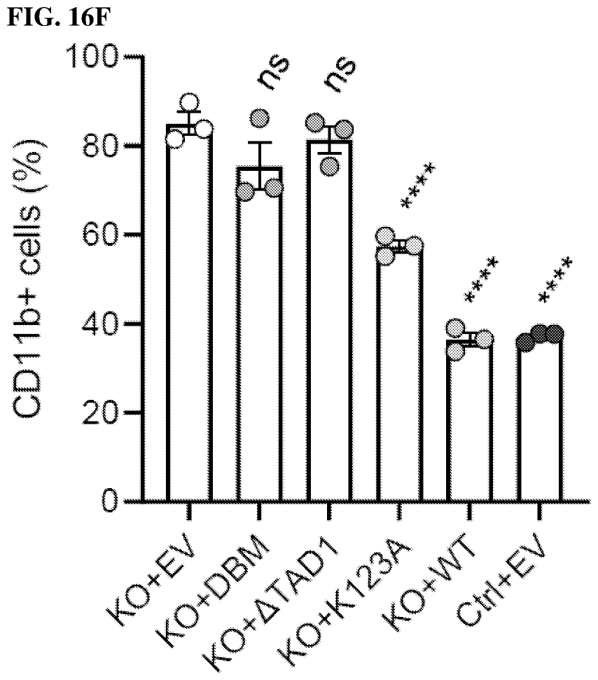

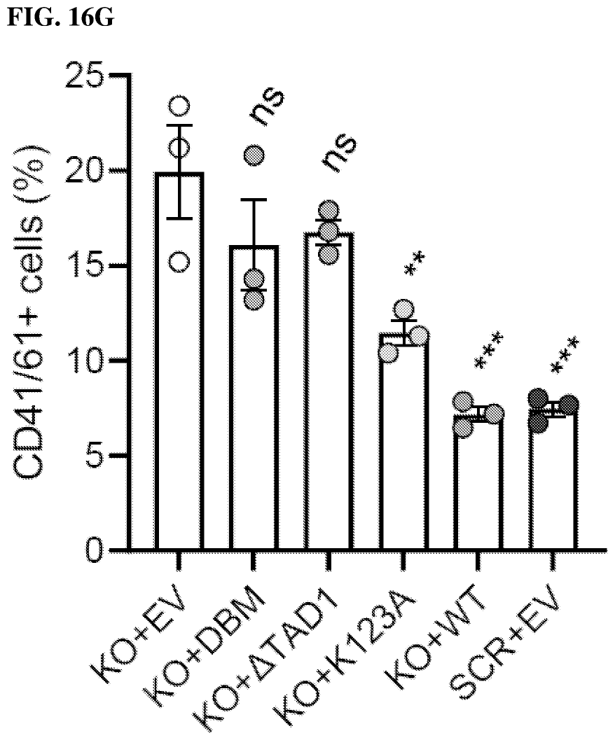

Given the integral roles of the DNA-binding and transactivation domains of RIOK2 in hematopoiesis (FIGS. 15E and 15F), the involvement of its kinase activity in hematopoietic lineage commitment was assessed next. To this end, the kinase-dead mutant of RIOK2 K123A with an N-terminal HA-tag was generated. Interestingly, the K123A mutant of RIOK2 did not affect transactivation of its targets, as opposed to the DBM and ΔTAD1 mutants which compromised RIOK2's transactivation functions (FIG. 16A). This suggests that the kinase activity of RIOK2 does not play a role in transcriptional functions.

Consistently, K123A RIOK2 displayed comparable DNA binding ability alongside WT, ΔTAD1 and ΔTAD2 RIOK2, whereas the DNA-binding mutant (DBM) failed to bind DNA (FIG. 16B). To confirm the roles of RIOK2's kinase activity in cytoplasmic protein translation, native protein synthesis was next assessed. The K123A mutant RIOK2 markedly compromised the recovery of protein translation in KO cells as compared to DBM and WT RIOK2 (FIG. 816D). Although the kinase-dead K123A mutant impaired erythroid differentiation on a comparable level with DBM and ΔTAD1 RIOK2, it significantly rescued myelopoiesis and megakaryopoiesis (16E-16G). This is consistent with existing literature that reported selective inhibition of erythroid differentiation with blockade of protein translation (Khajuria, R. K. et al. Ribosome Levels Selectively Regulate Translation and Lineage Commitment in Human Hematopoiesis. Cell 173, 90-103 el19 (2018)).

Correlation of RIOK2's mRNA Expression in MDS, AML and CKD

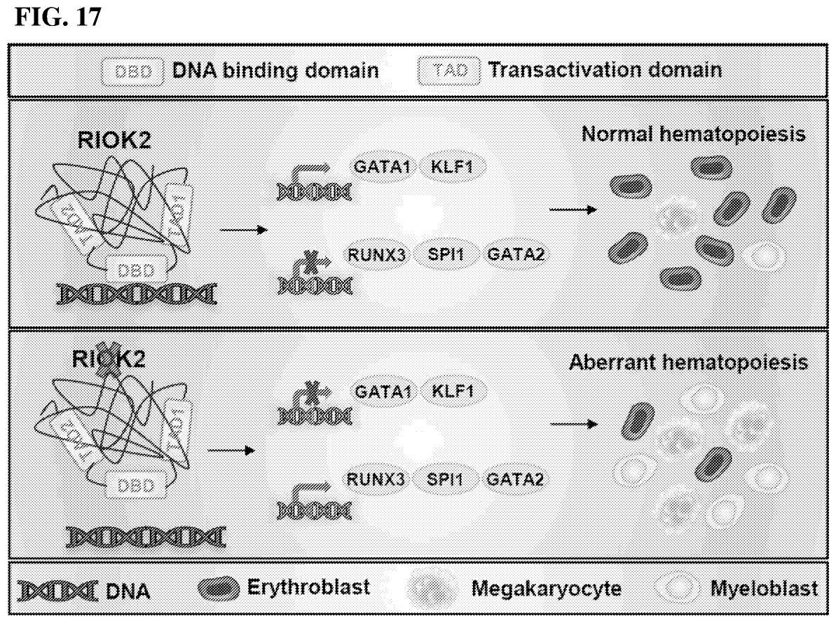

Importantly, the expression levels of RIOK2 in patients with hematologic disorders was assessed. Corroborating the observation that loss of RIOK2 dampens GATA1 and KLF1 mRNA levels while elevating GATA2, SPI1 and RUNX3 expression (FIGS. 3A and 3E), it was noted that RIOK2's mRNA expression positively correlated with GATA1 and KLF1, and negatively correlated with GATA2 and RUNX3 in MDS patients (GSE19429) (FIG. 8A). Additionally, RIOK2's expression positively correlated with erythroid gene expression and negatively correlated with myeloid and megakaryocytic gene expression (FIG. 8B). Similar correlations in acute myeloid leukemia (AML) patients (GSE131184) were obtained (FIGS. 8C and 8D). Furthermore, the mRNA expression of RIOK2 was significantly reduced in chronic kidney disease (CKD) patients as compared to healthy controls (GSE37171) (FIG. 8E), that also positively correlated with the expression of early erythroid genes (FIG. 8F). These findings strongly suggest that RIOK2-mediated effects on hematopoiesis may be physiologically relevant in MDS, AML and the anemia of CKD. Collectively, these data establish RIOK2 as a master regulator of hematopoietic differentiation (FIG. 17).

### Example 3: Loss of Function (LOF) Mutations in RIOK2 Attenuate Erythroid Differentiation

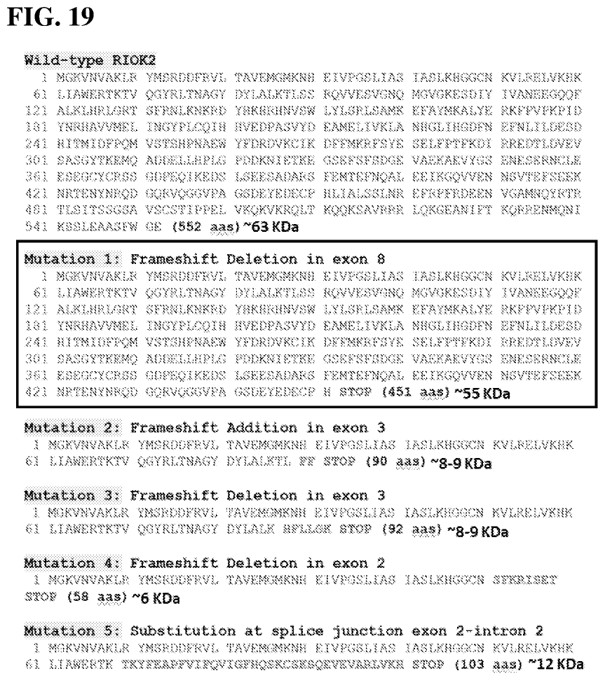

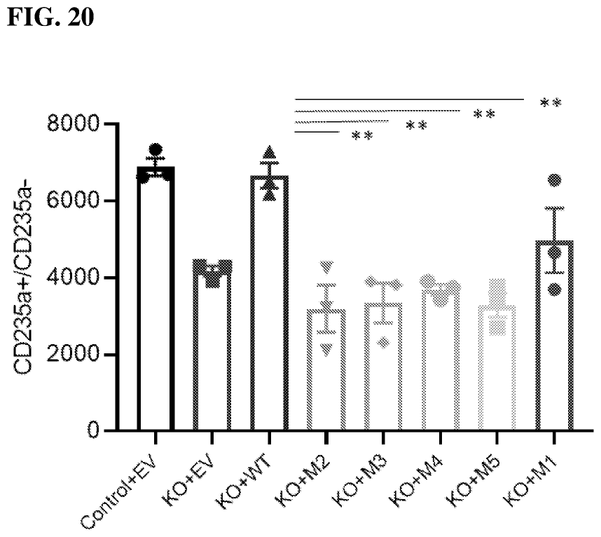

The inventors have identified 5 distinct mutations of RIOK2 (M1-M5) across 10 individuals in a cohort of consanguineous Pakistani nationals (FIG. 19). Since RIOK2 plays a critical role in erythroid differentiation, this prompted the assessment of whether the 5 identified RIOK2 mutants, M1-M5, behaved as LOF mutants in erythroid differentiation. To address this, mutants M1-M5 were cloned in the lentiviral vector that has been used previously to express wild-type (WT) RIOK2 with an N-terminal HA-tag (stated before). Next, endogenous RIOK2 in primary human stem and progenitor cells (HSPCs) was knocked out, followed by lentiviral expression of either empty vector (EV), WT or mutants M1-M5. As shown in FIG. 20, neither of the mutants were able to rescue the defects in erythroid differentiation in comparison to RIOK2 KO cells. This strongly suggests that the RIOK2 mutants M1-M5 display loss-of-function (LOF) phenotype while driving erythropoiesis. Collectively, these findings confirm that RIOK2 is a key player in driving erythropoiesis and deletion or point mutations in this protein blocks erythroid and ultimately hematopoietic differentiation. Whole exome sequencing in a large consanguineous family of over 100 Pakistani individuals will be proformed because one of the family members has a germline mutation in RIOK2. DNA, serum and viably frozen cells isolated from peripheral blood of these individuals will also be obtained to further characterize the transcriptomic and metabolic profiles associated with RIOK2 LOF mutations in this cohort.

### Example 4: Identification of RIOK2 as a Regulator of Telomere Shortening and Aging

Also provided herein is data to show loss of RIOK2 leads to significant telomere shortening. Telomere shortending was detected by PCR based amplification using primers amplifying telomeric copies (TTAGGG) and quantification with a single copy gene (Actin or B64u). Assays were done on primary human hematopoietic stem and progenitor cells (HSPCs). As shown in FIG. 21, CRISPR/Cas9-mediated knockdown or knockout of RIOK2 decreases telomere length in primary human HSPCs dose-dependently, thus signifying that RIOK2 is critical for telomere maintenance.

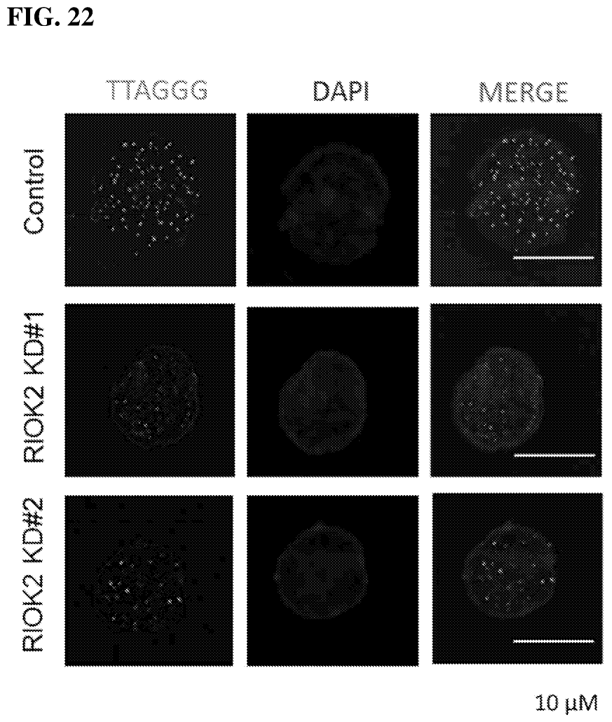

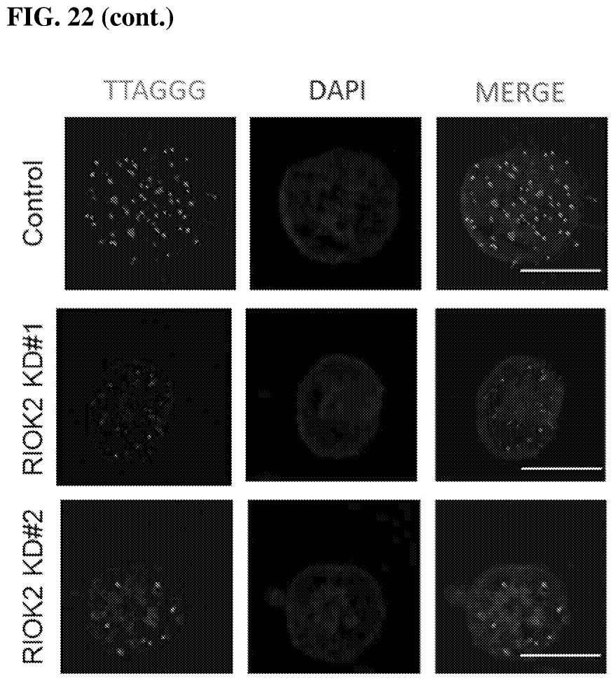

Detection of telomere shortening was also preformed by fluorescence in-situ hybridization (FISH) and on a TF-1 erythroleukemia cell line (FIG. 22). CRISPR/Cas9-mediated knockdown of RIOK2 decreases telomeric puncta (green dots) in TF-1 cells, thus signifying that loss of RIOK2 leads to significant telomere shortening.

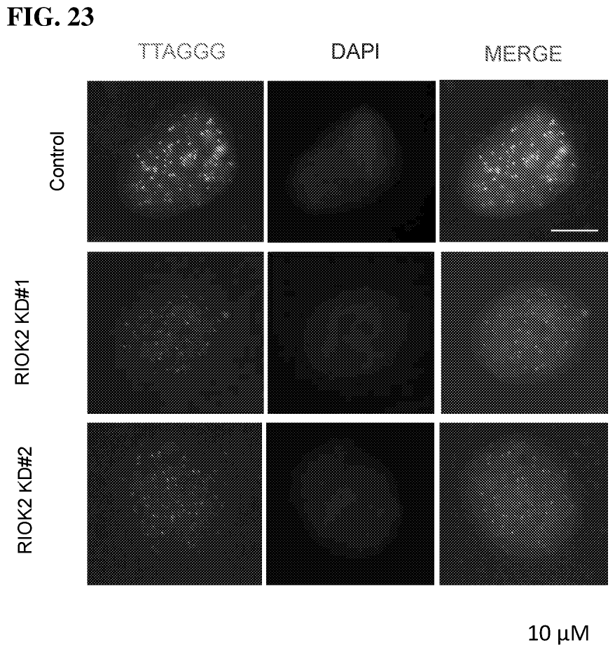

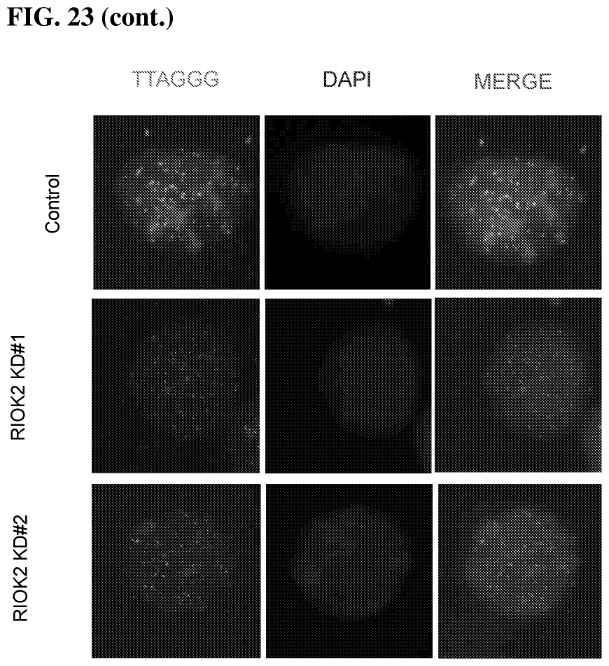

Additionally, telomere shortening is also detected by fluorescence in-situ hybridization (FISH) in a K562 erythroleukemia cell line following knock down/out of RIOK2. CRISPR/Cas9-mediated knockdown of RIOK2 decreases telomeric puncta (green dots) in K562 cells, thus signifying that loss of RIOK2 leads to significant telomere shortening (FIG. 23).

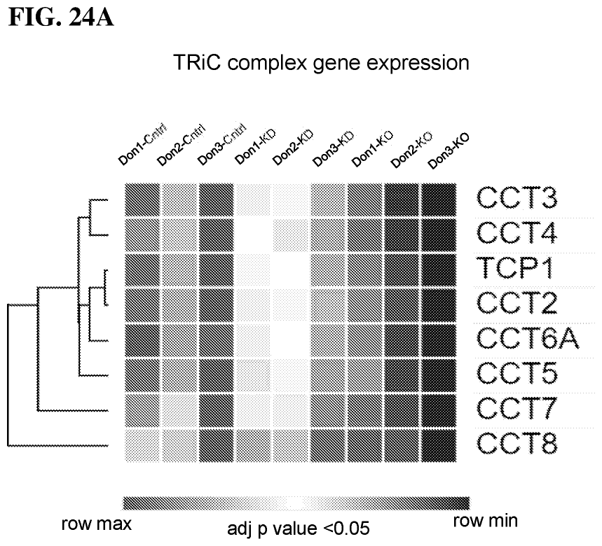

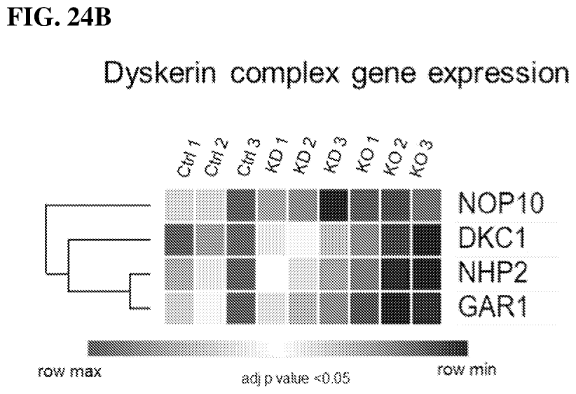

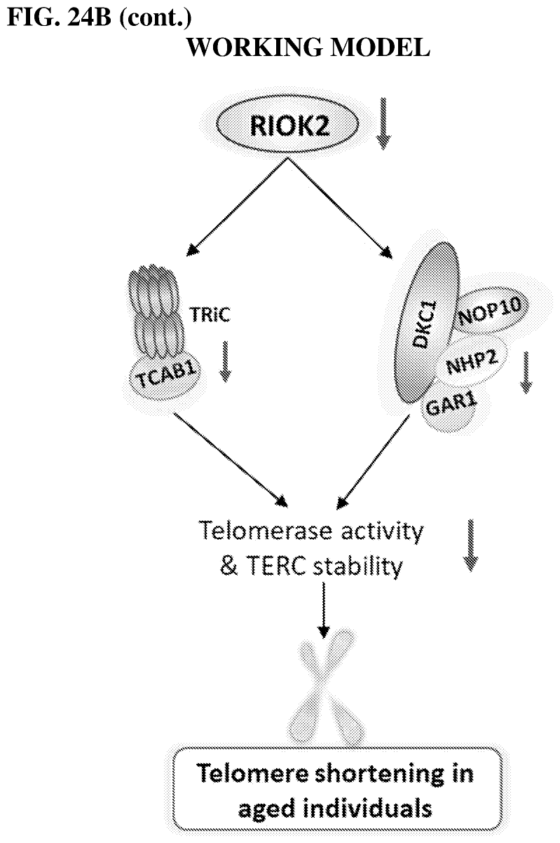

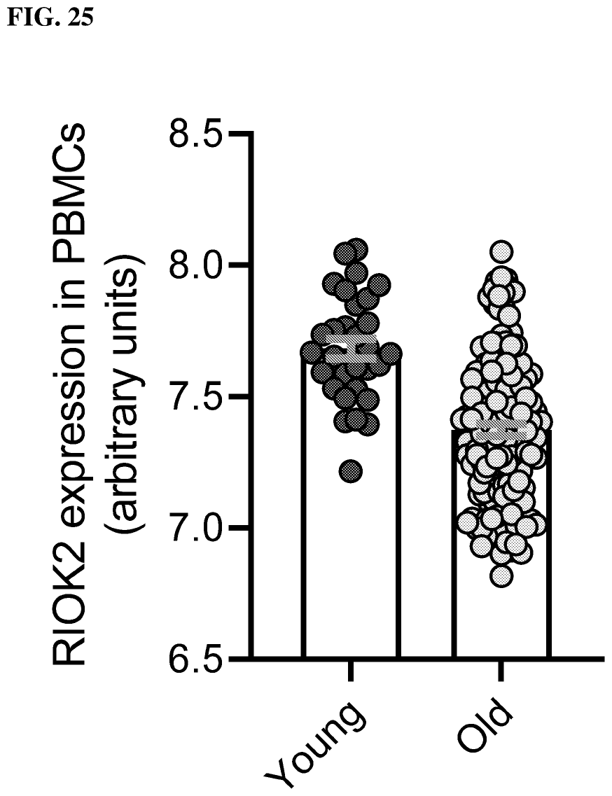

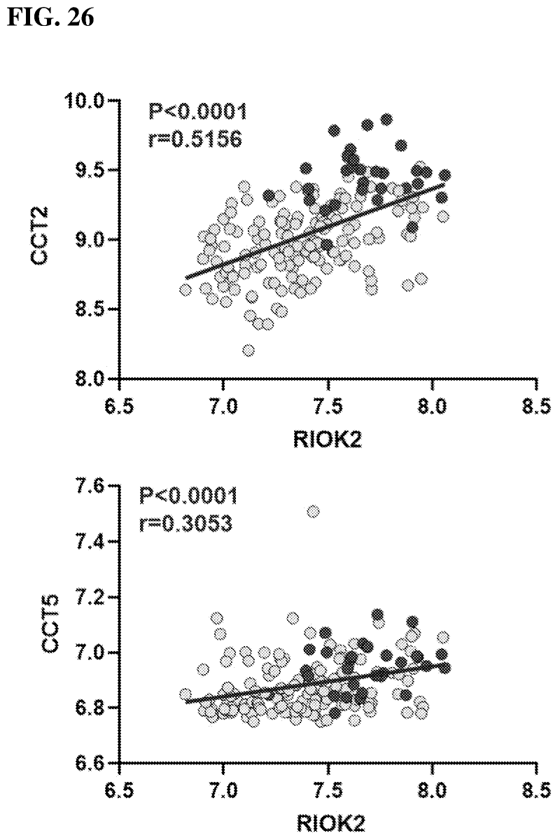

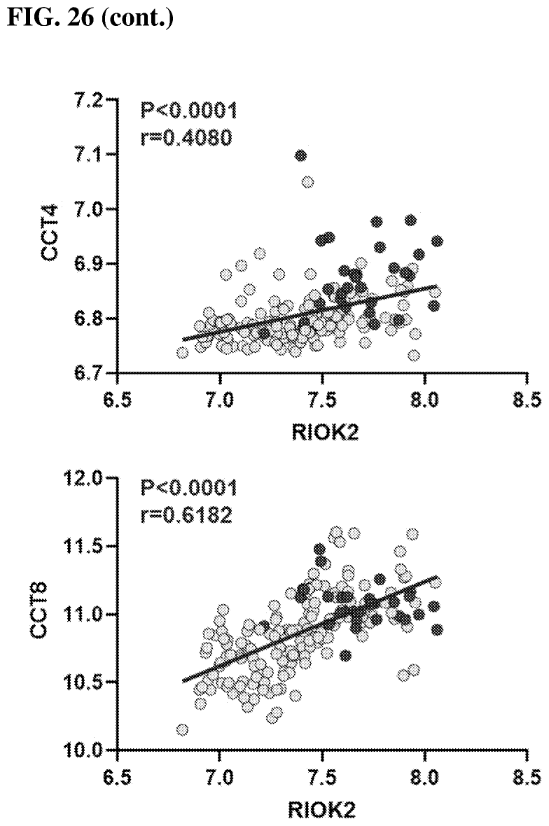

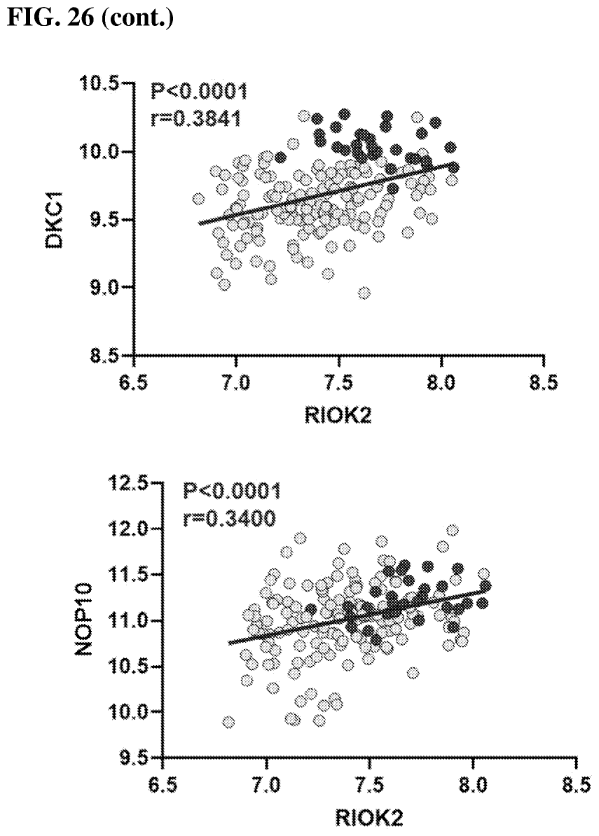

The inventors have also shown that TRiC and Dyskerin complex subunits are critical in maintaining telomerase activity and telomere length. RIOK2 transcriptionally regulates TRiC and Dyskerin complex subunit expression via its transcription factor (TF) activity. Bulk RNA-sequencing study performed on primary human hematopoietic stem and progenitor cells (HSPCs) isolated from n=3 healthy donors (FIG. 24). The data provided herewith also highlight the physiological relevance of RIOK2 in aging. mRNA expression of RIOK2 is significantly reduced in PBMCs of young vs old individuals, GEOdataset from Nevalainen et al., Age (Dordr) 2015 (FIG. 25). mRNA expression of RIOK2 also positively correlates with TRiC and Dyskerin complex subunits in PBMCs of young vs old individuals, i.e. with lesser expression of RIOK2 there is reduced expression of TRiC and Dyskerin complex subunits, GEOdataset from Nevalainen et al., Age (Dordr) 2015 (FIG. 26). Therefore, stabilization of the expression and/or activity of RIOK2 may be beneficial in alleviating telomere shortening in old individuals.

Telomere shortening is a major hallmark of aging (Mikhelson, V. M., and Gamaley, I. A. (2012). Telomere shortening is a sole mechanism of aging in mammals. Curr Aging Sci 5, 203-208; Zhu, Y., Liu, X., Ding, X., Wang, F., and Geng, X. (2019). Telomere and its role in the aging pathways: telomere shortening, cell senescence and mitochondria dysfunction. Biogerontology 20, 1-16.) and is emerging as a characteristic feature of hematological disorders, such as Myelodysplastic syndromes (MDS), chronic myeloid leukemia (AML), aplastic anemia and others (Lange, K., Holm, L., Vang Nielsen, K., Hahn, A., Hofmann, W., Kreipe, H., Schlegelberger, B., and Gohring, G. (2010). Telomere shortening and chromosomal instability in myelodysplastic syndromes. Genes Chromosomes Cancer 49, 260-269; Paiva, R. M., and Calado, R. T. (2014). Telomere dysfunction and hematologic disorders. Prog Mol Biol Transl Sci 125, 133-157). Optimal telomerase enzyme activity is central in subverting the progressive shortening of telomeric DNA ends that triggers a cascade of reactions leading to apoptosis or replicative senescence. Although expression of telomerase complexes remain tightly regulated to prevent a spectrum of disorders and malignancies (Roake, C. M., and Artandi, S.E. (2020). Regulation of human telomerase in homeostasis and disease. Nat Rev Mol Cell Biol 21, 384-397), the upstream regulation of telomerase core components remain poorly defined. Here, RIOK2 is identified as a key transcriptional regulator of telomere maintenance. Loss of RIOK2 significantly reduces the mRNA expression of both TRiC and dyskerin complex subunits and ultimately results in telomere shortening. Loss of the DNA-binding or transactivation functions of RIOK2 fails to rescue telomerase activity and telomere shortening. Notably, the mRNA expression of RIOK2 significantly correlates with that of TRiC and dyskerin complex subunits in patients with MDS, the telomere biology disorder Idiopathic pulmonary fibrosis (IPF) and in aging individuals from multiple cohorts. Furthermore, telomere lengths of MDS patient-derived bone marrow samples positively correlate with the corresponding mRNA levels of RIOK2. These data define RIOK2 as a key transcription factor regulating telomere biogenesis that functions as a novel molecular link between anemia and telomere shortening. These findings uncover key RIOK2-dependent transcriptional programs that concurrently regulate erythroid differentiation and telomere biogenesis in multiple hematological and age-associated human disorders.

### INTRODUCTION

Telomeres form the protective ends of chromosomal DNA which undergo progressive shortening with each cell division (Armanios, M., and Blackburn, E.H. (2012). The telomere syndromes. Nat Rev Genet 13, 693-704; Chen, L., Roake, C. M., Freund, A., Batista, P.J., Tian, S., Yin, Y. A., Gajera, C. R., Lin, S., Lee, B., Pech, M. F., et al. (2018). An Activity Switch in Human Telomerase Based on RNA Conformation and Shaped by TCAB1. Cell 174, 218-230 e213). The importance of telomere maintenance is highlighted by the spectrum of disorders resulting from telomere shortening, such as aplastic anemia, idiopathic pulmonary fibrosis, and also aging (Boddu, P. C., and Kadia, T. M. (2019). Molecular pathogenesis of acquired aplastic anemia. Eur J Haematol 102, 103-110; Zhang, K., Xu, L., and Cong, Y. S. (2021). Telomere Dysfunction in Idiopathic Pulmonary Fibrosis. Front Med (Lausanne) 8, 739810.; Zhu, Y., Liu, X., Ding, X., Wang, F., and Geng, X. (2019). Telomere and its role in the aging pathways: telomere shortening, cell senescence and mitochondria dysfunction. Biogerontology 20, 1-16). Telomere shortening is deemed particularly debilitating for the optimal renewal and functioning of stem cells, as evidenced by the disorder Dyskeratosis Congenita (DC) that stems for improper telomere maintenance in stem cells (AlSabbagh, M.M. (2020). Dyskeratosis congenita: a literature review. J Dtsch Dermatol Ges 18, 943-967). Telomere shortening is also emerging as a causative factor for myelodysplastic syndromes (MDS) (Schratz, K. E., and Armanios, M. (2020). Cancer and myeloid clonal evolution in the short telomere syndromes. Curr Opin Genet Dev 60, 112-118), a form of hematologic malignancy that is classically characterized by ineffective hematopoiesis and bone marrow failure (Pellagatti, A., and Boultwood, J. (2015). The molecular pathogenesis of the myelodysplastic syndromes. Eur J Haematol 95, 3-15). Hence, therapeutic approaches to stimulate telomere biogenesis largely requires meticulous understanding of the various cellular factors and pathways that intersect and interact to regulate telomere maintenance.

Telomerase enzyme is a ribonucleoprotein complex that is responsible for synthesizing telomeric DNA repeats. Human telomerase enzyme comprises of a reverse transcriptase known as TERT (the Telomerase Reverse Transcriptase), an integral RNA core called TERC (the Telomerase RNA Component or hTR) and several protein cofactors such as dyskerin complex and TCAB1 (Telomerase Cajal Body Protein 1) (Armanios, M., and Blackburn, E.H. (2012). The telomere syndromes. Nat Rev Genet 13, 693-704). Catalytically active telomerase enzyme elongates telomeres by reverse transcribing the RNA component TERC that functions as a template for TERT. Optimum enzymatic activity of telomerase is ensured by the proper processing and stability of its components (Roake, C. M., and Artandi, S.E. (2020). Regulation of human telomerase in homeostasis and disease. Nat Rev Mol Cell Biol 21, 384-397). In this regard, the TRiC (TCP-1 Ring Complex) chaperonin complex functions as an integral component of telomere maintenance by properly folding and stabilizing the telomerase cofactor TCAB1 (Freund, A., Zhong, F. L., Venteicher, A. S., Meng, Z., Veenstra, T.D., Frydman, J., and Artandi, S.E. (2014). Proteostatic control of telomerase function through TRiC-mediated folding of TCAB1. Cell 159, 1389-1403). The TRiC complex consists of 8 homologous subunits, CCT1-CCT8, forming two octameric rings that remain stacked together (Jin, M., Liu, C., Han, W., and Cong, Y. (2019). TRiC/CCT Chaperonin: Structure and Function. Subcell Biochem 93, 625-654). Depletion of even one TRiC complex subunit is shown to reduce TCAB1 protein stability, thus impairing telomerase activity (Chen, L., Roake, C. M., Freund, A., Batista, P. J., Tian, S., Yin, Y. A., Gajera, C. R., Lin, S., Lee, B., Pech, M. F., et al. (2018). An Activity Switch in Human Telomerase Based on RNA Conformation and Shaped by TCAB1. Cell 174, 218-230 e213). Presence of autosomal recessive mutations of TCAB1 in DC (Zhong, F., Savage, S. A., Shkreli, M., Giri, N., Jessop, L., Myers, T., Chen, R., Alter, B. P., and Artandi, S. E. (2011). Disruption of telomerase trafficking by TCAB1 mutation causes dyskeratosis congenita. Genes Dev 25, 11-16) further emphasize the vital roles of this telomerase holoenzyme subunit in telomere biogenesis.

The Dyskerin complex is another key component of the telomerase enzyme that is responsible for proper folding and assembly of TERC (MacNeil, D.E., Lambert-Lanteigne, P., and Autexier, C. (2019). N-terminal residues of human dyskerin are required for interactions with telomerase RNA that prevent RNA degradation. Nucleic Acids Res 47, 5368-5380; Shukla, S., Schmidt, J.C., Goldfarb, K.C., Cech, T. R., and Parker, R. (2016). Inhibition of telomerase RNA decay rescues telomerase deficiency caused by dyskerin or PARN defects. Nat Struct Mol Biol 23, 286-292). The Dyskerin complex is a 4-member group constituting dyskerin (encoded by DKC1), NHP2, NOP10 and GAR1. Mutations in dyskerin were identified as the first pathogenic mutations in DC, followed by NHP2 and NOP10 (Roake, C. M., and Artandi, S.E. (2020). Regulation of human telomerase in homeostasis and disease. Nat Rev Mol Cell Biol 21, 384-397). Loss or mutations in the dyskerin subunits decreases stability of TERC and induces telomere shortening (Vulliamy, T. J., and Dokal, I. (2008). Dyskeratosis congenita: the diverse clinical presentation of mutations in the telomerase complex. Biochimie 90, 122-130). Thus, the expression levels of telomerase subunits are under tight regulation to optimally reverse telomere shortening since hyper-stimulation of telomerase subunits are known to underlie cancer progression (Bernardes de Jesus and Blasco, 2013). However, the upstream regulation of telomerase subunit expression is inadequately understood, and the transcriptional control of TERT is exclusively studied in detail so far (Dogan and Forsyth, 2021). Systematic revelation of such regulatory mechanisms may shed light into the molecular underpinnings of reversing telomere shortening in not only aging and but also other hematological and telomere biology disorders.

It has been reported that RIOK2also functions as a master transcription factor to regulate human blood cell development (Ghosh, S., Raundhal, M., Myers, S.A., Carr, S.A., Chen, X., Petsko, G. A., and Glimcher, L. H. (2022). Identification of RIOK2 as a master regulator of human blood cell development. Nat Immunol 23, 109-121). Loss of RIOK2 in primary human hematopoietic stem and progenitor cells (HSPCs) and erythroblast cell lines, TF-1 and K562, severely dampens erythroid (red blood cell) differentiation. It was uncovered that RIOK2 binds to a specific nucleotide sequence at the promoter regions of its targets and controls their mRNA expression with the help of its DNA-binding (DBD) and transactivation (TAD) sub-domains contained within its winged helix-turn-helix (wHTH) domain at the N-terminus.

Here, RIOK2 is characterized as a key transcriptional regulator of human telomere maintenance and it is shown that loss of RIOK2 triggers significant telomere shortening in cells of both erythroid and non-erythroid origin. The RNA-sequencing, ATAC-sequencing, chromatin-immunoprecipitation, luciferase reporter assays and reconstitution experiments emphasize the essential roles of RIOK2 in transcriptionally regulating the mRNA expression of TRiC and Dyskerin complex subunits to sustain the enzymatic activity of human telomerase. This in-depth assessment of aging individuals and patients with MDS and IPF further highlight that RIOK2-mediated transcriptional control of TRiC and Dyskerin complexes underlie telomere shortening in these disorders. These findings shows that RIOK2-dependent transcriptional networks uniquely associate human blood cell development with telomere biogenesis.

### Results

Loss of RIOK2 triggers telomere shortening in erythroid and non-erythroid cells To gain deeper insights into RIOK2-dependent genetic programs, both knockdown (KD) and knockout (KO) of RIOK2 using CRISPR-Cas9 based gene editing were generated (FIG. 34A). Loss of RIOK2 substantially reduced cell proliferation in both TF-1 and K562 cells (FIG. 34B and FIG. 42A). Suppression of RIOK2 expression also arrested progression of cells at S and G2/M phases of cell cycle (FIG. 34C). Additionally, distinct upregulation of apoptosis was seen upon loss of RIOK2 (FIG. 34D). However, previously characterized functions of RIOK2 in erythroid differentiation could not explain these phenotypes. On the other hand, decline in cell growth, blockade of cell cycle at S and G2/M phases and increased apoptosis are all indicative of telomere shortening (Zhu, Y., Liu, X., Ding, X., Wang, F., and Geng, X. (2019). Telomere and its role in the aging pathways: telomere shortening, cell senescence and mitochondria dysfunction. Biogerontology 20, 1-16). Hence, the plausible roles of RIOK2 in telomere maintenance were investigated. Unbiased gene-set enrichment analysis also revealed a noteworthy decline in telomere maintenance upon RIOK2 deficiency (FIG. 34E). Indeed, loss of RIOK2 prominently induced telomere shortening in primary human hematopoietic stem and progenitor cells (HSPCs) as well as erythroblast cell lines, TF-1 and K562 (FIG. 34F-34H), as assessed by quantitative PCR-based methods. To further validate these findings, fluorescence in-situ hybridization (FISH) was performed to visualize telomeric puncta in RIOK2 proficient and deficient cells. Consistently, loss of RIOK2 markedly decreased formation of telomeric puncta in both TF-1 and K562 cells, thus demonstrating a key role of RIOK2 in telomere maintenance (FIG. 341 and FIG. 34J). This prompted us to explore roles of RIOK2 in telomere elongation in non-erythroid cells. To this end, Hela and HEK293 cells were examined and similarly observed a sharp decline in cell proliferation with dose-dependent loss of RIOK2 (FIG. 42B and FIG. 42C). Deficiency of RIOK2 analogously led to telomere shortening in both Hela and HEK293 cells (FIG. 34K, FIG. 34L, FIG. 42D and FIG. 42E), thus confirming that RIOK2 regulates telomere length homeostasis in both erythroid and non-erythroid cells.

### RIOK2 Transcriptionally Regulates TRiC Complex Subunits to Maintain TCAB1 Stability

The mechanisms underlying RIOK2-dependent telomere maintenance were investigated. RNA-sequencing analysis in primary human HSPCs showed a clear diminution in the mRNA expression of all TRiC complex subunits: TCP1 (CCT1), CCT2, CCT3, CCT4, CCT5, CCT6, CCT7 and CCT8 with depletion of RIOK2 (FIG. 35A). Using quantitative PCR analysis, it was validated that the mRNA expression of TRiC complex subunits were downregulated in both primary human HSPCs and TF-1 cells (FIG. 35B, FIG. 43A). Furthermore, loss of RIOK2 reduced the expression of TRiC subunits in Hela and HEK293 cells, thus signifying that RIOK2 controls TRiC complex expression in cells of both erythroid and non-erythroid origin (FIG. 43B and FIG. 43C). Given the recently identified transcriptional functions of RIOK2 (Ghosh, S., Raundhal, M., Myers, S. A., Carr, S. A., Chen, X., Petsko, G. A., and Glimcher, L. H. (2022). Identification of RIOK2 as a master regulator of human blood cell development. Nat Immunol 23, 109-121), these data led us to speculate whether RIOK2 acts as a transcription factor to regulate the expression of TRiC complex subunits. The ATAC-sequencing analysis showed dramatically reduced chromatin accessibility at the promoter regions of TRiC complex subunits upon loss of RIOK2 (FIG. 35C). Chromatin immunoprecipitation was then performed using both monoclonal and polyclonal antibodies against RIOK2 and observed that RIOK2 binds to the promoter regions of TCP1, CCT4, CCT6A and CCT8 (FIG. 35D). RIOK2 also transactivated the expression of TRiC complex subunits TCP1 and CCT8 in luciferase reporter assays (FIG. 43D). Next, the functional implications of RIOK2-mediated regulation of TRiC complex were performed. To interrogate this, the protein expression of TCAB1 was analyzed since TRiC chaperonin complex plays a central role in proper folding and stabilization of TCAB1 protein to sustain telomerase activity (Freund, A., Zhong, F. L., Venteicher, A. S., Meng, Z., Veenstra, T. D., Frydman, J., and Artandi, S. E. (2014). Proteostatic control of telomerase function through TRiC-mediated folding of TCAB1. Cell 159, 1389-1403). As speculated, a stark decrease in the protein expression of TCAB1 was observed at Cajal bodies upon RIOK2 deficiency in both TF-1 and K562 cells via immunofluorescence staining and immunoblotting (FIGS. 35E and 35F). These findings, thus, confirm that RIOK2 acts as a key transcriptional regulator of TRiC complex subunits to maintain TCAB1 expression.

### RIOK2-Mediated Regulation of TRiC and Dyskerin Complex Subunits Sustains Telomerase Activity

Shelterin and CTC complex subunits were then evaluated and no significant alteration in their mRNA levels with loss of RIOK2 was observed (FIGS. 44A and 44B). Interestingly, deficiency of RIOK2 led to a significant drop in the expression of Dyskerin complex subunits: DKC1, NHP2, NOP10 and GAR1 (FIG. 36A). These findings were also validated in primary human HSPCs, erythroid and non-erythroid cells (FIG. 36B, FIG. 44C-44E). Upon RIOK2 depletion, a stark decline in chromatin accessibility at the promoter regions of Dyskerin complex subunits was observed, indicating that RIOK2 may also function as a transcription factor for Dyskerin complex (FIG. 36C). As predicted, RIOK2 could not only bind with the promoter regions of Dyskerin complex subunits but also transactivate their expression (FIG. 36D and FIG. 44F). These results substantiate that RIOK2 transcriptionally controls the expression of Dyskerin complex subunits, along with TRiC complex. To confirm the functional outcome of RIOK2-driven expression of Dyskerin complex, the levels of TERC were analyzed, the RNA component of telomerase enzyme whose stability is maintained by the Dyskerin complex. Notably, the stability of TERC decayed with gradual loss of RIOK2 in primary human HSPCs (FIG. 36E). This RIOK2-mediated regulation of TERC stability was also observed in erythroid (TF-1, K562) and non-erythroid (Hela, HEK293) cells (FIG. 44G-FIG. 44J). Next, it was tested whether RIOK2-driven transcriptional regulation of TRiC and Dyskerin complexes reflects in the enzymatic activity of telomerase via telomerase repeated amplification protocol (TRAP) assay. Indeed, the telomerase activity of cells were prominently decreased with dose-dependent inhibition of RIOK2 (FIGS. 36F and 36G). Collectively, RIOK2 functions as a transcription factor to positively regulate the expression of TRiC and Dyskerin complex subunits, thereby maintaining telomere length homeostasis.

### The Transcriptional Abilities of RIOK2 are Critical in the Maintenance of Telomerase Activity

Besides the roles of RIOK2 as a transcription factor via its wHTH domain, this protein also plays a key role in cytoplasmic protein translation via its kinase domain (Ferreira-Cerca, S., Sagar, V., Schafer, T., Diop, M., Wesseling, A.M., Lu, H., Chai, E., Hurt, E., and LaRonde-LeBlanc, N. (2012). ATPase-dependent role of the atypical kinase Rio2 on the evolving pre-40S ribosomal subunit. Nat Struct Mol Biol 19, 1316-1323). Since globally obstructed cytoplasmic protein translation will inevitably affect telomere biogenesis, the contribution of RIOK2's transcriptional functions from its translational abilities in telomere maintenance was delineated. To achieve this, the DNA-binding mutant (DBM) and transactivation domain 1 and 2 deleted (ΔTAD1 and ΔTAD2) mutants of RIOK2 were used, as previously described (Ghosh, S., Raundhal, M., Myers, S.A., Carr, S.A., Chen, X., Petsko, G. A., and Glimcher, L. H. (2022). Identification of RIOK2 as a master regulator of human blood cell development. Nat Immunol 23, 109-121). Cellular proliferation in response to empty vector (EV), wild-type (WT) or the various mutants of RIOK2 was then tested, including the kinase-dead mutant K123A RIOK2 which blocks cytoplasmic protein translation. Reconstitution of WT RIOK2 almost completely rescued the inhibition of cellular proliferation in RIOK2-knockout (KO) cells, whereas the DNA-binding mutant (DBM) RIOK2 exhibited a significant decline in cell proliferation which was comparable to that of the kinase-dead mutant K123A RIOK2 (FIG. 37A). Intriguingly, depleting either of the transactivation domains rendered RIOK2 incapable of stimulating cell proliferation (FIG. 37A). Cell cycle analysis also indicated that perturbation of the DNA-binding domain or the transactivation domains of RIOK2 largely arrested cells at the S or G2/M phases of cell cycle (FIG. 37B). Chromatin immunoprecipitation further validated binding of wild-type (WT) RIOK2 to the promoter regions of TCP1 and DKC1, whereas the DNA-binding mutant (DBM) of RIOK2 showed marked reduction in its DNA-binding ability (FIG. 37C). The N-terminus extension (NTE) of RIOK2 comprising its wHTH domain was sufficient to interact with the promoter regions of its target genes (FIG. 37C). Luciferase reporter assays also confirmed the impeded transactivation functions of DBM, ΔTAD1 and ΔTAD2 RIOK2 as compared to WT RIOK2 (FIG. 37D). Corroborating this, the DBM, ΔTAD1 and ΔTAD2 mutants of RIOK2 failed to alleviate the expression of TRiC (TCP1, CCT6A) and dyskerin (DKC1, NHP2) complex subunits in RIOK2 knockout (KO) cells (FIG. 37E). In line with this, neither the DBM nor ΔTAD1 and ΔTAD2 RIOK2 mutants could rescue the expression of TCAB1 in RIOK2 KO cells (FIG. 37F, FIG. 45A). Furthermore, the DNA-binding mutant and transactivation domain deleted mutants of RIOK2 failed to rescue the expression of TERC in RIOK2 KO cells (FIG. 37G, FIG. 45B). Consequently, the DBM and ΔTAD mutants of RIOK2 could not revive the activity of telomerase enzyme as compared to WT RIOK2 in RIOK2 KO cells (FIG. 37H and 371, FIGS. 45C and 45D). The kinase dead mutant of RIOK2 K123A could also not rescue the expression of TERC or telomerase activity in RIOK2 knockout cells (FIG. 37G-37I). Taken together, these data demonstrate that both the transcriptional and translational functions of RIOK2 are pivotal in the maintenance of telomerase activity.

### Loss of the Transcriptional Functions of RIOK2 Results in Telomere Shortening

The involvement of RIOK2's transcriptional functions in telomere shortening was investigated next. To this end, fluorescence in-situ hybridization (FISH) was preformed of telomeric DNA in RIOK2 knockout (KO) cells with ectopic expression of the various mutants of RIOK2. Complete loss of RIOK2 substantially reduced telomeric puncta demonstrating acute telomere shortening, which was significantly rescued by the reconstitution of wild-type (WT) RIOK2 (FIG. 38A and FIG. 38B). However, the DNA-binding mutant and transactivation domain deleted mutants of RIOK2 could not restore telomere lengths in the absence of endogenous RIOK2, thus confirming key roles of the transcriptional functions of RIOK2 in telomere biogenesis (FIG. 38A and FIG. 38B). The kinase-dead mutant K123A RIOK2 could also not rescue telomere shortening as compared to WT reconstitution in RIOK2 KO cells, confirming that loss of cytoplasmic translation would eventually impair telomere biogenesis. WT RIOK2 also alleviated the DNA damage responses triggered by loss of RIOK2, whereas neither the DBM nor the ΔTAD mutants of RIOK2 could suppress it (FIG. 46). Taken together, these findings imply that the transcriptional functions of RIOK2 are as important as its translational activities in promoting telomere elongation.

Correlation of RIOK2's mRNA Expression in MDS Patients

It was recently reported that hematopoietic cell-specific heterozygous depletion of RIOK2 in mice results in MDS-associated anemia and myeloproliferation (Raundhal, M., Ghosh, S., Myers, S. A., Cuoco, M. S., Singer, M., Carr, S. A., Waikar, S. S., Bonventre, J. V., Ritz, J., Stone, R. M., et al. (2021). Blockade of IL-22 signaling reverses erythroid dysfunction in stress-induced anemias. Nat Immunol). It was also shown that the mRNA expression of RIOK2 significantly correlates with the expression of its target transcription factors in MDS patient-derived bone marrow samples, thus partly explaining the anemia observed in MDS patients (Ghosh, S., Raundhal, M., Myers, S. A., Carr, S. A., Chen, X., Petsko, G. A., and Glimcher, L. H. (2022). Identification of RIOK2 as a master regulator of human blood cell development. Nat Immunol 23, 109-121). These associations prompted evaluation of the expression of TRiC and Dyskerin complex subunits in the MDS patient-derived bone marrow samples. The independent assessment of a publicly available dataset (Pellagatti, A., Cazzola, M., Giagounidis, A., Perry, J., Malcovati, L., Della Porta, M. G., Jadersten, M., Killick, S., Verma, A., Norbury, C. J., et al. (2010). Deregulated gene expression pathways in myelodysplastic syndrome hematopoietic stem cells. Leukemia 24, 756-764) uncovered significant positive correlation between the mRNA levels of RIOK2 and all 8 subunits of the TRiC complex (FIG. 39A) in MDS patients. However, strong correlations between the mRNA expression of RIOK2 and shelterin complex subunits were not observed (FIG. 47A). Interestingly, the mRNA expression of RIOK2 also positively correlated with Dyskerin complex subunits (FIG. 39B). These correlations strongly indicate that loss of RIOK2-dependent telomere maintenance underlies MDS pathogenesis. This warranted further investigation into the telomere lengths of MDS patient-derived bone marrow cells. To support this hypothesis, bone marrow aspirates were procured from de-identified MDS patients and their genomic DNA and RNA was isolated for further analyses. Remarkably, the mRNA expression of RIOK2 positively correlated with the telomere lengths in MDS patient-derived bone marrow cells (FIG. 39C). Hence, these findings confirm that the telomere shortening in MDS is in part due to reduced expression and/or transcriptional abilities of RIOK2 to regulate the mRNA levels of TRiC and Dyskerin complexes.

The mRNA Expression of RIOK2 Declines with Aging and in IPF Patients

Given the integral roles of RIOK2 in the maintenance of telomerase activity and telomere length homeostasis, the expression levels of RIOK2 in young versus aging individuals was studied since telomere shortening is a predominant hallmark of aging. A publicly available microarray dataset of PBMCs derived from young (19-30 years) versus 146 nonagenarians (individuals with more than 90 years of age) was analyzed (Nevalainen, T., Kananen, L., Marttila, S., Jylha, M., Hervonen, A., Hurme, M., and Jylhava, J. (2015). Transcriptomic and epigenetic analyses reveal a gender difference in aging-associated inflammation: the Vitality 90+study. Age (Dordr) 37, 9814). Strikingly, the mRNA expression of RIOK2 was significantly reduced in nonagenarians as compared to young individuals (FIG. 40A). The expression patterns of TRiC and Dyskerin complex subunits was evaluated and a significant positive correlation between RIOK2 and several of the TRiC (CCT2, CCT4, CCT5, CCT8) and Dyskerin (DKC1, NHP2) complex subunits were observed (FIG. 40B and FIG. 40C). Of note, the young individuals exhibited higher expression of RIOK2 with correspondingly high expressions of both TRiC and Dyskerin complex subunits as compared to the nonagenarians (FIG. 40B and FIG. 40C). A strong correlation was not observed between the mRNA expressions of RIOK2 and shelterin complex subunits (FIG. 47B). Collectively, these findings strongly implicate that attenuated RIOK2-driven expression of TRiC and Dyskerin complexes underlie telomere shortening in aging individuals.

Since telomere shortening is also a key characteristic feature of telomere biology disorders, such as Idiopathic Pulmonary Fibrosis (IPF), the expression patterns of RIOK2 in a cohort of 70 IPF patients was studied (Huang, L. S., Berdyshev, E. V., Tran, J. T., Xie, L., Chen, J., Ebenezer, D. L., Mathew, B., Gorshkova, I., Zhang, W., Reddy, S. P., et al. (2015). Sphingosine-1-phosphate lyase is an endogenous suppressor of pulmonary fibrosis: role of SiP signalling and autophagy. Thorax 70, 1138-1148). The mechanistic basis of telomere shortening in the PBMCs of IPF patients is not clearly understood (Stuart, B. D., Lee, J. S., Kozlitina, J., Noth, I., Devine, M. S., Glazer, C. S., Torres, F., Kaza, V., Girod, C. E., Jones, K. D., et al. (2014). Effect of telomere length on survival in patients with idiopathic pulmonary fibrosis: an observational cohort study with independent validation. Lancet Respir Med 2, 557-565). These analyses revealed that the mRNA levels of RIOK2 were significantly reduced in IPF patients as compared to healthy individuals (FIG. 41A). More importantly, the mRNA expression of RIOK2 strongly correlated with the prognostic factors for IPF patients, such as diffusing capacity of the lung for carbon monoxide percent (dlco %) and forced vital capacity percent (FVC %) (FIG. 41B), which indicate that lesser expression of RIOK2 somehow correlate with poor prognosis in IPF patients. In addition, the mRNA expression of RIOK2 positively correlated with TRiC (CCT1, CCT2, CCT4, CCT6A) and Dyskerin (DKC1, NOP10) complex subunits in IPF patients (FIG. 41C and FIG. 41D). Since loss of RIOK2 also triggers telomere shortening in non-erythroid cells, the transcriptional landscape of lung explants from IPF patients was studied next (McDonough, J. E., Ahangari, F., Li, Q., Jain, S., Verleden, S.E., Herazo-Maya, J., Vukmirovic, M., DeIuliis, G., Tzouvelekis, A., Tanabe, N., et al. (2019). Transcriptional regulatory model of fibrosis progression in the human lung. JCI Insight 4). Surprisingly, the mRNA expression of RIOK2 was appreciably downregulated in the explant-derived lung tissues from IPF patients as compared to healthy controls (FIG. 48A). Consistently, the mRNA expression of RIOK2 positively correlated with TRiC and Dyskerin complex subunits in the lung tissues (FIG. 48B and FIG. 48C), whereas such strong association was not observed for the shelterin complex subunits (FIG. 48D). Taken together, these analyses strongly advocate that loss of RIOK2-driven transcription of TRiC and Dyskerin complex subunits underlie telomere shortening in aging individuals as well as in patients with MDS and IPF (FIG. 41E).

### MATERIALS AND METHODS for Example 4

Primary and secondary cell culture

CD34+ primary human hematopoietic stem and progenitor cells (HSPCs) were obtained from Fred-Hutchinson Cancer Research center, Seattle, USA. CD34+cells were isolated from G-CSF-mobilized peripheral blood of adult healthy donors by magnetic sorting and cryopreserved. The cells were thawed and washed with lx PBS constituting 1% human AB serum, followed by revival in expansion medium constituting StemSpan SFEM II medium supplemented with 1X CC100 (STEMCELL Technologies), 1% penicillin-streptomycin (P/S), 1% Glutamine and 10 ng/ml TPO. Post 3-5 days of genetic editing in the expansion phase, the cells were subjected to differentiation medium containing IMDM reconstituted with 3% human AB serum, 2% human AB plasma, 1% P/S, 0.06% heparin solution (STEMCELL Technologies), 1 ng/ml IL-3 (Peprotech, Inc.), 10 ng/ml SCF (Peprotech, Inc.), 200 μg/ml holo-transferrin (Sigma-Aldrich) and 3U/ml erythropoietin (DFCI pharmacy). Cells were cultured at a density of 0.1-0.2×10⁶ cells/ml and incubated at 37° C. with 5% CO2. Media were changed every alternate day, as required. These protocols have been previously described (Khajuria, R. K., Munschauer, M., Ulirsch, J. C., Fiorini, C., Ludwig, L. S., McFarland, S. K., Abdulhay, N. J., Specht, H., Keshishian, H., Mani, D. R., et al. (2018). Ribosome Levels Selectively Regulate Translation and Lineage Commitment in Human Hematopoiesis. Cell 173, 90-103 e119).

TF-1 human erythroblast cells were purchased from ATCC (ATCC® CRL-2003TM) and maintained in RPMI-1640 medium (ATCC® 30-2001TM) containing 10% fetal bovine serum (FBS), 2 ng/ml GM-CSF and 1% P/S. For differentiation, the cells were washed with lx PBS and resuspended in RPMI-1640 medium containing 10% FBS, 5 U/ml erythropoietin and 1% P/S. K562 human erythroid cells were purchased from ATCC (ATCC® CCL-243) and maintained in IMDM medium (ATCC® 30-2005™) containing 10% FBS and 1% P/S. For differentiation, the cells were washed with lx PBS and resuspended in IMDM medium containing 10% FBS, 40 μM hemin and 1% P/S. HEK293 cells were purchased from ATCC, and Hela cells were a kind gift from Dr. Kai Wucherpfennig's lab at DFCI. Both Hela and HEL293 cells were maintained in DMEM medium supplemented with 10% FBS and 1% P/S. Cells were incubated at 37° C. with 5% CO₂. Media were changed every alternate day, as required.

### Generation of Lentiviral Vectors and Infection

RIOK2 was cloned in the pHAGE-MCS-IRES-ZsGreen lentiviral vector with an N-terminal Hemagglutinin (HA) tag. DNA binding mutant (DBM), Transactivation domain deletion 1 and 2 (ΔTAD1 and ΔTAD2) vectors were generated by site-directed mutagenesis in N-terminal HA-tagged RIOK2 construct. N-terminal extension (NTE) construct was generated by cloning the first 92 amino acids of RIOK2 in the lentiviral vector mentioned above with an N-terminal HA-tag.

For lentiviral production, HEK293FT cells were transiently transfected with pVSV-G, pDelta8.9 and the required RIOK2 vectors using X-tremeGENE™ HP DNA transfection reagent as per the manufacturer's protocol. Viral supernatant was collected 48 hours post transfection. Viral supernatant was centrifuged at 1500 rpm for 5 mins to remove cellular debris, then filtered using 0.45 μm filter and stored at −80° C. for further usage. Primary HSPCs or secondary cells were transduced at a density of 0.1-0.2×10⁶ cells per well in 6-well plates. Spinfection was performed with viral supernatants added with 8 μg/ml polybrene (Millipore) at 2000 rpm for 1.5 hours at 32° C. and left overnight. The medium was changed the next morning. Lentiviral transduction efficiency reached 60-75% for primary human HSPCs and >95% for secondary cells after 48 hours of infection. The positively transduced cells (ZsGreen+) were FACS-sorted for further analysis.

### CRISPR/Cas9 Gene Editing

Primary human HSPCs and human erythroblast cells (TF-1) were electroporated using Lonza-Amaxa 4D nucleofector unit as per the manufacturer's protocols. For ribonucleoprotein (RNP) formation, tracer RNAs (IDT) and crRNAs (IDT) were incubated at 95° C. for 5 mins at equal molar ratios, brought to room temperature (RT) and mixed with equal molar concentration of Cas9 peptide (IDT Technologies). This was followed by incubation at 37° C. for 15 minutes and the RNP mix was kept at 4° C. until use within the next 2 hours. Cells were mixed with the RNP mix at a density of 0.1-0.2×10⁶ cells per well of a 16-well electroporation-strip and subsequently electroporated. Immediately after electroporation, fresh medium was added to the cells and incubated at RT for 10 mins before transferring the cells to 37° C. Genome editing efficiency was analyzed 48-72 hours after electroporation by nucleotide sequencing, quantitative RT-PCR, and Western blotting. The crRNAs used for knockdown (KD) of RIOK2: GAACGGCGGGTTTCTTACCG (SEQ ID NO: 33) and CATTTGTCAACCGATAGCCC (SEQ ID NO: 34), crRNAs used for knockout (KO) of RIOK2: TGACTTCAGGGTCTTGACCG (SEQ ID NO: 35) and TGATTACAATCGTCATGCAG (SEQ ID NO: 36). crRNA against LacZ (Control): TTCTCCGCGGGAACAAACGG (SEQ ID NO: 37).

### Luciferase Reporter Assay

For transactivation assays, the promoter regions of TCP1, CCT8, DKC1 and NHP2 (500-750 bps upstream of the ATG start codon) were cloned in pGL3.1 basic vector. Luminescence intensities were normalized using co-transfection of Renilla expression vector in HEK293 cells using Lipofectamine 3000 reagent. Dual luciferase assays (Promega) were performed as per manufacturer's instructions. Luminescence intensities were captured using EnVision™ Multimode plate reader (Perkin Elmer).

### Quantitative RT-PCR

RNA was isolated from cells using the RNeasy Plus Micro kit (Qiagen) using manufacturer's protocols. Genomic DNA (gDNA) was removed using gDNA eliminator spin columns, followed by isolation of total RNA using phenol-free RNeasy MinElute spin columns. Reverse transcription was performed using qScript™ cDNA Synthesis kit (QuantaBio). Quantitative real-time polymerase chain reaction (RT-PCR) was performed using Quantstudio6 RT-PCR system (Applied Biosciences) and PerfeCTa SYBR™ Green FastMix Reaction Mixes (QuantaBio). Comparative C_{T} method has been used for all quantifications using corresponding R-Actin mRNA levels for normalization.

### Western Blotting

For collection of whole cell lysates, cells were washed twice with ice cold 1× PBS after removal of medium, then resuspended in RIPA lysis buffer (Life Technologies) supplemented with 1× complete protease inhibitor cocktail and 1× phosphatase inhibitor cocktail (Thermo Scientific). Lysis was carried out on a rocker at 4° C. for 15-30 minutes followed by removal of cellular debris by centrifugation at 13,000 rpm for 20 minutes. The supernatant was collected in a fresh tube, mixed with Laemmli buffer and incubated at 95° C. for 10 minutes. Equal amounts of proteins were resolved by SDS-PAGE method using Novex Tris-Glycine minigels submerged in Tris-Glycine-SDS buffer in a mini gel tank (Life Technologies). The proteins were then transferred onto PVDF membranes (Thermo Fisher Scientific) using Tris-Glycine buffer followed by blocking in 5% skimmed milk solution in 1× PBST buffer at RT for 1 hour. The membranes were then probed with the required primary antibodies diluted in fresh blocking buffer at 4° C. for overnight (O/N): HA rabbit monoclonal antibody at 1:1000 (C29F4, 37245, Cell Signaling Technology), beta-Actin rabbit polyclonal antibody at 1:1000 (3967S, Cell Signaling Technology), RIOK2 mouse monoclonal antibody at 1:1000 (OTI3E11, TA505140, Origene). After O/N incubation, membranes were washed four times with PBST buffer for 5 minutes each on a rocker at RT, followed by incubation with HRP-linked anti-mouse IgG (7076S, Cell Signaling Technology) or HRP-linked anti-rabbit IgG (7076S, Cell Signaling Technology) at 1:3000 dilution in fresh blocking buffer for 1 hour at RT. After incubation with secondary antibodies, membranes were washed four times with PBST buffer for 5 minutes each on a rocker at RT, followed by incubation with Pierce Western blotting substrates (Thermo Scientific) mixed at 1:1 ratio for 2-5 mins at RT. The protein bands were then visualized using the ChemiDoc Touch™ Imaging system (Bio-Rad).

### Cell Cycle Analysis

For analysis of cell cycle stages, the cells were washed with 1× PBS and fixed using 4% PFA for 30 minutes or 70% ethanol for 1 hour at 4° C. The cells were washed twice using 1× PBS and resuspended in DAPI containing staining buffer followed by FACS analyses using CytoFLEX™ Flow Cytometer (Beckman Coulter). Data were analyzed using FlowJo 10.0.7 and plotted using GraphPad Prism.

### Apoptosis

Staining of apoptotic cells was performed using the Annexin V apoptosis detection kit from eBioscience (#88-8102-72) and visualized using CytoFLEX™ Flow Cytometer (Beckman Coulter). Data were analyzed using FlowJo 10.0.7 and plotted using GraphPad Prism.

### Telomerase Repeated Amplification Protocol (TRAP) Assay

Analysis of telomerase activity was performed using non-radioactive method with TRAPeze® Telomerase detection kit from Millipore (#S7700), as per manufacturer's protocols. Cells were lysed in the provided CHAPS lysis buffer and 0.5/0.25/0.1 μg total protein lysate was used in reaction mixture containing the provided TRAP reaction buffer, dNTP mix, TRAP and TS primers, and Taq polymerase enzyme. PCR reactions were performed in a thermal cycler using the following program:
Cycle 1 for 1 cycle: 30° C. for 30 mins;
Cycle 2 for 1 cycle: 95° C. for 2 mins;
Cycle 3 for 30 cycles: 94° C. for 15 secs, 59° C. for 30 secs, 72° C. for 1 min. 4° C. ∞ for 1 cycle.

The PCR samples and 10 bp DNA ladder were then loaded onto a 10% polyacrylamide TBE gel and run at 100V for 1 hr and 45 minutes. Post electrophoresis, the TBE gels were stained with GELRED nucleic acid gel stain (#10154-212) for 30 mins at RT in dark and visualized using ChemiDoc Touch Imaging system (Bio-Rad).

### Bulk RNA-Sequencing

RNA library preparations, sequencing reactions and initial bioinformatic analysis were conducted at GENEWIZ, LLC. (South Plainfield, NJ, USA) as follows:

Library Preparation with polyA selection and HiSeq Sequencing: RNA samples received were quantified using Qubit 2.0 Fluorometer (Life Technologies, Carlsbad, CA, USA) and RNA integrity was checked using Agilent TapeStation 4200 (Agilent Technologies, Palo Alto, CA, USA). RNA sequencing libraries were prepared using the NEBNext Ultra RNA Library Prep Kit for Illumina following manufacturer's instructions (NEB, Ipswich, MA, USA). Briefly, mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA). The sequencing libraries were clustered on 1 lane of a flowcell. After clustering, the flowcell was loaded on the Illumina HiSeq instrument (4000 or equivalent) according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

### Data Analysis

After investigating the quality of the raw data, sequence reads were trimmed to remove possible adapter sequences and nucleotides with poor quality using Trimmomatic v.0.36. The trimmed reads were mapped to the Homo sapiens reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. Only unique reads that fell within exon regions were counted.

Gene set enrichment analysis (GSEA) was performed using Broad Institute's GSEA Software. Sets of telomere-maintenance associated genes were derived by accounting for the differentially regulated genes that passed the cut off of adjusted p value <0.05 in control vs RIOK2-depleted HSPCs. Additionally, canonical pathways such as KEGG, REACTOME and BIOCARTA were consulted.

Assay of Transposase accessible Chromatin with high-throughput Sequencing (ATAC-Seq) ATAC-seq on equal number of snap-frozen control and RIOK2-depleted HSPCs was performed at Center for Functional Cancer Epigenetics (CFSE) at DFCI. Briefly, after high throughput Illumina sequencing of pair-ended reads, the raw reads were mapped to the reference genome (hg19) using alignment software (conFigureyaml). After alignment, the mapped reads were then normalized (by down-sampling) to 4 million reads. The uniquely mapped reads of the downsample were then piled-up. Nd is defined as the number of unique region locations. N1 is defined as the number of unique region locations with only one read. The PBC (a common method to determine sample complexity) is then simply calculated as N1/Nd. ATAC-seq samples with a good PBC score >=0.90 were analyzed. Peaks were called using the MACS peak calling software. Chromatin accessibility was analyzed using IGV software.

### Chromatin Immunoprecipitation (ChIP)

Chromatin Immunoprecipitation was performed using EZChIP™ kit (Millipore Sigma) according to the manufacturer's instructions. Briefly, 1×106 cells were incubated with growth medium supplemented with formaldehyde (methanol-free) at a final concentration of 1% for 10 minutes at RT for cross-linking. Then unreacted formaldehyde was quenched by adding Glycine at a final concentration of 0.125M. The cells were then washed twice with Ix PBS, followed by resuspension in lysis buffer supplemented with protease inhibitors and incubated at 4° C. for 15-30 minutes. The lysed samples were then sonicated at 25% amplitude for 5 mins at 1 sec on and 4 sec off cycles, followed by centrifugation at 12,000g for 10 mins at 4° C. To the supernatant, 60 μl of protein agarose G beads slurry was added for pre-clearing and incubated at 4° C. for 1 hour with rotation, followed by spinning at 1000 rpm for 1 min. 1% of the pre-cleared supernatant was saved as Input, and to the rest 2-4 μg primary antibodies: HA rabbit monoclonal antibody (C29F4, 3724S, Cell Signaling Technology), RIOK2 mouse monoclonal antibody (OTI3E11, TA505140, Origene); RIOK2 rabbit polyclonal antibody (NBP130098, Thermo Fisher Scientific)) or control IgGs were added followed by incubation at 4° C. with rotation overnight. After overnight incubation, 60 μl of protein agarose G beads slurry was added to each sample and incubated at 4° C. for 2 hours. The agarose beads were pelleted by spinning, and washed sequentially with low salt buffer, high salt buffer, LiCl wash buffer and TE buffer. After washing, elution of protein-DNA complexes was performed using elution buffer containing NaHCO₃ and SDS at RT for 15-30 mins. The eluted supernatant was treated with RNase A for 30 mins at 37° C. and reverse-crosslinked using a buffer containing EDTA, Tris-HCl and Proteinase K for 1-2 hours at 42° C. After reverse-crosslinking, DNA was purified using Spin columns provided by Millipore Sigma/Qiagen and the purified DNA was then used for quantitative RT-PCR using Quantstudio6 RT-PCR system (Applied Biosciences).

### Fluorescence In-Situ Hybridization (FISH)

Fluorescence in-situ hybridization was performed using probes from PNA Biosciences as per manufacturer's protocols. Cells were fixed on slides and washed twice with 1× PBS. They were treated with RNase solution and incubated at 37° C. for 20 minutes, followed by 1× PBS wash and pepsin digestion. The cells were then re-fixed with 70%, 85% and 100% cold ethanol solutions sequentially and air dried. Hybridization buffer (20 mM Tris, pH7.4, 60% formamide, 0.5% blocking reagent) containing 500 nM PNA probes (working concentration) was added to the air-dried slides, followed by heating the slides at 85° C. for 10 mins and incubation at room temperature for 2 hours in the dark. The slides were then sequentially washed with wash solution (2× SSc, 0.1% Tween-20), 2× SSc, 1× SSc and water, followed by quick drying the slides and mounting with anti-fade mounting reagent containing DAPI. The slides were then visualized using fluorescence microscopy.

### Immunofluorescence Staining

For immunofluorescence staining, the coverslips were coated with poly-L-Lysine solution (0.1 mg/ml) solution at 4° C. overnight. The coverslips were then washed with 1× PBS and cells were incubated on the coverslips. The cells on the coverslips were then fixed with 4% PFA in 1× PBS for 15 mins at RT, washed with lx PBS, permeabilized using 0.1% Triton in 1× PBS for 5 mins at RT and blocked with 4% BSA in 1× PBS for 1 hr at RT. The cells were then incubated with primary antibodies (TCAB1: Novus Biologicals #NB100-68252; Coilin: Abcam #ab11822; y-H2AX: Cell Signaling Technology #9718S) diluted in blocking buffer for 1 hour at RT, washed thrice with 1× PBS, and incubated with secondary antibodies (Abcam) for 1 hour at RT. The coverslips were similarly washed thrice with 1× PBS, mounted with anti-fade mounting reagent containing DAPI, and visualized using fluorescence microscopy.

### Telomere Length Measurement

Quantitative PCR-based telomere length measurement was performed as previously described (Cawthon, 2002; Hehar and Mychasiuk, 2016). The final telomere primer concentrations were: tell, 270 nM; tel2, 900 nM. The final 36B4 (single copy gene) primer concentrations were: 36B4u, 300 nM; 36B4d, 500 nM. The primer sequences were: tell, GGTTTTTGAGGGTGAGGGTGAGGGTGAGGGTGAGGGT (SEQ ID NO: 82); tel2, TCCCGACTATCCCTATCCCTATCCCTATCCCTATCCCTA (SEQ ID NO: 83); 36B4u, CAGCAAGTGGGAAGGTGTAATCC (SEQ ID NO: 84); 36B4d, CCCATTCTATCATCAACGGGTACAA (SEQ ID NO: 85). The housekeeping 36B4 gene encodes acidic ribosomal phosphoprotein PO and is located on chromosome 12.

### Statistical Tests

Data are presented as mean±SEM. Unpaired two-tailed t-test was used for comparing two groups. Analysis of variance (ANOVA) with Tukey's correction or Kruskal-Wallis test with Dunn's correction was used for comparisons amongst multiple groups, wherever applicable as per requirements of data and quantification. GraphPad Prism v8.0/9.0 (GraphPad Software Inc., San Diego, CA) was used to perform statistical analyses. Sample size was not predetermined.

### Example 5: Generation of Stapled Peptides to Activate and/or Stabilize or Inhibit RIOK2

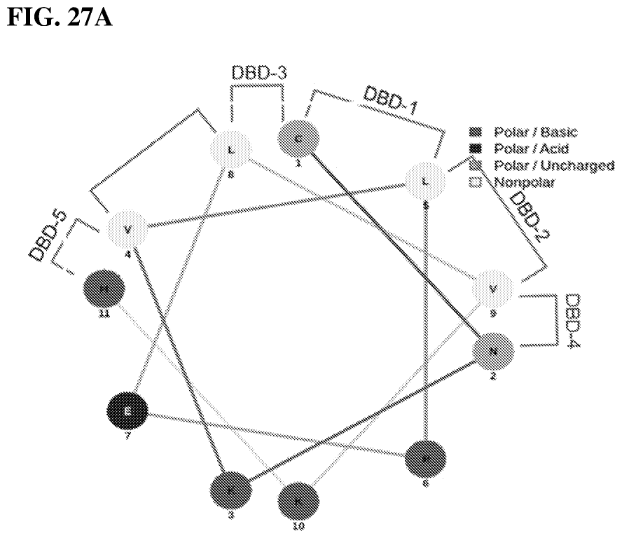

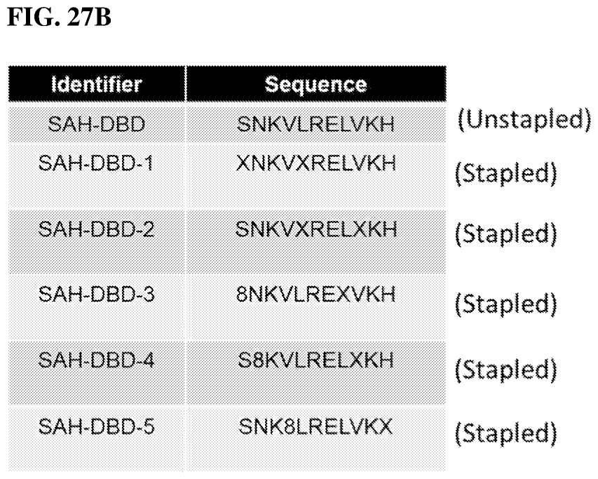

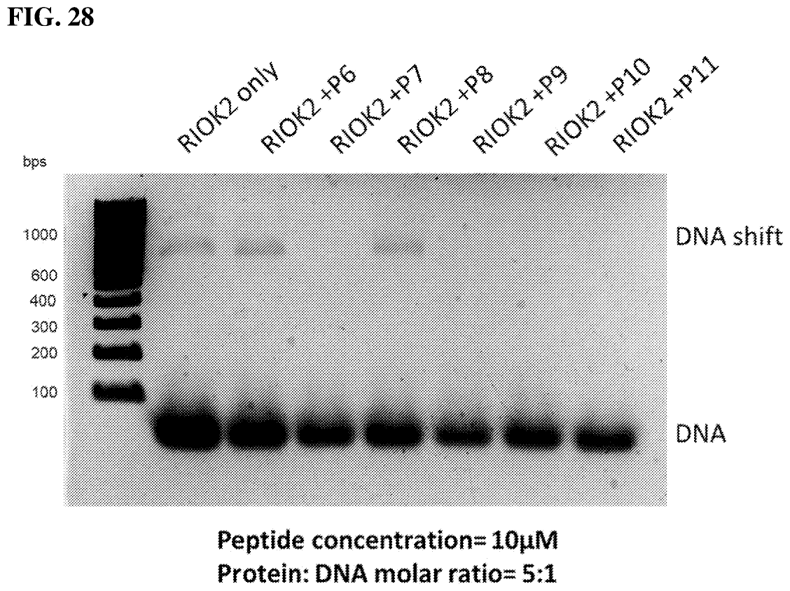

Provided herein are stapled peptides to stabilize/activate or inhibit RIOK2. Provided herein are internally cross-linked polypeptides that include (i) the amino acid sequence of GSLIASIAS (SEQ ID NO: 5) and/or (ii) a sequence containing between one to six amino acid substitutions in the sequence of SEQ ID NO: 5. These stapled peptides modelled after the transrepressor domain (TRD) of RIOK2 were generated to activate RIOK2 and/or stabilize its expression (FIG. 33). Specifically, stapled peptides may bind to an amino acid sequence in a corepressor that binds to RIOK2 may be used to activate or stabilize RIOK2. Stapled peptides modelled after the DNA-binding and transrepressor domains of RIOK2 are being tested in cell-free and cell-based assays. Internally cross-linked polypeptides may include (i) the amino acid sequence of SNKVLRELVKH (SEQ ID NO: 11) and/or (ii) a sequence containing between one to seven amino acid substitutions in the sequence of SEQ ID NO: 11. Stapled peptides modelled after the DNA-binding domain (DBD) of RIOK2 have been generated to inhibit RIOK2's activity (FIG. 27A-B).

### Example 6: Regulatory Roles of RIOK2 in Mitochondrial Metabolism

Myelodysplastic syndromes (MDS) are one of the most frequently occurring hematologic neoplasms in the U.S. (Bejar, R., and Steensma, D.P. (2014). Recent developments in myelodysplastic syndromes. Blood 124, 2793-2803.; Garcia-Manero, G., Chien, K. S., and Montalban-Bravo, G. (2020). Myelodysplastic syndromes: 2021 update on diagnosis, risk stratification and management. Am J Hematol 95, 1399-1420; Saygin, C., and Carraway, H. E. (2020). Current and emerging strategies for management of myelodysplastic syndromes. Blood Rev, 100791). It was recently reported that hematopoietic-cell specific heterozygous deletion of the atypical kinase RIOK2 results in anemia and myeloproliferation in mice (Raundhal, M., Ghosh, S., Myers, S. A., Cuoco, M. S., Singer, M., Carr, S. A., Waikar, S. S., Bonventre, J. V., Ritz, J., Stone, R. M., et al. (2021). Blockade of IL-22 signaling reverses erythroid dysfunction in stress-induced anemias. Nat Immunol.), thus presenting a novel mouse model for studying MDS. Remarkably, this comprehensive analysis in primary human HSPCs revealed that RIOK2 functions as a master transcription factor (TF) governing hematopoietic differentiation via its previously unexplored winged-helix-turn-helix (wHTH) domain incorporating DNA-binding (DBD), transrepressor (TRD) and transactivation (TAD) subdomains. It was discovered that RIOK2 drives erythropoiesis and concomitantly suppresses megakaryopoiesis and myelopoiesis by controlling the expression of key lineage-specific transcription factors for each of these pathways (Ghosh, S., Raundhal, M., Myers, S. A., Carr, S. A., Chen, X., Petsko, G. A., and Glimcher, L. H. (2022). Identification of RIOK2 as a master regulator of human blood cell development. Nat Immunol 23, 109-121). Mitochondrial dysfunction is an unappreciated hallmark of MDS patient-derived cells (Goncalves, A. C., Cortesao, E., Oliveiros, B., Alves, V., Espadana, A. I., Rito, L., Magalhaes, E., Lobao, M. J., Pereira, A., Nascimento Costa, J. M., et al. (2015). Oxidative stress and mitochondrial dysfunction play a role in myelodysplastic syndrome development, diagnosis, and prognosis: A pilot study. Free Radic Res 49, 1081-1094; Schildgen, V., Wulfert, M., and Gattermann, N. (2011). Impaired mitochondrial gene transcription in myelodysplastic syndromes and acute myeloid leukemia with myelodysplasia-related changes. Exp Hematol 39, 666-675 e661; Ward, G. A., McGraw, K. L., Abbas-Aghababazadeh, F., Meyer, B. S., McLemore, A. F., Vincelette, N. D., Lam, N. B., Aldrich, A. L., Al Ali, N. H., Padron, E., et al. (2021). Oxidized mitochondrial DNA released after inflammasome activation is a disease biomarker for myelodysplastic syndromes. Blood Adv 5, 2216-2228). Although metabolic profiles are widely recognized to influence stem cell fate (Agathocleous, M., Meacham, C. E., Burgess, R. J., Piskounova, E., Zhao, Z., Crane, G. M., Cowin, B. L., Bruner, E., Murphy, M. M., Chen, W., et al. (2017). Ascorbate regulates haematopoietic stem cell function and leukaemogenesis. Nature 549, 476-481; Ge, Y., and Fuchs, E. (2018). Stretching the limits: from homeostasis to stem cell plasticity in wound healing and cancer. Nat Rev Genet 19, 311-325; Jia, Y., Guo, J., Zhao, Y., Zhang, Z., Shi, L., Fang, Y., Wu, D., Wu, L., and Chang, C. (2021). AHR signaling pathway reshapes the metabolism of AML/MDS cells and potentially leads to cytarabine resistance. Acta Biochim Biophys Sin (Shanghai) 53, 492-500; Xie, J., Lou, Q., Zeng, Y., Liang, Y., Xie, S., Xu, Q., Yuan, L., Wang, J., Jiang, L., Mou, L., et al. (2021). Single-Cell Atlas Reveals Fatty Acid Metabolites Regulate the Functional Heterogeneity of Mesenchymal Stem Cells. Front Cell Dev Biol 9, 653308.) The molecular coupling of aberrant mitochondrial metabolism with MDS pathogenesis remains poorly defined. Here, it was identified that loss of RIOK2 in primary human HSPCs: (a) markedly inhibited mitochondrial translation (b) significantly reduced expression of mitochondrial ribosomal protein (MRP) coding genes, (c) differentially regulated genes involved in glycolysis, TCA cycle and electron transport chain reactions, and (d) deregulated metabolite profiles of TCA cycle intermediates. These findings led us to postulate that RIOK2 regulates mitochondrial metabolism to dictate the bioenergetics of hematopoietic differentiation, deregulation of which underlies anemia and other associated hematological pathologies.

These findings define RIOK2-driven genetic and metabolic networks and uncover a unique map correlating transcriptional and metabolic footprints that should facilitate a deeper understanding of the currently elusive etiology of MDS, which will in turn improve predictive and therapeutic approaches in a range of hematologic disorders.

### INTRODUCTION

### Metabolism and Myelodysplastic Syndromes (MDS)

MDS encompasses a heterogenous group of clonal neoplasms originating in hematopoietic stem cells, that are characterized by bone marrow failure, peripheral blood cytopenias, ineffective hematopoiesis, and an escalated risk of clonal evolution and progression to acute myeloid leukemia (Chang, Y. H. (2021). Myelodysplastic syndromes and overlap syndromes. Blood Res 56, S51—S64; Crisa, E., Boggione, P., Nicolosi, M., Mahmoud, A.M., Al Essa, W., Awikeh, B., Aspesi, A., Andorno, A., Boldorini, R., Dianzani, I., et al. (2021). Genetic Predisposition to Myelodysplastic Syndromes: A Challenge for Adult Hematologists. Int J Mol Sci 22; Lefevre, C., Bondu, S., Le Goff, S., Kosmider, O., and Fontenay, M. (2017). Dyserythropoiesis of myelodysplastic syndromes. Curr Opin Hematol 24, 191-197). The international prognostic scoring system (IPSS) and the Revised-IPSS (IPSS-R) are the benchmarks for MDS prognosis, based on marrow blast counts, number and degree of cytopenias, and metaphase karyotype (Greenberg, P. L., Tuechler, H., Schanz, J., Sanz, G., Garcia-Manero, G., Sole, F., Bennett, J. M., Bowen, D., Fenaux, P., Dreyfus, F., et al. (2012). Revised international prognostic scoring system for myelodysplastic syndromes. Blood 120, 2454-2465; Park, M. (2021). Myelodysplastic syndrome with genetic predisposition. Blood Res 56, S34—S38.). MDS is predominantly diagnosed in the elderly population aged >60, partly owing to a dearth of effective diagnostic tests for detection at early stages (Feld, J., Belasen, A., and Navada, S. C. (2020). Myelodysplastic syndromes: a review of therapeutic progress over the past 10 years. Expert Rev Anticancer Ther 20, 465-482; Garcia-Manero, G., Chien, K. S., and Montalban-Bravo, G. (2020). Myelodysplastic syndromes: 2021 update on diagnosis, risk stratification and management. Am J Hematol 95, 1399-1420; Steensma, D.P. (2019). Does early diagnosis and treatment of myelodysplastic syndromes make a difference?Best Pract Res Clin Haematol 32, 101099). Although cytogenetic analyses and mutational profiling add an effective stratum in diagnosis, patient risk stratification and therapy selection; allelic heterogeneity, presence of combinatorial or germline mutations, and minimal influence to decide treatment modalities dissuade clinicians from adopting complementary molecular and sequencing approaches (Steensma, D.P. (2016). New challenges in evaluating anemia in older persons in the era of molecular testing.Hematology Am Soc Hematol Educ Program 2016, 67-73). Consequently, MDS often remains misdiagnosed as idiopathic cytopenias of undetermined significance (ICUS) (Joosten, E. (2004). Strategies for the laboratory diagnosis of some common causes of anaemia in elderly patients. Gerontology 50, 49-56; Malcovati, L., and Cazzola, M. (2015). The shadowlands of MDS: idiopathic cytopenias of undetermined significance (ICUS) and clonal hematopoiesis of indeterminate potential (CHIP). Hematology Am Soc Hematol Educ Program 2015, 299-307.; Valent, P., Bain, B. J., Bennett, J. M., Wimazal, F., Sperr, W. R., Mufti, G., and Horny, H.P. (2012). Idiopathic cytopenia of undetermined significance (ICUS) and idiopathic dysplasia of uncertain significance (IDUS), and their distinction from low risk MDS. Leuk Res 36, 1-5) for years before becoming untreatable, since many patients become ineligible for the only curative treatment of allogeneic HSCT due to other age-related co-morbidities (Steensma, D. P., Brunner, A. M., DeZern, A. E., Garcia-Manero, G., Komrokji, R. S., Odenike, O. S., Roboz, G. J., Savona, M. R., Stone, R. M., and Sekeres, M. A. (2018). Low clinical trial accrual of patients with myelodysplastic syndromes: Causes and potential solutions. Cancer 124, 4601-4609). Only a handful of FDA-approved treatment regimens apart from HSCT, such as hypomethylating agents (Cheng, W. Y., Satija, A., Cheung, H. C., Hill, K., Wert, T., Laliberte, F., and Lefebvre, P. (2021). Persistence to hypomethylating agents and clinical and economic outcomes among patients with myelodysplastic syndromes. Hematology 26, 261-270; Kordella, C., Lamprianidou, E., and Kotsianidis, I. (2021). Mechanisms of Action of Hypomethylating Agents: Endogenous Retroelements at the Epicenter. Front Oncol 11, 650473.; Schiffer, M., Zhao, J., Johnson, A., Lee, J., Bewersdorf, J. P., and Zeidan, A. M. (2021). The development and clinical use of oral hypomethylating agents in acute myeloid leukemia and myelodysplastic syndromes: dawn of the total oral therapy era. Expert Rev Anticancer Ther, 1-13, lenalidomide (Hecht, A., Meyer, J. A., Jann, J. C., Sockel, K., Giagounidis, A., Gotze, K. S., Letsch, A., Haase, D., Schlenk, R. F., Haferlach, T., et al. (2021). Genome-wide DNA methylation analysis pre- and post-lenalidomide treatment in patients with myelodysplastic syndrome with isolated deletion (5q). Ann Hematol; List, A. F., Sun, Z., Verma, A., Bennett, J. M., Komrokji, R. S., McGraw, K., Maciejewski, J., Altman, J. K., Cheema, P. S., Claxton, D. F., et al. (2021). Lenalidomide-Epoetin Alfa Versus Lenalidomide Monotherapy in Myelodysplastic Syndromes Refractory to Recombinant Erythropoietin. J Clin Oncol 39, 1001-1009) or luspatercept (Chan, O., and Komrokji, R. S. (2021). Luspatercept in the treatment of lower-risk myelodysplastic syndromes. Future Oncol 17, 1473-1481; Kubasch, A. S., Fenaux, P., and Platzbecker, U. (2021). Development of luspatercept to treat ineffective erythropoiesis. Blood Adv 5, 1565-1575), and a paucity of druggable candidates further compound the treatment complications, resulting in a median survival of less than 3 years in MDS patients after diagnosis (Awada, H., Thapa, B., and Visconte, V. (2020). The Genomics of Myelodysplastic Syndromes: Origins of Disease Evolution, Biological Pathways, and Prognostic Implications. Cells 9; Bhatt, V. R., and Steensma, D. P. (2016). Hematopoietic Cell Transplantation for Myelodysplastic Syndromes. J Oncol Pract 12, 786-792; Schiffer, M., Zhao, J., Johnson, A., Lee, J., Bewersdorf, J. P., and Zeidan, A. M. (2021). The development and clinical use of oral hypomethylating agents in acute myeloid leukemia and myelodysplastic syndromes: dawn of the total oral therapy era. Expert Rev Anticancer Ther, 1-13; Steensma, D.P. (2018). Myelodysplastic syndromes current treatment algorithm 2018. Blood Cancer J 8, 47). There is thus a dire need to critically examine the pathogenic processes underlying MDS to identify additional targets for novel therapies.

### MDS and Mitochondrial Dysfunction

Mitochondrial dysfunction is coupled to MDS pathogenesis (Fontenay, M., Cathelin, S., Amiot, M., Gyan, E., and Solary, E. (2006). Mitochondria in hematopoiesis and hematological diseases. Oncogene 25, 4757-4767; Goncalves, A. C., Cortesao, E., Oliveiros, B., Alves, V., Espadana, A. I., Rito, L., Magalhaes, E., Lobao, M. J., Pereira, A., Nascimento Costa, J. M., et al. (2015). Oxidative stress and mitochondrial dysfunction play a role in myelodysplastic syndrome development, diagnosis, and prognosis: A pilot study. Free Radic Res 49, 1081-1094). Deregulated HIFlu expression (Liu, Z., Tian, M., Ding, K., Liu, H., Wang, Y., and Fu, R. (2019). High expression of PIM2 induces HSC proliferation in myelodysplastic syndromes via the IDH1/HIF1-alpha signaling pathway. Oncol Lett 17, 5395-5402; Stergiou, I.E., Kambas, K., Poulaki, A., Giannouli, S., Katsila, T., Dimitrakopoulou, A., Vidali, V., Mouchtouris, V., Kloukina, I., Xingi, E., et al. (2021). Exploiting the Role of Hypoxia-Inducible Factor 1 and Pseudohypoxia in the Myelodysplastic Syndrome Pathophysiology. Int J Mol Sci 22), presence of isocitrate dehydrogenase (IDH) mutations expressing the oncogenic metabolite 2-hydroxy-glutarate (2-HG) (Gonzalez-Menendez, P., Romano, M., Yan, H., Deshmukh, R., Papoin, J., Oburoglu, L., Daumur, M., Dume, A. S., Phadke, I., Mongellaz, C., et al. (2021). An IDH1-vitamin C crosstalk drives human erythroid development by inhibiting pro-oxidant mitochondrial metabolism. Cell Rep 34, 108723; Intlekofer, A. M., Shih, A. H., Wang, B., Nazir, A., Rustenburg, A. S., Albanese, S. K., Patel, M., Famulare, C., Correa, F. M., Takemoto, N., et al. (2018). Acquired resistance to IDH inhibition through trans or cis dimer-interface mutations. Nature 559, 125-129; Testa, U., Castelli, G., and Pelosi, E. (2020). Isocitrate Dehydrogenase Mutations in Myelodysplastic Syndromes and in Acute Myeloid Leukemias. Cancers (Basel) 12), elevation of mitochondrial oxidative stress markers (Stergiou, I. E., Kambas, K., Poulaki, A., Giannouli, S., Katsila, T., Dimitrakopoulou, A., Vidali, V., Mouchtouris, V., Kloukina, I., Xingi, E., et al. (2021). Exploiting the Role of Hypoxia-Inducible Factor 1 and Pseudohypoxia in the Myelodysplastic Syndrome Pathophysiology. Int J Mol Sci 22; Shimizu, N., Hasunuma, H., Watanabe, Y., Matsuzawa, Y., Iwashita, Y., Tatsuno, I., and Yokota, H. (2016). The Simultaneous Elevation of Oxidative Stress Markers and Wilms' Tumor 1 Gene during the Progression of Myelodysplastic Syndrome. Intern Med 55, 3661-3664), and presence of aberrant oxidation and mutations in mitochondrial DNA (Schildgen, V., Wulfert, M., and Gattermann, N. (2011). Impaired mitochondrial gene transcription in myelodysplastic syndromes and acute myeloid leukemia with myelodysplasia-related changes. Exp Hematol 39, 666-675 e661; Ward, G. A., McGraw, K. L., Abbas-Aghababazadeh, F., Meyer, B. S., McLemore, A. F., Vincelette, N. D., Lam, N. B., Aldrich, A. L., Al Ali, N. H., Padron, E., et al. (2021). Oxidized mitochondrial DNA released after inflammasome activation is a disease biomarker for myelodysplastic syndromes. Blood Adv 5, 2216-2228; Wulfert, M., Kupper, A. C., Tapprich, C., Bottomley, S. S., Bowen, D., Germing, U., Haas, R., and Gattermann, N. (2008). Analysis of mitochondrial DNA in 104 patients with myelodysplastic syndromes. Exp Hematol 36, 577-586) strongly implicate defective mitochondrial metabolism as a pathogenic process conducive to MDS pathogenesis. A recent study has correlated deregulation in metabolic genes with prognostic stratification of MDS patients (Coelho-Silva, J. L., Silveira, D. R. A., Pereira-Martins, D. A., Rojas, C. A. O., Lucena-Araujo, A. R., Rego, E. M., Machado-Neto, J. A., Bendit, I., Rocha, V., and Traina, F. (2021). Molecular-Based Score inspired on metabolic signature improves prognostic stratification for myelodysplastic syndrome. Sci Rep 11, 1675). Mitochondrial DNA encodes 13 polypeptides in conjunction with nuclear coded transcriptional and translational machineries that form indispensable components of the mitochondrial electron transport chain (ETC) responsible for oxidative phosphorylation (OXPHOS) (Itoh, Y., Andrell, J., Choi, A., Richter, U., Maiti, P., Best, R. B., Barrientos, A., Battersby, B. J., and Amunts, A. (2021). Mechanism of membrane-tethered mitochondrial protein synthesis. Science 371, 846-849; Kummer, E., and Ban, N. (2021). Mechanisms and regulation of protein synthesis in mitochondria. Nat Rev Mol Cell Biol.). Hence, mitochondrial and nuclear genomes remain interwoven in a tightly balanced relationship to dictate cellular metabolism, deregulation of which rewires metabolic programming and inevitably results in a multitude of alterations in metabolite levels (Alston, C. L., Stenton, S. L., Hudson, G., Prokisch, H., and Taylor, R. W. (2021). The genetics of mitochondrial disease: dissecting mitochondrial pathology using multi-omic pipelines. J Pathol.; Ito, K., and Ito, K. (2018). Hematopoietic stem cell fate through metabolic control. Exp Hematol 64, 1-11; Schildgen, V., Wulfert, M., and Gattermann, N. (2011). Impaired mitochondrial gene transcription in myelodysplastic syndromes and acute myeloid leukemia with myelodysplasia-related changes. Exp Hematol 39, 666-675 e661; Zheng, Q., Jiang, Y., Zhang, A., Cui, L., Xia, L., and Luo, M. (2017). The Mechanism of Mitochondria-Mediated Pathway in the Apoptosis of Platelets in Immune-Induced Bone Marrow Failure. Chin J Physiol 60, 338-344). Congruent with this notion, elevation of tryptophan catabolism metabolites in the serum of MDS patients (Berthon, C., Fontenay, M., Corm, S., Briche, I., Allorge, D., Hennart, B., Lhermitte, M., and Quesnel, B. (2013). Metabolites of tryptophan catabolism are elevated in sera of patients with myelodysplastic syndromes and inhibit hematopoietic progenitor amplification. Leuk Res 37, 573-579) and a positive correlation between reactive oxygen metabolites and karyotypic abnormalities in MDS patients (Fracchiolla, N. S., Bamonti Catena, F., Novembrino, C., Ippolito, S., Maisonneuve, P., and Cortelezzi, A. (2003). Possible association between reactive oxygen metabolites and karyotypic abnormalities in myelodysplastic syndromes. Haematologica 88, 594-597) further underscore the involvement of deregulated metabolites in MDS etiology. Although a few other studies have investigated the deregulation of metabolic pathways in MDS patient-derived cells, the patient numbers and stratification have remained inadequate to make definitive conclusions (Cano, K. E., Li, L., Bhatia, S., Bhatia, R., Forman, S. J., and Chen, Y. (2011). NMR-based metabolomic analysis of the molecular pathogenesis of therapy-related myelodysplasia/acute myeloid leukemia. J Proteome Res 10, 2873-2881; Poulaki, A., Katsila, T., Stergiou, I. E., Giannouli, S., Gomicronmez-Tamayo, J. C., Piperaki, E. T., Kambas, K., Dimitrakopoulou, A., Patrinos, G. P., Tzioufas, A. G., et al. (2020). Bioenergetic Profiling of the Differentiating Human MDS Myeloid Lineage with Low and High Bone Marrow Blast Counts. Cancers (Basel) 12; Zhong, P., Zhang, J., and Cui, X. (2015). Abnormal metabolites related to bone marrow failure in aplastic anemia patients. Genet Mol Res 14, 13709-13718), and metabolite profiling has thus remained an unmapped predictive approach in MDS and other hematological disorders.

### Result

### RIOK2 Transcriptionally Regulates Mitochondrial Translation.

To further investigate the cellular processes contributing to ineffective hematopoiesis upon loss of RIOK2, an unbiased gene-set enrichment analyses (GSEA) was performed on RNA-sequencing datasets. Interestingly, a profound downregulation of mitochondrial translation was observed in RIOK2-deficient primary human HSPCs (FIG. 49), indicative of defective mitochondrial functions. This further suggested that RIOK2 regulates mitochondrial translation at the transcriptional level. While probing deeper into the RNA-seq dataset, a significant downregulation in several mitochondrial ribosomal protein (MRP) coding genes was observed (FIG. 50), which form key components of the mitochondrial translation machinery (Kummer, E., and Ban, N. (2021). Mechanisms and regulation of protein synthesis in mitochondria. Nat Rev Mol Cell Biol.; Yousefi, R., Fornasiero, E. F., Cyganek, L., Montoya, J., Jakobs, S., Rizzoli, S. O., Rehling, P., and Pacheu-Grau, D. (2021). Monitoring mitochondrial translation in living cells. EMBO Rep, e51635). Next, it was ascertained whether the de novo nucleotide binding motif specific to RIOK2 (Ghosh, S., Raundhal, M., Myers, S. A., Carr, S. A., Chen, X., Petsko, G. A., and Glimcher, L. H. (2022). Identification of RIOK2 as a master regulator of human blood cell development. Nat Immunol 23, 109-121) was present in the promoter regions of mitochondria-associated genes.

Interestingly, the presence of this motif at the promoter regions of several MRP genes with differential transcriptional profiles upon dose-dependent loss of RIOK2 was noted (FIGS. 50 and 51), suggesting that RIOK2 binds to the promoter regions of MRP genes to transcriptionally regulate mitochondrial translation. Corroborating this, a marked inhibition of chromatin accessibility at the promoter regions of MRP genes upon loss of RIOK2 was noted (FIG. 52). Taken together, these shows that RIOK2 functions as a transcription factor to regulate the expression of MRP genes involved in mitochondrial translation.

Loss of RIOK2 impedes mitochondrial biogenesis and functions To validate the observations from sequencing analyses, mitochondrial translation upon knockdown (KD) and knockout (KO) of RIOK2 in erythroleukemia cell line TF-1 was examined employing O-propargyl-puromycin (OPP) incorporation assays that label nascent peptides. To distinguish between cytoplasmic and mitochondrial proteins, cycloheximide (CHX) treatment was utilized, which is known to block cytoplasmic translation without affecting mitochondrial protein synthesis (Zhang, S., Macias-Garcia, A., Ulirsch, J. C., Velazquez, J., Butty, V. L., Levine, S. S., Sankaran, V. G., and Chen, J. J. (2019). HRI coordinates translation necessary for protein homeostasis and mitochondrial function in erythropoiesis. Elife 8). Impeded OPP assimilation in RIOK2-deficient cells upon administration of cycloheximide was observed (FIG. 53A), underscoring that loss of RIOK2 indeed suppresses mitochondrial translation. Since mitochondrial translation produces key components of the Electron Transport Chain (ETC), any resultant alterations in mitochondrial membrane potential was investigated next. TMRE (Tetramethylrhodamine, ethyl ester) sequestration assays were used since this cell-permeant positively charged dye gets incorporated in polarized and active mitochondria. As speculated, loss of RIOK2 dramatically impaired mitochondrial membrane potential (FIG. 53B). Deficiency of RIOK2 also impeded incorporation of passively-diffusing MitoTracker dyes, indicative of impaired mitochondrial mass (FIG. 53C). To further assess if RIOK2 affects mitochondrial OXPHOS, Seahorse Respirometry assays were performed that determine real-time oxygen consumption rates (OCR) of live cells. Notably, downregulated OCR was observed in RIOK2-deficient viable TF-1 cells (FIG. 54).

Deficiency of genes implicated in MDS pathogenesis result in mitochondrial dysfunction Since loss of RIOK2 mirrors MDS-associated abnormalities and dysfunctional mitochondrial metabolism, these findings led to testing to determine whether other genes implicated in MDS, such as RPS14 (Ribosomal Protein S14) and APC (Adenomatosis Polyposis Coli) (Ebert, B. L., Pretz, J., Bosco, J., Chang, C. Y., Tamayo, P., Galili, N., Raza, A., Root, D. E., Attar, E., Ellis, S. R., et al. (2008). Identification of RPS14 as a 5q- syndrome gene by RNA interference screen. Nature 451, 335-339; Lane, S. W., Sykes, S. M., Al-Shahrour, F., Shterental, S., Paktinat, M., Lo Celso, C., Jesneck, J. L., Ebert, B. L., Williams, D. A., and Gilliland, D. G. (2010). The Apc(min) mouse has altered hematopoietic stem cell function and provides a model for MPD/MDS. Blood 115, 3489-3497), similarly affect mitochondrial functions. Surprisingly, knockdown of RPS14 or APC in primary human HSPCs significantly impeded mitochondrial mass and membrane potential (FIG. 55A and FIG. 55B). This further emphasizes that mitochondrial dysfunction underlies MDS pathogenesis.

### RIOK2 Regulates Metabolic Pathways

This data indicate that RIOK2 transcriptionally regulates the expression of MRP genes that are indispensable for mitochondrial translation and OXPHOS (Itoh, Y., Andrell, J., Choi, A., Richter, U., Maiti, P., Best, R. B., Barrientos, A., Battersby, B. J., and Amunts, A. (2021). Mechanism of membrane-tethered mitochondrial protein synthesis. Science 371, 846-849; Kummer, E., and Ban, N. (2021). Mechanisms and regulation of protein synthesis in mitochondria. Nat Rev Mol Cell Biol). This encouraged a deeper probe into the transcriptomic profiles of metabolic genes known to regulate mitochondrial nutrient and energy metabolism in stem cells (Ge, Y., and Fuchs, E. (2018). Stretching the limits: from homeostasis to stem cell plasticity in wound healing and cancer. Nat Rev Genet 19, 311-325; Intlekofer, A. M., and Finley, L. W. S. (2019). Metabolic signatures of cancer cells and stem cells. Nat Metab 1, 177-188). Investigation of RNA-seq datasets revealed that loss of RIOK2 led to downregulation of genes associated with the TCA cycle (pyruvate dehydrogenase: PDHA1, succinate dehydrogenase: SDHA/D), mitochondrial β-oxidation (hydroxyacyl-CoA dehydrogenase: HADH), and electron transport chain (ETC) complexes (NDUFAF4/5). In contrast, genes associated with glycolysis (hexokinase 3: HK3, pyruvate kinase: PKM) were upregulated in RIOK2-deficient HSPCs (FIG. 56). These results indicate that RIOK2-mediated metabolic regulation and an altered balance between glycolysis and OXPHOS could affect hematopoietic stem cell differentiation. However, alterations in metabolic gene profiles may not necessarily lead to alterations in the functional output of a pathway. Hence, the total pools of a targeted set of metabolites were initially quantified, including TCA cycle intermediates in RIOK2-proficient vs deficient primary human HSPCs (FIG. 57). These preliminary studies indicated reduced pools of the TCA cycle intermediates citrate, malate, aspartate and glutamate following RIOK2 knockdown (FIG. 57). Interestingly, these changes in TCA cycle metabolites are consistent with metabolite patterns reported in cells with reduced or complete loss of SDH (Cardaci, S., Zheng, L., MacKay, G., van den Broek, N. J., MacKenzie, E. D., Nixon, C., Stevenson, D., Tumanov, S., Bulusu, V., Kamphorst, J. J., et al. (2015). Pyruvate carboxylation enables growth of SDH-deficient cells by supporting aspartate biosynthesis. Nat Cell Biol 17, 1317-1326; Lussey-Lepoutre, C., Hollinshead, K. E., Ludwig, C., Menara, M., Morin, A., Castro-Vega, L.J., Parker, S. J., Janin, M., Martinelli, C., Ottolenghi, C., et al. (2015). Loss of succinate dehydrogenase activity results in dependency on pyruvate carboxylation for cellular anabolism. Nat Commun 6, 8784). In addition, total pyruvate levels were slightly but significantly higher in RIOK2 deficient cells, which is consistent with reduced PDH levels as observed in RNA-seq dataset (FIGS. 56 and 57). These findings give rise to the premise that RIOK2 deficiency leads to repatterning of metabolism in human HSPCs.

### MATERIALS AND METHODS for Example 6

### Primary and Secondary Cell Culture

CD34+primary human hematopoietic stem and progenitor cells (HSPCs) and TF-1 cells were procured and cultured, as previously described.

### CRISPR/Cas9 Gene Editing

Primary human HSPCs and human erythroblast cells (TF-1) were electroporated as previously described.

### Quantitative RT-PCR

Performed as previously described in other Examples.

### Flow Cytometry Analysis

Performed as previously described in other Examples.

### Mitochondrial Fitness Tests

TMRE (T669, Invitrogen) and Mitotracker (M22426, Life Technologies) staining were performed as per the manufacturer's instructions.

### Mitochondrial Seahorse Assay

Performed at the Beth Israel Deaconess Medical Center (BIDMC) metabolomics core.

### Bulk RNA-Sequencing

RNA library preparations, sequencing reactions and initial bioinformatic analysis were conducted at GENEWIZ, LLC. (South Plainfield, NJ, USA) as follows: Library Preparation with polyA selection and HiSeq Sequencing: RNA samples received were quantified using Qubit 2.0 Fluorometer (Life Technologies, Carlsbad, CA, USA) and RNA integrity was checked using Agilent TapeStation™ 4200 (Agilent Technologies, Palo Alto, CA, USA). RNA sequencing libraries were prepared using the NEBNext Ultra™ RNA Library Prep Kit for Illumina following manufacturer's instructions (NEB, Ipswich, MA, USA). Briefly, mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation™ (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA). The sequencing libraries were clustered on 1 lane of a flowcell. After clustering, the flowcell was loaded on the Illumina HiSeq™ instrument (4000 or equivalent) according to manufacturer's instructions. The samples were sequenced using a 2×150 bp Paired End (PE) configuration. Image analysis and base calling were conducted by the HiSeq™ Control Software (HCS). Raw sequence data (.bcl files) generated from Illumina HiSeq™ was converted into fastq files and de-multiplexed using Illumina's bcl2fastq 2.17 software. One mismatch was allowed for index sequence identification.

### Data Analysis

After investigating the quality of the raw data, sequence reads were trimmed to remove possible adapter sequences and nucleotides with poor quality using Trimmomatic v.0.36. The trimmed reads were mapped to the Homo sapiens reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. Only unique reads that fell within exon regions were counted.

Gene set enrichment analysis (GSEA) was performed using Broad Institute's GSEA Software. Sets of telomere-maintenance associated genes were derived by accounting for the differentially regulated genes that passed the cut off of adjusted p value <0.05 in control vs RIOK2-depleted HSPCs. Additionally, canonical pathways such as KEGG, REACTOME and BIOCARTA were consulted.

Assay of Transposase Accessible Chromatin with High-Throughput Sequencing (ATAC-Seq)

ATAC-seq on equal number of snap-frozen control and RIOK2-depleted HSPCs was performed at Center for Functional Cancer Epigenetics (CFSE) at DFCI. Briefly, after high throughput Illumina sequencing of pair-ended reads, the raw reads were mapped to the reference genome (hg19) using alignment software (conFigureyaml). After alignment, the mapped reads were then normalized (by down-sampling) to 4 million reads. The uniquely mapped reads of the downsample were then piled-up. Nd is defined as the number of unique region locations. N1 is defined as the number of unique region locations with only one read. The PBC (a common method to determine sample complexity) is then simply calculated as N1/Nd. ATAC-seq samples with a good PBC score >=0.90 were analyzed. Peaks were called using the MACS peak calling software. Chromatin accessibility was analyzed using IGV software.

### Nascent Protein Synthesis In Vitro

Measurement of nascent protein synthesis in vitro was done using Click-iT™ Plus OPP Alexa Fluor™ 647 Protein Synthesis Assay kit (Thermo Fisher Scientific) using manufacturer's instructions. Briefly, cells in culture (with or without cycloheximide 10/100 μM treated) were supplemented with Click-iTT™ OPP (o-propargyl puromycin) at a final working concentration of 20 μM and incubated for 30 minutes at 37° C. with 5% C02. The cells were then washed with PBS, stained with fixable viability dye, the cells fixed and permeabilized followed by incubation with Click-iT™ M reaction buffer containing Alexa Fluor™ 647 picolyl azide and incubated at RT for 30 minutes. The cells were then washed and analyzed via flow cytometry as described above.

### Statistical Tests

Data are presented as mean±SEM. Unpaired two-tailed t-test was used for comparing two groups. Analysis of variance (ANOVA) with Tukey's correction or Kruskal-Wallis test with Dunn's correction was used for comparisons amongst multiple groups, wherever applicable as per requirements of data and quantification. GraphPad Prism v8.0/9.0 (GraphPad Software Inc., San Diego, CA) was used to perform statistical analyses. Sample size was not predetermined.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of slowing aging and/or telomere shortening in a subject in need thereof, preventing or treating a metabolic disorder, or preventing or treating one or more red blood cell disorders, the method comprising administering to the subject an effective amount of at least one agent that increases and/or stabilizes the copy number, the expression level, and/or the activity of right open reading frame kinase 2 (RIOK2).

2. The method of claim 1, wherein the subject has dyskeratosis congenita (DC), myelodysplastic syndrome, or idiopathic pulmonary fibrosis (IPF).

3. (canceled)

4. The method of claim 1, wherein the metabolic disorder is diabetes, obesity, pre-diabetes, metabolic syndrome, or a mitochondriopathy or a metabolic disorder associated with a mitochondrial defect.

5. (canceled)

6. The method of claim 1, wherein the one or more red blood disorders comprise anemia, optionally wherein the anemia is selected from the group consisting of macrocytic anemia, anemia associated with chronic kidney disease (CKD), anemia caused by insufficiency of serine/threonine-protein kinase RIOK2, anemia caused by one or more mutations and/or deletions in human chromosome 5 or in an ortholog thereof, stress-induced anemia, Diamond Blackfan anemia, aplastic anemia, Schwachman-Diamond syndrome, an anemia associated with an inflammatory disease, such as rheumatoid arthritis or systemic lupus erythematosus and anemia associated with a bone marrow failure syndrome.

7. The method of claim 6, wherein the anemia is associated with a cancer, optionally wherein the cancer is a hematologic malignancy (e.g., myelodysplastic syndromes (MDS) or acute myeloid leukemia (AML)).

8. The method of claim 1, wherein the one or more red blood disorders comprises a red blood cell disorder associated with increased megakaryopoiesis and/or myelopoiesis.

9. The method of claim 6, further comprising administering to the subject an effective amount of an erythropoiesis-stimulating agent, wherein the erythropoiesis-stimulating agent comprises erythropoietin, epoetin alfa, epoetin beta, epoetin omega, epoetin zeta, IL-9, or darbepoetin alfa.

10-12. (canceled)

13. The method of claim 1, wherein the subject has a mutation that is associated with a decreased copy number, expression level, and/or the activity of RIOK2.

14. The method of claim 1, wherein the subject has a loss of function mutation in a RIOK2 gene.

15. The method of claim 1, wherein the subject expresses any one of the RIOK2 variants listed in Table 3.

16. The method of claim 15, wherein the at least one agent comprises an internally cross-linked peptide, small molecule, a peptide, a polypeptide, an aptamer, an antibody or a binding fragment thereof, an intrabody or a binding fragment thereof, and/or a nucleic acid.

17. The method of claim 16, wherein the at least one agent is an internally cross-linked peptide that
(i) specifically binds to an amino acid sequence in a corepressor that binds to a transrepressor domain (TRD) of RIOK2;
(ii) activates RIOK2, and/or
(iii) stabilizes the expression or activity of RIOK2.

18. (canceled)

19. The method of claim 16, wherein the internally cross-linked peptide comprises:
i) an amino acid sequence of XSLIXSIAS (SEQ ID NO: 1), wherein X is a non-natural amino acid and X1 and X5 are joined by an internal staple;
ii) an amino acid sequence of GSXIASXAS (SEQ ID NO: 2), wherein X is a non-natural amino acid, and X3 and X7 are joined by an internal staple;
iii) an amino acid sequence of GSLXASIXS (SEQ ID NO: 3), wherein X is a non-natural amino acid, and X4 and X8 are joined by an internal staple; or
iv) an amino acid sequence of 8GSLIASIXS (SEQ ID NO: 4), wherein X and 8 are non-natural amino acids, and 8 and X8 are joined by an internal staple.

20-25. (canceled)

26. The method of claim 1, wherein the at least one agent comprises a cell-based agent, wherein the cell that is modified to comprise an increased copy number, expression level, and/or activity of RIOK2 or a fragment thereof.

27-29. (canceled)

30. A method of preventing or treating polycythemia vera, the method comprising administering to the subject an effective amount of at least one agent that decreases the copy number, the expression level, and/or the activity of RIOK2.

31. The method of claim 30, wherein the at least one agent comprises an inhibitory internally cross-linked peptide, small molecule, a peptide, a polypeptide, an aptamer, an antibody or a binding fragment thereof, an intrabody or a binding fragment thereof, and/or a nucleic acid.

32. The method of claim 31, wherein the at least one agent is an inhibitory internally cross-linked peptide, wherein the inhibitory internally cross-linked peptide:
(i) blocks RIOK2 binding to DNA, and/or
(ii) decreases the copy number, expression, or activity of RIOK2.

33-51. (canceled)

52. An internally cross-linked polypeptide comprising:
i) an amino acid sequence of XSLIXSIAS (SEQ ID NO: 1), wherein X is a non-natural amino acid and X1 and X5 are joined by an internal staple;
ii) an amino acid sequence of GSXIASXAS (SEQ ID NO: 2), wherein X is a non-natural amino acid, and X3 and X7 are joined by an internal staple;
iii) an amino acid sequence of GSLXASIXS (SEQ ID NO: 3), wherein X is a non-natural amino acid, and X4 and X8 are joined by an internal staple; and/or
iv) an amino acid sequence of 8GSLIASIXS (SEQ ID NO: 4), wherein X and 8 are non-natural amino acids, and 8 and X8 are joined by an internal staple, v) an amino acid sequence of XNKVXRELVKH (SEQ ID NO: 6), wherein X is a non-natural amino acid and X1 and X5 are joined by an internal staple;
vi) an amino acid sequence of SNKVXRELXKH (SEQ ID NO: 7), wherein X is a non-natural amino acid, and X5 and X9 are joined by an internal staple;
vii) an amino acid sequence of 8NKVLREXVKH (SEQ ID NO: 8), wherein X and 8 are non-natural amino acids, and 8 and X8 are joined by an internal staple;
viii) an amino acid sequence of S8KVLRELXKH (SEQ ID NO: 9), wherein X and 8 are non-natural amino acids, and 8 and X9 are joined by an internal staple, and/or
ix) an amino acid sequence of SNK8LRELVKX(SEQ ID NO: 10), wherein X and 8 are non-natural amino acids, and 8 and X11 are joined by an internal staple.

53. (canceled)

54. The method of claim 52, wherein 8 is R5-octenyl alanine and X is S5-pentenyl alanine.

55. (canceled)
